(19) United States
(12) Patent Application Publication
(10) Pub. No.: US 2025/0249129 A1
(43) Pub. Date: Aug. 7, 2025

(54) A CONSTRUCT, VECTOR, AND SYSTEM AND USES THEREOF

(71) Applicant: UCL BUSINESS LTD, London, Greater London (GB)

(72) Inventors: Pietro FRATTA, London (GB); Oscar WILKINS, London (GB)

(73) Assignee: UCL BUSINESS LTD, London, Greater London (GB)

(21) Appl. No.: 18/855,688

(22) PCT Filed: Feb. 24, 2023
(86) PCT No.: PCT/EP2023/054670

§ 371 Date: Oct. 10, 2024

(30) Foreign Application Priority Data
Apr. 11, 2022 (GB) 2205282.3

(51) Int. Cl. A61K 48/00; C12N 15/85

(52) U.S. Cl. CPC A61K 48/0066; C12N 15/85; C12N 2320/33; C12N 2830/42

(57) ABSTRACT

A construct comprising a start codon, a regulatory domain comprising a first splice acceptor site and a first splice donor site, a binding domain for a splicing factor of the hnRNP family, located within 150 nucleotides of the first splice donor site and/or first splice acceptor site and/or located between the first splice acceptor site and first splice donor site; and a transgene sequence, wherein the construct is configured such that (i) if placed in a cell with nuclear depletion of the splicing factor, splicing of the first splice acceptor site and first donor site is not repressed, such that a functional protein is produced from the transgene sequence, and (ii) if placed in a cell without nuclear depletion of the splicing factor, splicing of the first splice acceptor site and/or first donor site is repressed such that no functional protein is produced from the transgene sequence. A vector comprising the construct, as well as a system comprising the constructs or vector and a cell are also described. The splicing factor of the hnRNP family may be TDP-43. The construct and vector may be used in therapy, for example, in diseases associated with depletion of a hnRNP splicing factor.

### BACKGROUND

Neurodegenerative diseases are often deadly and, with few exceptions, have no effective long-term treatments. There is thus an urgent need for new therapies and treatments for neurodegenerative diseases; however, progress has been slow due to a lack of understanding of the complex molecular mechanisms that underpin these diseases.

Although there is still much to learn about these disease mechanisms, it has been established that many neurodegenerative diseases involve dysregulation of RNA-binding proteins (RBPs), which include the heterogenous nuclear ribonucleoproteins (hnRNPs). hnRNPs are typically located in the nucleus and take part in many stages of RNA metabolism but have a role in regulation of alternative splicing leading to either exon skipping or intron retention.

One such protein of the hnRNP family is TAR DNA-binding protein (TDP-43). Although originally identified as a DNA-binding protein, TDP-43 is well characterised as a member of the hnRNP family of proteins, and has a prominent role in neurodegenerative diseases: TDP-43 is mislocalized in ˜97% of amyotrophic lateral sclerosis (ALS, a motor neuron disease) cases and around half of frontotemporal dementia cases and the majority of inclusion body myopathy (IBM). Furthermore, TDP-43 pathology has also been observed in Alzheimer's disease (AD), and other neurodegenerative diseases (including cases of Parkinson's disease (PD) and Perry syndrome), suggesting its role in neurodegeneration extends beyond ALS/FTD. Additionally, a small percentage of ALS cases are caused by mutations to the TARDBP gene which encodes TDP-43. TDP-43, in particular, has many roles in the regulation of RNA, ranging from RNA transcription to RNA decay. Perhaps its best characterised function is as a regulator of splicing, typically as a splicing repressor. When localised near splicing sites, TDP-43 binding is shown to repress and silence splicing. It was first shown to regulate splicing of the CFTR transcript in 2001; numerous subsequent studies have demonstrated that TDP-43 regulates a plethora of transcripts, including its own. In neurodegenerative diseases with TDP-43 pathology, cytoplasmic aggregation and nuclear depletion of the TDP-43 are typically both observed.

Although it is possible to target expression of proteins to specific cell types, for example by using local injection of viruses combined with cell-type-specific transcriptional promoters (such as the synapsin promoter), this has the disadvantage that expression occurs both in diseased cells and non-diseased cells. Transgenic expression of these proteins may therefore significantly damage otherwise healthy cells, increasing the risk of adverse events (e.g., during clinical trials), and would increase side effects for any treatment and reduce the likelihood of regulatory approval. While these risks can be lowered by decreasing the expression of the transgenic protein in patients, this would have the effect of decreasing efficacy within the diseased cells.

There is therefore a need to develop new tools to further understand, target, and correct dysregulated molecular mechanisms associated with neurodegenerative diseases which overcome some of the disadvantages associated with the prior art.

### SUMMARY OF INVENTION

In a first aspect there is provided, a construct comprising
a start codon,
a regulatory domain comprising:
   a first splice acceptor site and a first splice donor site,
   a binding domain for a splicing factor of the heterogenous nuclear ribonucleoprotein (hnRNP) family, located
   within 150 nucleotides of the first splice donor site and/or first splice acceptor site, and/or located between the first splice donor site and first splice acceptor site; and
a transgene sequence,
wherein the construct is configured such that
   (i) if placed in a cell with nuclear depletion of the splicing factor, splicing of the first splice acceptor site and first donor site is not repressed, such that a functional protein is produced from the transgene sequence
   (ii) if placed in a cell without nuclear depletion of the splicing factor, splicing of the first splice acceptor site and/or first donor site is repressed such that no functional protein is produced from the transgene sequence.

In a second aspect, or embodiment of the first aspect, there is provided, a construct comprising
a start codon,
a regulatory domain comprising:
   a first splice acceptor site and a first splice donor site, which define a cryptic exon sequence,
   an intronic region defined by a second splice acceptor site and a second splice donor site, wherein said cryptic exon sequence is located within the intronic region
   a binding domain for a splicing factor of the heterogenous nuclear ribonucleoprotein (hnRNP) family, located within 150 nucleotides of the first splice donor site and/or first splice acceptor site; and
a transgene sequence,
   configured such that
   (i) if placed in a cell that is depleted of the splicing factor, splicing of the first splice acceptor site and first donor site is not repressed and the cryptic exon sequence is present in the mRNA product of the construct, such that a functional protein is produced from the transgene sequence
   (ii) if placed in a cell that is not depleted of the splicing factor, splicing of the first splice acceptor site and/or first donor site is repressed and the cryptic exon sequence is absent in the mRNA product of the construct, such that a functional protein is not produced from the transgene sequence.

In an embodiment of the second aspect, the transgene sequence is completely downstream of the regulatory domain. These are described as “Design 1” embodiments described herein.

In an alternative embodiment of the second aspect, at least part of the transgene sequence is encoded by the cryptic exon sequence. These are described as “Design 2” embodiments described herein.

In a third aspect, or embodiment of the first aspect, there is provided, a construct comprising
a start codon,
a regulatory domain comprising
   a first splice donor site and a first acceptor donor site, which define a single regulatory intron,
   a binding domain for a splicing factor of the heterogenous nuclear ribonucleoprotein (hnRNP) family, located within 150 nucleotides of the first splice donor site and/or first splice acceptor site and/or located between the first splice donor site and first splice acceptor site;
   and
a transgene sequence,
   configured such that
   (i) if placed in a cell that is depleted of splicing factor, splicing of the first splice acceptor site and first donor site is not repressed and the single regulatory intron is spliced, such that a functional protein is produced from the transgene sequence
   (ii) if placed in a cell that is not depleted of splicing factor, splicing of the first splice acceptor site and/or first donor site is repressed, and the single regulatory intron is not or incorrectly spliced such that no functional protein is produced from the transgene sequence.

In a fourth aspect of the invention, there is provided a vector comprising the construct of the above aspects.

In a fifth aspect of the invention, there is provided a pharmaceutical composition comprising the construct of the above aspects, or the vector of the above aspect.

In a sixth aspect of the invention, there is provided a system comprising any construct described herein and a cell, or a system comprising any vector described herein and a cell wherein
(i) upon depletion of the splicing factor of the hnRNP family from the cell nucleus (i.e., in a diseased cell), the system produces a functional protein from the transgene sequence, and
(ii) wherein upon no depletion of the splicing factor of the hnRNP family from the cell nucleus, (i.e., in a healthy cell) the system does not produce a functional protein from the transgene sequence.

In a seventh aspect of the invention, there is provided any construct, vector or pharmaceutical composition described herein for use in therapy.

In an eighth aspect of the invention, there is provided any construct, vector or pharmaceutical composition described herein for use in the treatment of a disease associated with depletion of the splicing factor of the hnRNP family, wherein the treatment comprises contacting a cell with the construct, vector, or pharmaceutical composition such that
(i) in a cell with nuclear depletion of the splicing factor of the hnRNP family, the cell produces a functional protein,
(ii) in a cell without nuclear depletion of the splicing factor of the hnRNP family, the cell does not produce a functional protein.

In some embodiments, the disease is a neurodegenerative disease or a muscle disease. In some embodiments, the neurodegenerative disease is amyotrophic lateral sclerosis (ALS) or frontotemporal dementia (FTD). In preferred embodiments, the splicing factor of the hnRNP family is TDP-43.

In a ninth aspect of the present invention, is provided the use of any construct described herein, the use of any vector described herein, or the use of any pharmaceutical composition described herein, in a method of selectively producing functional protein in a diseased cell that has nuclear depletion of the splicing factor of the hnRNP family.

Also disclosed herein, is a construct comprising
a start codon,
a regulatory domain comprising:
   a first splice acceptor site and a first splice donor site,
   a binding domain for a splicing factor of the heterogenous nuclear ribonucleoprotein (hnRNP) family, located within 150 nucleotides of the first splice donor site or first splice acceptor site and/or located between the first splice donor site and first splice acceptor site; and
a transgene sequence,
wherein the construct is configured such that
   (i) if placed in an in vitro system with depletion (i.e., absence) of the splicing factor, splicing of the first splice acceptor site and first donor site is not repressed, such that a functional protein is produced from the transgene sequence
   (ii) if placed in a vitro system with without depletion (i.e., presence) of the splicing factor, splicing of the first splice acceptor site and/or first donor site is repressed such that no functional protein is produced from the transgene sequence.

The in vitro system must comprise components which enable transcription, splicing and translation. In some embodiments, these components are provided by a cell.

Also disclosed herein is a construct comprising
a start codon,
a regulatory domain comprising:
   a first splice acceptor site and a first splice donor site,
   a binding domain for a splicing factor of the heterogenous nuclear ribonucleoprotein (hnRNP) family, located
   within 150 nucleotides of the first splice donor site and/or first splice acceptor site, and/or located between the first splice donor site and first splice acceptor site; and
a transgene sequence encoding a functional protein,
wherein the construct is configured such that
   (i) if placed in a cell with nuclear depletion of the splicing factor, splicing of the first splice acceptor site and first donor site is not repressed, such that a functional protein is produced from the mRNA product of the construct
   (ii) if placed in a cell without nuclear depletion of the splicing factor, splicing of the first splice acceptor site and/or first donor site is repressed such that no functional protein is produced from the mRNA product of the construct.

Also described, there is provided, a construct comprising
a start codon,
a regulatory domain comprising:
   a first splice acceptor site and a first splice donor site, which define a cryptic exon sequence,
   an intronic region defined by a second splice acceptor site and a second splice donor site, wherein said cryptic exon sequence is located within the intronic region
   a binding domain for a splicing factor of the heterogenous nuclear ribonucleoprotein (hnRNP) family, located within 150 nucleotides of the first splice donor site and/or first splice acceptor site; and
a transgene sequence encoding a functional protein,
   configured such that
   (iii) if placed in a cell that is depleted of the splicing factor, splicing of the first splice acceptor site and first donor site is not repressed and the cryptic exon sequence is present in the mRNA product of the construct, such that a functional protein is produced from the mRNA product of the construct
   (iv) if placed in a cell that is not depleted of the splicing factor, splicing of the first splice acceptor site and/or first donor site is repressed and the cryptic exon sequence is absent in the mRNA product of the construct, such that a functional protein is not produced from the mRNA product of the construct.

Also described, there is provided, a construct comprising
a start codon,
a regulatory domain comprising
   a first splice donor site and a first acceptor donor site, which define a single regulatory intron,
   a binding domain for a splicing factor of the heterogenous nuclear ribonucleoprotein (hnRNP) family, located within 150 nucleotides of the first splice donor site and/or first splice acceptor site and/or located between the first splice donor site and first splice acceptor site;
   and
a transgene sequence encoding a functional protein,
   configured such that
   (iii) if placed in a cell that is depleted of splicing factor, splicing of the first splice acceptor site and first donor site is not repressed and the single regulatory intron is spliced, such that a functional protein is produced from the mRNA product of the construct
   (iv) if placed in a cell that is not depleted of splicing factor, splicing of the first splice acceptor site and/or first donor site is repressed, and the single regulatory intron is not or incorrectly spliced such that no functional protein is produced from the mRNA product of the construct.

Also described herein, there is provided a system comprising any construct described herein and a cell, or a system comprising any vector described herein and a cell wherein
(i) upon depletion of the splicing factor of the hnRNP family from the cell nucleus (i.e., in a diseased cell), the system produces a functional protein from the mRNA product of the construct, and
(ii) wherein upon no depletion of the splicing factor of the hnRNP family from the cell nucleus, (i.e., in a healthy cell) the system does not produce a functional protein from the mRNA product of the construct.

Also disclosed herein, as a further aspect or an embodiment of the first and second aspect, is a construct comprising
a transgene sequence and a regulatory domain, the regulatory domain comprising (from upstream to downstream)
   an exon immediately upstream of the splice donor site
   a splice donor site (i.e., a second splice donor site),
   a first part of an intronic region,
   a splice acceptor site (i.e., a first splice acceptor site),
   a cryptic exon sequence (i.e., which is embedded within the intronic region between the first splice acceptor site and the first splice donor site),
   a splice donor site (i.e., a first splice donor site),
   a second part of an intronic region, and
   a splice acceptor site (i.e., the second splice acceptor site), and
   an exon immediately downstream of the splice acceptor site
   wherein the regulatory domain comprises a binding site for a splicing factor of the hnRNP family which is within the first part of the intronic region, the cryptic exon sequence, and/or the second part of intronic region.

The splicing factor is preferably TDP-43. In some embodiments, the transgene sequence is completely downstream of the regulatory domain. In some embodiments, the transgene sequence is at least partly encoded by the cryptic exon sequence, and optionally encoded by the exon immediately upstream of the splice donor site and/or the exon immediately downstream of the splice acceptor site.

Also disclosed herein, as a further aspect or an embodiment of the first and second aspect, is
a construct comprising (from upstream to downstream)
an exonic sequence (i.e., immediately upstream of the splice donor site)
a splice donor site (i.e., a second splice donor site),
a first part of an intronic region (i.e., or a first intron)
a splice acceptor site (i.e., a first splice acceptor site),
a cryptic exon sequence (i.e., embedded within the intronic region between the first splice
acceptor site and the first splice donor site),
a splice donor site (i.e., a first splice donor site),
a second part of an intronic region,
a splice acceptor site (i.e., a second splice acceptor site), and
an exonic sequence immediately downstream of the splice acceptor site,
an optional protein cleavage or self-cleavage site,
and a transgene sequence (i.e., a complete transgene sequence),
wherein the construct comprises a binding domain for a splicing factor of the hnRNP family which is within the first part of the intronic region, the cryptic exon sequence, and/or the second part of intronic region.

The first splice acceptor site and first splice donor site are repressed by the splicing factor of the hnRNP family.

This construct may be described as a “Design 1” construct herein. The splicing factor is preferably TDP-43. In an embodiment, the start codon may be present in the exonic sequence upstream of the cryptic exon, and the cryptic exon of a length not divisible by three such that it introduces a frame-shift, with the construct configured such that only when the cryptic exon is included is the start codon in frame with the downstream transgene sequence. Alternatively, in another embodiment, the start codon (i.e., necessary for transgene expression) may be present within the cryptic exon itself.

Also disclosed herein, as a further aspect or an embodiment of the first and second aspect, is a construct comprising a transgene sequence (i.e., a transgene sequence encoding a functional protein) and a regulatory domain, the construct comprising (from upstream to downstream)
an exonic sequence (i.e., immediately upstream of the splice donor site, and optionally encoding for part of the transgene sequence)
a splice donor site (i.e., a second splice donor site),
a first part of an intronic region (i.e., a first intron),
a splice acceptor site (i.e., a first splice acceptor site),
a cryptic exon sequence encoding for at least a part of a transgene (i.e., embedded within the intronic region between the first splice acceptor site and the first splice donor site)
a splice donor site (i.e., a first splice donor site),
a second part of an intronic region (i.e., a second intron) and
a splice acceptor site (i.e., a second splice acceptor site), and
an exonic sequence (i.e., immediately downstream of the splice acceptor site, and optionally encoding for a part of the transgene), wherein the construct comprises a binding domain for a splicing factor of the hnRNP family which is within the first part of the intronic region, the cryptic exon sequence, and/or the second part of the intronic region.

The first splice acceptor site and first splice donor site are repressed by the splicing factor of the hnRNP family.

This construct may be described as a Design 2 construct herein. The splicing factor is preferably TDP-43.

Also disclosed herein, as a further aspect or an embodiment of the first and third aspect, is a construct comprising a transgene sequence (i.e., a transgene sequence encoding a functional protein) and a regulatory domain, the regulatory domain comprising (from upstream to downstream)
An exonic sequence (i.e., immediately upstream of the splice donor site)
A splice donor site (i.e., the first splice donor site),
A single regulatory intron,
A splice acceptor site (i.e., the first splice acceptor site) and
An exonic sequence (i.e., immediately downstream of the splice acceptor site), wherein the regulatory domain comprises a binding domain for a splicing factor of the hnRNP family which is within the exonic sequence upstream of the splice donor site, the single regulatory intron and/or the exonic sequence downstream of the splice acceptor site, and

The splicing factor is preferably TDP-43. In some embodiments, the transgene sequence is completely downstream of the regulatory domain. In some embodiments, the transgene sequence is encoded by the exonic sequence immediately upstream of the splice donor site and the exon immediately downstream of the splice acceptor site.

The first splice acceptor site and first splice donor site are repressed by the splicing factor of the hnRNP family. In some embodiments, the construct further comprises an alternative splice donor site and/or alternative splice acceptor site which is not repressed by the hnRNP splicing factor. The alternative splice acceptor site may be within the single regulatory intron or downstream of the first splice acceptor site. The alternative splice donor site may be within the single regulatory intron or upstream of the first splice donor site.

Aspects or embodiments of the present invention have one or more of the following advantages:

Aspects of the present invention provides a mechanism for expressing transgenic proteins selectively in diseased cells associated with depletion of a hnRNP splicing factor. This has immense therapeutic benefit, as therapeutic proteins such as chaperones, nuclear import receptors, or gene editing enzymes such as Cas9 nuclease, can be expressed specifically in cells with depletion of a hnRNP splicing factor (e.g., diseased cells depleted with TDP-43), with improved safety and efficacy, while leading to minimal, reduced or no expression in healthy cells. Furthermore, the construct and system can be used to express a diagnostic protein, such as a secreted luciferase, which can be used to aid detection of patients with cells with depleted hnRNP splicing factors, e.g., cells with TDP-43 pathology.

The present construct and system of the present invention also has utility to enable pre-emptive treatment, whereby the treatment is administered to at-risk patients before pathology is even detectable. Importantly, the construct and system will only be activated once pathology (e.g., significant TDP-43 pathology in neurons) occurs, and automatically deactivates once that pathology resolves in the cell.

The present invention therefore provides improved tools to specifically target diseased cells associated with hnRNP depletion, which can be used as a therapy for neurodegenerative disease. Since the constructs, vectors and pharmaceutical compositions described herein are designed to only express protein in diseased cells, selective administration of the construct to a specific cell type is not required. This means that more general and less invasive administration methods could be used.

In all the above aspects and embodiments described herein, the binding domain can be for TDP-43, and the splicing factor of the hnRNP family is TDP-43. This is useful for the study, detection, and treatment of cells with TDP-43 pathology, which is implicated in many neurodegenerative disorders and muscle diseases.

In all the above aspects and embodiments described herein, a distinction can be made between the transgene sequence and the coding sequence (CDS). By convention, the CDS refers to the entirety of the sequence between the start and stop codons. The CDS, by consequence of the regulatory nucleotide sequences used, may (in addition to encoding a functional protein) encode amino acid sequences with no clear protein function, that may be separated from the functional protein by a cleavage site. By contrast, the transgene sequence is defined herein as the region of the CDS which encodes the functional protein (i.e., the protein which one desires to express in diseased cells). For example, in a “Design 1” construct, the start codon may be present in or upstream of the cryptic exon, and in these cases the CDS would include at least a portion of the cryptic exon; however, the transgene region of the CDS (i.e., the region of the CDS encoding the functional protein) may be entirely downstream of the cryptic exon.

In all the above aspects and embodiments described herein, the transgene sequence may encode for a therapeutic protein. The construct can therefore be used to encode for a protein that is deficient or abnormal in a diseased cell. In some embodiments, the transgene sequence may encode for a regulatory protein. A regulatory protein is a protein that alters the expression of additional transgenes or endogenous genes. The construct can therefore be used to regulate expression of additional genes.

In all the above aspects and embodiments described herein, the transgene sequence may encode for a diagnostic protein. The construct can be used to further understand, probe, and diagnose cells with depletion of a hnRNP splicing factor.

In all the above aspects and embodiments described herein, depletion of a member of the hnRNP family of splicing factors activates expression of the transgene (i.e., results in expression of the protein product encoded by the transgene). Herein, the term “depletion” may, in some embodiments, refer to either the general depletion of the splicing factor from the cell (i.e., a “knockdown”, for example via expression of an shRNA targeting the mRNA of the splicing factor), and/or in some embodiments a nuclear depletion of the splicing factor (i.e., as occurs when TDP-43 aggregates in the cytoplasm). Both result in a loss of splicing regulation conferred by the splicing factor because splicing occurs primarily in the nucleus, and as such both would activate functional protein expression from the constructs described herein.

In the above aspects and embodiments described herein, the sequence defined by the first splice acceptor site and the first splice donor site may be a frame-shift inducing sequence. Depending on whether splicing occurs (i.e., diseased cells) or is repressed (i.e., in healthy cells), this dictates whether a frame-shifting inducing sequence is incorporated into the mRNA product of the construct, which introduces a frame-shift with respect to the start codon. In such embodiments, the construct may further comprise a premature termination codon (PTC) downstream of the regulatory domain, wherein the construct is configured such that wherein (i) in cells with nuclear depletion of the hnRNP splicing factor, the PTC is out of frame with the start codon in the mRNA product of the construct and (ii) in cells without nuclear depletion of the hnRNP splicing factor, the PTC is in frame with the start codon in the mRNA product of the construct. This results in the formation of a truncated protein in cells without depletion of the hnRNP splicing factor (i.e., healthy cells), but a functional protein is produced in cells with depletion of the hnRNP splicing factor, thereby providing one way to selectively express a protein in cells with hnRNP depletion. In such embodiments, the construct may further comprise a further intronic sequence (i.e., within an exonic sequence context), wherein the PTC is at least 40 nucleotides upstream of the further intronic sequence. Splicing of the further intron sequence promotes deposition of an exon junction complex (EJC) on the mRNA product, triggering nonsense-mediated decay of mRNA when a PTC has been encountered. In contrast, if no PTC is encountered, nonsense-mediated decay does not occur. The presence of a further intronic sequence therefore further improves the safety of the construct (as otherwise any peptide (e.g., truncated peptide) produced in healthy cells could build-up and could aggregate or be potentially toxic).

In some embodiments of the above aspects, the sequence between the first acceptor splice site and the first donor splice site is a cryptic exon sequence, wherein the regulatory domain further comprises an intronic region (i.e., defined by a second splice donor site and second splice acceptor site), wherein the cryptic exon sequence is located within said intronic region. In such constructs, the regulatory domain is therefore regulated by cryptic splicing, and the construct is configured such that the cryptic exon sequence is incorporated into the mRNA product of the construct in diseased cells (i.e., with nuclear depletion of hnRNP splicing factor), but is absent in the mRNA product of the construct in healthy cells (i.e., without nuclear depletion of hnRNP splicing factor). In some embodiments, the cryptic exon sequence may be a frame-shift inducing cryptic exon sequence, and can thereby regulate expression of the transgene as described above. Additionally, or alternatively, the cryptic exon sequence may encode for part of the transgene. This means that the complete transgene sequence is only fully present in the mature mRNA, enabling production of a functional protein, in diseased cells when the cryptic exon is incorporated into the mRNA product of the construct, but not in healthy cells when the cryptic exon is not incorporated. In some embodiments and examples herein, the intronic region is derived from the human AARS1 intronic region between exon 4 and exon 5. In some embodiments and examples herein, the intronic region is a synthetic sequence which is not derived from a naturally occurring intronic sequence.

Constructs comprising a cryptic exon sequence described herein may have a design according to “Design 1” or “Design 2” described herein, as demonstrated by FIG. 1 or FIG. 2 respectively. In Design 1 constructs, the transgene sequence is completely downstream of the regulatory domain. One of the benefits of this design is that it can be very easily modified to control the expression of various different proteins by including a different complete transgene or protein-coding sequence downstream of the regulatory sequence. Such embodiments may further comprise a protein cleavage site or self-cleaving site between the regulatory domain and the transgene sequence. The presence of this site has the advantage of ensuring that the transgene can be expressed without an extra N-terminal sequence, which in some cases may improve the functionality of the transgene's protein product.

In Design 2 constructs, the cryptic exon sequence encodes for at least a part of the transgene sequence. This may be an N-terminal part, internal part, or C-terminus part of the transgene sequence. Design 2 constructs also have many advantages. As compared with Design 1 constructs, the construct sequence can be smaller. Additionally, and unlike Design 1 where, in diseased cells, an unwanted peptide is produced from the upstream regulatory region, which may either be an N-terminal sequence attached to the transgene protein product, or a short released peptide, for Design 2 constructs no unwanted peptides are produced. Finally, there is reduced potential for “leaky expression”, for example via leaky scanning, of the full-length protein in healthy cells (i.e., in cells in which the cryptic exon is not expressed) because, unlike Design 1 constructs, the full transgene sequence is only present in the mature mRNA when the cryptic exon is included.

In some embodiments of the above aspects, the first splice donor site is upstream of the first acceptor site, and the first splice donor site and first splice acceptor site define a single regulatory intron. Constructs comprising a single regulatory intron sequence described herein may be as according to “Design 3”, as demonstrated by FIG. 3. The construct is configured such that in a cell that is depleted of splicing factor, the single regulatory intron is spliced, whereas in a cell that is not depleted of splicing factor, the single regulatory intron is either (i) not spliced or (ii) incorrectly spliced. This has the effect that only in cells that are depleted of the splicing factor (e.g., without TDP-43) is the intron spliced correctly, such that the start codon is in frame with an uninterrupted coding transgene sequence for the protein which is to be expressed. In some embodiments, the transgene sequence is completely downstream of the regulatory domain and/or single regulatory intron. In alternative embodiments, the transgene sequence may be encoded by exonic sequences which are upstream and downstream of the single regulatory intron.

In some embodiments, multiple regulatory domains may be contained within the same construct. For example, a transgene sequence may be split across multiple cryptic exons (i.e., may contain multiple regulatory domains in the style of “Design 2”), or may feature multiple regulatory introns (i.e., may contain multiple regulatory domains in the style of “Design 3”). In some embodiments, regulatory domains in the style of Design 1, 2, and/or 3 may be present in the same vector. The use of multiple regulatory domains may reduce the risk of leaky expression and improve safety.

### BRIEF DESCRIPTION OF FIGURES

The following disclosure will be described with reference to the following non-limiting examples and Figures.

FIG. 1 shows an example construct of the invention according to Design 1. This construct is designed such that repression of the first splice acceptor site (2) and first splice donor site (3), caused by binding of the splicing factor of the hnRNP family to the binding domain (4), leads to repression of splicing of the first splice acceptor site and/or first splice donor site. This is such that the cryptic exon is not included in the mRNA product in healthy cells. In diseased cells, splicing is not repressed, such that the cryptic exon is included in the mRNA product of the construct diseased cells. Inclusion or absence of the cryptic exon sequence can regulate expression of the transgene sequence (5).

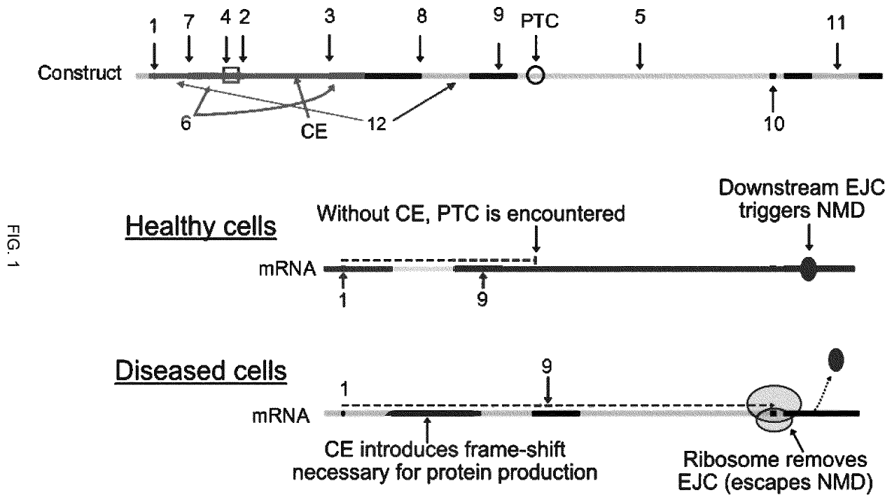

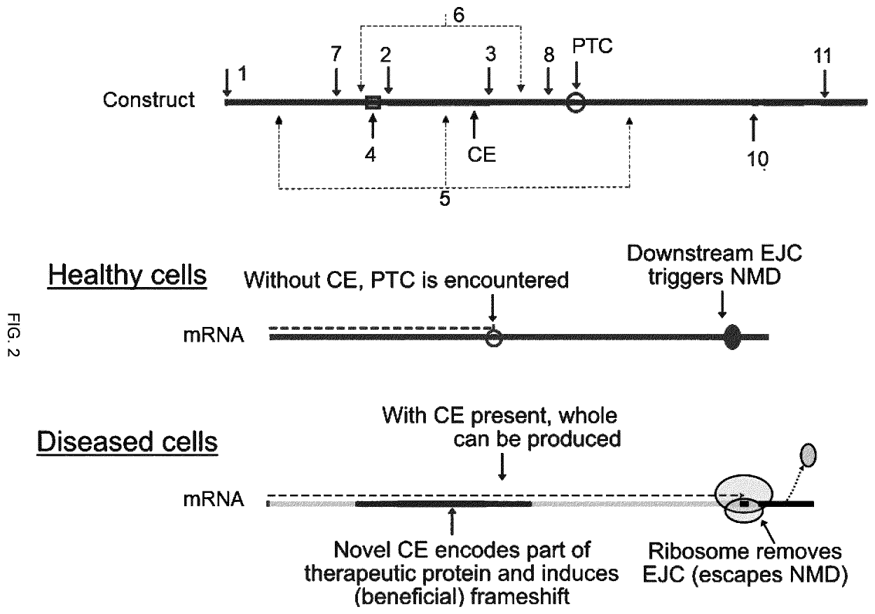

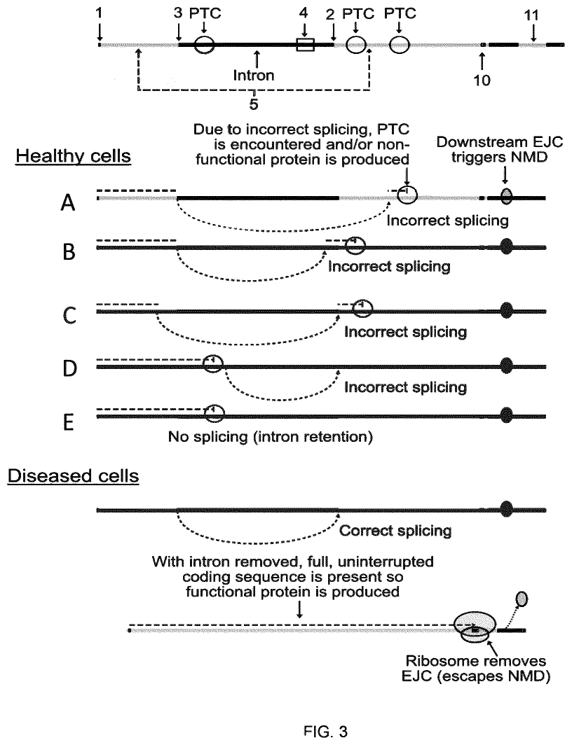

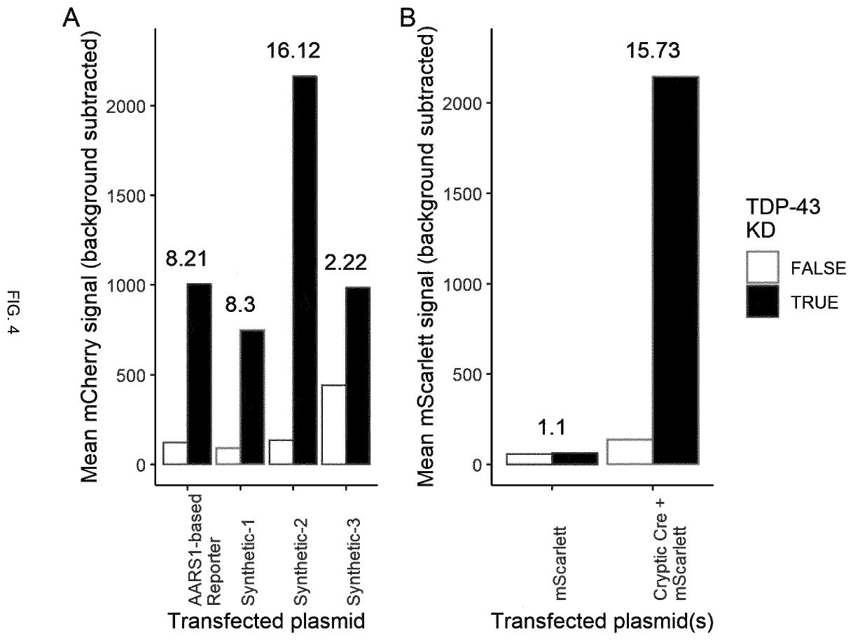

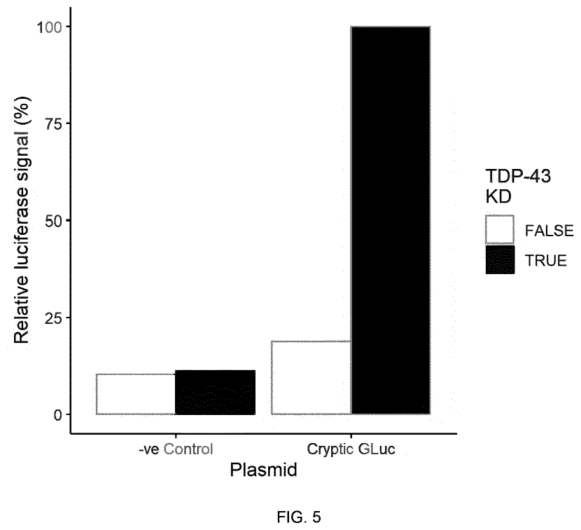

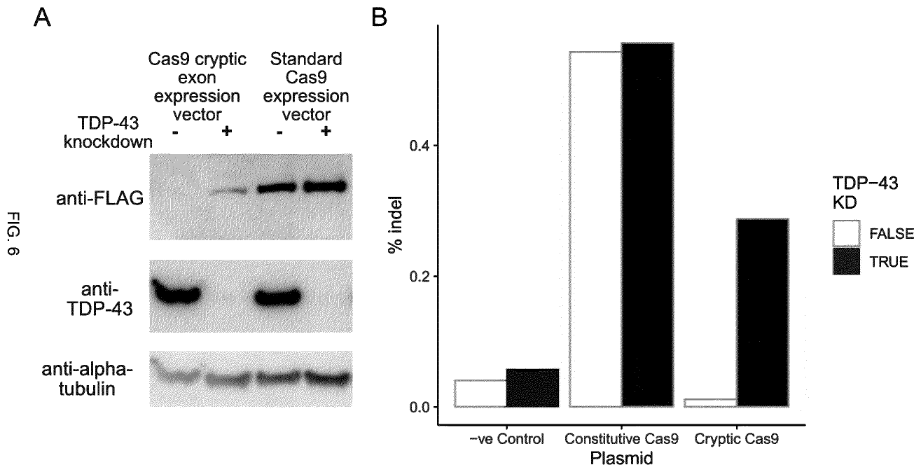

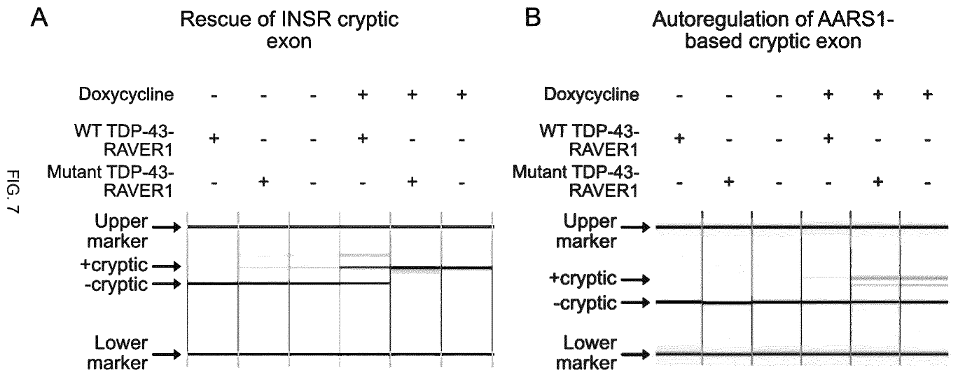

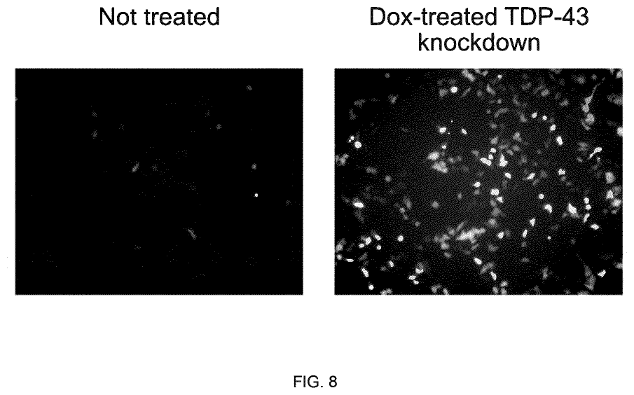

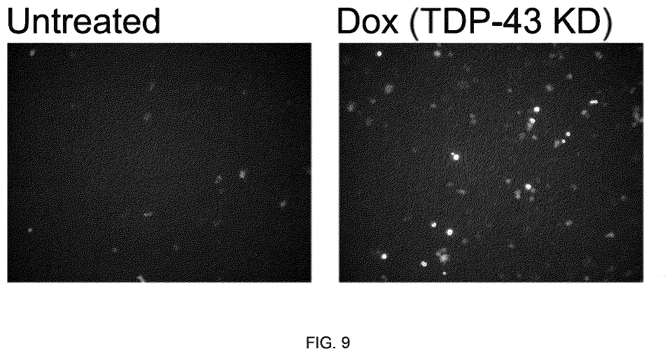

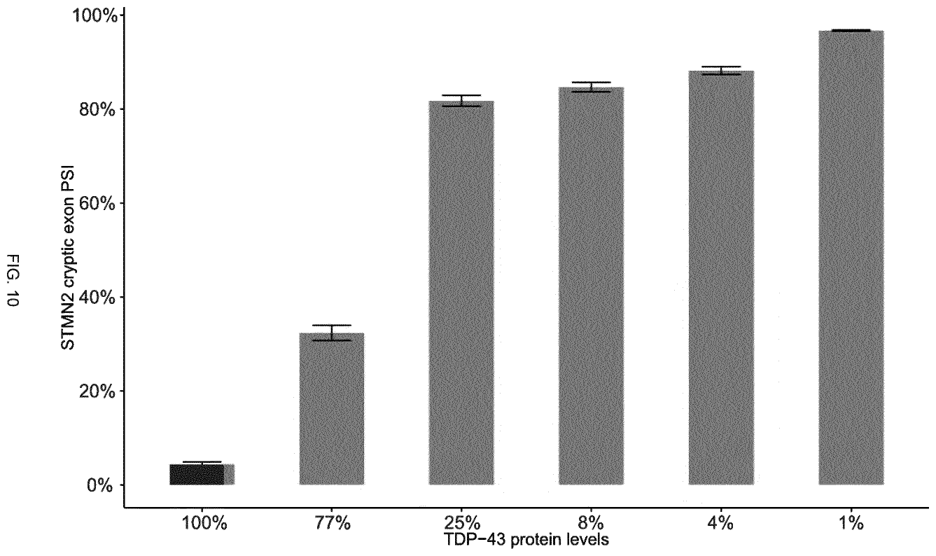

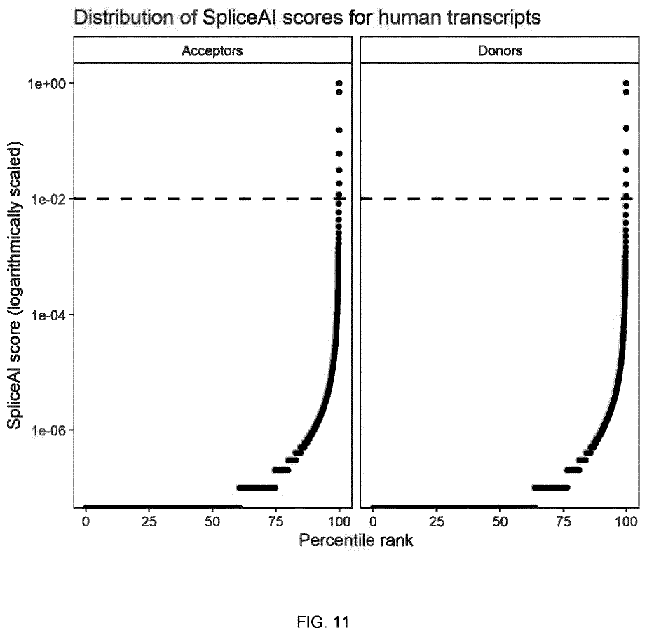

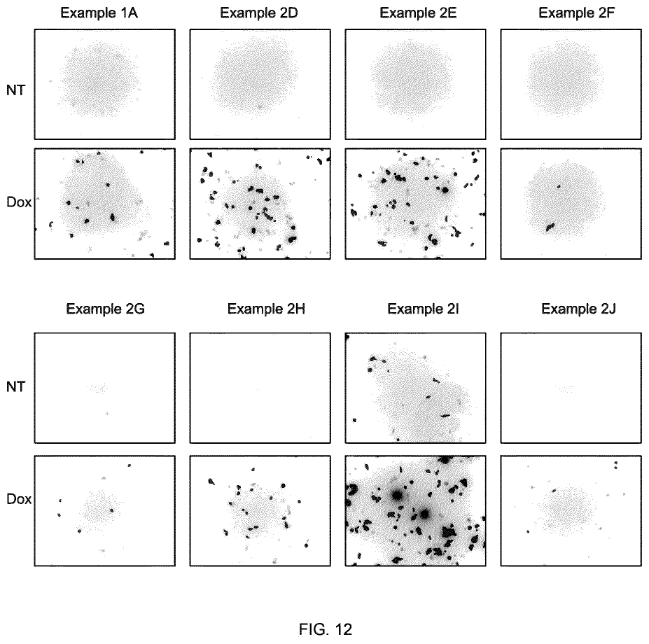

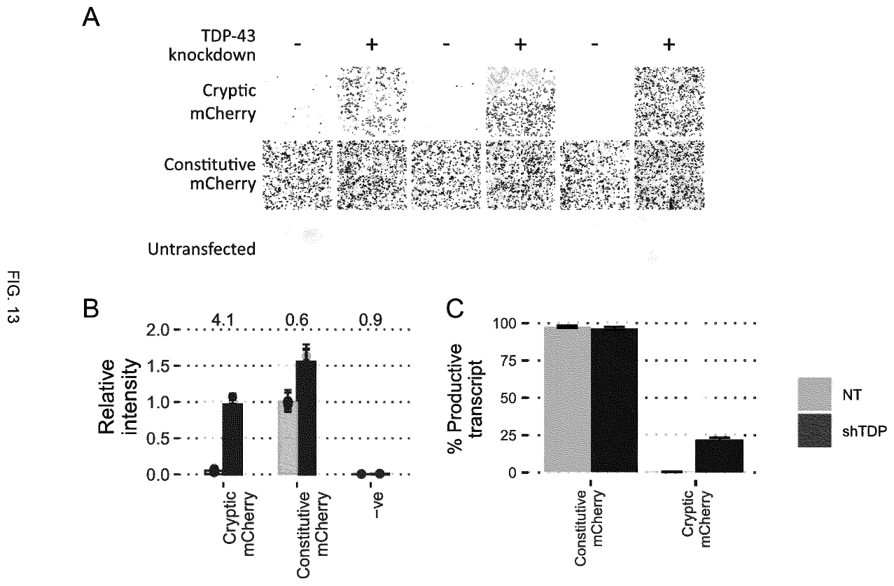

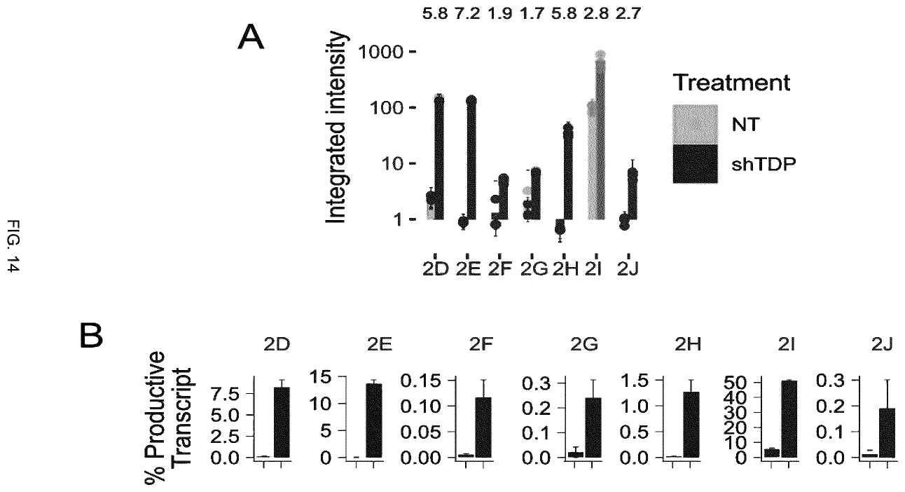

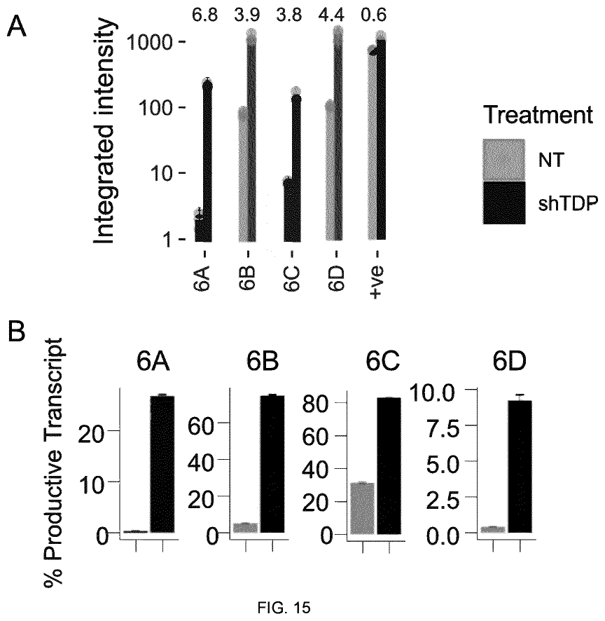

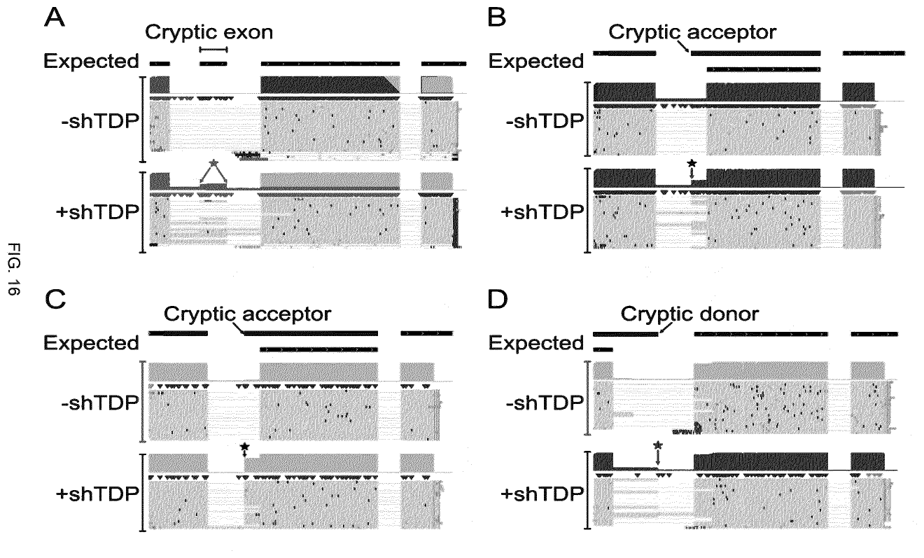

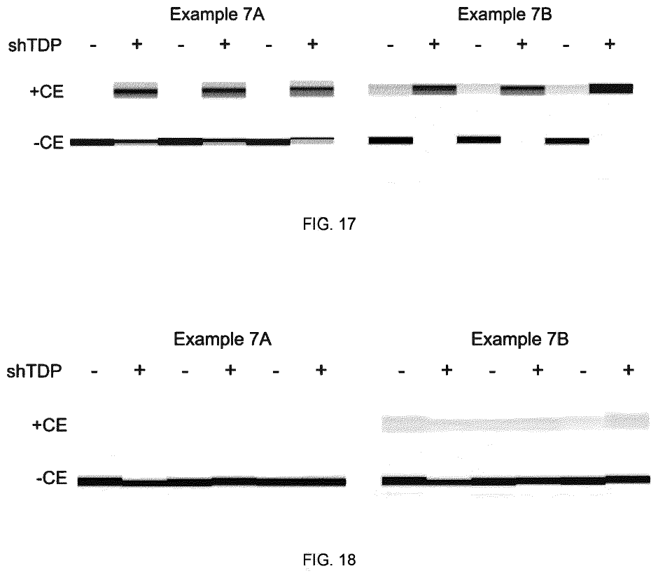

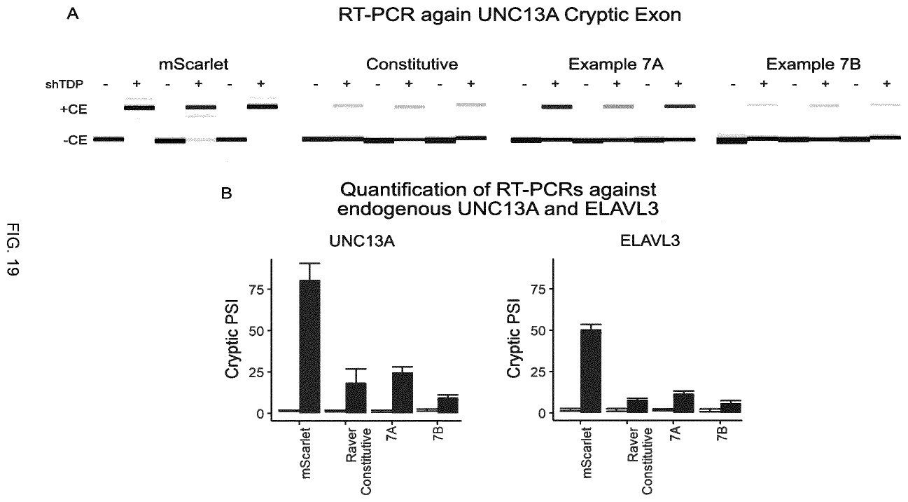

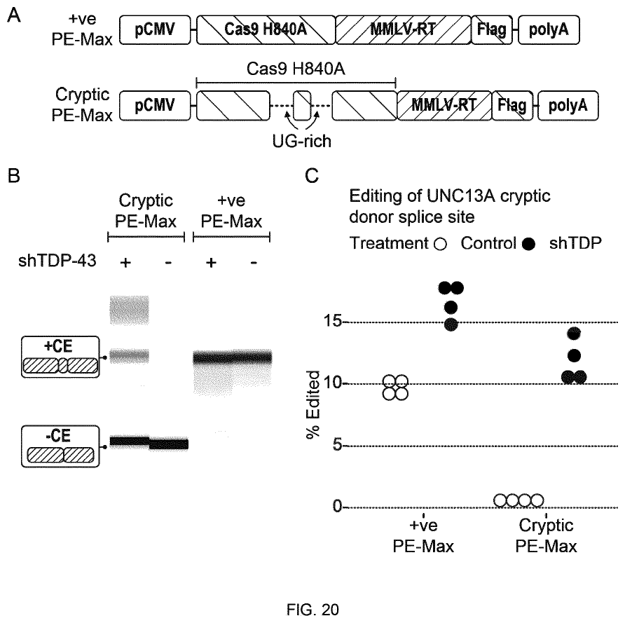

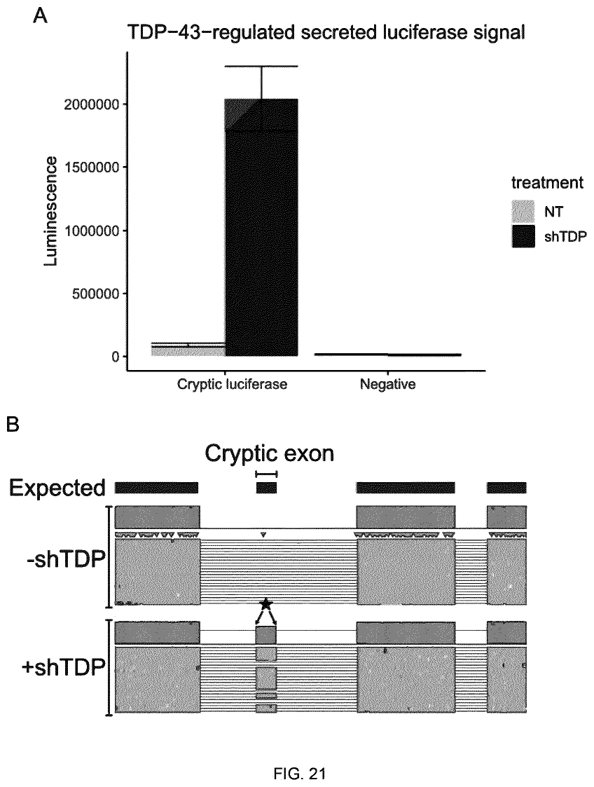

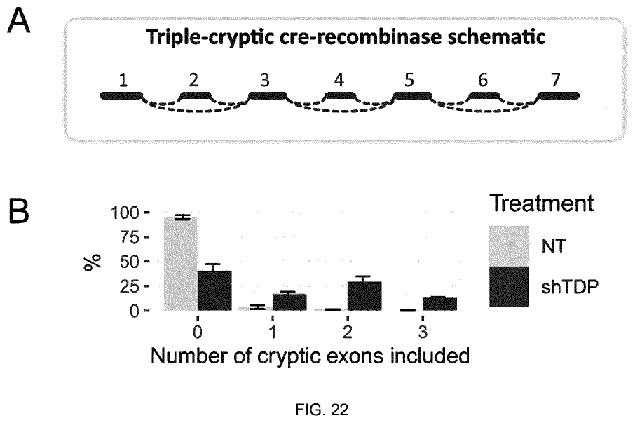

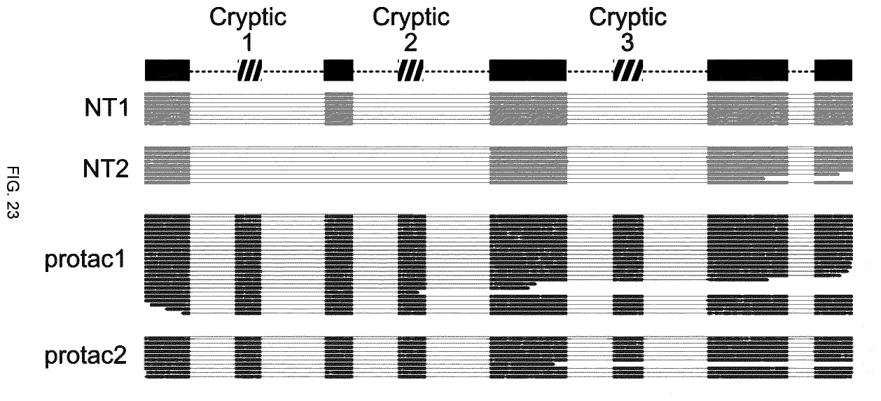

The example construct shown in FIG. 1 comprises a start codon (1), and a cryptic exon sequence (CE) defined by a first splice acceptor site (2) and a first splice donor (3) site. The construct comprises a binding domain for a hnRNP splicing factor (4) which regulates splicing of the first acceptor site (2) and/or the first splice donor site (3). The cryptic exon sequence (CE) is embedded within an intronic region (6), defined by a splice donor site (7) and splice acceptor site (8). A first part of the intronic region is upstream of the cryptic exon sequence, and a second part of the intronic region is downstream of the cryptic exon sequence. Exonic sequences (12) additionally flank the intronic region. A transgene sequence (5) is completely downstream of the regulatory domain and cryptic exon sequence (CE). The transgene sequence comprises a stop codon (10) at the end of the sequence.

An optional cleavage site (9) may be between the cryptic exon sequence and the transgene sequence (5). Optionally, the transgene sequence (5) further comprises a premature termination codon (PTC) at least part way through the sequence. Optionally, downstream of the transgene sequence is a further intronic sequence (11), within an exonic context. Optionally the cryptic exon sequence is a frame-shifting cryptic exon sequence.

In healthy cells, with no depletion of hnRNP splicing factor, splicing of the cryptic exon is repressed by binding of the splicing factor to the binding domain. The complete intronic region (6) is spliced (i.e., between 7 and 8), including the cryptic exon sequence (CE), such that no cryptic exon is included in the mRNA product of the construct. In this example, without a cryptic exon, the premature termination codon (PTC) is in frame with the start codon (1), leading to formation of a truncated protein. Furthermore, as a result of the further intronic sequence downstream of the transgene, an exon junction complex (EJC) is deposited on the mRNA product of the construct, which triggers nonsense mediated decay of the mRNA. Instead, in diseased cells, with depletion of the hnRNP splicing factor, splicing of the cryptic exon is not repressed. The first part of the intronic region is spliced (i.e., between 7 and 2) and the second part of the intronic region is spliced (i.e., between 3 and 8), such that the cryptic exon sequence (i.e., between 2 and 3) is included in the mRNA (i.e., mature mRNA) of the product. In this example, this introduces a frame-shift such that the PTC is no longer in frame with the start codon and the transgene can be fully translated such that functional protein can be produced. The cleavage site (9) releases the transgene protein separately from the peptide produced from exonic sequences (12) that flank the intronic region (6). Since no PTC is encountered in diseased cells, the ribosome removes any exon junction complex (EJC) meaning that NMD does not occur.

In alternative embodiments (not shown), the cryptic exon itself may contain the start codon.

FIG. 2 shows an example construct of the invention according to Design 2. Like Design 1, a cryptic exon is included in the mRNA product in diseased cells, but repression of the first splice acceptor site (2) and first splice donor site (3), caused by binding of the splicing factor of the hnRNP family to the binding domain (4), means that the cryptic exon is not included in the mRNA product in healthy cells. However, different from Design 1, the (CE) sequence itself encodes for part of the transgene sequence (5).

The construct comprises a start codon (1) and a cryptic exon sequence (CE) defined by a first splice acceptor site (2) and a first splice donor site (3). The construct also comprises a binding domain for a hnRNP splicing factor (4) which regulates splicing of the first splice acceptor site (2) and/or the first splice donor site (3). The cryptic exon sequence (CE) is also embedded within an intronic region (6), defined by a splice donor site (7) and splice acceptor site (8). In this example, a first part of the transgene sequence is encoded by an exonic sequence upstream intronic region, a second part of the transgene is the cryptic exon sequence, and a third part of the transgene is encoded by an exonic sequence downstream of the cryptic exon sequence. The part of the transgene downstream of the cryptic exon sequence optionally also comprises a premature termination codon (PTC) at least part way through the sequence, and the CE is a frame-shifting CE sequence. Optionally downstream of the transgene sequence is a further intronic sequence (11) in an exonic context.

Similar to Design 1, in healthy cells, with no depletion of hnRNP splicing factor, splicing of the cryptic exon is repressed and no cryptic exon is included in the mRNA product of the construct. This means that the full sequence encoding the protein to be expressed is not present in the mature mRNA product of the construct in healthy cells. In contrast, the diseased cells express mature mRNA that encode for the complete transgene protein product. Additionally, in this example, due to the frame-shifting CE sequence, a premature termination codon (PTC) is in frame with the start codon (1) in the mRNA product of the construct in healthy cells, but not in diseased cells, as with Design 1. Due to the presence of a further intronic sequence, an exon junction complex (EJC) triggers nonsense mediated decay of the mRNA product of healthy cells, but a ribosome removes the EJC in diseased cells such that no nonsense-mediated decay occurs.

In alternative embodiments (not shown), the cryptic exon may instead encode for the N- or C-terminal region of the protein product. Additionally, or alternatively, the PTC need not be present in the transgene downstream of the regulatory domain (not shown). This is because the absence of a cryptic exon in the mRNA product of the construct can lead to production of a non-functional protein product.

FIG. 3 shows an example construct of the invention according to Design 3. In healthy cells, this construct is designed such that repression of the first splice donor site (3) and first splice acceptor site (2), caused by binding of the splicing factor of the hnRNP family to the binding domain (4), leads to repression of splicing of the single regulatory intron, such that the single regulatory intron is either not spliced or incorrectly spliced. In diseased cells, splicing is not repressed, such that no part of the single regulatory intron is included in the mRNA product of the construct. In this example, the construct comprises a start codon (1) and a single regulatory intron sequence (intron) defined by a first splice donor site (3) and a first splice acceptor site (2). The construct comprises a binding domain for a hnRNP splicing factor (4) which regulates splicing of the first splice donor site (3) and/or the first splice acceptor site (2). In this example, the transgene sequence is encoded by exonic sequences both upstream and downstream of the single regulatory intron in two parts (5), although in alternative embodiments (not shown), the transgene (5) instead be completely downstream of the single regulatory intron. The construct may further comprise an alternative splice acceptor site and/or an alternative splice donor site (not shown). The alternative splice acceptor site and/or alternative splice donor site may otherwise be referred to as a “decoy” splice site herein. In some embodiments, the alternative splice site is configured such that it is spliced preferentially in cells that are not depleted of a splicing factor of the hnRNP family, (e.g., TDP-43), and wherein the first donor and/or acceptor splice site is spliced (i.e., the decoy splice site is not used) in cells that are depleted of the splicing factor of the hnRNP family (e.g., TDP-43). In some embodiments, the alternative splice site is a donor splice site located upstream of the first donor splice site. In some embodiments, the alternative splice site is a donor or acceptor splice site located between the first donor and first acceptor splice sites. In some embodiments, the alternative splice site is an acceptor splice site located downstream of the first acceptor splice site.

As described for Design 1 and Design 2 constructs, the construct may further comprise one or more premature termination codons (PTC) and the construct may optionally further comprise a further intronic sequence (11) downstream of the transgene (5). This promotes deposition of an EJC and NMD for the mRNA product in healthy cells.

In healthy cells, the intron is either retained fully (see e.g., E) or partially (see, e.g., B or D), due to the repression of both splice sites (e.g., E), or incorrectly spliced, due to the repression of one splice site (see, e.g., A and C). This means that a non-functional protein is produced in healthy cells, while a functional protein is produced in diseased cells. Optionally, a premature termination codon (PTC) is present in part of the transgene sequence (5) which is downstream of the single regulatory intron sequence. In certain embodiments, e.g., when either intron retention or incorrect splicing introduces a frame-shift (see, e.g., A, B and C), the construct is configured such that a PTC is in frame with the start codon when at least part of the intron is included in the mRNA product of the construct, but the PTC is not in frame with the start codon when the intron is absent in the mRNA product of the construct. This further leads to the formation of a truncated or non-functional protein for healthy cells, but a functional protein in diseased cells. Optionally, and additionally or alternatively, a PTC may instead be present in the intron, in frame with the start codon, such that full or partial intron retention results in this PTC being in frame with the start codon in the mRNA product of the construct (see. e.g., D and E). The combination of a PTC and a deposited EJC leads to NMD, preventing expression of truncated, non-functional protein, which could otherwise be toxic for the cell.

The presence of a PTC in the construct, and thereby in the mRNA product (i.e., mature mRNA product) of the construct in healthy cells, is not an essential part of the invention. This is because intron retention or incorrect splicing can produce a non-functional protein product (for example due to internal truncation due to incorrect splicing, or due to inclusion of disruptive amino acid sequence that impairs folding).

FIG. 4A shows mCherry fluorescence signal from four cryptic exon-containing vectors. “AARS1-based Reporter”, corresponds to Example 1A which is a Design 1 construct, and features a frame-shifting upstream AARS1-derived cryptic exon/intron regulatory sequence, and a downstream mCherry sequence. “Synthetic-1/2/3” feature computer-generated cryptic-exon sequences, corresponding to Examples 2A-2C which are Design 2 constructs, that encode an internal part of the mCherry sequence, flanked by computer-generated intronic sequences. Numbers show the ratio of signal in cells with TDP-43 knockdown versus control cells. FIG. 4B shows mScarlet fluorescence signal from cells transfected with an mScarlet-encoding plasmid containing a “poison exon” flanked by LoxP sites, co-transfected with a plasmid encoding Cre recombinase where part of the Cre recombinase sequence is encoded by a synthetic cryptic exon, flanked by AARS1-derived intronic sequences (i.e., the construct described in Example 3, another Design 2 construct). Numbers show the ratio of signal in cells with TDP-43 knockdown versus control cells. Y-axis values refer to “Scale Values” from Flow-Jo.

FIG. 5 shows the signal from secreted luciferase with construct Example 1B, an example Design 1 construct. “−ve Control” refers to cells transfected with a vector encoding mCherry.

FIG. 6 shows TDP-43-dependent genome editing. FIG. 6A shows western blot showing expression of FLAG-tagged Cas9, TDP-43 and alpha-tubulin in cells transfected with a Cas9 expression vector containing a cryptic exon (left), corresponding to Example 4 which is an Example Design 2 construct, or a constitutive Cas9 expression vector (right) with or without TDP-43 knockdown. FIG. 6B shows the fraction of Illumina reads with indels at the targeted CDK4 locus. “−ve Control”=cells transfected with a vector encoding mCherry.

FIG. 7 shows repression of cryptic exons and autoregulation. A: RT-PCR analysis of cells transfected with an INSR cryptic exon minigene, and optionally co-transfected with plasmid expressing cryptic TDP-43-RAVER1 fusion protein (i.e., according to Example 1C or a mutant 1C, which is an example Design 1 construct). The “mutant” protein is RNA-binding deficient. Doxycycline induces TDP-43 knockdown. B: Is as described for part A, except that the RT-PCR target is the AARS1-derived frame-shifting cryptic exon, thus demonstrating autoregulation for this construct.

FIG. 8 shows results using a Cas9/AARS1 mCherry reporter corresponding to Example 1D, which is an Example Design 1 construct: mCherry fluorescence, is assessed by fluorescence microscopy, from cells transfected with a construct containing a downstream mCherry transgene, regulated by an upstream frame-shifting cryptic exon; the cryptic exon is a novel sequence encoding part of S. pyogenes Cas9, flanked by intronic regions derived from AARS1. Left: cells without TDP-43 depletion; right: cells with TDP-43 depletion.

FIG. 9 shows the results of mCherry fluorescence assessed via fluorescence microscopy for SK-N-DZ cells transfected with the AARS1-mCherry-FLAG intron retention construct, which is a Design 3 construct corresponding to Example 5, with doxycycline inducible TDP-43 knockdown.

FIG. 10 shows STMN2 cryptic exon levels versus TDP-43 protein levels. The percentage inclusion (PSI) of the STMN2 cryptic exon, as assessed by RNA sequencing, is demonstrated against the level of remaining TDP-43, as assessed by western blot (% TDP-43 protein remaining is shown on the x-axis). Since these cells exhibit correctly localized TDP-43, the total level of TDP-43 protein is equivalent to the total level of nuclear TDP-43. This indicates that presence of STMN2 cryptic inclusion is indicative of nuclear TDP-43 depletion. This further demonstrates that a relative mild depletion of TDP-43 (eg., 23% depletion resulting in 77% remaining) can result in greatly increased levels of cryptic splicing.

FIG. 11 shows the distribution of Splice AI scores (logarithmically scaled) as determined by the SpliceAI algorithm in human transcripts for 500 genes, none of which were in the original training set for the Splice AI algorithm. The dashed line corresponds to a cut-off of 0.01, which corresponds to a ˜ 99.8th percentile rank of splicing sites.

FIG. 12 shows the fluorescence microscopy images of SK-N-DZ cells transfected with either a Design 1-style mCherry construct reporter (Example 1A), or various synthetic Design 2-style mScarlet construct reporters (Example 2D-2J). Doxycycline induces TDP-43 knockdown. The images shown have been inverted for clarity.

FIG. 13 shows A) fluorescence microscopy images, B) fluorescence microscopy quantification and C) nanopore sequencing of SK-N-DZ cells that were transfected with a constitutively expressing mCherry vector or Example 1A, both with or without TDP-43 knockdown. Numbers above bar graphs show the log 2-fold-increase with TDP-43 depletion.

FIG. 14 shows A) fluorescence microscopy quantification and B) nanopore sequencing of SK-N-DZ cells that were transfected with various Design 2 constructs (i.e., Examples 2D-2J) encoding mScarlet, both with or without TDP-43 knockdown. Numbers above bar graphs show the log 2-fold-increase with TDP-43 depletion.

FIG. 15 shows A) fluorescence microscopy quantification and B) nanopore sequencing of SK-N-DZ cells that were transfected with various Design 3 constructs (i.e., Examples 6A-6D) with or without TDP-43 knockdown. Numbers above bar graphs show the log 2-fold-increase with TDP-43 depletion.

FIG. 16 shows example nanopore traces derived from SK-N-DZ cells transfected with a Design 2 construct (i.e., Example 2E) and various Design 3 constructs, each of which encode mScarlet (i.e., Examples 6A, 6B and 6D). The star symbol highlights the usage of the cryptic splice site(s). The expected splicing pattern is shown above; for Design 3 constructs, the “decoy” splice site is also shown.

FIG. 17 shows RT-PCR analysis of SK-N-DZ cells, with or without TDP-43 knockdown, transfected with F2L mutants of Design 2 constructs (i.e., Examples 7A and 7B).

FIG. 18 shows RT-PCT analysis of SK-N-DZ cells, with or without TDP-43 knockdown, transfected with Design 2 constructs (i.e., Examples 7A and 7B) with functional TDP-43 sequences (i.e., without the F2L mutation).

FIG. 19 shows A) RT-PCR of the cryptic exon region for endogenously expressed UNC13A transcript for SK-N-DZ cells expressing Design 2 constructs (i.e., Examples 7A and 7B), and B) quantification of the above RT-PCRs against UNC13A, and equivalent RT-PCRs (not shown) performed against the ELAVL3 cryptic exon, with or without knockdown of endogenous TDP-43. For each sample, the bar on the left shows the quantification for untreated cells, and the bar on the right shows the quantification for dox-treated (i.e. shTDP-43) cells.

FIG. 20 shows A) diagrams of the Example 8 vector (bottom) and control (top), B) RT-PCR analysis of splicing of the vectors in part A with and without TDP-43 knockdown for SK-N-DZ cells, with or without TDP-43 knockdown and C) analysis of the genome editing at the expected locus via Nanopore amplicon sequencing for these cells.

FIG. 21 shows A) Luciferase activity from media of SK-N-DZ cells transfected with the Example 9 construct, with or without TDP-43 knockdown and B) Nanopore traces from these cells.

FIG. 22 shows A) a schematic of the Example 10 triple cryptic exon Cre-recombinase vector. Exons 2, 4 and 6 are “cryptic”, and B) Quantification of Nanopore reads for the number of cryptic exons included in each transcript for SK-N-DZ cells without (NT) or with doxycycline-induced knockdown of TDP-43. Error bars show standard error across three replicates.

FIG. 23 shows Nanopore traces derived from i3 iPSCs expressing the Example 10 triple cryptic exon Cre-recombinase vector, treated with or without a halo-tag based “protac” sequence that depletes endogenous halo-tagged TDP-43. The expected positions of the three cryptic exons are shown with the striped boxes.

### DETAILED DESCRIPTION

For any SEQ IDs disclosed herein, the complementary sequence is of each SEQ ID is also disclosed. Also disclosed herein is a construct with a complementary sequence to that described herein which may be used to encode for the constructs described herein.

The terms “treatment” and “treating” herein refer to an approach for obtaining beneficial or desired results in a subject and includes both a prophylactic benefit and a therapeutic benefit.

“Therapeutic benefit” refers to eradication, amelioration or slowing the progression of the underlying disorder being treated. Also, a therapeutic benefit is achieved with the eradication or amelioration of one or more of the physiological symptoms associated with the underlying disorder such that an improvement is observed in the subject, notwithstanding that the patient may still be afflicted with the underlying disorder.

“Prophylactic benefit” refers to delaying or eliminating the appearance of a disease or condition, delaying, or eliminating the onset of symptoms of a disease or condition, slowing, halting, or reversing the progression of a disease or condition, or any combination thereof. In the context of the present invention, the prophylactic benefit or effect may involve the prevention of the condition or disease. The construct, vector or pharmaceutical composition may be administered to a subject at risk of developing a particular disease, or to a subject reporting one or more of the physiological symptoms of a disease, even though a diagnosis of this disease may not have been made.

The term “subject” refers to any suitable subject, including any animal, such as a mammal. In preferred embodiments described herein, the subject is a human.

The term “comprising” (and related terms such as “comprise” or “comprises” or “having” or “including”) includes those embodiments, for example, an embodiment of any composition of matter, composition, method, or process, or the like, that “consist of” or “consist essentially of” the described features, unless context clearly dictates otherwise. The term “comprises” or “comprising” can be used interchangeably with “includes”.

The term “RNA-seq” referred to herein, otherwise known as “RNA sequencing”, refers to a next-generation sequencing technology which reveals the presence and quantity of RNA in a sample which can be used to analyse the cellular transcriptome.

A “construct” described herein has its normal meaning in the art and refers to a synthetic nucleic acid sequence which contains genetic material encoding for a gene of interest. A construct is intended not to be a complete naturally occurring nucleic acid sequence, i.e., as found in the genome of an organism (although the construct itself may comprise component parts that are derived from naturally occurring sequences). The construct may have a maximum length, i.e., the construct may comprise less than 50,000 nucleotides, or less than 40,000 nucleotides, or less than 30,000 nucleotides, or less than 20,000 nucleotides, or in some examples, less than 10,000 nucleotides or less than 5000 nucleotides, or less than 2500 nucleotides.

A “vector” has its normal meaning in the art and refers to a synthetic piece of nucleic acid which comprises a construct (i.e., as defined above), and which has the function of delivering the construct to a cell.

“Nucleotides” described herein describe the constituent parts of a nucleic acid sequence. Nucleotides comprise a nucleobase (e.g., A, G, T and C in DNA, or A, G, U and C in RNA, however other nucleobases may be used), linked to a sugar (e.g., deoxyribose in DNA, and ribose in RNA, however, other sugars may be used). In DNA and RNA, the sugars are linked by a phosphodiester backbone to form a nucleic acid sequence, however other backbones may be used.

“Nuclear depletion of the splicing factor” as described herein, may be defined as a cell with at least 20% loss of splicing factor, or at least 25% loss, or preferably at least 50% loss of splicing factor in the nucleus of a cell (or as an average (mean) of a population of cells) as compared to a healthy cell of the same type (or as an average (mean) of a population of healthy cells). Depletion of the splicing factor can be determined by standard methods, such as western blotting. In some examples, the term “nuclear depletion of the splicing factor” can be replaced with or is interchangeable with the term “absence of binding of splicing factor to the splicing factor binding domain”, and the term “without nuclear depletion of splicing factor” can be replaced with or is interchangeable with the term “presence of binding of splicing factor to the splicing factor binding domain”. When the splicing factor is TDP-43, nuclear depletion may be determined by determining the presence of a STMN2 cryptic splicing event (i.e., the presence of a STMN2 cryptic exon) in a cell transcript, which may be determined by RNA-sequencing. This is because the presence of a STMN2 cryptic exon in mRNA transcripts is indicative of nuclear depletion of TDP-43 (see FIG. 10). Depletion of TDP-43 refers to depletion of “normal” or wild-type TDP-43, and may not include pathological or mutated TDP-43. Pathological TDP-43 may be a hyper-phosphorylated, ubiquinated or cleaved form of TDP-43, a TDP-43 form with decreased solubility, or a misfolded form of TDP-43, a mutant form of TDP-43, or a TDP-43 with altered cellular location.

A cell with nuclear depletion of the splicing factor of the hnRNP family may be referred to as a “diseased cell” herein. A cell without nuclear depletion of the splicing factor of the hnRNP family may be referred to as “healthy cell” herein.

Any mention of splicing factor described herein is intended to refer to a splicing factor or splicing repressor protein of the hnRNP family. hnRNP as defined herein refers to a heterogenous nuclear ribonucleoprotein, which includes TDP-43 as a family member. The term hnRNP splicing factor may be used interchangeably with the term hnRNP splicing repressor protein. The term splicing factor of the hnRNP family may also be used interchangeably with the term hnRNP splicing factor.

TDP-43 as defined herein refers to TAR DNA Binding protein 43 (Transactive response DNA binding protein 43 kDa), which in humans is a protein encoded by the TARDBP gene. TDP-43 has been shown to bind both DNA and RNA and have multiple functions in transcriptional repression, pre-mRNA splicing and translational regulation, among other functions.

Splicing as defined herein refers to the process wherein pre-mRNAs are transformed into mature mRNAs, wherein introns are removed and exons are joined together.

Synonymous codons as described herein refer to different codons that encode for the same amino acid.

“In frame” defined herein refers to a situation where codons are spaced by a number of nucleotides that are divisible by 3. “Out of frame” refers to a situation where codons are spaced by a number of nucleotides that are not divisible by 3.

A cryptic exon as defined herein refers to a splicing variant that is incorporated into a mature mRNA (i.e., upon depletion of relevant splicing factor), introducing frameshifts or stop codons, among other changes in the resulting mRNA. In other words, the cryptic exon is a nucleotide sequence that is preferentially spliced upon depletion of the relevant splicing factor. A cryptic exon may otherwise be referred to as “CE”, “cryptic”, “cryptic exon sequence” or “cryptic event” herein or elsewhere in the art.

A single regulatory intron defined herein refers to a splicing variant that is incorporated, at least in part, into a mature mRNA, (i.e., due to alternative splicing of the intron), to introduce frameshifts or stop codons, among other changes in the resulting mRNA. In other words, the single regulatory intron is a nucleotide sequence that is spliced differently in cells with depletion of a relevant splicing factor; where alternative splicing of this intron may introduce frameshifts or stop codons, among other changes in the resulting mRNA.

Sequence complementarity disclosed herein refers to Watson-Crick base pairing in nucleic acids, e.g., wherein A binds with T (or U or modified variants thereof), and wherein C binds with G (or modified variants thereof).

Any genomic or chromosomal position described herein refers to the position on the human genome and associated transcriptome (hg38).

When ranges are used herein, all combinations and sub-combinations of ranges and specific embodiments therein are intended to be included. The term “about” or “˜” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale.

It must be noted that as used herein and in the appended claims, the singular forms “a”, “an”, and “the” include plural referents unless the context clearly dictates otherwise.

The binding domain for the splicing factor described herein refers to the sequence which encodes for the binding domain in the mRNA. For example, when referring to TG or UG rich motifs, for example, in the context of a TDP-43 binding domain, the TG rich motif is present in the DNA construct, while the UG-rich motif is present in the RNA.

Unless defined otherwise, all technical and scientific terms used herein have the same meanings as commonly understood by one of ordinary skill in the art to which this invention belongs. Abbreviations used herein have their conventional meaning within the chemical and biological arts, unless otherwise indicated.

Splice score as described herein refers to the splice score as determined by the Splice AI algorithm. The splice score as determined by the Splice AI algorithm is determined by calculating the probability of splicing of a given position, given a specific sequence context. The sequences flanking the splice site may comprise the entire construct, (i.e., from start to finish, or in a vector context from the end of the promoter to the start of the polyadenylation signal); this is because sequences in the flanking regions (e.g., up to 10,000 nucleotides apart) can impact the splicing prediction at a given position. The Splice AI algorithm can be found at the following link https://github.com/Illumina/SpliceAI, and can be used according to the instructions as described in Jaganathan et al., 2019, Cell, 176, 535-548, “Predicting Splicing from Primary Sequence with Deep Learning”, the contents of which is incorporated herein by reference. The version of the Splice AI algorithm and pretrained network weights used may be version 1.3.1. A score of 0.01 is in the 99.8^{th} percentile of scores generated by the Splice AI algorithm (see FIG. 11), and corresponds to a very high probability of splicing (i.e., as compared with random positions in the genome); as described in the Jaganathan et al reference and as shown in FIG. 11, a large fraction of bona fide naturally occurring splice sites obtain scores of far below 1. In particular, splice sites which are alternatively spliced in different tissues (for example, constitutively spliced in a neuronal cell, but not a hepatocyte), typically obtain lower SpliceAI scores, despite acting as strong splice sites in specific cell types.

A splice site, as understood in the art, is the boundary between an intron sequence and exon sequence. During splicing, the nucleotide sequence is cut at said splice sites, i.e., the nucleotide sequence is cut at the boundary between an intron sequence and exon sequence.

A splice acceptor site is a splicing site that occurs between and intron and exon, i.e., splice site immediately upstream of an exonic sequence wherein the intron is upstream of the exonic sequence. A splice acceptor site is characterised by any splice site that comprises the dinucleotides “AG” upstream of the splice site (i.e., at the end of the intron sequence which is upstream of the exon).

A splice donor site Is a splicing site that occurs between an exon and an intron, i.e., an exonic sequence wherein the exon is upstream of the intron. A splice donor site is characterised by any splice site that comprises the dinucleotides “GT” downstream of the splice site (i.e., at the start of the intron sequence which is downstream of the exon).

A splicing factor is a protein involved in splicing, i.e., the removal of introns from mRNA so that exons are bound together.

Unless context explicitly states otherwise, it is envisaged that any embodiment described herein may be combined with any other embodiment described herein. For example, embodiments described for the hnRNP binding domain, or more specifically TDP-43 binding domain, can be readily combined with other embodiments described herein and is not limited to construct design (e.g., Design 1, 2, or 3), cryptic exon sequence (if present), single regulatory intron (if present), first splice acceptor site, first splice donor site, PTC, further intronic sequence, intronic region (if present), etc. Similarly, the features of any dependent claim may be readily combined with the features of any of the independent claims or other dependent claims, unless context clearly dictates otherwise.

As described herein whether a functional protein is either produced or not produced from the transgene sequence, this refers to whether a functional protein is produced or not from the mRNA product of the construct.

### Construct

The construct as described herein is a synthetic nucleotide sequence. In some embodiments, the construct preferably comprises a DNA nucleotide sequence. The construct may comprise double-stranded DNA or single-stranded DNA. In some embodiments, the construct comprises linear DNA or circular DNA. The nucleotides may comprise or are formed from non-modified nucleobases (e.g., C, T, A or Gin DNA), but may also comprise modified nucleobases (e.g., but not limited to, 5-methylcytosine, 6-methyladenosine, deoxyuridine), provided the Watson-Crick base pairing, transcription and splicing, is not compromised. While a DNA nucleotide sequence is preferred, any other suitable nucleotide sequence may be used, i.e., comprising nucleotides with a different sugar, or a different backbone, provided the Watson-Crick base pairing, transcription, and splicing is not compromised

### Regulatory Domain

### First Splice Acceptor Site and First Splice Donor Site

The regulatory domain comprises a first splice acceptor site and the first splice donor site.

In some embodiments, the sequence surrounding the first splice acceptor site is HAG/N wherein/represents the splice site, wherein H=C, T or A and N is C, T, A or G. In some embodiments or examples, the construct comprises a polypyrimidine tract upstream of the first splice acceptor site (i.e., within the intronic region upstream of the splice acceptor site, e.g., upstream of HAG/N). In some embodiments, the polypyrimidine tract is upstream of the first splice acceptor site, more preferably up to 40 nucleotides upstream of the first splice acceptor site, or up to 20 nucleotides upstream of the first splice acceptor site. A polypyrimidine tract defined herein may be described as region that is pyrimidine rich, defined as a 20 nucleotide region comprising at least 70% pyrimidines or defined a 30 nucleotide region comprising at least 80% pyrimidines.

In some embodiments, the regulatory domain further comprises a branch site comprising an adenosine upstream of the first splice acceptor site and the polypyrimidine tract (i.e., within the intronic region upstream of the splice acceptor site). The branch site may comprise the sequence PTNAP, wherein N is any nucleotide, P is a pyrimidine (i.e., C or T), and wherein the underlined A is the branchpoint for example (e.g., CTGAC). The branch site may be located up to 45 nucleotides upstream of the first splice acceptor, preferably up to 35 nucleotides upstream of the first splice acceptor and preferably between 20 and 35 nucleotides upstream of the first splice acceptor.

In some embodiments, the sequence surrounding the first splice donor site is N/GT wherein/represents the splice site, and wherein N is C, T, A or G. In some examples described herein, the sequence surrounding the first donor splice site is CAG/GT wherein/represents the splice site.

In some embodiments, the first splice acceptor site and/or the first splice donor site have a splice score of 0.01 or above as determined by the Splice AI algorithm. In some embodiments, the first splice acceptor site and/or the first splice donor site have a splice score of 0.05 or above as determined by the Splice AI algorithm, or at least 0.1 or above, or at least 0.2 or above, or at least 0.3 or above, or at least 0.4 or above, or at least 0.5 or above, or at least 0.6 or above, or at least 0.7 or above, or at least 0.8 or above, or at least or equal to 0.9 or above as determined by the Splice AI algorithm.

The first splice acceptor site and first splice donor site define a sequence. In some embodiments, the sequence is a frame-shift inducing sequence, that is, a sequence comprising a number of nucleotides that is not divisible by 3. Splicing therefore leads to introduction of a frame-shift inducing sequence in the mRNA product of the construct, as compared to when no splicing occurs. In some embodiments, the construct further comprises a premature termination codon (PTC) downstream of the regulatory region, configured such that (i) in a cell that has nuclear depletion of the splicing factor, the PTC is out of frame with the start codon in the mRNA product of the construct, and (ii) in a cell without nuclear depletion of the splicing factor, the PTC is in frame with the start codon of the mRNA product of the construct. This can lead to formation of a truncated protein in cells without nuclear depletion of the splicing factor, but where a functional protein is selectively produced in cells with nuclear depletion of the splicing factor. In some embodiments, the construct comprises a further intronic sequence at least 40 nucleotides downstream of the PTC. The further intronic sequence is within an exonic context. The presence of a further intronic sequence downstream of the PTC promotes deposition of an exon junction complex (EJC) on the resultant mRNA when splicing of the first splice acceptor and/or first splice donor is repressed (i.e., since the PTC is in frame with the start codon), which promotes nonsense mediated decay. In cases where splicing is not repressed, the PTC codon is not in frame with the start codon in the mRNA product of the construct, and the ribosome therefore removes the EJC, such that no nonsense-mediated decay occurs. The presence of the further intronic sequence enhances the safety and selectivity of the construct.

In some embodiments or aspects, the first splice acceptor site is upstream of the first splice donor site and the first splice acceptor site and the first splice donor site define a cryptic exon sequence. In some embodiments, the cryptic exon sequence is a frame-shift inducing cryptic exon sequence, which therefore alters expression of the transgene as described above. In additional or alternative embodiments, the cryptic exon sequence encodes for at least a part of the transgene. Repression of splicing therefore can lead to a non-functional protein being produced in a cell without nuclear depletion of the splicing factor. In additional or alternative embodiments, the start codon is present in the cryptic exon sequence.

In some embodiments or aspects, the first splice donor site is upstream of the first splice acceptor site and the first splice donor site and the first acceptor donor site define a single regulatory intron. Repression of splicing therefore can lead to inclusion of at least part of an intron in the mRNA construct of a cell without nuclear depletion of the splicing factor, which can cause a frame-shift, which would block transgene expression as described above. Alternatively, or additionally, full, or partial intron retention could introduce a PTC into the sequence if the PTC were present within the intron itself. Alternatively, or additionally, incorrect splicing or (full or partial) intron retention could disrupt the function of a protein product without requiring a PTC or frame-shift, via introduction of a disruptive amino acid sequence, or via truncation of the amino acid sequence. In contrast, without depletion of the hnRNP splicing factor and with splicing, the intron sequence is removed in the mRNA product of the construct. This leads to a fully encoded and/or uninterrupted transgene sequence, and the production of protein in healthy cells. The above aspects and embodiments are described in more detail below.

In some embodiments, the construct comprises one single regulatory domain, however, the construct may comprise two or more, or three or more, or four or more regulatory domains as described herein. The presence of multiple regulatory domains may increase the selectivity of expression in diseased cells and/or minimise leaky expression in healthy cells. In some embodiments, the construct may comprise one, or at least two, or at least three, or at least four, or at least five, or at least six, or at least seven, or at least eight, or at least nine, or at least 10 cryptic exons and/or regulatory introns.

### Binding Domain

The regulatory domain comprises a binding domain for a splicing factor of the hnRNP family. The splicing factor of the hnRNP family may otherwise be referred to or restricted to a splicing repressor protein of the hnRNP family. Such proteins typically have a structure comprising at least one (e.g., two) RNA-recognition motif flanked by an N-terminus and C-terminal regions. The proteins typically comprise a nuclear-localisation sequence (NLS) which enables localisation in the nucleus. In some embodiments, the splicing factor of the hnRNP family may have a molecular weight between 30 kDa and 120 kDa, more preferably between 30 kDa and 50 kDa. In preferred embodiments, the splicing factor is an endogenous splicing factor, i.e., originating from within the cell.

In some embodiments, the splicing factor is any member of the hnRNP family which is associated with depletion in a disease, for example, a neurogenerative disease or a muscle disease.

In some embodiments, the binding domain is within 150 nucleotides of the first splice acceptor site and/or first splice donor site. In some embodiments, the binding domain is within 100 nucleotides of the first splice acceptor site and/or first splice donor site, or within 50 nucleotides of the first splice acceptor site or first splice donor site, or within 25 nucleotides of the first splice acceptor site or first splice donor site, or within 10 nucleotides of the first splice acceptor site or first splice donor site. Binding of the splicing factor of the hnRNP family to the binding domain leads to repression of the first splice acceptor site and/or first splice donor site and therefore regulates splicing (e.g., of the sequence between the first splice acceptor site and first splice donor site). Additionally, or alternatively, the binding domain may be between the first splice donor site and first splice acceptor site (e.g., within the single regulatory intron sequence in a Design 3 construct or within the cryptic exon sequence in a Design 1 or 2 construct).

In some embodiments, the binding domain comprises at least 6 nucleotides, more preferably at least 10 nucleotides. In some embodiments, the binding domain is from 6 to 700 nucleotides, or from 6 to 150 nucleotides, or from 10 nucleotides to 150 nucleotides, or from 15 to 50 nucleotides, or from 6 to 45 nucleotides, or from 10 to 45 nucleotides, or 10 to 20 nucleotides, and in some examples from 20 nucleotides to 45 nucleotides.

In some embodiments, the binding domain is upstream of the first splice acceptor site and/or the first splice donor site. In some embodiments, the binding domain is downstream of the first splice donor site and/or the first splice acceptor site. In some embodiments, the binding domain is between the first splice acceptor site and first splice donor site (i.e., within the sequence defined by the first splice acceptor site and first splice donor site, in some embodiments, the cryptic exon sequence, or in other embodiments, the single regulatory intron). In embodiments where the construct comprises a cryptic exon defined by the first splice acceptor site and the first splice donor site (e.g., Design 1 or Design 2 constructs), the binding domain may be upstream of the cryptic exon (i.e., in the first part of the intronic region), downstream of the cryptic exon (i.e., in the second part of the intronic region), or within the cryptic exon sequence.

In embodiments where the construct comprises a single regulatory intron defined by the first splice donor site and the first splice acceptor site (e.g., Design 3 constructs), the binding domain may be upstream or downstream of the single regulatory intron (i.e., in exonic regions flanking the single regulatory intron), or the binding domain may be within the single regulatory intron. In some embodiments, the construct comprises two binding domains for a splicing factor of the hnRNP family (e.g., one upstream of the first splice acceptor site and one downstream of the first splice donor site).

The binding domain in the construct may encode for any known binding site for the splicing factor in the RNA. For example, the sequence characteristics which promote binding of TDP-43 are described in Lukavsky et al., 2013 (NSMB, 20, pages 1443-1449) which is incorporated herein by reference. The known binding site for the splicing factor may have been identified by transcriptome mapping of the splicing factor, for example, as determined by immunoprecipitation, wherein the transcriptome mapping may have been performed on the human genome.

In preferred embodiments, the binding domain is a TDP-43 binding domain and the splicing factor of the hnRNP family is TDP-43.

In some embodiments, the TDP-43 binding domain comprises a region of at least 6 nucleotides, or preferably at least 10 nucleotides, or at least 20 nucleotides, with a statistically significant enrichment of TG dinucleotides and/or TGNNTG hexanucleotides, wherein N is A, T, C or G. In some embodiments, the TDP-43 binding domain comprises a region of from 6 nucleotides to 150 nucleotides, with a statistically significant enrichment of TG dinucleotides and/or TGNNTG hexanucleotides, wherein N is A, T, C or G, wherein statistically significant enrichment is defined as a probability of less than 0.2% that a random sequence of nucleotides of equal length would feature an equal number of TG dinucleotides and/or TGNNTG hexanucleotides. In some embodiments, the statistically significant enrichment is defined as a probability of less than or equal to 0.15% that a random sequence of nucleotides of equal length would feature an equal number of TG dinucleotides and/or TGNNTG hexanucleotides, or less than or equal to 0.1%, or less than or equal to 0.05%, or less than or equal to 0.01%, or less than or equal to 0.003%, or equal or less than 0.001%, or equal or less than 0.0003%, or equal or less than 0.0001%. These definitions cover both short sequences which are highly enriched for UG, and longer sequences which are broadly enriched for UG, both of which have been shown to be preferentially bound by TDP-43. In some embodiments and examples, the statistically significant enrichment is defined as a probability of less than or equal to 1×10⁻⁵, or of less than or equal to 1×10⁻⁶, or of less than or equal to 1×10⁻⁷, or of less than or equal to 1×10⁻⁸, or of less than or equal to 1×10⁻⁹, or less than or equal to 1×10⁻¹⁰.

Example TDP-43 binding domains include the TDP-43 binding region within UNC13A which represses UNC13A cryptic exon inclusion (SEQ ID NO: 1).

### SEQ ID NO: 1

TAGATAAAAGGATGGATGGAGAGATGGGTGAGTACATGGATGGATAGATGGATGAGTT GGTGGGTAGATTCGTGGCTAGATGGATGATGGATGGATGGACA, which has a probability score of ˜0.01% that a random sequence of nucleotides of equal length would feature an equal number of TG dinucleotides.

Other example TDP-43 binding domains include TGTGTG which has a probability score of 0.02% and TGNNTGTG which has a probability score of 0.15%. An example TDP-43 binding domain described herein is: SEQ ID NO: 2:

TGTGTGTGTGTGTGTGAATGTGTGTGTGTGTGTGTG, which has a probability of 5×10⁻²⁰ that a random sequence of nucleotides of equal length would feature an equal number of TG dinucleotides. This is a modified version (with over 90% sequence identity) of the binding domain found in the human AARS1 gene.

In some embodiments, the TDP-43 binding domain comprises a sequence that is enriched with TG dinucleotides. In some embodiments, an enrichment of TG dinucleotides is defined as a sequence comprising at least 6 nucleotides with 100% TG dinucleotides (i.e., TGTGTG), or one or more region with at least 6 nucleotides with 100% TG dinucleotides. In some embodiments, an enrichment of TG dinucleotides is defined as a sequence comprising at least 8 nucleotides (or one or more region with at least 8 nucleotides) with at least 80% TG dinucleotides (e.g., TGAATGTG), or at least 85%, or at least 90%, or at least 95%, or 100% TG dinucleotides (i.e., TGTGTGTG). In some embodiments, an enrichment of TG dinucleotides is defined as a sequence which comprises at least 10 nucleotides (or one or more region with at least 10 nucleotides) with at least 60% TG dinucleotides (e.g., TGAATGAATG (SEQ ID NO: 3)), or at least 65%, or at least 70%, or at least 75%, or at least 80%, or at least 85%, or at least 90%, or at least 95%, or at least 100% TG dinucleotides. In some embodiments, an enrichment of TG dinucleotides is defined as a sequence that comprises at least 15 nucleotides (or one or more region with at least 15 nucleotides) with at least 53% TG dinucleotides (e.g., TGAATGAAATGATG (SEQ ID NO: 4)), or at least 60%, or at least 65%, or at least 70%, or at least 75%, or at least 80%, or at least 85%, or at least 90%, or at least 95%, or 100% TG dinucleotides).

In some embodiments, the TDP-43 binding domain comprises a sequence that comprises at least one TGTGTG, or TGTGTGTGTG, or TGTGTGTGTG (SEQ ID NO: 5), or TGTGTGTGTGTG (SEQ ID NO: 6), or TGTGTGTGTGTGTG (SEQ ID NO: 7), or TGTGTGTGTGTGTGTG (SEQ ID NO: 8), or TGTGTGTGTGTGTGTGTG (SEQ ID NO: 9) or any combination thereof. In some examples, the TDP-43 binding domain comprises a sequence that has at least 80% sequence identity to SEQ ID NO: 2 or at least 85%, or at least 90% sequence identity, or at least 95% sequence identity, or 100% sequence identity to SEQ ID NO: 2-TGTGTGTGTGTGTGTGAATGTGTGTGTGTGTGTGTG.

In some examples, the TDP-43 binding domain has a sequence that has at least 80% sequence identity, or at least 85%, or at least 90%, or at least 95%, or at least 100% sequence identity with SEQ ID NO: 1-9, or SEQ ID NO: 115.

While TDP-43 is capable of binding a large variety of different sequences that are UG/TG-rich, the binding domain does not have to bind a pure UG/TG-repeat. This is in part due to the protein's lack of contact with some RNA residues within its binding footprint, and in part due to multivalent protein-protein interactions which enhance binding to large regions of UG-rich RNA. This means that in some embodiments, the TDP-43 binding domain may not require any pure UG-repeats. Example sequences include

SEQ ID NO: 159
TGTGTTTGATGAGTGTATGTGGTGTGTCTGAGAGTGTAGTGTATGAGTG
ATTGACGTGAGTGTTTGTAAGGCGTGTCTGTTTGAGTGACTGGTCGTGT
GATTG
SEQ ID NO: 160
TGGGTGCGTGCTGGGCGTGTCTGTCGGGTGAATGCACTGGAGTGCGTGT
CTGCGTGGGTGTTGAGTGGATGTAGGTGTGACTGCCTCGTGTGCTTGCG
AGAGTGAATGGAGTGTGCTTGATG

The construct is configured such that when placed in a cell with nuclear depletion of the the splicing factor, (e.g., in the absence of binding of the splicing factor to the binding domain) splicing of the first splice acceptor site or first donor site is not repressed, and when placed in a cell without nuclear depletion of the splicing factor (e.g., in the presence of binding of the splicing factor to the binding domain), splicing of the first splice acceptor site or first donor site is repressed. This alters the sequences that are incorporated into the mRNA product of the construct, and thereby regulates whether functional protein is produced from the mRNA product of the construct.

In some embodiments, the first splice acceptor site is upstream of the first splice donor site, and the first splice acceptor site and the first splice donor site define a cryptic exon sequence (e.g., Design 1 or 2 constructs described herein). In some embodiments, the cryptic exon sequence is a frame-shift inducing cryptic exon sequence, i.e., an exon comprising a length of nucleotides that is not divisible by 3 (e.g., Design 1 or 2 constructs described herein). Additionally, or alternatively, the cryptic exon sequence may comprise the start codon. Additionally, or alternatively, the cryptic exon sequence may encode for at least part of the transgene sequence (e.g., Design 2 construct described herein).

In alternative embodiments, the first splice donor site is upstream of the first splice acceptor site. In some embodiments, the sequence between the first splice donor site and the first splice acceptor site is a single regulatory intron (e.g., Design 3 construct described herein). In some embodiments, production of a functional protein from the transgene can be regulated (i.e., switched off or on) by the inclusion or exclusion of at least part of the intron in the mRNA product of the construct.

### Start Codon

The construct comprises a start codon, or a plurality or array of start codons (i.e., in frame with each other). In some embodiments, the start codon may be upstream of the regulatory domain. In some embodiments, the start codon may be present within the regulatory domain (e.g., in embodiments comprising a cryptic exon, the start codon may be present within the cryptic exon). In some embodiments, the start codon is provided in the form of a Kozak sequence or Kozak-like sequence. In preferred embodiments, the start codon comprises ATG. In some examples, the construct comprises a sequence encoding a start codon that has at least 80% sequence identity, or at least 85% sequence identity, or at least 90% sequence identity, or at least 95% sequence identity, or at least 100% sequence identity with SEQ ID NO: 28.

Approximately half of human mRNAs feature an upstream start codon in the 5′ untranslated region, which does not initiate translation of the mRNA's canonical coding sequence. Many such start codons initiate translation of upstream open reading fames. Despite the presence of upstream start codons, these mRNAs still result in the expression of the canonical protein from the downstream, canonical start codon, via a variety of proposed mechanisms including leaky scanning and re-initiation. As such, the start codon described in the embodiment above does not necessarily need to be the most-5′ start codon in the mRNA product.

### Transgene

The construct comprises a transgene sequence (e.g., a sequence that encodes for a protein). This may be formed of one or more exonic sequences (or parts) that together form a complete transgene sequence. In some embodiments, at least a part of the transgene sequence is downstream of the regulatory domain. In some embodiments, the complete transgene sequence may be uninterrupted. In some embodiments, the complete transgene sequence is downstream of the regulatory domain. In some embodiments, the transgene sequence may be interrupted (i.e., splice into parts). In some embodiments, the transgene sequence may be split into two or more parts, or three or more parts, or four or more parts, or five or more parts, or six or more parts, or seven or more parts, or eight or more parts, or nine or more parts, or ten or more parts. In some embodiments, at least part of the transgene sequence is upstream of the regulatory domain and downstream of the regulatory domain. In some embodiments, i.e., in embodiments comprising a cryptic exon defined by the first splice acceptor site and the first splice donor site, the cryptic exon may form part of the transgene sequence. In such embodiments, at least part of the transgene sequence may be upstream of the regulatory domain, at least part of the transgene sequence is encoded by the cryptic exon sequence and at least part of the transgene sequence may be downstream of the regulatory domain.

In some embodiments, the complete transgene is for (i.e., encodes for) a diagnostic protein. The diagnostic protein may be any suitable diagnostic protein known in the art. The construct can be used as a biomarker in this instance (e.g., to monitor depletion of the hnRNP splicing factor). In some embodiments, the diagnostic protein is a fluorescent protein, a luminescent protein, or a protein with a detectable antibody-binding tag (e.g., a protein with a peptide or polypeptide tag).

The fluorescent protein may be any suitable fluorescent protein known in the art. In some embodiments, the fluorescent protein is a monomeric red fluorescent protein (mRFP), for example, mCherry or mScarlet. In some embodiments, the fluorescent protein is a green fluorescent protein (GFP) or an enhanced derivative (eGFP). In some embodiments, the green fluorescent protein is mNeonGreen or mGreenLantern. In some embodiments, the fluorescent protein is a blue fluorescent protein. In some embodiments, the fluorescent protein is an orange fluorescent protein. In some embodiments, the fluorescent protein is a yellow fluorescent protein.

The luminescent protein may be any suitable luminescent protein known in the art. In some embodiments, the luminescent protein is a luciferase protein (e.g., firefly luciferase or Renilla luciferase). In some examples, the luciferase protein is Gaussia Luciferase (gLuc), i.e., Gaussia princeps Luciferase.

The protein with a detectable antibody-binding tag may have any suitable tag. In some embodiments, the tag is a peptide tag. In some embodiments, the peptide tag is a FLAG-tag (e.g., comprising DYKDDDDK (SEQ ID NO: 10) or DDDDK (SEQ ID NO: 11)), His-tag (HHHHHH, (SEQ ID NO: 12)), HA-tag (YPYDVPDYA, (SEQ ID NO: 13)), Myc-tag (EQKLISEEDL, (SEQ ID NO: 14)), V5 tag (GKPIPNPLLGLDST, (SEQ ID NO: 15)), S tag (KETAAAKFERQHMDS, (SEQ ID NO: 16)), E tag (GAPVPYPDPLEPR, (SEQ ID NO: 17)), T7 tag (MASMTGQQMG, (SEQ ID NO: 18)), VSV-G tag (YTDIEMNRLGK, (SEQ ID NO: 19)), Glu-Glu tag (EEEEYMPME, (SEQ ID NO: 20)), Strep-tag II (WSHPQFEK, (SEQ ID NO: 21)), HSV tag (QPELAPEDPED, (SEQ ID NO: 22)), a chitin binding domain (TTNPGVSAWQVNTAYTAGQLVIYNGKTYK, (SEQ ID NO: 23)), a calmodulin binding domain (KRRWKKNFIAVSAANRFKKISSSGAL, (SEQ ID NO: 24)). In some embodiments, the tag is a polypeptide tag. In some embodiments, the polypeptide tag is a Glutathione-S-transferase (GST) tag, a Maltose Binding Protein (MBP) tag or a Thioredoxin (Trx) tag).

In some embodiments, the transgene is for (i.e., encodes for) a therapeutic protein (i.e., a protein that has a therapeutic effect on the cell). The therapeutic protein may be a protein that is deficient or abnormal in a diseased cell. The therapeutic protein may be any suitable therapeutic protein known in the art. In some embodiments, the therapeutic protein is a neuroprotective protein. In some embodiments, the therapeutic protein may be a nuclease, a chaperone, a proteasomal protein, a recombinase protein, a splicing regulator, or a transcription factor or any combination thereof. In some embodiments, the therapeutic protein is a regulatory protein. The regulatory protein may be selected from a recombinase protein, a splicing regulator, a transcription factor, or any combination thereof.

The nuclease may be any suitable nuclease known in the art. In some embodiments, the nuclease is a Cas nuclease, for example a Cas9 or Cas13 nuclease, or a catalytically inactive derivative of a Cas nuclease, or a modified variant of a Cas-family nuclease with enhanced specificity or activity, or a nicking Cas9 nuclease. In some embodiments, the Cas-family nuclease, or variant thereof, is fused to a second protein (for example a nicking Cas9 nuclease fused to a reverse transcriptase to enable “prime editing”).

The chaperone protein may be any suitable chaperone protein known in the art. In some embodiments, the chaperone protein is a foldase protein. In some embodiments, the chaperone protein is a heat-shock protein. In some embodiments the heat shock protein is selected from, but not limited to, HSPB1, HSP104, HSP40, or HSP70. In some embodiments, the chaperone is a cyclophilin, e.g., cyclophilin A. In some embodiments, the chaperone is any protein from the DnaJ family.

The recombinase protein may be any suitable recombinase protein used in the art. In some examples, the recombinase protein is Cre recombinase. In some examples, the recombinase protein is Flp recombinase. In some examples, the recombinase protein is Vika recombinase. In some examples, the recombinase protein is Dre recombinase.

The proteasomal protein may be any suitable proteasomal protein known in the art.

The transcription factor may be any suitable transcription factor known in the art. In some embodiments, the transcription factor may be, or may derive from (e.g., as a truncation or a fusion protein), a human or mammalian transcription factor. In some embodiments, the transcription factor could be a synthetic engineered transcription factor, for example with a DNA binding domain based on a transcription activator-like effector (TALE), or a zinc finger domain, or a modified Cas-family enzyme (e.g., the CRISPRa system). In some embodiments the transcription factor could be an activator or a repressor of transcription. In some embodiments, the transcription factor may feature a characterised transcriptional regulatory domain, for example a VP16 domain, or a KRAB domain

The splicing regulator may be any suitable splicing regulator known in the art. In some embodiments, the splicing regulator is or comprises a splicing inhibitor. In some embodiments, the splicing regulator is hnRNPA1 or RAVER1. In some embodiments, the splicing regulator further comprises a binding domain of the hnRNP family (i.e., fused to a splicing regulator), (e.g., an RNA binding domain of the hnRNP family), for example, a TDP-43 binding domain fused to a splicing regulator, such as TDP-43 binding domain fused to RAVER1 (e.g., a TDP-43 RNA binding domain fused to RAVER1). In some embodiments, the transgene is configured such that it can autoregulate and/or suppress cryptic splicing (i.e., upon depletion of the endogenous hnRNP splicing factor, such as TDP-43).

In some embodiments, the construct may comprise a single transgene. In other embodiments, the construct may comprise at least two transgenes. The at least two transgenes may comprise a first transgene which encodes for a first therapeutic protein and a second transgene that encodes for a diagnostic protein, or a first transgene which encodes for a first therapeutic protein and a second transgene that encodes for a second therapeutic protein. The two transgenes may be separated by a protein cleavage site or self cleavage site, for example, comprising any sequence of a protein-cleavage site or self-cleavage site described elsewhere herein. In some examples described herein, two transgenes are separated by a T2A cleavage site.

The transgene sequence may comprise a stop codon at the end of the transgene sequence, (i.e., unless linked to a further downstream transgene). In embodiments wherein the construct comprises a further intronic sequence (e.g., a constitutively spliced intron), the stop codon is no more than 55 nucleotides, preferably no more than 50 nucleotides, or no more than 40 nucleotides upstream of the further intronic sequence, or the stop codon is downstream of the further intron sequence.

In some embodiments, the transgene is a known sequence encoding for a protein, i.e., a naturally occurring sequence. In some embodiments, the known sequence is modified by replacing naturally occurring codons with synonymous codons.

### Optional Features of the Construct

In some embodiments, the sequence defined by the first acceptor splice site and the first donor splice site is a frame-shift inducing sequence. In such embodiments (e.g., when the sequence between the first splice acceptor site and the first splice donor site is a frame-shift inducing sequence), the construct may further comprise a premature termination codon (PTC). The premature termination codon may be selected from TAG, TAA or TGA. The PTC may be downstream of the regulatory domain but upstream of at least part of the transgene sequence. In some embodiments, the PTC may be positioned within at least part of the transgene which is located downstream of the regulatory domain. In alternative embodiments, the PTC may not be present in at least part of the transgene, for example, the PTC may be present within a separate sequence comprising a PTC. In some embodiments, i.e., in embodiment comprising a single regulatory intron, the PTC may be present within the single regulatory intron.

The PTC is positioned and configured such it is in frame with the start codon in the mRNA product of the construct when splicing is repressed (i.e., in a healthy cell), but out of frame in the mRNA product of the construct when splicing is not repressed (i.e., in a diseased cell). A PTC in frame with the start codon leads to production of a truncated protein. This leads to a functional protein being produced upon nuclear depletion of the splicing factor, but no functional protein being produced without nuclear depletion of the splicing factor. This selectively leads to formation of a truncated protein in cells without nuclear depletion.

### Further Intronic Sequence (e.g., Constitutively Spliced Intron Sequence)

In some embodiments, the construct may further comprise a further intronic sequence downstream of the regulatory domain. The further intronic sequence is within or surrounded by exonic context (e.g., flanked by exonic sequences). In preferred embodiments, the further intronic sequence comprises a constitutively spliced intron sequence. The further intronic sequence is at least 40 nucleotides downstream of the PTC, but in preferred embodiments, the PTC is at least 50 nucleotides upstream of the further intronic sequence, or at least 55 nucleotides, upstream of the further intronic sequence. In some embodiments, the PTC is between 40 to 55 nucleotides upstream of the further intronic sequence, or 50 to 55 nucleotides upstream of the further intronic sequence. In some embodiments, the further intronic sequence is downstream of the complete transgene sequence. In alternative embodiments, the further intronic sequence is downstream of the regulatory domain but upstream of at least part of the transgene sequence.

The presence of a further intronic sequence downstream of the PTC promotes deposition of an exon junction complex (EJC) on the resultant mRNA when splicing of the first splice acceptor and/or first splice donor is repressed (i.e., resulting in the PTC being in frame with the start codon), which promotes nonsense mediated decay. In cases where splicing is not repressed, the PTC codon is not in frame with the start codon in the mRNA product of the construct, and the ribosome therefore removes the EJC, such that no nonsense-mediated decay occurs.

In the examples described herein the further intronic sequence and surrounding exonic context is derived from human RPS24, however, any suitable intron and exon sequence may be used. In some embodiments, the further intronic sequence comprises any naturally occurring intron and exon sequence (e.g., any intron and exon from the human genome). In alternative embodiments, the further intronic sequence and exon are formed of or from a synthetic sequence. The sequences may be designed using the Splice AI algorithm, i.e., wherein the splicing sites defining the further intronic sequence have a splice score of at least 0.01, or at least 0.05, preferably at least 0.1, or at least 0.5, or more preferably at least 0.9. Further, the synthetic sequences may be designed using “algorithm 1” described herein.

### Protease Cleavage Site or Self-Cleaving Cleavage Site

In some embodiments (e.g., in certain Design 1 and Design 3 constructs described herein), the construct further comprises a protease-cleavage site or self-cleavage site. In some embodiments, the protease-cleavage site or self-cleavage site may be downstream of the regulatory domain but upstream of at least part of the transgene sequence. In alternative embodiments, the protease cleavage site or self-cleavage site may be between transgene sequences. The protease cleavage site or self-cleavage site may be selected from P2A, T2A, F2A, E2A, furin, PCSK1, PCSK6, PCSK7, cathepsin B, granzyme B, factor XA, enterokinase, genenase, sortase, precission protease, thrombin, TEV protease or elastase 1. In some examples described herein, the cleavage site is P2A or T2A. The protease cleavage site enables cleavage of the protein encoded by the transgene from any peptides encoded by the regulatory domain, or cleavage of a protein encoded by a first transgene with a protein encoded by a second transgene, if required.

### Regulation of the Construct

The construct and regulatory domain are configured such that (i) if placed in a cell with nuclear depletion of the splicing factor of the hnRNP family, (e.g., in the absence of binding of the splicing factor to the binding domain) splicing of the first splice acceptor site and first donor site is not repressed, such that functional protein is produced from the transgene sequence. In other words, functional protein is produced from the mRNA product of the construct (i.e., the functional protein encoded by the complete, uninterrupted transgene sequence). A functional protein may be defined herein as a protein produced when the complete, uninterrupted transgene sequence is present in the mRNA product, and in frame with the start codon, and with no in-frame stop codon between the start codon and the transgene sequence. A functional protein may additionally or alternatively be defined herein as a polypeptide chain of at least 30, preferably 50, further preferably 100 amino acids, which can perform a therapeutic, diagnostic, or regulatory role within the cell, either alone or acting in tandem with one or more additional proteins (for example as a heterodimer). For example, a functional protein could be a full length GFP protein capable of intrinsic fluorescence, or one component of a split-GFP system capable of fluorescence upon binding to the second component of the split-GFP system, or a mutated or truncated GFP fragment with no fluorescence that could be detected via an assay such as western blotting.

The construct and regulatory domain are also configured such that (ii) if placed in a cell without nuclear depletion of the splicing factor of the hnRNP family (e.g., in the presence of binding of the splicing factor to the binding domain), splicing of the first splice acceptor site and/or first donor site is repressed, such that no functional protein is produced from the complete transgene sequence. In some embodiments, this may arise because at least part of the transgene sequence is not in frame with the start codon (e.g., wherein the sequence defined by the first splice acceptor site and first splice donor site is a frame-shift inducing sequence). In some embodiments, this may arise because at least part of the transgene sequence is absent in the mRNA product of the construct (i.e., the transgene sequence is not fully transcribed, e.g., in embodiments where the cryptic exon sequence encodes for part of the transgene, and the cryptic exon sequence is absent in the mRNA product of the construct in healthy cells). In some embodiments, this may arise because a sequence is introduced in the mRNA product of the construct which interrupts the transgene sequence (e.g., in embodiments where the first splice donor site and first splice acceptor site define a single regulatory intron, and wherein without depletion of the splicing factor, at least part of the intron is incorporated into the mRNA product of the construct in healthy cells, or alternatively part of the transgene sequence is not included in the mRNA product of the construct in healthy cells). In this last embodiment, this interruption may involve introduction of a PTC, and/or introduction of a disruptive amino acid sequence that inhibits protein function.

The cell may be any suitable cell. In some embodiments, the cell is a mammalian cell, more preferably a human cell. In preferred embodiments, the cell has nuclear depletion of the hnRNP splicing factor (e.g., depletion of TDP-43). In some embodiments, the cell is a brain cell. In some embodiments, the cell is a neuron or neuronal cell. In some embodiments, the cell is a microglial cell or astrocyte cell. In some embodiments, the cell is a muscle cell.

In a first embodiment of the first aspect, or according to the second aspect of the present invention, the regulatory sequence is regulated by cryptic splicing. In such embodiments, the regulatory sequence comprises a cryptic exon sequence between the first splice acceptor site and the first splice donor site and the cryptic exon is embedded within the intronic region. This embodiment is described in more detail below, and is demonstrated by the embodiments shown in FIGS. 1 and 2. The construct is configured such that
(i) if placed in a cell with nuclear depletion of the splicing factor of the hnRNP family, the cryptic exon sequence is present in the mRNA product of the construct, and
(ii) if placed in a cell without nuclear depletion of the splicing factor of the hnRNP family the cryptic exon is not present in the mRNA product of the construct.

In an embodiment of the first aspect, or according to the third aspect of the present invention, the regulatory sequence is regulated by splicing of a single regulatory intron.

In such embodiments, an intronic sequence is between the first splice donor site and first splice acceptor site. The construct is configured such that
(i) if placed in a cell with nuclear depletion of the splicing factor, the single regulatory intron is spliced such that a functional protein is produced.
(ii) if placed in a cell without nuclear depletion of the splicing factor, the single regulatory intron is incorrectly spliced, or not spliced, such that functional protein is not produced.

Each of the above embodiments or aspects are described in more detail below. All such embodiments importantly comprise a binding domain for a splicing factor of the hnRNP family, a first splice acceptor site, a first splice donor site, and a transgene sequence (i.e., a transgene sequence encoding a functional protein). The construct is configured such that binding of the splicing factor to the binding domain regulates splicing of the first splice acceptor site or the first splice donor site. Splicing is not repressed in cells depleted of splicing factor, but repressed in cells without depletion of the splicing factor. This in turn regulates whether the transgene is fully expressed and encoded to produce a functional protein.

Constructs where Regulatory Domain is Regulated by Cryptic Splicing

In a second aspect, or embodiment of the first aspect, there is provided, a construct comprising
a start codon,
a regulatory domain comprising:
   a first splice acceptor site and a first splice donor site, which define a cryptic exon sequence,
   an intronic region defined by a second splice donor site and a second splice acceptor site, wherein the cryptic exon sequence is located within the intronic region, and
   a binding domain for a splicing factor of the heterogenous nuclear ribonucleoprotein (hnRNP) family, located within 150 nucleotides of the first splice donor site or first splice acceptor site; and
a transgene sequence,
   configured such that
   (i) if placed in a cell that is depleted of the splicing factor, splicing of the first splice acceptor site and first donor site is not repressed and the cryptic exon sequence is present in the mRNA product of the construct, such that a functional protein is produced from the transgene sequence (i.e., functional protein is produced from the mRNA product where the functional protein is encoded by the complete, uninterrupted transgene sequence)
   (ii) if placed in a cell that is not depleted of the splicing factor, splicing of the first splice acceptor site and/or first donor site is repressed and the cryptic exon sequence is absent in the mRNA product of the construct, such that a functional protein is not produced from the transgene sequence (i.e., functional protein is produced from the mRNA product where the functional protein is encoded by the complete, uninterrupted transgene sequence)

The binding domain for a splicing factor of the heterogenous nuclear ribonucleoprotein (hnRNP) family, the premature termination codon, the first splice acceptor site, the first splice donor site and the transgene sequence are as otherwise described herein. In embodiments where the regulatory domain comprises a cryptic exon, the first splice acceptor site and the first splice donor site may be termed “cryptic splice sites”.

### Intronic Region

The intronic region is defined by a second splice donor site and a second splice acceptor site. The intronic region comprises (from upstream to downstream) a first part of the intronic region, a cryptic exon sequence, and a second part of the intronic region. The intronic region comprises the binding domain for the splicing factor of the hnRNP family, which is located at most 150 nucleotides upstream or downstream from the first splice acceptor and/or first splice donor site (as described above). The binding domain may be within the first part of the intronic region, in the cryptic exon sequence, or the second part of the intronic region.

The first part of the intronic region may be described as a “first intron”, and the second part of the intronic region may be described as a “second intron”. In some embodiments, the first part of the intronic region and/or second part of the intronic region each comprises at least 50 nucleotides, preferably at least 70 nucleotides, or at least 100 nucleotides, or at least 150 nucleotides. In some embodiments, the first part of the intronic region and/or second part of the intronic region comprises from 70 nucleotides to 5000 nucleotides, or from 70 to 1000 nucleotides, or from 70 to 500 nucleotides, and in some examples, from 125 nucleotides to 250 nucleotides.

In some embodiments, the second splice donor site and/or the second splice acceptor site have a splice score of 0.01 (the 99.8th percentile of SpliceAI scores, see FIG. 11) or above as determined by the Splice AI algorithm. In preferred embodiments, the second splice donor site and/or the second splice acceptor site have a splice score of 0.05 or above as determined by the Splice AI algorithm, or at least 0.1 or above, or at least 0.2 or above, or at least 0.3 or above, or at least 0.4 or above, or at least 0.5 or above, or at least 0.6 or above, or at least 0.7 or above, or at least 0.8 or above, or at least or equal to 0.9 or above as determined by Splice AI algorithm, more preferably at least 0.95, or at least 0.96, or at least 0.97, or at least 0.98, or at least 0.99 or above as determined by the Splice AI algorithm.

In some embodiments, the intronic region may derive from a naturally occurring intronic region comprising a cryptic exon (e.g., from the human genome), wherein the cryptic exon is regulated by a splicing factor of the hnRNP family (e.g., TDP-43). In some embodiments, the intronic region may be at least 80% identical to at least a part of a naturally occurring intronic region comprising a cryptic exon (e.g., from the human genome), or at least 85% identical, or at least 90% identical, or at least 95% identical, or at least 100% identical to at least a part of a naturally occurring intronic region comprising a cryptic exon (e.g., from the human genome). In some embodiments, the intronic region may have been modified by truncation (i.e., parts of the intronic regions upstream and downstream of the cryptic exon may comprise less nucleotides than as found in the human genome). The intronic region may have been modified by insertion, deletion, or substitution of one or more nucleotides, for example, two nucleotides, three nucleotides, four nucleotides, five nucleotides, or six or more nucleotides. In some embodiments, the intronic region may have been modified by (i) mutating a nucleotide in the intronic region to remove one or more premature termination codon(s), and/or (ii) inserting or deleting one or two nucleotides in the cryptic exon sequence to introduce a frame-shift. In some embodiments, the intronic region derives from at least part of AACSP1, AARS1, ABCB1, ABCD1, AC002310.11, AC002310.7, AC002456.2, AC008543.1, AC008676.3, AC009133.12, AC010531.1, AC015712.1, AC015712.6, AC022387.2, AC022966.1, AC025165.6, AC064807.1, AC092073.1, AC138932.1, AC245041.2, ACSF2, ACTL6B, ACTR1A, ADARB1, ADARB2, ADCY1, ADCY7, ADCY8, ADGRB1, ADGRL1, ADSSL1, AGK, AGRN, AHNAK, AKT3, AL023775.2, AL031282.2, AL035461.3, AL121845.3, AL157392.3, AL157392.5, AL354696.2, AL360181.3, AL645568.1, AL669831.3, AL672142.1, ALDH3B1, AMPD2, ANKRD19P, ANKRD44, ANOS2P, AP000662.4, AP006621.8, AP4M1, ARAP3, ARF1, ARHGAP22, ARHGAP23, ARHGEF16, ARHGEF19, ASGR1, ATAD5, ATG4B, ATP5MG, ATP8A2, ATXN1, ATXN10, BCL2L11, BCL2L13, BLCAP, BMP8B, BNIP3P11, BRD1, BTN3A3, C16orf95, C20orf194, C2orf81, C4orf36, C5orf66, CACNB2, CACNG5, CAMK2B, CAMTA1, CASP8, CASTOR1, CBY1, CCDC102B, CCDC150, CCDC183-AS1, CCDC33, CCT2, CDHR2, CDK11A, CDKAL1, CDON, CELF5, CENPBD1P1, CENPK, CENPS-CORT, CEP152, CEP290, CEP72, CEP83, CH17-189H20.1, CH507-154B10.1, CHD8, CHFR, CHGB, CHRNA5, CHRNB3, CLCN6, CLSPN, CLTCL1, CNGA3, CNPY1, CORO6, CPVL, CREB3L4, CRLS1, CRTC1, CSMD2, CTC-490E21.12, CTD-2014B16.3, CTD-2054N24.2, CTD-2162K18.4, CTD-2554C21.2, CTD-2561J22.3, CU634019.6, CUL9, CYFIP2, CYP2C8, DACH2, DACT3-AS1, DAGLA, DAPK1, DELE1, DENND2B, DGKA, DLG5, DLGAP1, DNAJC12, DNAJC25-GNG10, DNMT3A, DNMT3B, DOCK1, DPF1, DUXAP9, EBF1, ECEL1, EHD2, EIF2A, EIF2AK1, EIF4ENIF1, ELAVL3, EML6, ENAH, ENTPD6, EP300, EP400, EPB41L1, EPB41L4A, EPS8L2, ETV5, F12, FADS2, FAM114A2, FAM156A, FAM182B, FAM66D, FAM66E, FBL, FBXL19, FGFR4, FIRRE, FKBP14-AS1, FOXK1, FRYL, G2E3, G3BP1, GALNT12, GAS6, GATA2, GLIPR2, GMPPA, GOLGA7B, GOLGA8A, GPHN, GPSM2, GPX7, GRAMD1A, GREB1, GRIN2D, GSTCD, GTF2H2, GTF2IP13, HAUS2, HDAC6, HDGFL2, HDLBP, HECTD4, HERC2P2, HIPK1, HROB, HULC, ICA1, IFT122, IGSF21, IGSF9, IK, IL15, INPP4A, INSR, INTS11, IQCE, IQCK, ISL2, ISYNA1, ITGA3, ITGA7, ITPR3, KALRN, KATNA1, KCNIP1, KCNIP2, KCNK15-AS1, KCNQ2, KCNT1, KDM1B, KDM4D, KIAA1211, KIAA1217, KIF14, KIF21A, KLC1, KMT5A, KNDC1, KRT8, L3MBTL1, LCOR, LIAS, LINC00265, LINC00342, LINC00475, LINC01002, LINC01224, LINC01322, LINC01503, LINC01572, LINC01684, LINC02082, LINC02202, LINC02506, LINGO1, LMNA, LRP1B, LRP8, LSM12, LSS, LTBP2, MACROD1, MADD, MANBAL, MAP2K6, MAPKAPK5, MATK, MBP, MC1R, MCM9, MDC1, MED12, MED13L, MEIS2, METTL8, MGAT5B, MIER3, MMAA, MRPL34, MTRR, MTX1P1, NAA38, NADSYN1, NAT1, NBEA, NBPF9, NDUFB9, NFKBIZ, NFYC, NIPSNAP3B, NPIPB11, NPLOC4, NSFL1C, NTRK2, NTRK3, NUP188, NUP210, OBSCN, OPCML, PAOX, PATJ, PCBP3, PCBP4, PCDH11X, PCSKIN, PDCD2L, PDCD6, PDE2A, PDE9A, PER3, PHF2, PHF5A, PI4KA, PIGG, PIGU, PKD1P3, PKN1, PLCE1, PLEKHA1, PLEKHA6, PLEKHG2, PLEKHG4, PLEKHM2, POLD1, POLR2F, POU2F2, PPCDC, PPIP5K1, PPMIN, PPP1R14B-AS1, PRDM8, PRELID3A, PREX1, PRKG2, PROX1-AS1, PRPF40B, PRRT4, PRUNE2, PSPC1, PTK2, PTPN13, PTPN21, PTPRN2, PTPRT, PUDP, PUS7L, PWWP3A, PXDN, RAB20, RAB27A, RALGAPA2, RANBP17, RASGRP2, RBMXL1, RC3H1, RCAN3, RET, RFLNA, RGMA, RHOQ, RP1-120G22.12, RP1-138B7.8, RP1-283E3.8, RP1-59M18.2, RP11-101E3.5, RP11-108K14.8, RP11-108L7.4, RP11-124N2.1, RP11-155D18.12, RP11-155G14.5, RP11-155G14.6, RP11-206L10.2, RP11-30K9.6, RP11-345P4.10, RP11-411B6.6, RP11-436D23.1, RP11-465B22.3, RP11-47909.4, RP11-505D17.1, RP11-511P7.6, RP11-566K11.4, RP11-613M10.9, RP11-61L23.2, RP11-718011.1, RP11-739N20.2, RP11-73M18.2, RP11-761B3.1, RP11-795F19.5, RP11-977G19.10, RP4-583P15.15, RP5-967N21.13, RPGRIP1L, RSF1, RTL1, SCN9A, SCUBE3, SDAD1, SEC14L1, SEC31B, SEMA4D, SEMA6C, SEMA6D, SEPT11, SEPT7P2, SEPTIN11, SEPTIN3, SEPTIN6, SEPTIN7P2, SERGEF, SERP1, SETD5, SFXN2, SGMS1, SH2B1, SH3BP5-AS1, SH3PXD2B, SHANK1, SHLD2, SIPA1L3, SIX1, SLC12A5, SLC1A6, SLC24A3, SLC25A14, SLC25A22, SLC2A11, SLC35G1, SLC38A7, SLC41A2, SLC4A3, SMAD4, SMG1P7, SPATA17, SPATS2, SPEG, SPIN1, SRRM4, ST5, STMN2, STOX2, STRA6, STXBP5L, SUPT3H, SVEP1, SYDE1, SYNE1, SYNGR3, SYNJ2, SYT7, TAF6, TAFA2, TBCD, TBL1XR1, TENM3, TEX9, TGFB3, THUMPD3-AS1, TM6SF2, TMEM117, TMEM175, TMEM189, TMEM191A, TMEM198B, TMEM214, TMEM230, TMEM88, TPRA1, TRAF3, TRAPPC12, TRIM16, TRIM6, TRIO, TRRAP, TSHZ3, TSPAN3, TTC39C-AS1, TTLL4, TTTY14, TUBB3, TUBB6, TUBGCP6, TXLNGY, UNC13A, UNK, USP10, USP28, USP36, VAX2, VPS29, VPS50, VPS53, WARS2, WASL, WDFY2, WDR19, WDR37, WDR4, WWOX, ZBTB18, ZC2HC1C, ZCCHC4, ZDHHC1, ZFAT, ZFP91, ZFP91-CNTF, ZGPAT, ZNF195, ZNF202, ZNF236, ZNF320, ZNF382, ZNF394, ZNF420, ZNF423, ZNF429, ZNF43, ZNF48, ZNF527, ZNF571-AS1, ZNF583, ZNF594-DT, ZNF598, ZNF692, ZNF696, ZNF700, ZNF737, ZNF785, ZNF789, ZNF81, ZNF814, ZNF826P, ZNF875, ZNHIT1, ZRANB3, ZSCAN12.

In some embodiments and examples described herein, at least part of the intronic region is derived from AARS1, i.e., the intronic region between exon 4 and exon 5 of AARS1. In some embodiments, the first part and second part of the intronic region is derived from AARS1, i.e., the intronic region between exon 4 and exon 5 in the human genome. The first part of the intronic region deriving from AARS1 may correspond to at least part of the intronic region between exon 4 and exon 5 of AARS1 in the human genome which is upstream of the AARS1 cryptic exon. The second part of the intronic region deriving from AARS1 may correspond to at least part of the intronic region between exon 4 and exon 5 of AARS1 in the human genome that is downstream of the AARS1 cryptic exon.

In some embodiments, the first part of the intronic region may comprise a sequence which is at least 80% identical to one of SEQ ID NO: 30, SEQ ID NO: 70, SEQ ID NO: 76, SEQ ID NO: 82, SEQ ID NO: 119, SEQ ID NO: 125, SEQ ID NO: 131, SEQ ID NO: 137, SEQ ID NO: 143, SEQ ID NO: 149 or SEQ ID NO: 155, or at least 85%, or at least 90%, or at least 95%, or at least 100% identical to one of SEQ ID NO: 30, SEQ ID NO: 70, SEQ ID NO: 76, SEQ ID NO: 82, SEQ ID NO: 119, SEQ ID NO: 125, SEQ ID NO: 131, SEQ ID NO: 137, SEQ ID NO: 143, SEQ ID NO: 149 or SEQ ID NO: 155 or SEQ ID NO 179, or SEQ ID NO 185 or SEQ ID NO 191, or SEQ ID NO 197. In some embodiments, the second part of the intronic region may comprise a sequence which is at least 80% identical to one of SEQ ID NO: 32 or SEQ ID NO: 72, or SEQ ID NO: 78, or SEQ ID NO: 84, or SEQ ID NO: 121, or SEQ ID NO: 127, or SEQ ID NO: 133, or SEQ ID NO 139, or SEQ ID NO: 145, or SEQ ID NO: 151 or SEQ ID NO: 157, or at least 85%, or at least 90%, or at least 100% identical to SEQ ID NO: 32 SEQ ID NO: 72, or SEQ ID NO: 78, or SEQ ID NO: 84, or SEQ ID NO: 121, or SEQ ID NO: 127, or SEQ ID NO: 133, or SEQ ID NO 139, or SEQ ID NO: 145, or SEQ ID NO: 151 or SEQ ID NO: 157, or SEQ ID NO: 181, or SEQ ID NO: 187, or SEQ ID NO: 193, or SEQ ID NO: 199. In some examples, the first part of the intronic sequence is at least 80%, or at least 85%, or at least 90%, or at least 95%, or identical SEQ ID NO 30 and the second part of the intronic sequence is at least 80%, or at least 85%, or at least 90%, or at least 95%, or identical 32 are derived from AARS1 intronic region between exon 4 and exon 5 in the human genome.

In other examples, the first part and second part of the intronic region are synthetic. In some embodiments, the intronic region is designed such that the intronic region begins with GT (AAG) and ends with (C) AG. In some embodiments and examples, the first part and second part of the intronic region may be selected such that the first acceptor splice site and/or first donor splice site have a splice score of at least 0.01, or at least 0.05, or at least 0.1, or at least 0.3, or between 0.01 and 0.8 (as determined by the Splice AI algorithm), and/or wherein the second acceptor splice site and/or second splice donor site have a splice score of at least 0.01, but preferably at least 0.5, or at least 0.9, or at least 0.95 as determined by the Splice AI algorithm. In some embodiments, the intronic region (i.e., the first part of the intronic region, the cryptic exon sequence, or the second part of the intronic region) is designed to comprise a binding domain for the splicing factor of the hnRNP family (e.g., TDP-43). In some embodiments, the binding domain is for TDP-43 and the intronic sequence comprises a sequence which is at least 80% identical, or at least 85% identical, or at least 90% identical or at least 95% identical or at least 100% identical with SEQ ID NO: 2 or SEQ ID NO: 115, or comprises a TDP-43 binding domain as otherwise described herein. In preferred embodiments or examples, the intronic region is designed such that the intronic region (e.g., first part of the intronic region) comprises a polypyrimidine tract. A polypyrimidine tract defined herein may be described as a 20 nucleotide region that is pyrimidine rich, defined as a 20 nucleotide region with at least 70% pyrimidines, or a 30 nucleotide region with at least 80% pyrimidines.

As indicated above, the intronic region is defined by a second splice donor site and a second splice acceptor site. The second splice donor site and the second splice donor site are typically at least 150 nucleotides apart, more preferably at least 200 nucleotides apart. In some embodiments, the sequence surrounding the second splice acceptor site is HAG/N wherein/represents the splice site, wherein H=C, T or A and N is C, T, A or G. In some embodiments or examples, the construct comprises a polypyrimidine tract upstream of the second splice acceptor site (i.e., within the cryptic exon sequence upstream of the second splice acceptor site, e.g., upstream of HAG/N). In some embodiments, the polypyrimidine tract is upstream of the second splice acceptor site, more preferably up to 40 nucleotides upstream of the second splice acceptor site, or up to 20 nucleotides upstream of the first splice acceptor site. A polypyrimidine tract defined herein may be described as a region that is pyrimidine rich, defined as a 20 nucleotide region with at least 70% pyrimidines and a 30 nucleotide region with at least 80% pyrimidines.

In some examples, the sequence surrounding the second donor splice is CAG/GT wherein/represents the splice site.

In some embodiments, the intronic region comprises one or more branch sites comprising an adenosine upstream of the first and/or second splice acceptor site and the polypyrimidine tract (i.e., within the intronic region upstream of the second splice acceptor site). The branch site(s) may comprise the sequence PTNAP, wherein N is any nucleotide, P is a pyrimidine (i.e., C or T), and wherein the underlined A is the branchpoint for example (e.g., CTGAC). The branch site(s) may be located up to 45 nucleotides upstream of the first and/or second splice acceptor, preferably up to 35 nucleotides upstream of the first and/or second splice acceptor and preferably between 20 and 35 nucleotides upstream of the first and/or second splice acceptor

### Cryptic Exon

The cryptic exon sequence is defined (i.e., between) the first splice acceptor site and the first splice donor site. In some embodiments, the first splice donor site and/or the first splice acceptor site have a splice score of 0.01 (the 99.8th percentile of SpliceAI scores) or above as determined by the Splice AI algorithm, or in some embodiments, 0.05 or above, or in some embodiments, 0.1 or above. In some embodiments, the first splice donor site and/or the first splice acceptor site, defining the cryptic exon, have a splice score of 0.01 to 0.7, or from 0.05 to 0.7, or from 0.1 to 0.7. In preferred embodiments, the splice score(s) for the first splice acceptor site and first splice donor site may be lower than the splice score(s) for the second splice acceptor site and second splice donor site. In preferred embodiments, the intronic region (i.e., defined by the second splice donor site and second splice acceptor site) comprises no other splice site identified as having a splice score of 0.2 or above. In preferred embodiments, the first splice acceptor site and the first splice donor site have the highest splice AI score in the intronic region (i.e., defined by the second splice donor site and second splice acceptor site, but not including the second splice donor site and second splice acceptor site). In preferred embodiments, the first splice acceptor site and the first splice donor site have the highest splice AI score in the cryptic exon sequence. In some embodiments, the first splice acceptor site and the first splice donor site have the highest Splice AI score within 100 nucleotides, or within 50 nucleotides, or within 25 nucleotides of the first acceptor and first splice donor sites.

In some embodiments, the cryptic exon sequence comprises from about 10 nucleotides to about 2000 nucleotides, preferably 30 to 500 nucleotides, or in some examples, from 44 nucleotides to about 200 nucleotides.

In some embodiments, the cryptic exon sequence is a frame-shift inducing cryptic exon sequence, i.e., the exon sequence comprises a number of nucleotides that is not divisible by 3. The construct is configured such that (i.e., for the mRNA product of the construct):
(i) if placed in a cell that that is depleted of the splicing factor of the hnRNP family, the complete transgene sequence is in frame with the start codon, and
(ii) if placed in a cell that is not depleted of splicing factor of the hnRNP family, at least part of the transgene sequence is out of frame with the start codon.

In such embodiments, the construct may further comprise a premature termination codon downstream of the regulatory domain and cryptic exon sequence. If placed in a cell with nuclear depletion of the splicing factor, the cryptic exon sequence is included in the mRNA of the construct such that the start codon is out of frame with the premature termination codon. If placed in a cell without nuclear depletion of the splicing factor, the cryptic exon sequence is not included in the mRNA of the construct such that the start codon is in frame with the premature termination codon. In such embodiments, the construct may further comprise a further intronic sequence downstream of the regulatory domain and transgene sequence as described elsewhere herein.

In alternative embodiments, the cryptic exon sequence is not a frame-shift inducing cryptic exon sequence, i.e., the nucleotide sequence comprises a number of nucleotides that is divisible by 3. Such embodiments may be used, for example, wherein the cryptic exon comprises the start codon. Such embodiments may be used if the cryptic exon encodes for at least part of the transgene. In such constructs, the construct or transgene sequence may not comprise a PTC (i.e., that is relevant for the regulation of protein expression).

In some embodiments, the cryptic exon sequence is a known cryptic exon that is regulated by a splicing factor of the hnRNP family, such as TDP-43. In some embodiments, the cryptic exon sequence derives from the cryptic exon sequences in human genes at least part of AACSP1, AARS1, ABCB1, ABCD1, AC002310.11, AC002310.7, AC002456.2, AC008543.1, AC008676.3, AC009133.12, AC010531.1, AC015712.1, AC015712.6, AC022387.2, AC022966.1, AC025165.6, AC064807.1, AC092073.1, AC138932.1, AC245041.2, ACSF2, ACTL6B, ACTR1A, ADARB1, ADARB2, ADCY1, ADCY7, ADCY8, ADGRB1, ADGRL1, ADSSL1, AGK, AGRN, AHNAK, AKT3, AL023775.2, AL031282.2, AL035461.3, AL121845.3, AL157392.3, AL157392.5, AL354696.2, AL360181.3, AL645568.1, AL669831.3, AL672142.1, ALDH3B1, AMPD2, ANKRD19P, ANKRD44, ANOS2P, AP000662.4, AP006621.8, AP4M1, ARAP3, ARF1, ARHGAP22, ARHGAP23, ARHGEF 16, ARHGEF19, ASGR1, ATAD5, ATG4B, ATP5MG, ATP8A2, ATXN1, ATXN10, BCL2L11, BCL2L13, BLCAP, BMP8B, BNIP3P11, BRD1, BTN3A3, C16orf95, C20orf194, C2orf81, C4orf36, C5orf66, CACNB2, CACNG5, CAMK2B, CAMTA1, CASP8, CASTOR1, CBY1, CCDC102B, CCDC150, CCDC183-AS1, CCDC33, CCT2, CDHR2, CDK11A, CDKAL1, CDON, CELF5, CENPBD1P1, CENPK, CENPS-CORT, CEP152, CEP290, CEP72, CEP83, CH17-189H20.1, CH507-154B10.1, CHD8, CHFR, CHGB, CHRNA5, CHRNB3, CLCN6, CLSPN, CLTCL1, CNGA3, CNPY1, CORO6, CPVL, CREB3L4, CRLS1, CRTC1, CSMD2, CTC-490E21.12, CTD-2014B16.3, CTD-2054N24.2, CTD-2162K18.4, CTD-2554C21.2, CTD-2561J22.3, CU634019.6, CUL9, CYFIP2, CYP2C8, DACH2, DACT3-AS1, DAGLA, DAPK1, DELE1, DENND2B, DGKA, DLG5, DLGAP1, DNAJC12, DNAJC25-GNG10, DNMT3A, DNMT3B, DOCK1, DPF1, DUXAP9, EBF1, ECEL1, EHD2, EIF2A, EIF2AK1, EIF4ENIF1, ELAVL3, EML6, ENAH, ENTPD6, EP300, EP400, EPB41L1, EPB41L4A, EPS8L2, ETV5, F12, FADS2, FAM114A2, FAM156A, FAM182B, FAM66D, FAM66E, FBL, FBXL19, FGFR4, FIRRE, FKBP14-AS1, FOXK1, FRYL, G2E3, G3BP1, GALNT12, GAS6, GATA2, GLIPR2, GMPPA, GOLGA7B, GOLGA8A, GPHN, GPSM2, GPX7, GRAMD1A, GREB1, GRIN2D, GSTCD, GTF2H2, GTF2IP13, HAUS2, HDAC6, HDGFL2, HDLBP, HECTD4, HERC2P2, HIPK1, HROB, HULC, ICA1, IFT122, IGSF21, IGSF9, IK, IL15, INPP4A, INSR, INTS11, IQCE, IQCK, ISL2, ISYNA1, ITGA3, ITGA7, ITPR3, KALRN, KATNA1, KCNIP1, KCNIP2, KCNK15-AS1, KCNQ2, KCNT1, KDM1B, KDM4D, KIAA1211, KIAA1217, KIF14, KIF21A, KLC1, KMT5A, KNDC1, KRT8, L3MBTL1, LCOR, LIAS, LINC00265, LINC00342, LINC00475, LINC01002, LINC01224, LINC01322, LINC01503, LINC01572, LINC01684, LINC02082, LINC02202, LINC02506, LINGO1, LMNA, LRP1B, LRP8, LSM12, LSS, LTBP2, MACROD1, MADD, MANBAL, MAP2K6, MAPKAPK5, MATK, MBP, MC1R, MCM9, MDC1, MED12, MED13L, MEIS2, METTL8, MGAT5B, MIER3, MMAA, MRPL34, MTRR, MTX1P1, NAA38, NADSYN1, NAT1, NBEA, NBPF9, NDUFB9, NFKBIZ, NFYC, NIPSNAP3B, NPIPB11, NPLOC4, NSFL1C, NTRK2, NTRK3, NUP188, NUP210, OBSCN, OPCML, PAOX, PATJ, PCBP3, PCBP4, PCDH11X, PCSKIN, PDCD2L, PDCD6, PDE2A, PDE9A, PER3, PHF2, PHF5A, PI4KA, PIGG, PIGU, PKD1P3, PKN1, PLCE1, PLEKHA1, PLEKHA6, PLEKHG2, PLEKHG4, PLEKHM2, POLD1, POLR2F, POU2F2, PPCDC, PPIP5K1, PPMIN, PPP1R14B-AS1, PRDM8, PRELID3A, PREX1, PRKG2, PROX1-AS1, PRPF40B, PRRT4, PRUNE2, PSPC1, PTK2, PTPN13, PTPN21, PTPRN2, PTPRT, PUDP, PUS7L, PWWP3A, PXDN, RAB20, RAB27A, RALGAPA2, RANBP17, RASGRP2, RBMXL1, RC3H1, RCAN3, RET, RFLNA, RGMA, RHOQ, RP1-120G22.12, RP1-138B7.8, RP1-283E3.8, RP1-59M18.2, RP11-101E3.5, RP11-108K14.8, RP11-108L7.4, RP11-124N2.1, RP11-155D18.12, RP11-155G14.5, RP11-155G14.6, RP11-206L10.2, RP11-30K9.6, RP11-345P4.10, RP11-411B6.6, RP11-436D23.1, RP11-465B22.3, RP11-47909.4, RP11-505D17.1, RP11-511P7.6, RP11-566K11.4, RP11-613M10.9, RP11-61L23.2, RP11-718011.1, RP11-739N20.2, RP11-73M18.2, RP11-761B3.1, RP11-795F19.5, RP11-977G19.10, RP4-583P15.15, RP5-967N21.13, RPGRIP1L, RSF1, RTL1, SCN9A, SCUBE3, SDAD1, SEC14L1, SEC31B, SEMA4D, SEMA6C, SEMA6D, SEPT11, SEPT7P2, SEPTIN11, SEPTIN3, SEPTIN6, SEPTIN7P2, SERGEF, SERP1, SETD5, SFXN2, SGMS1, SH2B1, SH3BP5-AS1, SH3PXD2B, SHANK1, SHLD2, SIPA1L3, SIX1, SLC12A5, SLC1A6, SLC24A3, SLC25A14, SLC25A22, SLC2A11, SLC35G1, SLC38A7, SLC41A2, SLC4A3, SMAD4, SMG1P7, SPATA17, SPATS2, SPEG, SPIN1, SRRM4, ST5, STMN2, STOX2, STRA6, STXBP5L, SUPT3H, SVEP1, SYDE1, SYNE1, SYNGR3, SYNJ2, SYT7, TAF6, TAFA2, TBCD, TBL1XR1, TENM3, TEX9, TGFB3, THUMPD3-AS1, TM6SF2, TMEM117, TMEM175, TMEM189, TMEM191A, TMEM198B, TMEM214, TMEM230, TMEM88, TPRA1, TRAF3, TRAPPC12, TRIM16, TRIM6, TRIO, TRRAP, TSHZ3, TSPAN3, TTC39C-AS1, TTLL4, TTTY14, TUBB3, TUBB6, TUBGCP6, TXLNGY, UNC13A, UNK, USP10, USP28, USP36, VAX2, VPS29, VPS50, VPS53, WARS2, WASL, WDFY2, WDR19, WDR37, WDR4, WWOX, ZBTB18, ZC2HC1C, ZCCHC4, ZDHHC1, ZFAT, ZFP91, ZFP91-CNTF, ZGPAT, ZNF195, ZNF202, ZNF236, ZNF320, ZNF382, ZNF394, ZNF420, ZNF423, ZNF429, ZNF43, ZNF48, ZNF527, ZNF571-AS1, ZNF583, ZNF594-DT, ZNF598, ZNF692, ZNF696, ZNF700, ZNF737, ZNF785, ZNF789, ZNF81, ZNF814, ZNF826P, ZNF875, ZNHIT1, ZRANB3, ZSCAN12. In some embodiments, the known cryptic exon may have been mutated by insertion or deletion of nucleotides (e.g., addition or deletion of any number of nucleotides that is not divisible by three, e.g., preferably addition or deletion of one or two nucleotides) such that the cryptic exon is a frame-shift inducing cryptic exon. In one of the examples described herein, the cryptic exon is derived from the human AARS1 cryptic exon sequence but which comprises an additional nucleotide, e.g., an additional adenosine nucleotide, increasing its length from 87 to 88 nucleotides.

In some embodiments, the cryptic exon sequence has a sequence that has at least 80% sequence identity, or at least 85%, or at least 90%, or at least 95%, or at least 100% sequence identity with SEQ ID NO: 31. This sequence derives from the cryptic exon sequence in the human AARS1 gene, between exons 4 and 5, but with insertion of an additional nucleotide. In the example described herein, the additional nucleotide is an adenosine. In alternative embodiments, the cryptic exon sequence is a synthetic exon sequence. The cryptic exon sequence may be designed using Splice AI algorithm (i.e., comprise a sequence such that the splice site(s) flanking the cryptic exon sequence have a probability score of at least 0.01, or at least 0.05, or at least 0.1 as determined by the Splice AI algorithm), as described above and/or using “algorithm 1” as described herein. Note that the cryptic exon splice sites are expected to be weaker than constitutively spliced splice sites, and thus may be selected to have lower SpliceAI scores. In some embodiments, the synthetic cryptic exon sequence encodes for a part of the transgene, and the part of the transgene is modified to comprise synonymous codons.

In some examples, the cryptic exon sequence has at least 80% sequence identity, or at least 85%, or at least 90%, or at least 95%, or at least 100% sequence identity with SEQ ID NO: 31, SEQ ID NO: 49, SEQ ID NO: 51-64, SEQ ID NO: 71, SEQ ID NO: 77, SEQ ID NO: 83, SEQ ID NO: 88, SEQ ID NO: 92, SEQ ID NO: 120, SEQ ID NO: 126, SEQ ID NO: 132, SEQ ID NO: 138, SEQ ID NO: 14q

In some embodiments, the regulatory domain may comprise the following features from upstream to downstream:
a splice donor site (i.e., the second splice donor site),
a first part of the intronic region
a splice acceptor site (i.e., the first splice acceptor site),
a cryptic exon sequence,
a splice donor site (i.e., the first splice donor site),
a second part of the intronic region and
a splice acceptor site (i.e., the second splice acceptor site), and

The binding domain for the splicing factor (i.e., of the hnRNP family) may be within the first part of the intronic region, the cryptic exon sequence, or the second part of the intronic region.

In some embodiments, the construct may further comprise an exon sequence or exonic region immediately upstream of the second splice donor site and/or an exon sequence or exonic region immediately downstream of the second splice acceptor site. In some embodiments, the exon immediately upstream of the first splice acceptor site and/or the exon immediately downstream of the first splice donor site may encode for at least part of the transgene sequence. In other embodiments, the exon immediately upstream of the first splice acceptor site and/or the exon immediately downstream of the first splice donor site may encode for a peptide sequence which does not encode for part of the transgene sequence.

In some embodiments, regulatory domain may comprise the following features from upstream to downstream:
An exonic sequence immediately upstream of the splice donor site
a splice donor site (i.e., the second splice donor site),
a first part of the intronic region
a splice acceptor site (i.e., the first splice acceptor site),
a cryptic exon sequence embedded within the intronic region,
a splice donor site (i.e., the first splice donor site),
a second part of the intronic region and
a splice acceptor site (i.e., the second splice acceptor site), and
an exonic sequence immediately downstream of the splice acceptor site.

The binding domain for the splicing factor (i.e., of the hnRNP family) may be within the first part of the intronic region, cryptic exon sequence, or the second part of intronic region. In some embodiments, the exonic sequence immediately upstream of the splice donor site and the exonic sequence immediately downstream of the splice acceptor site may encode for part of the transgene sequence. In alternative embodiments, the exonic sequences immediately upstream of the splice donor site and the exonic sequence immediately downstream of the splice acceptor site may encode for a peptide, different to the protein produced by the transgene.

### Constructs Containing a Cryptic Exon Sequence According to “Design 1”

In some embodiments of the construct, the one or more exons that encode for the transgene are all downstream of the cryptic exon sequence and/or regulatory domain. Such constructs are described herein as “Design 1” constructs which are shown schematically in FIG. 1.

An example construct may comprise a regulatory domain and a transgene sequence (i.e., a transgene sequence encoding a functional protein), wherein the regulatory domain comprises, from upstream to downstream:
an exonic sequence immediately upstream of the splice donor site
a splice donor site (i.e., the second splice donor site),
a first part of the intronic region,
a splice acceptor site (i.e., the first splice acceptor site),
a cryptic exon sequence embedded within the intronic region,
a splice donor site (i.e., the first splice donor site),
a second part of the intronic region, and
a splice acceptor site (i.e., the second splice acceptor site), and
an exonic sequence immediately downstream of the splice acceptor site

These features may all be as described elsewhere herein. The binding domain for the splicing factor of the hnRNP family may be within the first part of the intronic region, cryptic exon sequence, or the second part of the intronic region. The transgene can be encoded by at least part of the exonic sequence downstream of the second splice acceptor site (i.e., downstream of the regulatory domain). In some embodiments, the transgene may be downstream of the regulatory domain. In some embodiments, the transgene may be encoded by the cryptic exon sequence. In such embodiments, the transgene may be encoded by the cryptic exon sequence and the exonic sequence immediately upstream of the splice donor site and/or the exonic sequence immediately downstream of the splice acceptor site.

The construct of Design 1 may further comprise one or more optional features.
a sequence comprising a start codon upstream of the regulatory domain
a premature termination codon (PTC), downstream of the cryptic exon sequence, which may be present in (but out of frame with) the transgene sequence.
a further intronic sequence downstream of the PTC
a sequence for a protease cleavage site or self-cleaving cleavage site, (e.g., upstream of the transgene sequence and downstream of the regulatory domain).

In such embodiments, the construct comprises the following features from upstream to downstream.
an optional sequence comprising a start codon,
an exonic sequence immediately upstream of the splice donor site
a splice donor site (i.e., the second splice donor site),
a first part of the intronic region
a splice acceptor site (i.e., the first splice acceptor site),
a cryptic exon sequence (i.e., embedded within the intronic region between the first splice acceptor site and the first splice donor site),
a splice donor site (i.e., the first splice donor site),
a second part of the intronic region,
a splice acceptor site (i.e., the second splice acceptor site), and
an exonic sequence immediately downstream of the splice acceptor site,
an optional protein cleavage or self-cleavage site,
a transgene sequence (i.e., a complete transgene sequence), optionally comprising a PTC
an optional further intronic sequence (i.e., downstream of the transgene sequence and within an exonic context).

The binding domain for the splicing factor of the hnRNP family may be within the first part of the intronic region, cryptic exon sequence, or the second part of the intronic region.

In some embodiments, the start codon is upstream of the regulatory domain. In other embodiments, the start codon is within the regulatory domain (e.g., in the exonic sequence immediately upstream of the second splice donor site), and in some embodiments, the start codon is within the cryptic exon sequence.

The above features may have any of the same features as described elsewhere herein. In some examples described herein, the exon immediately upstream of the splice donor site, first part of the intronic region, cryptic exon sequence, second part of the intronic region, and the exon immediately downstream of the splice donor site, all derive from the human AARS1 gene or a modified variant thereof. In other examples, the exon immediately upstream of the splice donor site, first part of the intronic region, cryptic exon sequence, second part of the intronic region, and the exon immediately downstream of the splice donor site are alternatively synthetic sequences. In some examples, the further intronic sequence and surrounding exonic context derives from RPS24. In some examples, the self-cleavage site is P2A. In some examples, the transgene encodes for a diagnostic protein (e.g., mCherry, or Gaussia Luciferase). In other examples, the transgene encodes for a therapeutic protein (e.g., a splicing regulator, such as TDP-43 binding domain fused to RAVER 1, more particularly the TDP-43 RNA binding domain fused to RAVER 1). In some examples described herein, the binding domain for the hnRNP family is TDP-43, and the splicing factor is TDP-43. In some embodiments, the binding domain is a functional binding domain or a mutant binding domain.

In some examples, the construct has a sequence that has at least 80% or at least 85%, or at least 90%, or at least 95%, or at least 100% sequence identity with SEQ ID NO: 25 or SEQ ID NO: 47.

In some examples, the first part of the intronic region has a sequence that has at least 80% sequence identity, or at least 85%, or at least 90%, or at least 95%, or at least 100% sequence identity with SEQ ID NO: 30 or SEQ ID NO: 70, or SEQ ID NO: 76, or SEQ ID NO:82, or SEQ ID NO: 119, or SEQ ID NO: 125, or SEQ ID NO: 131, or SEQ ID NO: 137, or SEQ ID NO: 143, or SEQ ID NO 149, or SEQ ID NO: 155, or SEQ ID NO 179, or SEQ ID NO 185 or SEQ ID NO 191, or SEQ ID NO 197.

In some examples, the second part of the intronic region has a sequence that has at least 80% sequence identity, or at least 85%, or at least 90%, or at least 95%, or at least 100% sequence identity with SEQ ID NO: 32 or SEQ ID NO: 72, or SEQ ID NO: 78, or SEQ ID NO: 84, or SEQ ID NO: 121, or SEQ ID NO: 127, or SEQ ID NO: 133, or SEQ ID NO: 139, or SEQ ID NO: 145, or SEQ ID NO: 151, or SEQ ID NO: 157 or SEQ ID NO: 181, or SEQ ID NO: 187, or SEQ ID NO: 193, or SEQ ID NO: 199.

In some examples, the TDP-43 binding domain has a sequence that has at least 80% sequence identity, or at least 85%, or at least 90%, or at least 95%, or at least 100% sequence identity with SEQ ID NO: 1-9, or SEQ ID NO: 115, SEQ ID NO: 159 or SEQ ID NO: 160.

In some examples, the further intronic sequence has a sequence that has at least 80% sequence identity, or at least 85%, or at least 90%, or at least 95%, or at least 100% sequence identity with SEQ ID NO: 36.

In some examples, the cryptic exon sequence has a sequence that has at least 80% sequence identity, or at least 85%, or at least 90%, or at least 95%, or at least 100% sequence identity with SEQ ID NO: 31, SEQ ID NO: 49 or SEQ ID NO: 51-64, or SEQ ID NO: 71, or SEQ ID NO: 77, or SEQ ID NO: 83, or SEQ ID NO: 120, or SEQ ID NO: 126, or SEQ ID NO: 132, or SEQ ID NO: 138, or SEQ ID NO: 144, or SEQ ID NO: 150 or SEQ ID NO: 156

In some examples, the self-cleavage site has a sequence that has at least 80% sequence identity, or at least 85%, or at least 90%, or at least 95%, or at least 100% sequence identity with SEQ ID NO: 34.

In some examples, the exonic sequence immediately upstream of the first splice acceptor site has at least 80% sequence identity, or at least 85%, or at least 90%, or at least 95%, or at least 100% sequence identity with SEQ ID NO: 29 or SEQ ID NO: 48.

In some examples, the exonic sequence immediately downstream of the first splice donor site has at least 80% sequence identity, or at least 85%, or at least 90%, or at least 95%, or at least 100% sequence identity with SEQ ID NO: 33, or SEQ ID NO: 50.

### Constructs According to “Design 2”

In alternative embodiments, the cryptic exon sequence may encode for at least part of the transgene. The cryptic exon sequence may encode for an internal part of a protein, the N-terminal part of the protein, or a C-terminal part of the protein. Such constructs are described herein as “Design 2” constructs and are shown schematically in FIG. 2. The construct may comprise further exonic sequences that encode for another part of the transgene protein. In some embodiments, the construct may comprise another part of the transgene sequence downstream of the cryptic exon and/or upstream of the cryptic exon. In some examples, described herein, the transgene sequence is formed from at least three parts that together form a complete transgene sequence. In some embodiments, the transgene sequence may be split into two or more parts, or three or more parts, or four or more parts, or five or more parts, or six or more parts, or seven or more parts, or eight or more parts, or nine or more parts, or ten or more parts. The transgene may be split into parts such that the first donor acceptor site, first splice acceptor site, second splice acceptor site and second splice donor site have a splicing score of at least 0.01 as determined by the Splice AI algorithm, or according to other splicing scores determined by the Splice AI algorithm as described herein. In some embodiments, the transgene sequence may be modified to include synonymous codon sequences.

In some embodiments, the regulatory domain may comprise the following features from upstream to downstream:
A splice donor site (i.e., the second splice donor site),
a first part of the intronic region
a splice acceptor site (i.e., the first splice acceptor site),
a cryptic exon sequence which encodes for at least part of the transgene,
a splice donor site (i.e., the first splice donor site),
a second part of the intronic region and
a splice acceptor site (i.e., the second splice acceptor site).

The binding domain for the splicing factor (i.e., of the hnRNP family) may be within the first part of the intronic region, cryptic exon sequence, or the second part of the intronic region. These features may all be as described elsewhere herein.

An example construct may comprise a transgene and a regulatory domain, the regulatory domain comprising the following features, from upstream to downstream.
an exon immediately upstream of the splice donor site (i.e., optionally encoding for part of the transgene)
a splice donor site (i.e., the second splice donor site),
a first part of the intronic region
a splice acceptor site (i.e., the first splice acceptor site),
a cryptic exon sequence embedded within the intronic region, encoding for at least a part of the transgene, and optionally the first or the second part of the transgene,
a splice donor site (i.e., the first splice donor site),
a second part of the intronic region,
a splice acceptor site (i.e., the second splice acceptor site), and
an exon immediately downstream of the splice acceptor site, optionally encoding for a part of the transgene.

The binding domain for the splicing factor of the hnRNP family may be within the first part of the intronic region, cryptic exon sequence, or the second part of the intronic region.

The construct of Design 2 may also further comprise one or more optional features.
a sequence comprising a start codon upstream of the regulatory domain
a premature termination codon (PTC), downstream of the cryptic exon sequence, which may be present in the transgene sequence.
a further intronic sequence downstream of the PT
a sequence for a protease cleavage site or self-cleaving cleavage site, (e.g., between two different transgene sequences).

An example construct may therefore have the following features, from upstream to downstream.

An optional start codon sequence
an exon immediately upstream of the splice donor site (i.e., optionally encoding for part of the transgene, (e.g., a first part of the transgene)
a splice donor site (i.e., the second splice donor site),
a first part of the intronic region (i.e., or first intron),
a splice acceptor site (i.e., the first splice acceptor site),
a cryptic exon sequence embedded within the intronic region, encoding for at least a part of the transgene, (e.g., a second part of the transgene),
a splice donor site (i.e., the first splice donor site),
a second part of the intronic region (i.e., a second intron) and
a splice acceptor site (i.e., the second splice acceptor site), and
an exon immediately downstream of the splice acceptor site, optionally encoding for a part of the transgene, (e.g., a third part of the transgene),
an optional further intron sequence downstream of the transgene.

The binding domain for the splicing factor of the hnRNP family may be within the first part of the intronic region, cryptic exon sequence, or the second part of the intronic region.

In some embodiments, the start codon is upstream of the regulatory domain. In other embodiments, the start codon is within the regulatory domain, and in some embodiments, the start codon is within the cryptic exon sequence.

These features may be as described elsewhere herein. In some examples described herein, the exon immediately upstream of the splice donor site, first part of the intronic region, and the second part of the intronic region, derive from the human AARS1 gene or a modified variant thereof. In some examples, the exons that encode for the transgene together encode for a diagnostic protein (e.g., mCherry), or a therapeutic protein (e.g., a nuclease, such as Cas 9), or a recombinase protein (e.g., Cre recombinase). In some examples, the optional intron sequence and optional exon sequence downstream of the one or more exons that together encode for the transgene derive from RPS24. In the examples described herein, the binding domain is for TDP-43, and the splicing factor (i.e., of the hnRNP family) is TDP-43.

In some examples, the construct has a sequence has a sequence that has at least 80% or at least 85%, or at least 90%, or at least 95%, or at least 100% sequence identity with SEQ ID NO: 68, SEQ ID NO: 74, SEQ ID NO: 80, SEQ ID NO: 86, SEQ ID NO: 90, SEQ ID NO: 117, SEQ ID NO: 123, SEQ ID NO: 129, SEQ ID NO: 135, SEQ ID NO: 141, SEQ ID NO: 147, SEQ ID NO: 153.

In some examples, the first part of the intronic region has a sequence that has at least 80% sequence identity, or at least 85%, or at least 90%, or at least 95%, or at least 100% sequence identity with SEQ ID NO: 30 or SEQ ID NO: 70, or SEQ ID NO: 76, or SEQ ID NO: 82, or SEQ ID NO: 119, or SEQ ID NO: 125, or SEQ ID NO: 131, or SEQ ID NO: 137, or SEQ ID NO: 143, or SEQ ID NO 149, or SEQ ID NO: 155, or SEQ ID NO 179, or SEQ ID NO 185 or SEQ ID NO 191, or SEQ ID NO 197.

In some examples, the second part of the intronic region has a sequence that has at least 80% sequence identity, or at least 85%, or at least 90%, or at least 95%, or at least 100% sequence identity with SEQ ID NO: 32 or SEQ ID NO: 72, or SEQ ID NO: 78, or SEQ ID NO: 84 or SEQ ID NO: 121, or SEQ ID NO: 127, or SEQ ID NO: 133, or SEQ ID NO: 139, or SEQ ID NO: 145, or SEQ ID NO: 151, or SEQ ID NO: 157 or SEQ ID NO: 181, or SEQ ID NO: 187, or SEQ ID NO: 193, or SEQ ID NO: 199.

In some examples, the TDP-43 binding domain has a sequence that has at least 80% sequence identity, or at least 85%, or at least 90%, or at least 95%, or at least 100% sequence identity with SEQ ID NO: 1-9, or SEQ ID NO: 115, or SEQ ID NO: 159 or SEQ ID NO: 160.

In some examples, the further intronic sequence has a sequence that has at least 80% sequence identity, or at least 85%, or at least 90%, or at least 95%, or at least 100% sequence identity with SEQ ID NO: 36.

In some examples, the cryptic exon is placed within a prime editing vector. In some embodiments, the prime editing vector uses a H840A mutant S. pyogenes Cas9. In some embodiments, the first part of the intron has a sequence that has at least 80% sequence identity, or at least 85%, or at least 90%, or at least 95%, or at least 100% sequence identity with SEQ ID NO: 191. has a sequence that has at least 80% sequence identity, or at least 85%, or at least 90%, or at least 95%, or at least 100% sequence identity with SEQ ID NO: 193

Constructs where Regulatory Domain is Regulated by Splicing of a Single Regulatory Intron

In a third aspect, or embodiment of the first aspect, there is provided, a construct comprising
a start codon,
a regulatory domain comprising:
   a first splice donor site and a first acceptor donor site, which define a single regulatory intron,
   a binding domain for a splicing factor of the heterogenous nuclear ribonucleoprotein (hnRNP) family, located within 150 nucleotides of the first splice donor site or first splice acceptor site and/or located between the first splice donor site and first splice acceptor site; and
a transgene sequence (i.e., a transgene sequence encoding a functional protein),
   configured such that
   (i) if placed in a cell that is depleted of splicing factor, splicing of the first splice acceptor site and/or first donor site is not repressed and the single regulatory intron is spliced, such that a functional protein is produced from the transgene sequence (i.e., a functional product is produced in the mRNA product of the construct where the functional protein is encoded by the complete, uninterrupted transgene sequence)
   (ii) if placed in a cell that is not depleted of splicing factor, the single regulatory intron is not or incorrectly spliced such that no functional protein is produced from the transgene sequence (i.e., a functional product is produced in the mRNA product of the construct where the functional protein is encoded by the complete, uninterrupted transgene sequence).

Such constructs are described herein as “Design 3” constructs and are shown schematically in FIG. 3. Design 3 constructs are configured such that only in cells with nuclear depletion of the hnRNP splicing factor is the intron spliced correctly. This has the effect that no part of the intron sequence is present in the mRNA product of the construct in cells with depletion of the hnRNP splicing factor. In contrast, in cells without nuclear depletion of the hnRNP splicing factor, the intron is not or incorrectly spliced. This has the effect that at least part of the intron is present in the mRNA product of the construct, which interrupts the transgene sequence and leads to a non-functional protein, and/or that an essential part of the transgene sequence is not included in the mature mRNA (see, e.g., FIG. 3, A to D). Additionally or alternatively, inclusion of all or part of the intron in the mature mRNA, and/or exclusion of part of the transgene sequence in the mature mRNA, induces a frame-shift, and the transgene comprises a premature termination codon which is only in frame with the start codon in the mRNA product of the construct when at least part of the single regulatory intron is incorporated into the mRNA product of the construct and/or a part of the transgene sequence is not included in the mature mRNA. Additionally, or alternatively, the part of the single regulatory intron incorporated into the mRNA product comprises a premature stop codon in frame with the start codon in the mRNA product of the construct (see, e.g., FIGS. 3, D and E). Additionally or alternatively, the part of the single regulatory intron incorporated into the mRNA product comprises a disruptive amino acid sequence.

In some embodiments, at least part of the transgene sequence is downstream of the single regulatory intron. In some embodiments, the complete transgene sequence is downstream of the regulatory domain. In some embodiments, part of the transgene sequence is upstream of the single regulatory intron, and part of the transgene sequence is downstream of the single regulatory intron. Other embodiments of the transgene sequence are as described herein. The transgene may be split into parts such that the first donor acceptor site and first splice acceptor site have a splicing score of at least 0.01 as determined by the Splice AI algorithm, or according to other splicing scores determined by the Splice AI algorithm as described herein. In some embodiments, the transgene sequence may be modified to include synonymous codon sequences.

In some embodiments, the binding domain for the splicing factor of the hnRNP family is within the single regulatory intron. In some embodiments, the binding domain for the splicing factor of the hnRNP family is upstream of the single regulatory intron (i.e., in the exonic sequence upstream of the first splice donor site). In some embodiments, the binding domain for the splicing factor of the hnRNP family is downstream of the single regulatory intron (i.e., in the exonic sequence downstream of the first splice acceptor site). In some examples, the binding domain is a TDP-43 binding domain and the hnRNP splicing factor is TDP-43. Other aspects of the hnRNP binding domain and/or TDP-43 binding domain are as elsewhere described herein. Other aspects of the first splice donor site, first splice acceptor site and transgene are as described herein.

In some embodiments, the first splice acceptor site and/or the first splice donor site have a splice score of 0.01 or above as determined by the Splice AI algorithm. In some embodiments, the first splice acceptor site and/or the first splice donor site have a splice score of 0.05 or above as determined by the Splice AI algorithm, or at least 0.1 or above, or at least 0.2 or above, or at least 0.3 or above, or at least 0.4 or above, or at least 0.5 or above, or at least 0.6 or above, or at least 0.7 or above, or at least 0.8 or above, or at least or equal to 0.9 or above as determined by Splice AI algorithm.

In some examples, the construct that has a sequence that has at least 80% sequence identity with SEQ ID NO: 95

In some examples, the single regulatory intron sequence has a sequence that has at least 80% sequence identity, or at least 85%, or at least 90%, or at least 95%, or at least 100% sequence identity with SEQ ID NO: 97, or SEQ ID NO: 163, or SEQ ID NO: 168, or SEQ ID NO: 171, or SEQ ID NO: 175.

In some examples, the exonic sequence upstream of the first splice donor site has a sequence that has at least 80% sequence identity, or at least 85%, or at least 90%, or at least 95%, or at least 100% sequence identity with SEQ ID NO: 29.

In some examples, the exonic sequence downstream of the first splice acceptor site has a sequence that has at least 80% sequence identity, or at least 85%, or at least 90%, or at least 95%, or at least 100% sequence identity with SEQ ID NO: 33.

In some examples, the TDP-43 binding domain has a sequence that has at least 80% sequence identity, or at least 85%, or at least 90%, or at least 95%, or at least 100% sequence identity with SEQ ID NO: 1-9, or SEQ ID NO: 115, or SEQ ID NO: 159 or SEQ ID NO: 160.

In some examples, the further intronic sequence has a sequence that has at least 80% sequence identity, or at least 85%, or at least 90%, or at least 95%, or at least 100% sequence identity with SEQ ID NO: 36.

In some embodiments, no splicing occurs in cells with no nuclear depletion of the hnRNP splicing factor, leading to intron retention in the mRNA product of the construct. The construct is configured such that the entire single regulatory intron is incorporated in the mRNA product of the construct in cells without depletion of the hnRNP splicing factor (i.e., wherein splicing of the first splice donor site and/or first splice acceptor site is repressed), but is not incorporated in the mRNA product of the construct in cells with depletion of the hnRNP splicing factor (i.e., wherein splicing of the first splice donor site and/or first splice acceptor site is not repressed).

In some examples, the regulatory domain comprises:
A splice donor site (i.e., the first splice donor site),
A single regulatory intron, and
A splice acceptor site (i.e., the first splice acceptor site).

In some examples, the construct comprises a transgene sequence (i.e., a transgene sequence encoding a functional protein) and a regulatory domain, the regulatory domain comprising (from upstream to downstream):

An optional coding sequence comprising a start codon,

An exonic sequence (i.e., immediately upstream of the splice donor site), A splice donor site (i.e., the first splice donor site),

A single regulatory intron,

A splice acceptor site (i.e., the first splice donor site) and

An exonic sequence (i.e., immediately downstream of the splice acceptor site).

The transgene sequence may be completely downstream of the regulatory domain. In other embodiments, the transgene sequence may be encoded by the exonic sequence

In some examples, the construct further comprises a further intronic sequence downstream of the exonic sequence. The binding domain for the hnRNP splicing factor may be within the single regulatory intron, upstream of the single regulatory intron in the exonic sequence immediately upstream of the splice donor site or downstream of the single regulatory intron immediately downstream of the splice acceptor site.

In some examples, the construct comprises (from upstream to downstream):
An optional coding sequence comprising a start codon,
An exonic sequence (i.e., optionally coding for at least part of the transgene),
A splice donor site (i.e., the first splice donor site),
A single regulatory intron,
A splice acceptor site (i.e., the first splice donor site) and
An exonic sequence,
A protein cleavage or self-cleaving site, and
A complete transgene sequence.

In some examples, the construct further comprises a further intronic sequence downstream of the exonic sequence. The binding domain for the hnRNP splicing factor may be within the single regulatory intron, upstream of the single regulatory intron in the exonic sequence immediately upstream of the splice donor site, or downstream of the single regulatory intron immediately downstream of the splice acceptor site.

In some examples, the construct comprises (from upstream to downstream):
An optional coding sequence comprising a start codon,
An exonic sequence (i.e., coding for a first part of the transgene),
A splice donor site (i.e., the first splice donor site),
A single regulatory intron,
A splice acceptor site (i.e., the first splice donor site) and
An exonic sequence (i.e., coding for a second part of the transgene).

In some examples, the construct further comprises a further intronic sequence downstream of the exonic sequence. The binding domain for the hnRNP splicing factor may be within the single regulatory intron, upstream of the single regulatory intron in the exonic sequence immediately upstream of the splice donor site, or downstream of the single regulatory intron immediately downstream of the splice acceptor site.

In some examples, the construct comprises (from upstream to downstream):
An optional coding sequence comprising a start codon,
An exonic sequence (i.e., coding for at least part of the transgene),
A splice donor site (i.e., the first splice donor site),
A single regulatory intron,
A splice acceptor site (i.e., the first splice acceptor site) and
An exonic sequence (i.e., coding for at least part of the transgene).

In alternative embodiments, incorrect or alternative splicing occurs in cells without nuclear depletion of the hnRNP splicing factor. In such embodiments, the construct and regulatory domain may comprise an alternative splice donor site and/or alternative splice acceptor site. In some embodiments, the alternative splice donor site may be upstream of the first splice donor site or may be within the single regulatory intron sequence (i.e., between the first splice donor site and the first splice acceptor site). In some embodiments, the alternative splice acceptor site may be downstream of the first acceptor site or may be within the single regulatory intron sequence (i.e., between the first splice donor site and the first splice acceptor site). An alternative splice acceptor site and/or alternative splice donor site may be any splice donor site that has a median splice SpliceAI score of at least 0.01 (99.8th percentile SpliceAI score), or at least 0.05, or at least 0.1, or at least 0.5, or least 0.9 as determined by the Splice AI algorithm as described elsewhere herein. The alternative splicing acceptor site and/or alternative splice donor site is not repressed by the hnRNP splicing factor (e.g., TDP-43). In some embodiments, the alternative splice acceptor site and/or alternative splice donor site is further away from the binding domain than the first splice acceptor site and the first splice donor site. In some embodiments, the alternative splice acceptor site and/or alternative splice donor site may be at least 20 nucleotides away from the binding domain, or at least 50 nucleotides away, or at least 100 nucleotides away from the binding domain, or at least 150 nucleotides away from the binding domain, or at least 200 nucleotides away from the binding domain.

In some embodiments, the construct is configured such that in cells without nuclear depletion of the hnRNP splicing factor (i.e., wherein splicing of the first splice donor site or first splice acceptor site is repressed), at least a part of the single regulatory intron is incorporated in the mRNA product, but in cells with nuclear depletion of the hnRNP splicing factor (i.e., wherein splicing of the first splice donor site or first splice acceptor site is not repressed), no part of the single regulatory intron is incorporated in the mRNA product of the construct.

Additionally or alternatively, the construct is configured such that in cells without nuclear depletion of the hnRNP splicing factor (i.e., wherein splicing of the first splice donor site or first splice acceptor site is repressed), at least part of the transgene sequence is not included in the mRNA product, but in cells with nuclear depletion of the hnRNP splicing factor (i.e., wherein splicing of the first splice donor site or first splice acceptor site is not repressed), all of the transgene sequence is present in the mRNA product of the construct.

In cells with nuclear depletion of the hnRNP splicing factor, the intron is fully spliced and removed to provide a complete and uninterrupted transgene sequence, in frame with the start codon and with no premature stop codons in frame with the start codon in the mRNA product of the construct such that a functional protein is produced.

In some examples, the regulatory domain comprises:
A splice donor site (i.e., the first splice donor site),
A single regulatory intron, i.e., defined by the first splice donor site and the first splice acceptor site,
A splice acceptor site (i.e., the first splice acceptor site), and
An alternative splice donor and/or an alternative splice acceptor site, which may be located within the single regulatory intron, upstream of the splice donor site or downstream of the splice acceptor site.

In some examples, the construct comprises (from upstream to downstream):
An optional coding sequence comprising a start codon,
An exonic sequence (i.e., immediately upstream of the splice donor site),
A splice donor site (i.e., the first splice donor site),
A single regulatory intron, (i.e., defined by the first splice donor site and the first splice acceptor site),
A splice acceptor site (i.e., the first splice acceptor site) and
An exonic sequence (immediately downstream of the splice acceptor site).

The binding domain for the hnRNP splicing factor may be within the single regulatory intron, or upstream or downstream of the single regulatory intron (i.e., in the exonic sequences flanking the single regulatory intron). The transgene may be completely downstream of the regulatory domain, or may be encoded by the exonic sequences upstream and downstream of the single regulatory intron. The alternative splice acceptor site may be within the single regulatory intron or downstream of the first splice acceptor site. The alternative splice donor site may be within the single regulatory intron or upstream of the first splice donor site. In some examples, the construct further comprises a further intronic sequence downstream of the exonic sequence.

In some examples, the construct comprises (from upstream to downstream):
An optional coding sequence comprising a start codon,
An exonic sequence,
A splice donor site (i.e., the first splice donor site),
A single regulatory intron, (i.e., defined the first splice donor site and the first splice acceptor site),
A splice acceptor site (i.e., the first splice acceptor site),
An exonic sequence,
An optional protein cleavage or self-cleaving site,
A complete transgene sequence

The binding domain for the hnRNP splicing factor which may be within the single regulatory intron, or upstream or downstream of the single regulatory intron (i.e., in the exonic sequences flanking the single regulatory intron. The alternative splice acceptor site may be within the single regulatory intron or downstream of the first splice acceptor site. The alternative splice donor site may be within the single regulatory intron or upstream of the first splice donor site. In some examples, the construct further comprises a further intronic sequence downstream of the exonic sequence

In some examples, the construct comprises (from upstream to downstream):
An optional coding sequence comprising a start codon,
An exonic sequence (i.e., coding for a first part of the transgene),
A splice donor site (i.e., the first splice donor site),
A single regulatory intron, (i.e., defined the first splice donor site and the first splice acceptor site),
A splice acceptor site (i.e., the first splice acceptor site) and
An exonic sequence (coding for a second part of the transgene).

The binding domain for the hnRNP splicing factor may be within the single regulatory intron, or upstream or downstream of the single regulatory intron (i.e., in the exonic sequences flanking the single regulatory intron). The alternative splice acceptor site may be within the single regulatory intron or downstream of the first splice acceptor site. The alternative splice donor site may be within the single regulatory intron or upstream of the first splice donor site. In some examples, the construct further comprises a further intronic sequence downstream of the exonic sequence.

### Optional Features

In all the above embodiments, the single regulatory intron, or at least part of the single regulatory intron (i.e., the part of the single regulatory intron that is incorrectly spliced and thus included in the mRNA product in cells without nuclear depletion of the hnRNP splicing factor) may comprise a premature termination codon (PTC) that is in frame with the start codon. This has the effect that in cells without nuclear depletion of hnRNP splicing factor, at least part of the intron may be present in the mRNA product of the construct, and a PTC is encountered, while in cells with nuclear depletion of the hnRNP splicing factor, the intron is not present in the mRNA product of the construct, such that no PTC is encountered.

In some embodiments, at least part of the transgene sequence downstream of the single regulatory intron comprises a PTC that is out of frame with the start codon when the intron is correctly spliced, but in frame with the start codon when the intron is not spliced or incorrectly spliced.

In some embodiments, the length of the single regulatory intron is not divisible by 3, i.e., such that incorporation of the single regulatory intron into the mRNA product of the construct introduces a frame-shift. In such embodiments, the construct may comprise a PTC downstream of the regulatory domain configured such that the PTC is out of frame with the start codon when no part of the single regulatory intron is incorporated into the mRNA product of the construct (i.e., when the intron is “correctly” spliced), but wherein the PTC is in frame with the start codon when the single regulatory intron is incorporated into the mRNA product of the construct (i.e., when the intron is not spliced).

In some embodiments, the single regulatory intron comprises a disruptive amino acid sequence.

In some embodiments, the construct further comprises a further intronic sequence which is at least 40 nucleotides downstream of the PTC. This leads to deposition of an EJC complex and promotes NMD of the mRNA when the PTC is in frame with the start codon.

In some embodiments, i.e., in embodiments where the transgene is completely downstream of the regulatory domain, the construct may further comprise a protease cleavage site or self-cleaving site.

### Vector

Disclosed herein is a vector comprising the construct according to any of the aspects or embodiments disclosed herein. In some embodiments, the vector is a DNA vector. In some embodiments, the vector is a circular vector, for example, in the form of a plasmid. In some embodiments, the vector is a single-stranded or double stranded vector, for example, double-stranded

In some embodiments, the vector is a viral vector. In some embodiments, the viral vector is a retrovirus, lentivirus, adenovirus (AV), or adeno-associated virus (AAV), chimeric AAV vector, or a herpes simplex viral vector. The viral vectors may be derived from any suitable serotype or subgroup. The viral vector may be a human viral vector or a non-human viral vector. In some embodiments, the AAV vector is a recombinant AAV vector.

In some embodiments, the viral vector comprises the construct described herein and one or more regions comprising inverted terminal repeat (ITR) sequences flanking the construct. In some embodiments, the sequence is operably linked to a promoter. Any suitable promoter may be used. In some examples, the promoter is a cytomegalovirus (CMV) promoter, a CMV enhancer, the CAG promoter, the SV40 promoter, the JeT promoter, the PGK promoter, and the chicken beta-actin promoter (CBA) promoter, eEF1A promoter, synapsin promoter, ChAT promoter, TRE promoter, calcium/calmodulin-dependent protein kinase II promoter, tubulin alpha I promoter, neuron-specific enolase promoter, or platelet-derived growth factor beta chain promoter, or fusions of the above.

In some embodiments, the promoter is a tissue-specific (e.g., CNS-specific) promoter. In some embodiments, the neuron specific promoter is derived from neuron-specific enolase (NSE) (see, e.g., EMBL HSEN02, X51956); an aromatic amino acid decarboxylase (MDC) promoter; a neurofilament promoter (see, e.g., GenBank HUMNFL, L04147); a synapsin promoter (see, e.g., GenBank HUMSYNIB, M55301); athy-1 promoter; a serotonin receptor promoter (see, e.g., GenBank S62283); a tyrosine hydroxylase promoter (TH); an L7 promoter; a DNMT promoter; an enkephalin promoter; a myelin basic protein (MBP) promoter; a Ca2+-calmodulin-dependent protein kinase II-alpha (CamKIM) promoter; a CMV enhancer/platelet-derived growth factor-p promoter.

In some embodiments, the vector comprises a polyadenylation site downstream of the construct. In some embodiments, the vector may comprise a post-transcriptional regulatory element (PRE) downstream of the construct.

### Pharmaceutical Composition

In one aspect of the present invention, there is provided a pharmaceutical composition comprising the construct or vector disclosed herein and a pharmaceutically acceptable excipient.

### System

In one aspect of the present invention, there is provided a system comprising a cell and any construct, vector or pharmaceutical composition described herein, wherein the system is configured such that
(i) upon depletion of the splicing factor of the hnRNP family from the cell nucleus, the system produces a functional protein, and
(ii) without depletion of the splicing factor of the hnRNP family from the cell nucleus, the system does not produce a functional protein

The system is such that cells only selectively express a functional protein upon depletion of the splicing factor from the nucleus (e.g., in a diseased cell), while functional protein is not produced without depletion of the splicing factor from the nucleus (e.g., in a healthy cell).

The cell may be any suitable cell. In some embodiments, the cell is a mammalian cell, more preferably a human cell. In preferred embodiments, the cell has nuclear depletion of the hnRNP splicing factor (e.g., depletion of TDP-43). In some embodiments, the cell is a brain cell. In some embodiments, the cell is a neuron or neuronal cell. In some embodiments, the cell is a microglial cell or astrocyte cell. In some embodiments, the cell is a muscle cell.

### Constructs, Vectors and Pharmaceutical Compositions for Use in Therapy and Related Methods

In a further aspect, there is provided the construct described herein, the vector described herein, or the pharmaceutical composition described herein, for use in therapy.

Also described herein, there is provided the construct described herein, the vector described herein, or the pharmaceutical composition described herein, for use in the treatment of a disease associated with depletion of a splicing factor of the hnRNP family. In some embodiments, the disease is a neurodegenerative disease. In some embodiments, the disease is a muscular disease or myopathy, e.g., a neuromuscular disease.

In a further aspect, there is provided the construct described herein, the vector described herein, or the pharmaceutical composition described herein, for use in the treatment of a disease associated with depletion of the TDP-43. In some embodiments, the disease is a neurodegenerative disease. In some embodiments, the disease is a muscular disease, e.g., a neuromuscular disease.

In some embodiments, the disease (e.g., neurodegenerative disease) is selected from amyotrophic lateral sclerosis (ALS), frontotemporal dementia (FTD), Parkinson's disease, Alzheimer's disease, inclusion body myopathy, or Perry syndrome.

In a further aspect, there is provided the construct described herein, the vector described herein, or the pharmaceutical composition described herein, for use in the treatment of a neuromuscular disease is associated with depletion of the splicing factor of the hnRNP family. In some embodiments, the splicing factor of the hnRNP family is TDP-43.

The construct, vector or pharmaceutical composition described herein may be administered using any suitable method.

In some embodiments, the treatment of the disease comprises contacting a cell with the construct, vector, or pharmaceutical composition disclosed herein. The treatment is such that
(i) in a cell with nuclear depletion of the splicing factor (i.e., when the cell nucleus is depleted of splicing factor), the cell produces a functional protein,
(ii) In a cell without nuclear depletion of the splicing factor (i.e., when the cell nucleus is depleted of the splicing factor), the cell produces does not produce a functional protein.

Also disclosed herein, is a method of treatment for a disease associated with depletion of the hnRNP splicing factor (e.g., a neurodegenerative or muscular disease, for example, associated with depletion of TDP-43), the method of treatment comprising contacting the cell with the construct, vector, or pharmaceutical composition disclosed herein. In preferred embodiments, the disease is associated with depletion of TDP-43. The method of treatment is such that
(i) in a cell with nuclear depletion of the splicing factor, the cell produces a functional protein,
(ii) In a cell without nuclear depletion of the splicing factor, the cell produces does not produce a functional protein.

Also disclosed herein, is the construct described herein, vector described herein, or pharmaceutical composition described herein for use in the manufacture of a medicament. The medicament may be used for the treatment of a disease associated with depletion of a hnRNP splicing factor (e.g., a neurodegenerative disease or neuromuscular disease, e.g., associated with depletion of TDP-43), and wherein the treatment comprises contacting the cell with the construct, vector, or pharmaceutical composition disclosed herein. In preferred embodiments, the disease is associated with depletion of TDP-43.

The method of treatment is such that
(i) in a cell with nuclear depletion of the splicing factor, the cell produces a functional protein,
(ii) In a cell without nuclear depletion of the splicing factor, the cell produces does not produce a functional protein.

In a further aspect, there is provided the use of the construct, use of the vector, or use of the pharmaceutical composition disclosed herein, in a method of selectively producing functional protein in a diseased cell that has nuclear depletion of a splicing factor of the hnRNP family. In preferred embodiments, the splicing factor of the hnRNP family is TDP-43. The cells may be in vivo or in vitro.

### In Vitro System

Also disclosed herein, is a construct comprising
a start codon,
a regulatory domain comprising:
   a first splice acceptor site and a first splice donor site,
   a binding domain for a splicing factor of the heterogenous nuclear ribonucleoprotein (hnRNP) family, located within 150 nucleotides of the first splice donor site or first splice acceptor site and/or located between the first splice donor site and first splice acceptor site; and
a transgene sequence (i.e., a transgene sequence encoding a functional protein),
wherein the construct is configured such that
   (i) if placed in an in vitro system with depletion of the splicing factor, splicing of the first splice acceptor site and first donor site is not repressed, such that a functional protein is produced from the transgene sequence (i.e., a functional protein is produced from the transgene sequence from the mRNA product of the construct where the functional protein is encoded by the complete, uninterrupted transgene sequence)
   (ii) if placed in a vitro system with without depletion of the splicing factor, splicing of the first splice acceptor site and/or first donor site is repressed such that no functional protein is produced from the transgene sequence (i.e., a functional protein is produced from the transgene sequence from the from the mRNA product of the construct where the functional protein is encoded by the complete, uninterrupted transgene sequence)

The in vitro system must comprise components which enable transcription, splicing and translation. In some embodiments, the components are provided by a cell.

In some embodiments, there is provided the use of the construct in an in vitro system for selectively producing functional protein in the absence of a splicing factor of the hnRNP family. In preferred embodiments, the splicing factor of the hnRNP family is TDP-43

### EXAMPLES

### Design 1

### Example 1

An example construct of the present invention has a structure according to “Design 1” as shown in FIG. 1. Constructs of Design 1 comprise a regulatory domain comprising an intronic sequence comprising a TDP-43 binding domain, and a cryptic exon sequence embedded within the intronic region. The cryptic exon sequence is defined by a first splice acceptor site and a first splice donor site (i.e., “cryptic splice sites”), and the intronic region is defined by a second splice donor site and second splice acceptor. The construct further comprises a transgene sequence downstream of the regulatory domain which encodes for a protein (e.g., a functional or diagnostic protein).

For this construct, binding of TDP-43 to the binding domain represses splicing of the cryptic splice acceptor and/or cryptic splice donor site. Due to the role that exon definition plays in determining splicing, repression of one cryptic splice site can also repress the other. This has the result that in healthy cells (i.e., not depleted of splicing factor), the cryptic exon sequence is not present in the mRNA product of the construct. In contrast, in diseased cells (i.e., depleted of splicing factor), the cryptic exon sequence is present in the mRNA product of the construct. This can be used to control the expression of downstream transgene.

### Example 1A

In this Example, the regulatory domain is based on a modified portion of the AARS1 sequence between exon 4 and exon 5, and the transgene is a sequence that encodes for mCherry (a red fluorescent protein).

The first example construct (SEQ ID NO: 25) comprises the following features, listed from 5′→3′
Sequence encoding a start codon
A regulatory domain (SEQ ID NO: 26) comprising:
A 3′ exonic sequence (here, based on exon 4 of AARS1)
A cryptic exon sequence embedded within an intronic region. The cryptic exon sequence is defined by a splice acceptor site and splice donor site, where at least one of these splice sites is repressed by TDP-43 binding. The intronic region itself is defined by a second splice donor site and second splice acceptor site. The intronic region comprises a first intronic part upstream of the cryptic exon sequence and a second intronic part downstream of the cryptic exon sequence, and comprises a TDP-43 binding domain. The full intronic sequence, when the cryptic exon is not included, contains, from 5′ to 3′, the first intronic part, the cryptic exon, and the second intronic part.
A 5′ exonic sequence (here, based on exon 5 of AARS1, with a single point mutation)
Sequence for a protease cleavage site or self-cleaving site (here, a P2A self-cleaving site)
A complete transgene sequence (here, encoding for mCherry)·
A further intron sequence comprising a downstream intron in an exonic context (here, based on human RPS24)

In this example, the regulatory domain was based on a modified AARS1 gene. As compared with the naturally occurring sequence, large sections of intronic region were removed (reduced from 6.5 kb to 0.6 kb) such that the intronic regions only comprise the cryptic exon, regions flanking the cryptic exon sequence and cryptic splice sites (i.e., which form the first splice acceptor and first splice donor sites in the construct) and constitutive splice sites (i.e., which form the second splice acceptor and second splice donor sites). Additionally, the TG-repeat region (i.e., the TDP-43 binding sequence) was slightly modified to perform more effective gene synthesis, where an “AA” was inserted into the middle TG-repeat to make it less repetitive. Next, the 5′ exonic sequence based on exon 5 of AARS1 was mutated to avoid a premature stop codon. The cryptic exon sequence was also modified as compared with what occurs naturally to include an additional adenosine within the sequence. This gave the cryptic exon (CE) a total length of 88 nucleotides (rather than 87 nucleotides), which is not divisible by 3. This had the effect that the cryptic exon can perform a frame-shifting function when included in the mRNA product of the construct. In diseased cells, inclusion of the cryptic exon sequence means that the premature stop codon, downstream of the cryptic exon, is no longer in frame with the start codon; this leads to the production of a functional protein. In healthy cells, the cryptic exon sequence is not included, and the premature termination codon is encountered because it is in frame with the start codon. This leads to the formation of a truncated and non-functional protein, with no amino acid similarity to mCherry due to the frame shift.

In this example, the cryptic splice acceptor site (i.e., the first acceptor splice site) has a splice score of 0.05 as determined by the Splice AI algorithm and the cryptic splice donor site (i.e., the first splice donor site) has a splice score of 0.19 as determined by the Splice AI algorithm.

Sequences used in the example construct are tabulated below:

| | SEQ ID | |
|---|---|---|
| | NO: | Sequence |
| Construct 1A | 25 | GGTTTAGTGAACCGTCAGATCAGATCTTTGTCGATCCTACCATCCACTCG |
| | | ACACACCCGCCAGCGGCCGCTTCTTGGTGCCAGCTTATCATAGCGCTAC |
| | | CGGTCGCCACCATGGCGAGAACCATGGTAGCCATGGAGACCATGGGGCT |
| | | CATGACAACAGATCTGGCAAAATTTGGGGTAAGAATGCACATCACTTCTT |
| | | GAGAGTATGGAGGAGTGAAATGACACTCAGTGCCAGAGTTACTGTATATC |
| | | TACACTTTAAAAGTGTAGCTTTTAAAAGATAAGCAAGCACAATCTTTTGTGT |
| | | GTGTGTGTGTGAATGTGTGTGTGTGTGTGTGTCACCCAGGCTGGAGTGC |
| | | AGTGGCATGATCACAGCTCACTGCAGCCTCAAACTTCCTGGGCTCAAGTG |
| | | ATCCTCTCCCGAGTAGCTGGGACTACAGGTATGCATCACCCCCCCAGCTA |
| | | ATTTTTTTTTGTATTTTTTACCGAGTCGGGGTTTCGCAATGTTGCCCAGGC |
| | | TGGTCTCAGAGTCTCGCTCTGTTGTCTACGCTGGAGTGCAGTAACATGAG |
| | | CCACTGTGCCCGGCCAATCCTAAGAATTTCTTTTGCGGTGGTTGCAAGTC |
| | | TGGGCAGAACTCTTGTCAGGGGCTGTAACTGGACTTATCTTTACTCCTTT |
| | | GTCAGGCTGGATGCCACCAAAATCCTCCCAGGCAACATACGGCAGCGGC |
| | | GCCACCAACTTTTCCCTGCTCAAGCAAGCCGGCGACGTGGAAGAGAATC |
| | | CCGGCCCCGTCAGCAAAGGGGAAGAGGACAACATGGCCATCATTAAGGA |
| | | GTTTATGCGATTCAAAGTACACATGGAGGGATCTGTTAATGGCCATGAATT |
| | | TGAGATAGAGGGGGAAGGTGAGGGTCGCCCTTACGAAGGCACGCAGAC |
| | | GGCTAAGCTGAAGGTCACGAAAGGGGGACCCTTGCCCTTCGCATGGGAC |
| | | ATACTCTCCCCACAGTTTATGTATGGTTCTAAGGCATATGTTAAGCACCCT |
| | | GCAGACATCCCAGACTATCTGAAGCTCTCCTTTCCTGAGGGGTTTAAGTG |
| | | GGAACGCGTTATGAACTTTGAGGATGGAGGGGTCGTGACTGTTACCCAG |
| | | GATTCTTCCCTGCAAGATGGAGAGTTCATATACAAAGTGAAACTTCGGGG |
| | | AACGAATTTCCCATCAGACGGGCCAGTGATGCAGAAAAAGACGATGGGG |
| | | TGGGAGGCTTCATCCGAGAGGATGTATCCCGAGGACGGAGCATTGAAAG |
| | | GCGAAATAAAACAAAGGCTGAAGTTGAAGGATGGGGGCCACTACGACGC |
| | | GGAGGTTAAAACAACGTATAAAGCTAAAAAGCCAGTACAGCTCCCAGGCG |
| | | CATATAACGTGAATATAAAGCTTGACATAACGAGTCATAACGAGGATTACA |
| | | CAATCGTAGAACAGTACGAAAGAGCTGAAGGACGGCACTCCACCGGTGG |
| | | GATGGATGAACTCTATAAATAAACAAATGGTAAGGAAGGGCACATCAATC |
| | | TTTGCTTAATTGTCCTTTACTCTAAAGATGTATTTTATCATACTGAATGCTA |
| | | AACTTGATATCTCCTTTTAGGTCATTGATGTCCTTCACCCCGGGAAGGCG |
| | | ACAGTGCCTAAGACAGAAATTCGGGAAAAACTAGCCAAAATGTACAAGAC |
| | | CACACCGGATGTCATCTTTGTATTTGGATTCAGAACTCA |
| Regulatory | 26 | ATGACAACAGATCTGGCAAAATTTGGGGTAAGAATGCACATCACTTCTTG |
| Domain (cryptic | | AGAGTATGGAGGAGTGAAATGACACTCAGTGCCAGAGTTACTGTATATCT |
| exon, intronic | | ACACTTTAAAAGTGTAGCTTTTAAAAGATAAGCAAGCACAATCTTTTGTGT |
| regions and | | GTGTGTGTGTGAATGTGTGTGTGTGTGTGTGTCACCCAGGCTGGAGTGC |
| flanking exons) | | AGTGGCATGATCACAGCTCACTGCAGCCTCAAACTTCCTGGGCTCAAGTG |
| | | ATCCTCTCCCGAGTAGCTGGGACTACAGGTATGCATCACCCCCCCAGCTA |
| | | ATTTTTTTTTGTATTTTTTACCGAGTCGGGGTTTCGCAATGTTGCCCAGGC |
| | | TGGTCTCAGAGTCTCGCTCTGTTGTCTACGCTGGAGTGCAGTAACATGAG |
| | | CCACTGTGCCCGGCCAATCCTAAGAATTTCTTTTGCGGTGGTTGCAAGTC |
| | | TGGGCAGAACTCTTGTCAGGGGCTGTAACTGGACTTATCTTTACTCCTTT |
| | | GTCAGGCTGGATGCCACCAAAATCCTCCCAGGCAACATAC |
| Intronic region | 27 | GTAAGAATGCACATCACTTCTTGAGAGTATGGAGGAGTGAAATGACACTC |
| (including first part | | AGTGCCAGAGTTACTGTATATCTACACTTTAAAAGTGTAGCTTTTAAAAGA |
| of intronic region, | | TAAGCAAGCACAATCTTTTGTGTGTGTGTGTGTGAATGTGTGTGTGTGTGT |
| cryptic exon, and | | GTGTCACCCAGGCTGGAGTGCAGTGGCATGATCACAGCTCACTGCAGCC |
| second part of | | TCAAACTTCCTGGGCTCAAGTGATCCTCTCCCGAGTAGCTGGGACTACAG |
| intronic region) | | GTATGCATCACCCCCCCAGCTAATTTTTTTTTGTATTTTTTACCGAGTCGG |
| | | GGTTTCGCAATGTTGCCCAGGCTGGTCTCAGAGTCTCGCTCTGTTGTCTA |
| | | CGCTGGAGTGCAGTAACATGAGCCACTGTGCCCGGCCAATCCTAAGAAT |
| | | TTCTTTTGCGGTGGTTGCAAGTCTGGGCAGAACTCTTGTCAGGGGCTGTA |
| | | ACTGGACTTATCTTTACTCCTTTGTCAG |
| Sequence | 28 | GGTTTAGTGAACCGTCAGATCAGATCTTTGTCGATCCTACCATCCACTCG |
| encoding a start | | ACACACCCGCCAGCGGCCGCTTCTTGGTGCCAGCTTATCATAGCGCTAC |
| codon (start | | CGGTCGCCACCATGGCGAGAACCATGGTAGCCATGGAGACCATGGGGC |
| codon underlined | | TCATGACA |
| and in bold) | | |
| 3′ exonic | 29 | ACAGATCTGGCAAAATTTGGG |
| sequence | | |
| (AARS1 exon 4) | | |
| First part of | 30 | GTAAGAATGCACATCACTTCTTGAGAGTATGGAGGAGTGAAATGACACTC |
| intronic region | | AGTGCCAGAGTTACTGTATATCTACACTTTAAAAGTGTAGCTTTTAAAAGA |
| (derived from | | TAAGCAAGCACAATCTTTTGTGTGTGTGTGTGTGAATGTGTGTGTGTGTGT |
| AARS1 and | | GTGTCACCCAG |
| proceeding cryptic | | |
| exon sequence | | |
| (TDP-43 binding | | |
| domain | | |
| underlined)) | | |
| Cryptic exon | 31 | GCTGGAGTGCAGTGGCATGATCACAGCTCACTGCAGCCTCAAACTTCCT |
| sequence (based | | GGGCTCAAGTGATCCTCTCCCGAGTAGCTGGGACTACAG |
| on AARS1, with | | |
| inserted | | |
| nucleotide in bold | | |
| and underlined) | | |
| Second part of | 32 | GTATGCATCACCCCCCCAGCTAATTTTTTTTTGTATTTTTTACCGAGTCG |
| intronic region, | | GGGTTTCGCAATGTTGCCCAGGCTGGTCTCAGAGTCTCGCTCTGTTGTCT |
| (derived from | | ACGCTGGAGTGCAGTAACATGAGCCACTGTGCCCGGCCAATCCTAAGAAT |
| AARS1 and | | TTCTTTTGCGGTGGTTGCAAGTCTGGGCAGAACTCTTGTCAGGGGCTGTA |
| following cryptic | | ACTGGACTTATCTTTACTCCTTTGTCAG |
| exon sequence) | | |
| 5′ exonic | 33 | GCTGGATGCCACCAAAATCCTCCCAGGCAACAT |
| sequence | | |
| (Sequence based | | |
| on 5′ region | | |
| AARS1 exon 5, | | |
| shown with | | |
| mutated | | |
| nucleotide A→C | | |
| in bold and | | |
| underlined) | | |
| P2A cleavage site | 34 | GGCAGCGGCGCCACCAACTTTTCCCTGCTCAAGCAAGCCGGCGACGTGG |
| | | AAGAGAATCCCGGCCCC |
| Transgene | 35 | GTCAGCAAAGGGGAAGAGGACAACATGGCCATCATTAAGGAGTTTATGC |
| sequence for | | GATTCAAAGTACACATGGAGGGATCTGTTAATGGCCATGAATTTGAGAT |
| mCherry | | AGAGGGGGAAGGTGAGGGTCGCCCTTACGAAGGCACGCAGACGGCTAAG |
| (premature stop | | CTGAAGGTCACGAAAGGGGGACCCTTGCCCTTCGCATGGGACATACTCT |
| codon shown in | | CCCCACAGTTTATGTATGGTTCTAAGGCATATGTTAAGCACCCTGCAGAC |
| bold and | | ATCCCAGACTATCTGAAGCTCTCCTTTCCTGAGGGGTTTAAGTGGGAACG |
| underlined) | | CGTTATGAACTTTGAGGATGGAGGGGTCGTGACTGTTACCCAGGATTCTT |
| | | CCCTGCAAGATGGAGAGTTCATATACAAAGTGAAACTTCGGGGAACGAAT |
| | | TTCCCATCAGACGGGCCAGTGATGCAGAAAAAGACGATGGGGTGGGAGG |
| | | CTTCATCCGAGAGGATGTATCCCGAGGACGGAGCATTGAAAGGCGAAAT |
| | | AAAACAAAGGCTGAAGTTGAAGGATGGGGGCCACTACGACGCGGAGGTT |
| | | AAAACAACGTATAAAGCTAAAAAGCCAGTACAGCTCCCAGGCGCATATA |
| | | ACGTGAATATAAAGCTTGACATAACGAGTCATAACGAGGATTACACAAT |
| | | CGTAGAACAGTACGAAAGAGCTGAAGGACGGCACTCCACCGGTGGGATG |
| | | GATGAACTCTATAAA |
| Further intronic | 36 | ACAAATGGTAAGGAAGGGCACATCAATCTTTGCTTAATTGTCCTTTACTCT |
| sequence | | AAAGATGTATTTTATCATACTGAATGCTAAACTTGATATCTCCTTTTAGGT |
| (comprising a | | CATTGATGTCCTTCACCCCGGGAAGGCGACAGTGCCTAAGACAGAAATTCG |
| downstream | | GGAAAAACTAGCCAAAATGTACAAGACCACACCGGATGTCATCTTTGTATT |
| constitutively | | TGGATTCAGAACTCA |
| spliced intron | | |
| within exonic | | |
| context, based on | | |
| RPS24, intronic | | |
| sequence is | | |
| shown″ | | |
| underlined) | | |
| Coding sequence | 37 | ATGGCGAGAACCATGGTAGCCATGGAGACCATGGGGCTCATGACAACAGATCTGG |
| without cryptic | | CAAAATTTGGGGCTGGATGCCACCAAAATCCTCCCAGGCAACATACGGCAGCGGC |
| exon (premature | | GCCACCAACTTTTCCCTGCTCAAGCAAGCCGGCGACGTGGAAGAGAATCCCGGCC |
| stop codon in bold | | CCGTCAGCAAAGGGGAAGAGGACAACATGGCCATCATTAAGGAGTTTATGCGATT |
| and underlined) | | CAAAGTACACATGGAGGGATCTGTTAATGGCCATGAATTTGAGATAG |
| Encoded amino | 38 | MARTMVAMETMGLMTTDLAKFGAGCHQNPPRQHTAAAPPTFPCSSKPATW |
| acid sequence | | KRIPAPSAKGKRTTWPSLRSLCDSKYTWRDLLMAMNLR* |
| without cryptic | | |
| exon | | |
| Coding sequence | 39 | ATGGCGAGAACCATGGTAGCCATGGAGACCATGGGGCTCATGACAACAG |
| with the cryptic | | ATCTGGCAAAATTTGGGGCTGGAGTGCAGTGGCATGATCACAGCTCACT |
| exon | | GCAGCCTCAAACTTCCTGGGCTCAAGTGATCCTCTCCCGAGTAGCTGGG |
| | | ACTACAGGCTGGATGCCACCAAAATCCTCCCAGGCAACATACGGCAGCG |
| | | GCGCCACCAACTTTTCCCTGCTCAAGCAAGCCGGCGACGTGGAAGAGAA |
| | | TCCCGGCCCCGTCAGCAAAGGGGAAGAGGACAACATGGCCATCATTAAG |
| | | GAGTTTATGCGATTCAAAGTACACATGGAGGGATCTGTTAATGGCCATGA |
| | | ATTTGAGATAGAGGGGGAAGGTGAGGGTCGCCCTTACGAAGGCACGCAG |
| | | ACGGCTAAGCTGAAGGTCACGAAAGGGGGACCCTTGCCCTTCGCATGGGA |
| | | CATACTCTCCCCACAGTTTATGTATGGTTCTAAGGCATATGTTAAGCACC |
| | | CTGCAGACATCCCAGACTATCTGAAGCTCTCCTTTCCTGAGGGGTTTAAG |
| | | TGGGAACGCGTTATGAACTTTGAGGATGGAGGGGTCGTGACTGTTACCC |
| | | AGGATTCTTCCCTGCAAGATGGAGAGTTCATATACAAAGTGAAACTTCGG |
| | | GGAACGAATTTCCCATCAGACGGGCCAGTGATGCAGAAAAAGACGATGG |
| | | GGTGGGAGGCTTCATCCGAGAGGATGTATCCCGAGGACGGAGCATTGAA |
| | | AGGCGAAATAAAACAAAGGCTGAAGTTGAAGGATGGGGGCCACTACGAC |
| | | GCGGAGGTTAAAACAACGTATAAAGCTAAAAAGCCAGTACAGCTCCCAGG |
| | | CGCATATAACGTGAATATAAAGCTTGACATAACGAGTCATAACGAGGATT |
| | | ACACAATCGTAGAACAGTACGAAAGAGCTGAAGGACGGCACTCCACCGGT |
| | | GGGATGGATGAACTCTATAAATAA |
| Encoded amino | 40 | MARTMVAMETMGLMTTDLAKFGAGVQWHDHSSLQPQTSWAQVILSRVAGTT |
| acid sequence | | GWMPPKSSQATYGSGATNFSLLKQAGDVEENPGPVSKGEEDNMAIIKEFMR |
| with the cryptic | | FKVHMEGSVNGHEFEIEGEGEGRPYEGTQTAKLKVTKGGPLPFAWDILSP |
| exon (mCherry | | QFMYGSKAYVKHPADIPDYLKLSFPEGFKWERVMNFEDGGVVTVTQDSSL |
| sequence in bold, | | QDGEFIYKVKLRGTNFPSDGPVMQKKTMGWEASSERMYPEDGALKGEIKQ |
| self-cleaving P2A | | RLKLKDGGHYDAEVKTTYKAKKPVQLPGAYNVNIKLDITSHNEDYTIVEQ |
| sequence in | | YERAEGRHSTGGMDELYK* |
| italics) | | |

The above example construct was incorporated into a plasmid. In addition to the features described above, the plasmid further comprises an enhancer sequence and a promoter sequence upstream of the construct (here, a CMV enhancer and CMV promoter respectively) and polyadenylation site downstream of the construct (here an SV40 late polyA site).

This example plasmid also contained sequence elements for propagation in bacteria, namely an origin of replication (in this case ColE1 origin) and an antibiotic selection gene (in this case AmpR for ampicillin resistance). These features would not be relevant for use in mammalian cells and therefore can be omitted.

The plasmid had the following sequence, SEQ ID NO: 41

| | |
|---|---|
| 1 | ATATATGGAG TTCCGCGTTA CATAACTTAC GGTAAATGGC CCGCCTGGCT GACCGCCCAA |
| 61 | CGACCCCCGC CCATTGACGT CAATAATGAC GTATGTTCCC ATAGTAACGC CAATAGGGAC |
| 121 | TTTCCATTGA CGTCAATGGG TGGAGTATTT ACGGTAAACT GCCCACTTGG CAGTACATCA |
| 181 | AGTGTATCAT ATGCCAAGTA CGCCCCCTAT TGACGTCAAT GACGGTAAAT GGCCCGCCTG |
| 241 | GCATTATGCC CAGTACATGA CCTTATGGGA CTTTCCTACT TGGCAGTACA TCTACGTATT |
| 301 | AGTCATCGCT ATTACCATGC TGATGCGGTT TTGGCAGTAC ATCAATGGGC GTGGATAGCG |
| 361 | GTTTGACTCA CGGGGATTTC CAAGTCTCCA CCCCATTGAC GTCAATGGGA GTTTGTTTTG |
| 421 | GCACCAAAAT CAACGGGACT TTCCAAAATG TCGTAACAAC TCCGCCCCAT TGACGCAAAT |
| 481 | GGGCGGTAGG CGTGTACGGT GGGAGGTCTA TATAAGCAGA GCTGGTTTAG TGAACCGTCA |
| 541 | GATCAGATCT TTGTCGATCC TACCATCCAC TCGACACACC CGCCAGCGGC CGCTTCTTGG |
| 601 | TGCCAGCTTA TCAtagcgct accggtcgcc accatggCga gaACCATGGT AGCCATGGAG |
| 661 | accATGgggc tCATGACAAC AGATCTGGCA AAATTTGGGG TAAGAATGCA CATCACTTCT |
| 721 | TGAGAGTATG GAGGAGTGAA ATGACACTCA GTGCCAGAGT TACTGTATAT CTACACTTTA |
| 781 | AAAGTGTAGC TTTTAAAAGA TAAGCAAGCA CAATCTTTTG TGTGTGTGTG TGTGAATGTG |
| 841 | TGTGTGTGTG TGTGTCACCC AGGCTGGAGT GCAGTGGCAT GATCACAGCT CACTGCAGCC |
| 901 | TCAAACTTCC TGGGCTCAAG TGATCCTCTC CCGAGTAGCT GGGACTACAG GTATGCATCA |
| 961 | CCCCCCCAGC TAATTTTTTT TTGTATTTTT TACCGAGTCG GGGTTTCGCA ATGTTGCCCA |
| 1021 | GGCTGGTCTC AGAGTCTCGC TCTGTTGTCT ACGCTGGAGT GCAGTAACAT GAGCCACTGT |
| 1081 | GCCCGGCCAA TCCTAAGAAT TTCTTTTGCG GTGGTTGCAA GTCTGGGCAG AACTCTTGTC |
| 1141 | AGGGGCTGTA ACTGGACTTA TCTTTACTCC TTTGTCAGGC TGGATGCCAC CAAAATCCTC |
| 1201 | CCAGGCAACA TACggcagcg gcgccaccaa cttttccctg ctcaagcaag ccggcgacgt |
| 1261 | ggaagagaat cccggccccG TCAGCAAAGG GGAAGAGGAC AACATGGCCA TCATTAAGGA |
| 1321 | GTTTATGCGA TTCAAAGTAC ACATGGAGGG ATCTGTTAAT GGCCATGAAT TTGAGATAGA |
| 1381 | GGGGGAAGGT GAGGGTCGCC CTTACGAAGG CACGCAGACG GCTAAGCTGA AGGTCACGAA |
| 1441 | AGGGGGACCC TTGCCCTTCG CATGGGACAT ACTCTCCCCA CAGTTTATGT ATGGTTCTAA |
| 1501 | GGCATATGTT AAGCACCCTG CAGACATCCC AGACTATCTG AAGCTCTCCT TTCCTGAGGG |
| 1561 | GTTTAAGTGG GAACGCGTTA TGAACTTTGA GGATGGAGGG GTCGTGACTG TTACCCAGGA |
| 1621 | TTCTTCCCTG CAAGATGGAG AGTTCATATA CAAAGTGAAA CTTCGGGGAA CGAATTTCCC |
| 1681 | ATCAGACGGG CCAGTGATGC AGAAAAAGAC GATGGGGTGG GAGGCTTCAT CCGAGAGGAT |
| 1741 | GTATCCCGAG GACGGAGCAT TGAAAGGCGA AATAAAACAA AGGCTGAAGT TGAAGGATGG |
| 1801 | GGGCCACTAC GACGCGGAGG TTAAAACAAC GTATAAAGCT AAAAAGCCAG TACAGCTCCC |
| 1861 | AGGCGCATAT AACGTGAATA TAAAGCTTGA CATAACGAGT CATAACGAGG ATTACACAAT |
| 1921 | CGTAGAACAG TACGAAAGAG CTGAAGGACG GCACTCCACC GGTGGGATGG ATGAACTCTA |
| 1981 | TAAATAAACA AATGGTAAGG AAGGGCACAT CAATCTTTGC TTAATTGTCC TTTACTCTAA |
| 2041 | AGATGTATTT TATCATACTG AATGCTAAAC TTGATATCTC CTTTTAGGTC ATTGATGTCC |
| 2101 | TTCACCCCGG GAAGGCGACA GTGCCTAAGA CAGAAATTCG GGAAAAACTA GCCAAAATGT |
| 2161 | ACAAGACCAC ACCGGATGTC ATCTTTGTAT TTGGATTCAG AACTCAGTAA ACTGGATCCG |
| 2221 | CAGGCCTCTG CTAGCTTGAC TGACTGAGAT ACAGCGTACC TTCAGCTCAC AGACATGATA |
| 2281 | AGATACATTG ATGAGTTTGG ACAAACCACA ACTAGAATGC AGTGAAAAAA ATGCTTTATT |
| 2341 | TGTGAAATTT GTGATGCTAT TGCTTTATTT GTAACCATTA TAAGCTGCAA TAAACAAGTT |
| 2401 | AACAACAACA ATTGCATTCA TTTTATGTTT CAGGTTCAGG GGGAGGTGTG GGAGGTTTTT |
| 2461 | TAAAGCAAGT AAAACCTCTA CAAATGTGGT ATTGGCCCAT CTCTATCGGT ATCGTAGCAT |
| 2521 | AACCCCTTGG GGCCTCTAAA CGGGTCTTGA GGGGTTTTTT GTGCCCCTCG GGCCGGATTG |
| 2581 | CTATCTACCG GCATTGGCGC AGAAAAAAAT GCCTGATGCG ACGCTGCGCG TCTTATACTC |
| 2641 | CCACATATGC CAGATTCAGC AACGGATACG GCTTCCCCAA CTTGCCCACT TCCATACGTG |
| 2701 | TCCTCCTTAC CAGAAATTTA TCCTTAAGGT CGTCAGCTAT CCTGCAGGCG ATCTCTCGAT |
| 2761 | TTCGATCAAG ACATTCCTTT AATGGTCTTT TCTGGACACC ACTAGGGGTC AGAAGTAGTT |
| 2821 | CATCAAACTT TCTTCCCTCC CTAATCTCAT TGGTTACCTT GGGCTATCGA AACTTAATTA |
| 2881 | ACCAGTCAAG TCAGCTACTT GGCGAGATCG ACTTGTCTGG GTTTCGACTA CGCTCAGAAT |
| 2941 | TGCGTCAGTC AAGTTCGATC TGGTCCTTGC TATTGCACCC GTTCTCCGAT TACGAGTTTC |
| 3001 | ATTTAAATCA TGTGAGCAAA AGGCCAGCAA AAGGCCAGGA ACCGTAAAAA GGCCGCGTTG |
| 3061 | CTGGCGTTTT TCCATAGGCT CCGCCCCCCT GACGAGCATC ACAAAAATCG ACGCTCAAGT |
| 3121 | CAGAGGTGGC GAAACCCGAC AGGACTATAA AGATACCAGG CGTTTCCCCC TGGAAGCTCC |
| 3181 | CTCGTGCGCT CTCCTGTTCC GACCCTGCCG CTTACCGGAT ACCTGTCCGC CTTTCTCCCT |
| 3241 | TCGGGAAGCG TGGCGCTTTC TCATAGCTCA CGCTGTAGGT ATCTCAGTTC GGTGTAGGTC |
| 3301 | GTTCGCTCCA AGCTGGGCTG TGTGCACGAA CCCCCCGTTC AGCCCGACCG CTGCGCCTTA |
| 3361 | TCCGGTAACT ATCGTCTTGA GTCCAACCCG GTAAGACACG ACTTATCGCC ACTGGCAGCA |
| 3421 | GCCACTGGTA ACAGGATTAG CAGAGCGAGG TATGTAGGCG GTGCTACAGA GTTCTTGAAG |
| 3481 | TGGTGGCCTA ACTACGGCTA CACTAGAAGA ACAGTATTTG GTATCTGCGC TCTGCTGAAG |
| 3541 | CCAGTTACCT TCGGAAAAAG AGTTGGTAGC TCTTGATCCG GCAAACAAAC CACCGCTGGT |
| 3601 | AGCGGTGGTT TTTTTGTTTG CAAGCAGCAG ATTACGCGCA GAAAAAAAGG ATCTCAAGAA |
| 3661 | GATCCTTTGA TCTTTTCTAC GGGGTCTGAC GCTCAGTGGA ACGAAAACTC ACGTTAAGGG |
| 3721 | ATTTTGGTCA TGAGATTATC AAAAAGGATC TTCACCTAGA TCCTTTTAAA TTAAAAATGA |
| 3781 | AGTTTTAAAT CAATCTAAAG TATATATGAG TAAACTTGGT CTGACAGTTA CCAATGCTTA |
| 3841 | ATCAGTGAGG CACCTATCTC AGCGATCTGT CTATTTCGTT CATCCATAGT TGCATTTAAA |
| 3901 | TTTCCGAACT CTCCAAGGCC CTCGTCGGAA AATCTTCAAA CCTTTCGTCC GATCCATCTT |
| 3961 | GCAGGCTACC TCTCGAACGA ACTATCGCAA GTCTCTTGGC CGGCCTTGCG CCTTGGCTAT |
| 4021 | TGCTTGGCAG CGCCTATCGC CAGGTATTAC TCCAATCCCG AATATCCGAG ATCGGGATCA |
| 4081 | CCCGAGAGAA GTTCAACCTA CATCCTCAAT CCCGATCTAT CCGAGATCCG AGGAATATCG |
| 4141 | AAATCGGGGC GCGCCTGGTG TACCGAGAAC GATCCTCTCA GTGCGAGTCT CGACGATCCA |
| 4201 | TATCGTTGCT TGGCAGTCAG CCAGTCGGAA TCCAGCTTGG GACCCAGGAA GTCCAATCGT |
| 4261 | CAGATATTGT ACTCAAGCCT GGTCACGGCA GCGTACCGAT CTGTTTAAAC CTAGATATTG |
| 4321 | ATAGTCTGAT CGGTCAACGT ATAATCGAGT CCTAGCTTTT GCAAACATCT ATCAAGAGAC |
| 4381 | AGGATCAGCA GGAGGCTTTC GCATGAGTAT TCAACATTTC CGTGTCGCCC TTATTCCCTT |
| 4441 | TTTTGCGGCA TTTTGCCTTC CTGTTTTTGC TCACCCAGAA ACGCTGGTGA AAGTAAAAGA |
| 4501 | TGCTGAAGAT CAGTTGGGTG CGCGAGTGGG TTACATCGAA CTGGATCTCA ACAGCGGTAA |
| 4561 | GATCCTTGAG AGTTTTCGCC CCGAAGAACG CTTTCCAATG ATGAGCACTT TTAAAGTTCT |
| 4621 | GCTATGTGGC GCGGTATTAT CCCGTATTGA CGCCGGGCAA GAGCAACTCG GTCGCCGCAT |
| 4681 | ACACTATTCT CAGAATGACT TGGTTGAGTA TTCACCAGTC ACAGAAAAGC ATCTTACGGA |
| 4741 | TGGCATGACA GTAAGAGAAT TATGCAGTGC TGCCATAACC ATGAGTGATA ACACTGCGGC |
| 4801 | CAACTTACTT CTGACAACGA TTGGAGGACC GAAGGAGCTA ACCGCTTTTT TGCACAACAT |
| 4861 | GGGGGATCAT GTAACTCGCC TTGATCGTTG GGAACCGGAG CTGAATGAAG CCATACCAAA |
| 4921 | CGACGAGCGT GACACCACGA TGCCTGTAGC AATGGCAACA ACCTTGCGTA AACTATTAAC |
| 4981 | TGGCGAACTA CTTACTCTAG CTTCCCGGCA ACAGTTGATA GACTGGATGG AGGCGGATAA |
| 5041 | AGTTGCAGGA CCACTTCTGC GCTCGGCCCT TCCGGCTGGC TGGTTTATTG CTGATAAATC |
| 5101 | TGGAGCCGGT GAGCGTGGGT CTCGCGGTAT CATTGCAGCA CTGGGGCCAG ATGGTAAGCC |
| 5161 | CTCCCGTATC GTAGTTATCT ACACGACGGG GAGTCAGGCA ACTATGGATG AACGAAATAG |
| 5221 | ACAGATCGCT GAGATAGGTG CCTCACTGAT TAAGCATTGG TAACCGATTC TAGGTGCATT |
| 5281 | GGCGCAGAAA AAAATGCCTG ATGCGACGCT GCGCGTCTTA TACTCCCACA TATGCCAGAT |
| 5341 | TCAGCAACGG ATACGGCTTC CCCAACTTGC CCACTTCCAT ACGTGTCCTC CTTACCAGAA |
| 5401 | ATTTATCCTT AAGATCCCGA ATCGTTTAAA CTCGACTCTG GCTCTATCGA ATCTCCGTCG |
| 5461 | TTTCGAGCTT ACGCGAACAG CCGTGGCGCT CATTTGCTCG TCGGGCATCG AATCTCGTCA |
| 5521 | GCTATCGTCA GCTTACCTTT TTGGCAGCGA TCGCGGCTCC CGACATCTTG GACCATTAGC |
| 5581 | TCCACAGGTA TCTTCTTCCC TCTAGTGGTC ATAACAGCAG CTTCAGCTAC CTCTCAATTC |
| 5641 | AAAAAACCCC TCAAGACCCG TTTAGAGGCC CCAAGGGGTT ATGCTATCAA TCGTTGCGTT |
| 5701 | ACACACACAA AAAACCAACA CACATCCATC TTCGATGGAT AGCGATTTTA TTATCTAACT |
| 5761 | GCTGATCGAG TGTAGCCAGA TCTAGTAATC AATTACGGGG TCATTAGTTC ATAGCCC |

### Example 1B

A construct was prepared exactly as described for Example 1A, apart from the transgene sequence instead encoded for Gaussia princeps luciferase (Gluc), which was codon-optimized for mammalian cells and with two methionines changed to leucines, the sequence of which is described below.

| | SEQ ID | |
|---|---|---|
| | NO: | Sequence |
| Gluc | 42 | ATGGGAGTCAAAGTTCTGTTTGCCCTGATCTGCATCGCTGTGGCCGAGGCCA |
| | | AGCCCACCGAGAACAACGAAGACTTCAACATCGTGGCCGTGGCCAGCAACT |
| transgene | | TCGCGACCACGGATCTCGATGCTGACCGCGGGAAGTTGCCCGGCAAGAAGC |
| sequence | | TGCCGCTGGAGGTGCTCAAAGAGTTGGAAGCCAATGCCCGGAAAGCTGGCT |
| | | GCACCAGGGGCTGTCTGATCTGCCTGTCCCACATCAAGTGCACGCCCAAGA |
| | | TGAAGAAGTTCATCCCAGGACGCTGCCACACCTACGAAGGCGACAAAGAGT |
| | | CCGCACAGGGGGGCATAGGCGAGGCGATCGTCGACATTCCTGAGATTCCTG |
| | | GGTTCAAGGACTTGGAGCCCTTGGAGCAGTTCATCGCACAGGTCGATCTGT |
| | | GTGTGGACTGCACAACTGGCTGCCTCAAAGGGCTTGCCAACGTGCAGTGTT |
| | | CTGACCTGCTCAAGAAGTGGCTGCCGCAACGCTGTGCGACCTTTGCCAGCA |
| | | AGATCCAGGGCCAGGTGGACAAGATCAAGGGGGCCGGTGGTGAC |
| Coding | 43 | ATGGCGAGAACCATGGTAGCCATGGAGACCATGGGGCTCATGACAACAGAT |
| sequence | | CTGGCAAAATTTGGGGCTGGATGCCACCAAAATCCTCCCAGGCAACATACGG |
| without the | | CAGCGGCGAGGGCAGAGGAAGTCTGCTAA |
| cryptic exon | | |
| Coding | 44 | ATGGCGAGAACCATGGTAGCCATGGAGACCATGGGGCTCATGACAACAGAT |
| sequence | | CTGGCAAAATTTGGGGCTGGAGTGCAGTGGCATGATCACAGCTCACTGCAG |
| with the | | CCTCAAACTTCCTGGGCTCAAGTGATCCTCTCCCGAGTAGCTGGGACTACAG |
| cryptic exon | | GCTGGATGCCACCAAAATCCTCCCAGGCAACATACGGCAGCGGCGAGGGCA |
| | | GAGGAAGTCTGCTAACATGCGGTGACGTCGAGGAGAATCCTGGCCCAATGG |
| | | GAGTCAAAGTTCTGTTTGCCCTGATCTGCATCGCTGTGGCCGAGGCCAAGCC |
| | | CACCGAGAACAACGAAGACTTCAACATCGTGGCCGTGGCCAGCAACTTCGC |
| | | GACCACGGATCTCGATGCTGACCGCGGGAAGTTGCCCGGCAAGAAGCTGCC |
| | | GCTGGAGGTGCTCAAAGAGTTGGAAGCCAATGCCCGGAAAGCTGGCTGCAC |
| | | CAGGGGCTGTCTGATCTGCCTGTCCCACATCAAGTGCACGCCCAAGATGAAG |
| | | AAGTTCATCCCAGGACGCTGCCACACCTACGAAGGCGACAAAGAGTCCGCA |
| | | CAGGGGGGCATAGGCGAGGCGATCGTCGACATTCCTGAGATTCCTGGGTTC |
| | | AAGGACTTGGAGCCCTTGGAGCAGTTCATCGCACAGGTCGATCTGTGTGTGG |
| | | ACTGCACAACTGGCTGCCTCAAAGGGCTTGCCAACGTGCAGTGTTCTGACCT |
| | | GCTCAAGAAGTGGCTGCCGCAACGCTGTGCGACCTTTGCCAGCAAGATCCA |
| | | GGGCCAGGTGGACAAGATCAAGGGGGCCGGTGGTGACTAA |

### Example 1C

Next, a construct was prepared as described for Example 1A, but wherein the transgene encoded for a TDP-43 based fusion protein, that is, TDP-43/Raver 1. We also generated an RNA-binding deficient mutant of the same construct, in which two phenylalanines in the RNA-recognition domain 1 of TDP-43 were mutated to leucine. The sequences of both are provided below.

| | SEQ | |
|---|---|---|
| | ID | |
| | NO: | Sequence |
| TDP- | 45 | GTCAGCAAAGGGGAAGAGCCAAAAAAGAAGAGAAAGGTAGAAGACCCCGGCGGA |
| 43/Raver 1 | | CCGGCGGCGAAACGCGTGAAACTGGATGGAGGTTACCCATACGATGTTCCAGATT |
| Transgene | | ACGCTGGTGGTATGTCAGAATATATTCGGGTCACCGAGGACGAGAACGACGAGCC |
| Sequence | | TATCGAGATACCATCCGAAGACGACGGAACAGTCCTCCTGAGTACCGTGACAGCA |
| | | CAATTCCCAGGGGCCTGCGGCCTCCGTTACAGAAACCCTGTTAGCCAGTGTATGA |
| | | GGGGTGTGCGGCTCGTGGAAGGCATACTCCACGCTCCGGACGCCGGGTGGGGTA |
| | | ACTTGGTTTATGTCGTAAATTACCCTAAGGACAATAAACGAAAGATGGACGAAACC |
| | | GACGCTAGTAGCGCCGTGAAAGTAAAACGGGCAGTGCAGAAGACATCTGACCTCA |
| | | TCGTCTTAGGTCTGCCTTGGAAGACCACAGAGCAGGATCTGAAAGAATATTTCTCT |
| | | ACTTTTGGCGAAGTCCTGATGGTGCAGGTGAAAAAGGATCTGAAGACAGGGCATA |
| | | GCAAAGGGTTCGGATTTGTCAGGTTCACTGAGTATGAGACCCAGGTGAAAGTGAT |
| | | GTCCCAGCGACATATGATCGATGGGCGGTGGTGCGATTGTAAGCTGCCTAATAGC |
| | | AAGCAGTCTCAGGACGAACCCTTAAGATCCCGCAAGGTGTTCGTGGGTCGCTGCA |
| | | CGGAGGATATGACCGAGGACGAACTCAGGGAATTTTTTTCACAATACGGAGACGT |
| | | AATGGACGTCTTTATCCCCAAGCCTTTTCGGGCCTTTGCCTTCGTTACTTTCGCTG |
| | | ATGATCAGATTGCTCAATCCTTGTGCGGCGAGGATCTTATTATTAAGGGCATCTCT |
| | | GTACACATCAGCAATGCAGAGCCCAAGCATAATTCTAACCTGCCACCTTTACTGG |
| | | GCCCCTCAGGCGGCGACCGGGAGCCAATGGGACTAGGCCCACCAGCAACGCAGCT |
| | | GACTCCACCACCCGCCCCAGTTGGCTTGCGTGGATCCAACCACCGTGGACTTCCC |
| | | AAAGATAGTGGCCCCTTGCCTACGCCACCCGGCGTGAGCCTGCTAGGCGAGCCAC |
| | | CAAAGGATTACAGGATACCCCTGAACCCTTACCTTAATCTCCACAGCCTGCTGCC |
| | | CTCTAGCAATCTTGCGGGAAAAGAGACCAGGGGCTGGGGCGGAAGCGGGAGAGG |
| | | GCGAAGACCAGCTGAGCCGCCACTGCCTTCGCCAGCAGTTCCTGGAGGAGGGTCA |
| | | GGCAGTAACAATGGCAACAAAGCGTTCCAAATGAAAAGTCGACTCTTGTCTCCCA |
| | | TTGCCTCTAACCGCCTGCCTCCCGAACCCGGGCTGCCAGACTCCTATGGATTTGA |
| | | TTACCCGACAGATGTGGGTCCTCGCCGCTTGTTCAGCCATCCCAGAGAACCTACT |
| | | CTAGGAGCCCACGGGCCGAGTAGGCACAAAATGTCGCCTCCGCCGTCCTCATTCA |
| | | ACGAGCCTAGATCCGGCGGTGGGTCCGGAGGCCCACTTTCGCACTTCTGA |
| Mutant | 46 | GTCAGCAAAGGGGAAGAGCCAAAAAAGAAGAGAAAGGTAGAAGACCCCGGCGGA |
| TDP- | | CCGGCGGCGAAACGCGTGAAACTGGATGGAGGTTACCCATACGATGTTCCAGATT |
| 43/Raver 1 | | ACGCTGGTGGTATGTCAGAATATATTCGGGTCACCGAGGACGAGAACGACGAGCC |
| Transgene | | TATCGAGATACCATCCGAAGACGACGGAACAGTCCTCCTGAGTACCGTGACAGCA |
| Sequence | | CAATTCCCAGGGGCCTGCGGCCTCCGTTACAGAAACCCTGTTAGCCAGTGTATGA |
| (mutations | | GGGGTGTGCGGCTCGTGGAAGGCATACTCCACGCTCCGGACGCCGGGTGGGGTA |
| in bold and | | ACTTGGTTTATGTCGTAAATTACCCTAAGGACAATAAACGAAAGATGGACGAAACC |
| underlined) | | GACGCTAGTAGCGCCGTGAAAGTAAAACGGGCAGTGCAGAAGACATCTGACCTCA |
| | | TCGTCTTAGGTCTGCCTTGGAAGACCACAGAGCAGGATCTGAAAGAATATTTCTCT |
| | | ACTTTTGGCGAAGTCCTGATGGTGCAGGTGAAAAAGGATCTGAAGACAGGGCATA |
| | | GCAAAGGGCTCGGACTTGTCAGGTTCACTGAGTATGAGACCCAGGTGAAAGTGAT |
| | | GTCCCAGCGACATATGATCGATGGGCGGTGGTGCGATTGTAAGCTGCCTAATAGC |
| | | AAGCAGTCTCAGGACGAACCCTTAAGATCCCGCAAGGTGTTCGTGGGTCGCTGCA |
| | | CGGAGGATATGACCGAGGACGAACTCAGGGAATTTTTTTCACAATACGGAGACGT |
| | | AATGGACGTCTTTATCCCCAAGCCTTTTCGGGCCTTTGCCTTCGTTACTTTCGCTG |
| | | ATGATCAGATTGCTCAATCCTTGTGCGGCGAGGATCTTATTATTAAGGGCATCTCT |
| | | GTACACATCAGCAATGCAGAGCCCAAGCATAATTCTAACCTGCCACCTTTACTGG |
| | | GCCCCTCAGGCGGCGACCGGGAGCCAATGGGACTAGGCCCACCAGCAACGCAGCT |
| | | GACTCCACCACCCGCCCCAGTTGGCTTGCGTGGATCCAACCACCGTGGACTTCCC |
| | | AAAGATAGTGGCCCCTTGCCTACGCCACCCGGCGTGAGCCTGCTAGGCGAGCCAC |
| | | CAAAGGATTACAGGATACCCCTGAACCCTTACCTTAATCTCCACAGCCTGCTGCC |
| | | CTCTAGCAATCTTGCGGGAAAAGAGACCAGGGGCTGGGGCGGAAGCGGGAGAGG |
| | | GCGAAGACCAGCTGAGCCGCCACTGCCTTCGCCAGCAGTTCCTGGAGGAGGGTCA |
| | | GGCAGTAACAATGGCAACAAAGCGTTCCAAATGAAAAGTCGACTCTTGTCTCCCA |
| | | TTGCCTCTAACCGCCTGCCTCCCGAACCCGGGCTGCCAGACTCCTATGGATTTGA |
| | | TTACCCGACAGATGTGGGTCCTCGCCGCTTGTTCAGCCATCCCAGAGAACCTACT |
| | | CTAGGAGCCCACGGGCCGAGTAGGCACAAAATGTCGCCTCCGCCGTCCTCATTCA |
| | | ACGAGCCTAGATCCGGCGGTGGGTCCGGAGGCCCACTTTCGCACTTCTGA |

### Example 1D

It was found that the Example 1A construct could also be modified by using different sequences for both the cryptic exon and flanking exonic context. In this case, the cryptic exon sequence and flanking exonic sequences instead encoded a fragment of Streptococcus pyogenes Cas9 enzyme. The construct was otherwise as described in Example 1A, and comprised a transgene sequence for mCherry.

To help design this construct, we used computational splicing prediction programs (i.e. Splice AI, see https://github.com/Illumina/SpliceAI), to identify sequences that demonstrate a high probability of splicing. Cryptic exon sequences with various synonymous codons were identified which gave moderate (i.e., >0.01 and <0.5) SpliceAI scores for the cryptic donor and acceptor, and no other predicted splice sites within the cryptic exon. The following synthetic sequence, for example, had scores of 0.31 for the cryptic acceptor and 0.42 for the cryptic donor.

| | Seq ID | |
|---|---|---|
| | No: | Sequence |
| Full 1D | 47 | GGTTTAGTGAACCGTCAGATCAGATCTTTGTCGATCCTACCATCCACTCG |
| construct | | ACACACCCGCCAGCGGCCGCTTCTTGGTGCCAGCTTATCATAGCGCTAC |
| | | CGGTCGCCACCATGGCGAGAACCATGGTAGCCATGGAGACCATGGGGCT |
| | | CATGACAACAGATCTGGCAAAATTTGGGAGATACACCGGCTGGGGCAGG |
| | | TAAGAATGCACATCACTTCTTGAGAGTATGGAGGAGTGAAATGACACTCA |
| | | GTGCCAGAGTTACTGTATATCTACACTTTAAAAGTGTAGCTTTTAAAAGAT |
| | | AAGCAAGCACAATCTTTTGTGTGTGTGTGTGTGAATGTGTGTGTGTGTGT |
| | | GTGTCACCCAGATTATCACGCAAATTGATCAATGGAATAAGAGATAAACAG |
| | | TCCGGAAAAACAATCCTTGATTTTTTAAAAAGTGATGGGTTCGCAAATAGA |
| | | AATTTTATGCAACTCATACATGATGACAGCTTGACATTCAAAGAGGACATT |
| | | CAGAAGGCGCAGGTATGCATCACCCCCCCAGCTAATTTTTTTTTGTATTTT |
| | | TTACCGAGTCGGGGTTTCGCAATGTTGCCCAGGCTGGTCTCAGAGTCTC |
| | | GCTCTGTTGTCTACGCTGGAGTGCAGTAACATGAGCCACTGTGCCCGGC |
| | | CAATCCTAAGAATTTCTTTTGCGGTGGTTGCAAGTCTGGGCAGAACTCTT |
| | | GTCAGGGGCTGTAACTGGACTTATCTTTACTCCTTTGTCAGGTATCCGGC |
| | | CAGGGCGATAGCCTGCAATCCTCCCAGGCAACATACGGCAGCGGCGCCA |
| | | CCAACTTTTCCCTGCTCAAGCAAGCCGGCGACGTGGAAGAGAATCCCGG |
| | | CCCCGTCAGCAAAGGGGAAGAGGACAACATGGCCATCATTAAGGAGTTT |
| | | ATGCGATTCAAAGTACACATGGAGGGATCTGTTAATGGCCATGAATTTG |
| | | AGATAGAGGGGGAAGGTGAGGGTCGCCCTTACGAAGGCACGCAGACGGC |
| | | TAAGCTGAAGGTCACGAAAGGGGGACCCTTGCCCTTCGCATGGGACATAC |
| | | TCTCCCCACAGTTTATGTATGGTTCTAAGGCATATGTTAAGCACCCTGCA |
| | | GACATCCCAGACTATCTGAAGCTCTCCTTTCCTGAGGGGTTTAAGTGGGA |
| | | ACGCGTTATGAACTTTGAGGATGGAGGGGTCGTGACTGTTACCCAGGATT |
| | | CTTCCCTGCAAGATGGAGAGTTCATATACAAAGTGAAACTTCGGGGAACG |
| | | AATTTCCCATCAGACGGGCCAGTGATGCAGAAAAAGACGATGGGGTGGG |
| | | AGGCTTCATCCGAGAGGATGTATCCCGAGGACGGAGCATTGAAAGGCGA |
| | | AATAAAACAAAGGCTGAAGTTGAAGGATGGGGGCCACTACGACGCGGAG |
| | | GTTAAAACAACGTATAAAGCTAAAAAGCCAGTACAGCTCCCAGGCGCATA |
| | | TAACGTGAATATAAAGCTTGACATAACGAGTCATAACGAGGATTACACAA |
| | | TCGTAGAACAGTACGAAAGAGCTGAAGGACGGCACTCCACCGGTGGGATG |
| | | GATGAACTCTATAAAGACTACAAGGACGATGATGACAAGTAAACAAATGG |
| | | TAAGGAAGGGCACATCAATCTTTGCTTAATTGTCCTTTACTCTAAAGATGT |
| | | ATTTTATCATACTGAATGCTAAACTTGATATCTCCTTTTAGGTCATTGATG |
| | | TCCTTCACCCCGGGAAGGCGACAGTGCCTAAGACAGAAATTCGGGAAAAA |
| | | CTAGCCAAAATGTACAAGACCACACCGGATGTCATCTTTGTATTTGGATTC |
| | | AGAACTCA |
| Upstream | 48 | AGATACACCGGCTGGGGCAG |
| exonic | | |
| sequence | | |
| Cryptic exon | 49 | ATTATCACGCAAATTGATCAATGGAATAAGAGATAAACAGTCCGGAAAAAC |
| sequence | | AATCCTTGATTTTTTAAAAAGTGATGGGTTCGCAAATAGAAATTTTATGCA |
| | | ACTCATACATGATGACAGCTTGACATTCAAAGAGGACATTCAGAAGGCGCA |
| | | G |
| Downstream | 50 | GTATCCGGCCAGGGCGATAGCCTGC |
| exonic | | |
| sequence | | |

### Example 1E

To further examine whether different regulatory sequences could be used for a Design 1 reporter, we designed a high-throughput assay to test the splicing behaviour of large numbers of different synthetic cryptic exons, in the context of a Design 1-style regulatory upstream sequence. To enable this, we generated a library of plasmids featuring different cryptic exon sequences: each cryptic exon encoded the same amino acid sequence (a fragment of Cas9) but featured different combinations of synonymous codons. The surrounding sequence was the same as the upstream regulatory sequence from Example 1D.

We then performed high-throughput RNA-sequencing to determine the splicing behaviour of each cryptic exon sequence. We found that different sequences in this context also showed increased cryptic exon expression upon TDP-43 knockdown, with the majority of these having no detectable leaky expression in normal cells (i.e., those without TDP-43 knockdown). A selection of these sequences are detailed below, in addition to the SpliceAI scores assigned to the cryptic splice sites of each, and the percentage inclusion of the cryptic exon upon TDP-43 knockdown (KD). While the percentage inclusion is low for some examples, this is still enough to give good protein expression selectively in diseased cells.

| | | | | Cryptic |
|---|---|---|---|---|
| | | Acceptor | Donor | inclusion |
| SEQ | | Splice | Splice | (%) |
| ID | | AI | AI | TDP-43 |
| NO: | Cryptic Exon Sequence | score | score | KD |
| 51 | GCTATCGCGTAAACTTATTAATGGCATCCGGGATAAGCAGTCC | 0.98 | 1.00 | 3.30 |
| | GGGAAGACTATTCTCGATTTCCTGAAGTCTGATGGCTTTGCGA | | | |
| | ACCGGAACTTCATGCAGCTGATCCATGACGACTCTCTAACGTT | | | |
| | CAAGGAGGACATTCAGAAGGCGCAG | | | |
| 52 | ACTCTCTCGAAAGCTGATCAATGGAATACGGGATAAACAATCG | 0.88 | 0.92 | 47.10 |
| | GGGAAAACAATTCTAGATTTTCTCAAGTCGGATGGCTTTGCGA | | | |
| | ATCGCAATTTCATGCAACTTATTCATGATGATTCGCTTACATT | | | |
| | TAAGGAGGATATACAGAAGGCTCAG | | | |
| 53 | ACTTTCTCGAAAGCTGATTAACGGTATACGCGATAAGCAGTCTG | 0.90 | 0.97 | 4.80 |
| | GAAAAACGATTCTGGATTTCCTGAAGTCCGATGGGTTTGCGAAC | | | |
| | CGCAATTTTATGCAACTTATACACGATGATTCACTGACATTTAA | | | |
| | GGAGGATATACAGAAAGCGCAG | | | |
| 54 | ACTGTCTCGAAAGCTCATTAATGGTATCCGCGACAAGCAATCTG | 0.94 | 0.97 | 62.50 |
| | GGAAAACTATCCTTGATTTCCTCAAGTCCGATGGCTTTGCAAAT | | | |
| | CGGAACTTTATGCAACTCATTCATGACGACTCGCTAACTTTTAA | | | |
| | AGAAGATATTCAAAAGGCGCAG | | | |
| 55 | ACTATCTCGCAAGCTCATTAACGGTATACGAGACAAACAGTC | 0.59 | 0.59 | 9.10 |
| | GGGGAAAACGATACTCGATTTCCTCAAGTCTGACGGCTTCGC | | | |
| | TAATCGTAATTTCATGCAACTGATTCACGACGACTCTCTCAC | | | |
| | ATTCAAAGAAGACATACAAAAGGCACAA | | | |
| 56 | GCTGTCTCGTAAGCTAATCAACGGAATCCGTGACAAGCAATCT | 0.91 | 0.95 | 4.50 |
| | GGGAAAACAATACTTGACTTCCTAAAGTCAGATGGTTTCGCTA | | | |
| | ACCGTAATTTCATGCAGCTAATTCATGACGACTCACTTACGTT | | | |
| | TAAGGAAGATATCCAGAAGGCGCAG | | | |
| 57 | GCTTTCGCGAAAACTAATCAATCGGATTCGCGATAAGCAATCGGG | 0.95 | 0.99 | 5.60 |
| | AAAAACAATACTTGATTTTCTAAAGTCTGATGGGTTTGCAAATCG | | | |
| | GAATTTTATGCAACTGATTCATGATGATTCGCTGACTTTCAAAGA | | | |
| | GGATATTCAGAAGGCACAG | | | |
| 58 | ACTATCTCGTAAACTGATTAATGGGATACGAGATAAGCAATCGGGA | 0.50 | 0.54 | 66.70 |
| | AAAACGATCCTGGACTTCCTGAAATCAGACGGGTTTGCTAATCGAA | | | |
| | ATTTCATGCAACTTATCCACGACGATTCGCTTACGTTTAAGGAGGA | | | |
| | TATTCAAAAAGCGCAA | | | |
| 59 | GCTTTCCCGTAAACTTATAAATGGTATTCGTGATAAACAGTCTGGCA | 0.10 | 0.63 | 7.10 |
| | AGACTATTCTTGATTTCCTAAAGTCAGATGGTTTCGCTAACCGGAAC | | | |
| | TTTATGCAACTTATTCATGATGACTCTCTAACCTTTAAGGAGGACAT | | | |
| | ACAGAAAGCGCAG | | | |
| 60 | ACTCTCACGTAAACTGATCAACGGGATACGGGATAAACAGTCGGGCA | 0.29 | 0.49 | 5.30 |
| | AAACTATACTAGATTTCCTGAAGTCAGATGGGTTTGCGAACCGTAAT | | | |
| | TTTATGCAGCTTATTCATGACGATTCCCTAACTTTTAAGGAAGACAT | | | |
| | ACAGAAAGCACAG | | | |
| 61 | GCTGTCTCGAAAACTGATAAATGGTATCCGCGACAAGCAATCAGGGA | 0.39 | 0.44 | 3.80 |
| | AGACGATTCTTGATTTTCTTAAATCTGATGGCTTTGCTAATCGTAAC | | | |
| | TTTATGCAGCTTATCCACGACGATTCCCTGACCTTCAAAGAAGATAT | | | |
| | ACAGAAGGCCCAA | | | |
| 62 | ACTTTCACGAAAACTGATAAACGGTATTCGAGATAAGCAATCCGGTAA | 0.50 | 0.69 | 5.00 |
| | GACCATACTGGATTTCCTTAAATCTGATGGTTTTGCGAATCGCAATTTT | | | |
| | ATGCAGCTAATCCATGACGATTCTCTGACCTTTAAAGAAGATATCCAGA | | | |
| | AGGCCCAG | | | |
| 63 | ACTTTCTCGGAAGCTTATCAATGGGATCCGAGATAAGCAATCAGGCA | 0.13 | 0.11 | 3.70 |
| | AAACGATCCTTGATTTTCTTAAGTCCGATGGATTTGCTAACCGGAATT | | | |
| | TTATGCAACTTATCCACGATGACTCTCTCACTTTTAAAGAGGATATCC | | | |
| | AAAAGGCACAA | | | |
| 64 | GCTCTCCCGCAAGCTTATAAATGGAATTCGGGACAAACAGTCTGGGA | 0.84 | 0.95 | 4.30 |
| | AGACAATCCTGGACTTTCTAAAGTCTGATGGCTTTGCTAATCGTAAC | | | |
| | TTTATGCAACTAATACATGACGATTCTCTTACGTTTAAAGAAGACAT | | | |
| | ACAAAAGGCACAG | | | |

We noted that, as demonstrated by Example 1E, cryptic exon splicing was possible with various synthetic cryptic exon sequences with a wide range of different SpliceAI predicted splice scores.

### Comments on Design 1 Constructs

Examples 1A-1D all had a construct according to “Design 1” as shown in FIG. 1. Example 1E featured the same AARS1-based intronic sequences as examples 1A-1D, but did not feature a downstream transgene, and instead featured a 12 nt barcode sequence.

A construct of Design 1 has many advantages. The main benefit of this design is that it can be very easily modified to control the expression of various different proteins by simply including a different complete transgene or protein-coding sequence downstream of the regulatory sequence. This is demonstrated by looking to Examples 1A-1C above. As demonstrated in Example 1D-1E, a range of different cryptic exon sequences and intronic sequence contexts can be used.

The above “Design 1” examples contain many preferred or optional features.

For example, while the above “Design 1” example construct comprises a P2A cleavage site downstream of the cryptic exon, this feature is not essential because some transgenes may function correctly with an additional N-terminal sequence encoded by the upstream regulatory domain. Presence of a cleavage site (e.g., such as P2A) nevertheless has the advantage of ensuring that the transgene can be expressed without an extra N-terminal sequence, which in some cases may improve the functionality of the transgene's protein product. It is envisaged that the P2A cleavage site can be replaced with a range of alternative protein cleavage or self-cleaving sites, as described above, which would confer the same benefits.

Additionally, although each Design 1 construct described above contains intronic regions based on AARS1, we show below (for example in Examples 2A-2C) that different intronic sequences, based on no pre-existing sequence, can successfully be designed that harbour cryptic exons. In fact, the synthetic intronic/cryptic exon sequences in Examples 2A-2C could directly be used as the regulatory domain of a Design 1 construct, as the cryptic exons cause frame shifts. Thus, the intronic sequences of a Design 1 construct are not limited to AARS1-derived sequences, but could be any suitable intronic sequence, which may or may not be based on a naturally occurring cryptic exon/intronic context.

In the above example “Design 1” construct, the protein-coding sequence itself comprises a premature termination codon (PTC) in-frame with the start codon when the cryptic exon sequence is not included in the mRNA product, but out of frame with the start codon when the cryptic exon sequence is included in the mRNA product. A PTC sequence is any sequence selected from TGA, TAA and TAG, in frame with, and downstream of, the start codon. However, the construct need not contain a premature termination codon if the cryptic exon itself comprises the start codon. This would mean that only in diseased cells (i.e., with depletion of hnRNP splicing factor) is the full downstream transgene translated; in cells without depletion of the hnRNP splicing factor, the translated protein could be an out-of-frame peptide, or an N-terminally truncated version of the protein encoded by the transgene, depending on the position of the start codon in mRNA products without the cryptic exon.

The above example constructs comprise a further intronic sequence downstream of the cryptic exon (in this example, derived from RPS24). While not essential, the presence of a downstream intron is preferred, since it promotes deposition of an exon junction complex (EJC) on the resultant mRNA. When the cryptic exon sequence is not included in the mRNA transcript and thus the premature termination codon is encountered, this triggers nonsense-mediated decay (NMD) of the transcript, which further improves the safety of the construct in healthy cells (as otherwise the peptide produced in healthy cells could build-up and could aggregate or even be potentially toxic). In contrast, in cells that are absent of hnRNP splicing factor (i.e., diseased cells, e.g., with TDP-43 depletion), splicing is not repressed, and the cryptic exon is included in the mRNA product. In these cases, the PTC codon (e.g., the PTC or a stop codon within a transgene sequence) is not in frame with the start codon, and the ribosome therefore removes the EJC, such that no nonsense-mediated decay occurs. In this example, a further intronic sequence within an exonic context is downstream of the transgene, however, the further intronic sequence could instead be present within the transgene itself. In this example, while the intron immediate flanking sequence is derived from the human RPS24 gene (which was selected since it is highly expressed, constitutively spliced, and short in length), it is envisaged that numerous alternative suitable introns and flanking sequences could be used, as there exist hundreds of short, constitutively spliced mammalian introns that could be readily selected by the skilled person and used in the same way.

Further, while the above example constructs comprise a frame-shift inducing cryptic exon (e.g., a sequence with a number of nucleotides that is not divisible by 3), regulation can still be achieved without requiring a frame-shift if the cryptic exon were to itself contain the start codon that is required for transgene expression.

In the constructs described above, the TDP-43 binding domain comprises a TG/UG repeat (with a small “AA” interruption). However, it is known in the art that TDP-43 is capable of binding to other TG/UG-rich sequences which are not pure repeats. Structural biology studies have demonstrated that many bases within the TDP-43 binding footprint can be degenerate, and have shown that TDP-43 can bind “UG-rich” sequences such as SEQ ID NO: 65 GUGUGAAUGAAU with similar affinity to pure UG-repeats. Furthermore, there are well characterized examples of TDP-43 regulated cryptic exons that feature TDP-43-binding domains that are UG-rich, but do not contain extended UG repeats. A clear example is the TDP-43 regulated cryptic exon in UNC13A (see SEQ ID NO: 66): although a significant enrichment of UG is observed in the region near the cryptic exon which TDP-43 binds (as shown via iCLIP studies), there are no UG-repeats of 3 (UGUGUG) or longer within 400 nt of the cryptic exon, and no TG-repeats of 4 (UGUGUGUG) or longer anywhere within the annotated intron that harbors this cryptic exon. A TDP-43 binding domain may therefore include any TG/UG-rich region.

UNC13A intron with cryptic (cryptic in bold,
TG-rich region (SEQ ID NO: 67) in italics)
SEQ ID NO: 66
GTGAGGGTCATTGCTCGGCCCCTCCCATGCCACTTCCACTCACCA
TTCCTGCCTGCCCAGCTCTTCCTCTTTCTGGCCACACCATCCACA
CTCTCCTGGCCCTCTGAGACTGCCCGCCATGCCATTCCCTTTACC
TGGAAAACTCCTCCCTATCCATCAAAGTCCAGATTCAGGGTCACC
TCCTCTGGGAAGCCCACCTTGGCCTCCAGGTTGACTCTCACTACT
CATCATCAGGTTCTTCCTTCTATTCCAGCCCTAACCACTCAGGAT
TGGGCCGTTTGTGTCTGGGTATGTCTCTTCCAGCTGCCTGGGTT
TCCTGGAAAGAACTCTTATCCCCAGGAACTAGTTTGTTGAATAAA
TGCTGGTGAATGAATGAATGATTGAACAGATGAATGAGTGATGAG
TAGATAAAAGGATGGATGGAGAGATGGGTGAGTACATGGATGGAT
AGATGGATGAGTTGGTGGGTAGATTCGTGGCTAGATGGATGATGG
ATGGATGGACAGATGGATGGATATATGATTGAACTATTGAAAGTA
TAGATGTATGGATGGGTGAATTTGGGGGTAATTGTTAGATGATGG
ATGAGTATAGATGAATGATGGATGGATAACTTGATGAGTGGATAG
ATAGATTGCTGGATAGATGATTGACTGGGTGGATAGATGAAATGT
TGGATGAGCAGATTAAGTTGTATTGGATGGGATGGATGGAAGTGT
GGTTGAGTTATTAGAAGGAAGATTGAGTAGATAGGTGAATTTGTT
GATAGTCAGATGGGTAGATAGGTAGATGGATGGATGGATGGATGG
ATGTATAGGCAGATGGACAAATGGATGAATGGGTGGGTGGATGAA
TGGAAGGATGTGTGGTTGAACTATTGCAAGTATTGATAATTGGGT
TCATAATTTCTGAATATTTAGATGGATGGTTGTGAGTGGCTGGTG
GACAGACGAAAAATGGATGGTTGGATAAATTGATGGGTGGATGGA
TGGTTGGTTGTATGAAAGAATGAATGATTGGGTAGGTGGATTAAG
TTGCGGATCAATGTATGGGATGGATGAATGGATGGATGGATGGAT
GTGTGGTTGAATTACTGAAAGGTTGGAAGAGTGGATGGGTGAAAT
TTGGGGTAGTTAGATGGGTGGGTGTGTGGATGGATAAAAGAGTAG
ATGAATGAATTAATGAATAAACAGGCAGATGGATGATGTAAGCTG
CCCCAGACCCTGGGACCTCTGACCCCCGGCGACCCCTTGCACTCT
CCATGACACTTTCTCTCCCATGGTGGCAG

While the constructs described herein comprise TDP-43 binding domains and are regulated by TDP-43, the binding domain can be switched for any other hnRNP splicing factor. Binding domains for other hnRNP splicing factors are known in the art.

### Design 2

We next designed a construct having a different design to the constructs shown in Example 1. Design 2 constructs are exemplified by FIG. 2.

Constructs of Design 2 comprise a regulatory domain comprising an intronic sequence comprising a TDP-43 binding domain, and a cryptic exon sequence embedded within the intronic region (defined by a splice acceptor site and splice donor site), but where the cryptic exon sequence itself encodes for part of a transgene which encodes for a protein (e.g., a functional or diagnostic protein).

### Example 2

The construct contains (from 5′→3′):
A sequence comprising a start codon
A first exon, encoding for a first part of the transgene (here, mCherry),
A regulatory domain comprising:
   A cryptic exon sequence embedded within an intronic region, wherein the cryptic exon sequence encodes for a second part of the transgene (here, mCherry). The cryptic exon sequence is defined by a splice acceptor site and splice donor site, where one of these splice sites is repressed by TDP-43 binding. The intronic region itself is defined by a second splice donor and acceptor site and is split into two parts, a first part upstream of the cryptic exon sequence and a second part downstream of the cryptic exon sequence. The intronic region comprises a TDP-43 binding domain A third exon, encoding for a third part of the transgene (here, mCherry).

A further intronic sequence, comprising an intron in an exonic context (here, derived from RPS24).

In Example 2A-C constructs, the exonic sequences all together encoded for mCherry. The cryptic exon sequence encoded for the internal part of mCherry, and the N- and C-terminal sequences of mCherry were encoded by the upstream exon (i.e., first exon) and downstream exon (i.e., third exon) respectively.

Different to the Design 1 constructs, the cryptic exon sequence encoded for part of the transgene. Different to the Design 1 constructs, the cryptic exons and, in some examples, surrounding intronic regions forming the regulatory domain, were also completely synthetic. These were designed using computational splicing prediction programs (i.e., Splice AI, see https://github.com/Illumina/SpliceAI).

An algorithm was used and developed to design these entirely synthetic cryptic exons and surrounding introns (see Materials and Methods). To generate the introns, randomised sequences were generated, where each base had an equal chance of being A, C, G or T; and GT (AAG) and (C) AG were added to the 5′ and 3′ ends respectively; additionally, TG-rich regions (e.g., a sequence with at least 80% identity to SEQ ID NO: 2 and/or SEQ ID NO: 115) and or randomised pyrimidine-rich regions (defined as a 30 nucleotide region with 80% chance of a pyrimidine) were added, to form a TDP-43 binding site or polypyrimidine tract respectively. As a result, the resultant intronic sequences were entirely synthetic and were not derived from any existing intronic sequence. To generate the cryptic exon sequence, a section of the mCherry transgene sequence was selected and reverse translated. The introns and cryptic exon were then joined together and combined with the upstream and downstream mCherry coding sequences, to form an initial sequence.

Next, SpliceAI was used to predict and modify the splicing characteristics of the initial sequence. The sequence was randomly mutated; but wherein for the coding regions, only synonymous mutations (i.e., mutations that did not change the encoded amino acid sequence) were allowed. After each round of mutations, SpliceAI was used to predict the splicing behaviour. The splicing predictions were compared to the presumed ideal scenario (where the intronic upstream and downstream splice sites (i.e., the second splice donor site and second splice acceptor site) have high scores of ˜1.00 (e.g., >0.95), and the splice sites defining the cryptic exon had slightly lower splicing scores (e.g., 0.8), and where there were no other predicted splice sites with scores of >0.01). If the predicted splicing of the mutated sequence was closer to the ideal scenario than the previous best sequence, then the new mutated sequence was used as the template for subsequent rounds of mutation; if it was no better, or worse, than the previous best sequence, the mutated sequence was discarded. As such, the algorithm can be viewed as a Darwinian, directed evolution approach to generating optimised sequences.

Three different constructs were prepared, all of which encoded for mCherry. The first two examples (Example 2A and 2B) featured a TDP-43 binding domain (i.e., a TG rich region) upstream of the cryptic exon. In Example 2C, the TDP-43 binding domain (i.e., a TG rich region) was downstream of the cryptic exon. The Splice AI scores for the cryptic splice sites were as follows:

| | Cryptic Acceptor SpliceAI | Cryptic Donor SpliceAI |
|---|---|---|
| Example | score | score |
| 2A | 0.82 | 0.79 |
| 2B | 0.77 | 0.65 |
| 2C | 0.93 | 0.9 |

The sequences and component parts of the example constructs were as follows:

### Example 2A

| | SEQ ID | |
|---|---|---|
| | NO: | |
| Example 2A | 68 | ATGGTCAGCAAAGGGGAAGAGGACAACATGGCCATCATTAAGGAGTTTATG |
| construct | | CGATTCAAAGTACACATGGAGGGATCTGTTAATGGCCATGAATTTGAGATA |
| | | GAGGGGGAAGGTGAGGGTCGCCCTTACGAAGGCACGCAGACGGCTAAGC |
| | | TGAAGGTCACGAAAGGGGGACCCTTGCCCTTCGCATGGGACATACTCTCC |
| | | CCACAGGTAAGAGCGTTCGGCCTTATTTACTGCTGCCTGGGCTCAAGCACT |
| | | CGATAGTACCGTAATATTGGTTAGACAGTTACACGGTAGTGAGCTGGAAGA |
| | | TTGTAAATGTGTGTGTGTGTGTGTGAGTGTGTGTGTGTGTGTGTGTTTCTAG |
| | | TTCATGTACGGGAGCAAGGCCTACGTTAAACATCCGGCCGACATTCCAGGT |
| | | AAGTTCAACTCACTGCACATGATCGCATAGCGTAATAGGCCTCACTTCTTTT |
| | | GAGCTAGGGATAGAGACGCTTAAGTTATATGTTGAGGCGCTAAGTACCGAT |
| | | GGATGCTTTTCACTTTGATCTCTCCTCCCCAGACTATCTGAAGCTCTCCTTT |
| | | CCTGAGGGGTTTAAGTGGGAACGCGTTATGAACTTTGAGGATGGAGGGGTC |
| | | GTGACTGTTACCCAGGATTCTTCCCTGCAAGATGGAGAGTTCATATACAAA |
| | | GTGAAACTTCGGGGAACGAATTTCCCATCAGACGGGCCAGTGATGCAGAAA |
| | | AAGACGATGGGGTGGGAGGCTTCATCCGAGAGGATGTATCCCGAGGACGG |
| | | AGCATTGAAAGGCGAAATAAAACAAAGGCTGAAGTTGAAGGATGGGGGCC |
| | | ACTACGACGCGGAGGTTAAAACAACGTATAAAGCTAAAAAGCCAGTACAGC |
| | | TCCCAGGCGCATATAACGTGAATATAAAGCTTGACATAACGAGTCATAACGA |
| | | GGATTACACAATCGTAGAACAGTACGAAAGAGCTGAAGGACGGCACTCCAC |
| | | CGGTGGGATGGATGAACTCTATAAAGACTACAAGGACGATGATGACAAGTA |
| | | AACAAATGGTAAGGAAGGGCACATCAATCTTTGCTTAATTGTCCTTTACTCT |
| | | AAAGATGTATTTTATCATACTGAATGCTAAACTTGATATCTCCTTTTAGGTC |
| | | ATTGATGTCCTTCACCCCGGGAAGGCGACAGTGCCTAAGACAGAAATTCGG |
| | | GAAAAACTAGCCAAAATGTACAAGACCACACCGGATGTCATCTTTGTATTTG |
| | | GATTCAGAACTCA |
| First mCherry | 69 | ATGGTCAGCAAAGGGGAAGAGGACAACATGGCCATCATTAAGGAGTTTATG |
| exon | | CGATTCAAAGTACACATGGAGGGATCTGTTAATGGCCATGAATTTGAGATA |
| sequence | | GAGGGGGAAGGTGAGGGTCGCCCTTACGAAGGCACGCAGACGGCTAAGCT |
| encoding for | | GAAGGTCACGAAAGGGGGACCCTTGCCCTTCGCATGGGACATACTCTCC |
| first part of | | CCACAG |
| transgene | | |
| First part of | 70 | GTAAGAGCGTTCGGCCTTATTTACTGCTGCCTGGGCTCAAGCACTCGATAG |
| intronic region | | TACCGTAATATTGGTTAGACAGTTACACGGTAGTGAGCTGGAAGATTGTAA |
| (TDP-43 | | ATGTGTGTGTGTGTGTGTGAGTGTGTGTGTGTGTGTGTGTTTCTAG |
| binding | | |
| domain in bold | | |
| and | | |
| underlined) | | |
| CE and | 71 | TTCATGTACGGGAGCAAGGCCTACGTTAAACATCCGGCCGACATTCCAG |
| second | | |
| mCherry exon | | |
| sequence | | |
| encoding for | | |
| second part of | | |
| transgene | | |
| Second part of | 72 | GTAAGTTCAACTCACTGCACATGATCGCATAGCGTAATAGGCCTCACTTCTT |
| intronic region | | TTGAGCTAGGGATAGAGACGCTTAAGTTATATGTTGAGGCGCTAAGTACCG |
| | | ATGGATGCTTTTCACTTTGATCTCTCCTCCCCAG |
| Third mCherry | 73 | ACTATCTGAAGCTCTCCTTTCCTGAGGGGTTTAAGTGGGAACGCGTTATGA |
| exon | | ACTTTGAGGATGGAGGGGTCGTGACTGTTACCCAGGATTCTTCCCTGCAAG |
| sequence | | ATGGAGAGTTCATATACAAAGTGAAACTTCGGGGAACGAATTTCCCATCAG |
| encoding for | | ACGGGCCAGTGATGCAGAAAAAGACGATGGGGTGGGAGGCTTCATCCGAG |
| third part of | | AGGATGTATCCCGAGGACGGAGCATTGAAAGGCGAAATAAAACAAAGGCT |
| transgene | | GAAGTTGAAGGATGGGGGCCACTACGACGCGGAGGTTAAAACAACGTATA |
| (PTC shown in | | AAGCTAAAAAGCCAGTACAGCTCCCAGGCGCATATAACGTGAATATAAAGC |
| bold) | | TTGACATAACGAGTCATAACGAGGATTACACAATCGTAGAACAGTACGAAA |
| | | GAGCTGAAGGACGGCACTCCACCGGTGGGATGGATGAACTCTATAAAGAC |
| | | TACAAGGACGATGATGACAAGTAA |

The sequence comprising the start codon and further intronic sequence (e.g., based on RPS24) was as the same as described in Example 1A.

### Example 2B

| | SEQ ID | |
|---|---|---|
| | NO: | |
| Construct | 74 | ATGGTCAGCAAAGGGGAAGAGGACAACATGGCCATCATTAAGGAGTTTATGCGA |
| 2B | | TTCAAAGTACACATGGAGGGATCTGTTAATGGCCATGAATTTGAGATAGAGGGG |
| | | GAAGGTGAGGGTCGCCCTTACGAAGGCACGCAGACGGCTAAGCTGAAGGTCACG |
| | | AAAGGGGGACCCTTGCCCTTCGCATGGGACATACTCTCCCCACAGGTAAGTATT |
| | | GACTTTCTCGCCATCTCCTCCTCCCATCGTGTGCCGTTATAGATCATAGGGTCT |
| | | GGGCTTCTGCGTCGAGGACATCCAATCTGTCGAGTTACTAAGGCTCATGAGTCT |
| | | GTGTTGGGTCAGCCCTGCGCGACCCGTAAAATGTCCATTGTGTGTGTGTGTGTG |
| | | TTTGTGTGTGTGTGTGTGTGCTGTCAGTTCATGTACGGATCGAAGGCCTACGTGA |
| | | AGCATCCGGCGGACATACCAGGTAAGCATGTTGCGGGGATTCAAAGCAGTTACT |
| | | GATCAGTACCGCCCAACTTTGGTTACTGGCGTGAACTCTCGGCTCAGTTATCTAT |
| | | TGAAACCTCGCACCTTATAGATATCAATGCGTTGTTAGTATCCCATATCGAGGAT |
| | | GCGTAGTGTAGGGCGAAAGCTAATTGCTTCTCTTTATCCTGTAGACTATCTGAAG |
| | | CTCTCCTTTCCTGAGGGGTTTAAGTGGGAACGCGTTATGAACTTTGAGGATGGA |
| | | GGGGTCGTGACTGTTACCCAGGATTCTTCCCTGCAAGATGGAGAGTTCATATAC |
| | | AAAGTGAAACTTCGGGGAACGAATTTCCCATCAGACGGGCCAGTGATGCAGAAA |
| | | AAGACGATGGGGTGGGAGGCTTCATCCGAGAGGATGTATCCCGAGGACGGAGC |
| | | ATTGAAAGGCGAAATAAAACAAAGGCTGAAGTTGAAGGATGGGGGCCACTACGA |
| | | CGCGGAGGTTAAAACAACGTATAAAGCTAAAAAGCCAGTACAGCTCCCAGGCGC |
| | | ATATAACGTGAATATAAAGCTTGACATAACGAGTCATAACGAGGATTACACAATC |
| | | GTAGAACAGTACGAAAGAGCTGAAGGACGGCACTCCACCGGTGGGATGGATGA |
| | | ACTCTATAAAGACTACAAGGACGATGATGACAAGTAAACAAATGGTAAGGAAGGG |
| | | CACATCAATCTTTGCTTAATTGTCCTTTACTCTAAAGATGTATTTTATCATACTG |
| | | AATGCTAAACTTGATATCTCCTTTTAGGTCATTGATGTCCTTCACCCCGGGAAGG |
| | | CGACAGTGCCTAAGACAGAAATTCGGGAAAAACTAGCCAAAATGTACAAGACCAC |
| | | ACCGGATGTCATCTTTGTATTTGGATTCAGAACTCA |
| First | 75 | ATGGTCAGCAAAGGGGAAGAGGACAACATGGCCATCATTAAGGAGTTTATGCGA |
| mCherry | | TTCAAAGTACACATGGAGGGATCTGTTAATGGCCATGAATTTGAGATAGAGGGG |
| exon | | GAAGGTGAGGGTCGCCCTTACGAAGGCACGCAGACGGCTAAGCTGAAGGTCAC |
| sequence | | GAAAGGGGGACCCTTGCCCTTCGCATGGGACATACTCTCCCCACAG |
| encoding | | |
| for first part | | |
| of | | |
| transgene | | |
| First part of | 76 | GTAAGTATTGACTTTCTCGCCATCTCCTCCTCCCATCGTGTGCCGTTATAGATCA |
| intronic | | TAGGGTCTGGGCTTCTGCGTCGAGGACATCCAATCTGTCGAGTTACTAAGGCTC |
| region | | ATGAGTCTGTGTTGGGTCAGCCCTGCGCGACCCGTAAAATGTCCATTGTGTGTG |
| (with TDP- | | TGTGTGTGTTTGTGTGTGTGTGTGTGTGCTGTCAG |
| 43 binding | | |
| domain in | | |
| bold and | | |
| underlined) | | |
| CE and | 77 | TTCATGTACGGATCGAAGGCCTACGTGAAGCATCCGGCGGACATACCAG |
| second | | |
| mCherry | | |
| exon | | |
| sequence | | |
| encoding | | |
| for second | | |
| part of | | |
| transgene | | |
| Second | 78 | GTAAGCATGTTGCGGGGATTCAAAGCAGTTACTGATCAGTACCGCCCAACTTTG |
| part of | | GTTACTGGCGTGAACTCTCGGCTCAGTTATCTATTGAAACCTCGCACCTTATAGA |
| intronic | | TATCAATGCGTTGTTAGTATCCCATATCGAGGATGCGTAGTGTAGGGCGAAAGCT |
| region | | AATTGCTTCTCTTTATCCTGTAG |
| Third | 79 | ACTATCTGAAGCTCTCCTTTCCTGAGGGGTTTAAGTGGGAACGCGTTATGAACTT |
| mCherry | | TGAGGATGGAGGGGTCGTGACTGTTACCCAGGATTCTTCCCTGCAAGATGGAGA |
| exon | | GTTCATATACAAAGTGAAACTTCGGGGAACGAATTTCCCATCAGACGGGCCAGT |
| sequence | | GATGCAGAAAAAGACGATGGGGTGGGAGGCTTCATCCGAGAGGATGTATCCCGA |
| encoding | | GGACGGAGCATTGAAAGGCGAAATAAAACAAAGGCTGAAGTTGAAGGATGGGGG |
| for third | | CCACTACGACGCGGAGGTTAAAACAACGTATAAAGCTAAAAAGCCAGTACAGCT |
| part of | | CCCAGGCGCATATAACGTGAATATAAAGCTTGACATAACGAGTCATAACGAGGA |
| transgene | | TTACACAATCGTAGAACAGTACGAAAGAGCTGAAGGACGGCACTCCACCGGTGG |
| (PTC | | GATGGATGAACTCTATAAAGACTACAAGGACGATGATGACAAGTAA |
| shown in | | |
| bold) | | |

The sequence comprising the start codon and further intronic sequence (e.g., based on RPS24) was as the same as described in Example 1A.

### Example 2C

| | SEQ ID | |
|---|---|---|
| | NO: | |
| Construct 2C | 80 | ATGGTCAGCAAAGGGGAAGAGGACAACATGGCCATCATTAAGGAGTTTATGCG |
| | | ATTCAAAGTACACATGGAGGGATCTGTTAATGGCCATGAATTTGAGATAGAGGG |
| | | GGAAGGTGAGGGTCGCCCTTACGAAGGCACGCAGACGGCTAAGCTGAAGGTC |
| | | ACGAAAGGGGGACCCTTGCCCTTCGCATGGGACATACTCTCCCCACAGGTAAG |
| | | AGCGGGGTGATAAGAGCCTCAGGGTTATTTCCCAGACTTTGAATTTGCTAATTA |
| | | TCTCATACGCAACCTAGCGAATCTCATAGGGGTCCGGGCTACTTGTCTGAGCT |
| | | TCTTCTCTTGTGCCCTATGCTCTGTTCCTCTTTTGACGCCCTTAGTTCATGTACG |
| | | GTTCGAAGGCTTACGTCAAACATCCCGCCGACATTCCGGGTAAGTGTGTGTGT |
| | | GTGTGTGTTTGTGTGTGTGTGTGTGTGAGTAACTCCAGGGCCTGGCCCCTCTG |
| | | GATCCGTGAAGTAGCATGGGGTTAAGGCACGGCGGAAGCGCATTATCTATGAA |
| | | TTTAGGGCCAATGCGAGTCCTGTTAGTTCAAAGCCTTCTGTTTACCCTTTTCCG |
| | | TTTCCTTCTTATCTACGCAGACTATCTGAAGCTCTCCTTTCCTGAGGGGTTTAA |
| | | GTGGGAACGCGTTATGAACTTTGAGGATGGAGGGGTCGTGACTGTTACCCAG |
| | | GATTCTTCCCTGCAAGATGGAGAGTTCATATACAAAGTGAAACTTCGGGGAAC |
| | | GAATTTCCCATCAGACGGGCCAGTGATGCAGAAAAAGACGATGGGGTGGGAG |
| | | GCTTCATCCGAGAGGATGTATCCCGAGGACGGAGCATTGAAAGGCGAAATAAA |
| | | ACAAAGGCTGAAGTTGAAGGATGGGGGCCACTACGACGCGGAGGTTAAAACA |
| | | ACGTATAAAGCTAAAAAGCCAGTACAGCTCCCAGGCGCATATAACGTGAATATA |
| | | AAGCTTGACATAACGAGTCATAACGAGGATTACACAATCGTAGAACAGTACGAA |
| | | AGAGCTGAAGGACGGCACTCCACCGGTGGGATGGATGAACTCTATAAAGACTA |
| | | CAAGGACGATGATGACAAGTAAACAAATGGTAAGGAAGGGCACATCAATCTTT |
| | | GCTTAATTGTCCTTTACTCTAAAGATGTATTTTATCATACTGAATGCTAAACTTGA |
| | | TATCTCCTTTTAGGTCATTGATGTCCTTCACCCCGGGAAGGCGACAGTGCCTAA |
| | | GACAGAAATTCGGGAAAAACTAGCCAAAATGTACAAGACCACACCGGATGTCA |
| | | TCTTTGTATTTGGATTCAGAACTCA |
| First | 81 | ATGGTCAGCAAAGGGGAAGAGGACAACATGGCCATCATTAAGGAGTTTATGCG |
| mCherry | | ATTCAAAGTACACATGGAGGGATCTGTTAATGGCCATGAATTTGAGATAGAGGG |
| exon | | GGAAGGTGAGGGTCGCCCTTACGAAGGCACGCAGACGGCTAAGCTGAAGGTC |
| sequence | | ACGAAAGGGGGACCCTTGCCCTTCGCATGGGACATACTCTCCCCACAG |
| encoding for | | |
| first part of | | |
| transgene | | |
| First part of | 82 | GTAAGAGCGGGGTGATAAGAGCCTCAGGGTTATTTCCCAGACTTTGAATTTGC |
| intronic | | TAATTATCTCATACGCAACCTAGCGAATCTCATAGGGGTCCGGGCTACTTGTCT |
| region | | GAGCTTCTTCTCTTGTGCCCTATGCTCTGTTCCTCTTTTGACGCCCTTAG |
| CE and | 83 | TTCATGTACGGTTCGAAGGCTTACGTCAAACATCCCGCCGACATTCCGG |
| second | | |
| mCherry | | |
| exon | | |
| sequence | | |
| encoding for | | |
| second part | | |
| of transgene | | |
| Second part | 84 | GTAAGTGTGTGTGTGTGTGTGTTTGTGTGTGTGTGTGTGTGAGTAACTCCAGG |
| of intronic | | GCCTGGCCCCTCTGGATCCGTGAAGTAGCATGGGGTTAAGGCACGGCGGAAG |
| region (TDP- | | CGCATTATCTATGAATTTAGGGCCAATGCGAGTCCTGTTAGTTCAAAGCCTTCT |
| 43 binding | | GTTTACCCTTTTCCGTTTCCTTCTTATCTACGCAG |
| domain | | |
| shown in | | |
| bold) | | |
| Third | 85 | ACTATCTGAAGCTCTCCTTTCCTGAGGGGTTTAAGTGGGAACGCGTTATGAACT |
| mCherry | | TTGAGGATGGAGGGGTCGTGACTGTTACCCAGGATTCTTCCCTGCAAGATGGA |
| exon | | GAGTTCATATACAAAGTGAAACTTCGGGGAACGAATTTCCCATCAGACGGGCC |
| encoding for | | AGTGATGCAGAAAAAGACGATGGGGTGGGAGGCTTCATCCGAGAGGATGTAT |
| third part of | | CCCGAGGACGGAGCATTGAAAGGCGAAATAAAACAAAGGCTGAAGTTGAAGGA |
| transgene | | TGGGGGCCACTACGACGCGGAGGTTAAAACAACGTATAAAGCTAAAAAGCCAG |
| (PTC shown | | TACAGCTCCCAGGCGCATATAACGTGAATATAAAGCTTGACATAACGAGTCATA |
| in bold) | | ACGAGGATTACACAATCGTAGAACAGTACGAAAGAGCTGAAGGACGGCACTCC |
| | | ACCGGTGGGATGGATGAACTCTATAAAGACTACAAGGACGATGATGACAAGTA |
| | | A |

The sequence comprising the start codon and further intronic sequence (e.g., based on RPS24) was as the same as described in Example 1A.

### Examples 2D-2J

The following examples are all Design 2 style constructs which express mScarlet (i.e., part of the mScarlet coding sequence is within the cryptic exon). Importantly, they have different TDP-43 binding domains, with shorter TG repeats than shown in other Examples (e.g., Example 1A) comprising intronic regions based on AARS1.

For all of the Examples 2D-2J, the construct further comprised a C-terminal FLAG tag, with sequence

SEQ ID NO: 116
GACTACAAGGACGATGATGACAAG.

Each 2D-2J example construct further features the constitutive downstream intron, with an identical sequence to that described for the Example 1A construct.

### Example 2D

This example construct contains short TG repeats on each side of the cryptic exon

| | SEQ ID | Sequence |
|---|---|---|
| | NO: | |
| Construct 2D | 117 | CGGCCGCTTCTTGGTGCCAGCTTATCATAGCGCTACCGGTCGCCAC |
| | | CATGGCGCGGACAATGGTTGCTATGGTGTCCAAAGGCGAAGCAGGT |
| | | AAGAGAGATCTGTTGCTCTGGAGGGGTGTGAATGCTGCGGCATGAG |
| | | TGAATGTCTCGATGATTGACTGAATGGATGCTTGCGTGTGTGTGTGT |
| | | GGTCTAGTTATCAAGGAATTCATGAGGTTCAAAGTCCACATGGAAGG |
| | | TTCAATGAACGGCCATGAATTCGAGATTGAAGGCGAGGGTGAAGGC |
| | | CGACCTTACGAAGGAACACAAACTGCAAAGGTGGTTGTGTGTGTGT |
| | | GCATGAATGCATGTTTGTGTGATTAAAGCGTGCCTGGTTTATCGACG |
| | | TGTGTATGAACGATGGGTGCCTGCCTTCGCCGTTGTTTCTTTCTTTC |
| | | CCGCCTCCAGCTCAAGGTGACGAAGGGCGGGCCTCTGCCCTTCTCT |
| | | TGGGATATCCTGAGCCCGCAGTTTATGTACGGCAGCCGGGCTTTCA |
| | | CCAAACACCCTGCCGATATCCCAGACTACTATAAACAGTCCTTTCCA |
| | | GAAGGATTTAAGTGGGAGCGAGTCATGAATTTCGAGGACGGAGGTG |
| | | CCGTGACGGTTACTCAGGACACCAGCCTGGAGGACGGCACCCTGAT |
| | | CTACAAGGTGAAGCTGAGGGGCACCAACTTCCCCCCCGACGGCCC |
| | | CGTGATGCAGAAGAAGACCATGGGCTGGGAGGCCAGCACCGAGAG |
| | | GCTGTACCCCGAGGACGGCGTGCTGAAGGGCGACATCAAGATGGC |
| | | CCTGAGGCTGAAGGACGGCGGCAGGTACCTGGCCGACTTCAAGAC |
| | | CACCTACAAGGCCAAGAAGCCCGTGCAGATGCCCGGCGCCTACAAC |
| | | GTGGACAGGAAGCTGGACATCACCAGCCACAACGAGGACTACACCG |
| | | TGGTGGAGCAGTACGAGAGGAGCGAGGGCAGGCACAGCACCGGCG |
| | | GCATGGACGAGCTGTACAAGGACTACAAGGACGATGATGACAAGTG |
| | | ATAAACAAATGGTAAGGAAGGGCACATCAATCTTTGCTTAATTGTCCT |
| | | TTACTCTAAAGATGTATTTTATCATACTGAATGCTAAACTTGATATC |
| | | CTCTTTTAGGTCATTGATGTCCTTCACCCCGGGAAGGCGACAGTGCC |
| | | TAAGACAGAAATTCGGGAAAAACTAGCCAAAATGTACAAGACCACAC |
| | | CGGATGTCATCTTTGTATTTGGATTCAGAACTCA |
| Exon sequence | 118 | ATGGCGCGGACAATGGTTGCTATGGTGTCCAAAGGCGAAGCAG |
| encoding for first | | |
| part of mScarlet | | |
| First part of | 119 | GTAAGAGAGATCTGTTGCTCTGGAGGGGTGTGAATGCTGCGGCATG |
| intronic region (TG | | AGTGAATGTCTCGATGATTGACTGAATGGATGCTTGCGTGTGTGTG |
| repeats in bold) | | TGTGGTCTAG |
| Cryptic exon | 120 | TTATCAAGGAATTCATGAGGTTCAAAGTCCACATGGAAGGTTCAATG |
| sequence | | AACGGCCATGAATTCGAGATTGAAGGCGAGGGTGAAGGCCGACCTT |
| encoding for | | ACGAAGGAACACAAACTGCAAAG |
| second part of | | |
| mScarlet | | |
| Second part of | 121 | GTGGTTGTGTGTGTGTGCATGAATGCATGTTTGTGTGATTAAAGCGT |
| intronic region (TG | | GCCTGGTTTATCGACGTGTGTATGAACGATGGGTGCCTGCCTTCGC |
| repeats in bold) | | CGTTGTTTCTTTCTTTCCCGCCTCCAG |
| Exon sequence | 122 | CTCAAGGTGACGAAGGGCGGGCCTCTGCCCTTCTCTTGGGATATCC |
| encoding for third | | TGAGCCCGCAGTTTATGTACGGCAGCCGGGCTTTCACCAAACACCC |
| part of mScarlet | | TGCCGATATCCCAGACTACTATAAACAGTCCTTTCCAGAAGGATTTA |
| | | AGTGGGAGCGAGTCATGAATTTCGAGGACGGAGGTGCCGTGACGGTT |
| | | ACTCAGGACACCAGCCTGGAGGACGGCACCCTGATCTACAAGGTGA |
| | | AGCTGAGGGGCACCAACTTCCCCCCCGACGGCCCCGTGATGCAGA |
| | | AGAAGACCATGGGCTGGGAGGCCAGCACCGAGAGGCTGTACCCCG |
| | | AGGACGGCGTGCTGAAGGGCGACATCAAGATGGCCCTGAGGCTGA |
| | | AGGACGGCGGCAGGTACCTGGCCGACTTCAAGACCACCTACAAGG |
| | | CCAAGAAGCCCGTGCAGATGCCCGGCGCCTACAACGTGGACAGGA |
| | | AGCTGGACATCACCAGCCACAACGAGGACTACACCGTGGTGGAGCA |
| | | GTACGAGAGGAGCGAGGGCAGGCACAGCACCGGCGGCATGGACGA |
| | | GCTGTACAAGGACTACAAGGACGATGATGACAAGTGA |

### Example 2E

Similar to Example 2D, this construct also contains short TG repeats on each side of the cryptic.

| | SEQ ID NO: | Sequence |
|---|---|---|
| Example 2E | 123 | CGGCCGCTTCTTGGTGCCAGCTTATCATAGCGCTACCGGTCGCCAC |
| (start codon in | | CATGGCGAGAACAATGGTTGCTATGGTGTCCAAGGGTGAGGCAGGT |
| bold) | | AAGAATCGTAGCATACAAAATTATAGGAGTGGCTGTGTGAATTGGTC |
| | | ACTGGCAATGTCCGTGCGTGAGTGGTGCGATCAGTGGTGGTTGAAT |
| | | GCCTGGATGACTGAGTGTGTGTGTGTGCTTCAGTCATCAAGGAGTTT |
| | | ATGCGCTTCAAGGTGCACATGGAAGGATCAATGAATGGCCACGAGT |
| | | TCGAAATTGAAGGCGAGGGCGAGGGCCGCCCCTATGAAGGGACAC |
| | | AGACTGCCAAGGTGTGTGTGTGTGTGTGAGTGTGTGGTTGATTGTCT |
| | | GACAGGCAGGTGATTAGTGAGTGCTTGAAGACGTTATCAAGCGTGA |
| | | TTGTTCCTTGGGAGACTGAAGTGTGGTTGGAAAACGAATTATCATTG |
| | | TTCTTCCCCGCTACAGCTCAAGGTGACGAAGGGCGGGCCTCTGCCC |
| | | TTCTCTTGGGATATCCTGAGCCCGCAGTTTATGTACGGCAGCCGGG |
| | | CTTTCACCAAACACCCTGCCGATATCCCAGACTACTATAAACAGTCC |
| | | TTTCCAGAAGGATTTAAGTGGGAGCGAGTCATGAATTTCGAGGACG |
| | | GAGGTGCCGTGACGGTTACTCAGGACACCAGCCTGGAGGACGGCA |
| | | CCCTGATCTACAAGGTGAAGCTGAGGGGCACCAACTTCCCCCCCGA |
| | | CGGCCCCGTGATGCAGAAGAAGACCATGGGCTGGGAGGCCAGCAC |
| | | CGAGAGGCTGTACCCCGAGGACGGCGTGCTGAAGGGCGACATCAA |
| | | GATGGCCCTGAGGCTGAAGGACGGCGGCAGGTACCTGGCCGACTT |
| | | CAAGACCACCTACAAGGCCAAGAAGCCCGTGCAGATGCCCGGCGC |
| | | CTACAACGTGGACAGGAAGCTGGACATCACCAGCCACAACGAGGAC |
| | | TACACCGTGGTGGAGCAGTACGAGAGGAGCGAGGGCAGGCACAGC |
| | | ACCGGCGGCATGGACGAGCTGTACAAGGACTACAAGGACGATGATG |
| | | ACAAGTGATAAACAAATGGTAAGGAAGGGCACATCAATCTTTGCTTA |
| | | ATTGTCCTTTACTCTAAAGATGTATTTTATCATACTGAATGCTAAACTT |
| | | GATATCTCCTTTTAGGTCATTGATGTCCTTCACCCCGGGAAGGCGAC |
| | | AGTGCCTAAGACAGAAATTCGGGAAAAACTAGCCAAAATGTACAAGA |
| | | CCACACCGGATGTCATCTTTGTATTTGGATTCAGAACTCA |
| Exon encoding | 124 | ATGGCGAGAACAATGGTTGCTATGGTGTCCAAGGGTGAGGCAG |
| for first part of | | |
| mScarlet | | |
| First part of | 125 | GTAAGAATCGTAGCATACAAAATTATAGGAGTGGCTGTGTGAATTGG |
| intronic region | | TCACTGGCAATGTCCGTGCGTGAGTGGTGCGATCAGTGGTGGTTGA |
| (TDP-43 binding | | ATGCCTGGATGACTGAGTGTGTGTGTGTGCTTCAG |
| domain in bold) | | |
| Cryptic exon | 126 | TCATCAAGGAGTTTATGCGCTTCAAGGTGCACATGGAAGGATCAATG |
| encoding for | | AATGGCCACGAGTTCGAAATTGAAGGCGAGGGCGAGGGCCGCCCC |
| second part of | | TATGAAGGGACACAGACTGCCAAG |
| mScarlet | | |
| Second part of | 127 | GTGTGTGTGTGTGTGTGAGTGTGTGGTTGATTGTCTGACAGGCAGG |
| intronic region | | TGATTAGTGAGTGCTTGAAGACGTTATCAAGCGTGATTGTTCCTTGG |
| (TG repeats in | | GAGACTGAAGTGTGGTTGGAAAACGAATTATCATTGTTCTTCCCCGC |
| bold) | | TACAG |
| Exon encoding | 128 | CTCAAGGTGACGAAGGGCGGGCCTCTGCCCTTCTCTTGGGATATCC |
| for third part of | | TGAGCCCGCAGTTTATGTACGGCAGCCGGGCTTTCACCAAACACCC |
| mScarlet | | TGCCGATATCCCAGACTACTATAAACAGTCCTTTCCAGAAGGATTTA |
| | | AGTGGGAGCGAGTCATGAATTTCGAGGACGGAGGTGCCGTGACGGTT |
| | | ACTCAGGACACCAGCCTGGAGGACGGCACCCTGATCTACAAGGTGA |
| | | AGCTGAGGGGCACCAACTTCCCCCCCGACGGCCCCGTGATGCAGA |
| | | AGAAGACCATGGGCTGGGAGGCCAGCACCGAGAGGCTGTACCCCG |
| | | AGGACGGCGTGCTGAAGGGCGACATCAAGATGGCCCTGAGGCTGA |
| | | AGGACGGCGGCAGGTACCTGGCCGACTTCAAGACCACCTACAAGG |
| | | CCAAGAAGCCCGTGCAGATGCCCGGCGCCTACAACGTGGACAGGA |
| | | AGCTGGACATCACCAGCCACAACGAGGACTACACCGTGGTGGAGCA |
| | | GTACGAGAGGAGCGAGGGCAGGCACAGCACCGGCGGCATGGACGA |
| | | GCTGTACAAGGACTACAAGGACGATGATGACAAGTGA |

### Example 2F

This Example construct had a downstream TDP-43 binding domain.

| | SEQ ID | Sequence |
|---|---|---|
| | NO: | |
| Construct- | 129 | CGGCCGCTTCTTGGTGCCAGCTTATCATAGCGCTACCGGTCGCCACC |
| Example 2F | | ATGGCGAGAACGATGGTGGCTATGGTCTCCAAAGGCGAGGCAGGTAA |
| (start codon | | GTCTTACCCTATTGAATGATTACTTAAATGGGGGTGTGGCTGAGCCGA |
| shown in bold) | | TGTAGCGTGATTGCTAGCTACGAGTGCGTGTTGTATTAACAATGGCTC |
| | | CTCCGTGTGGCTGGCCACTCCAGTGATAAAGGAATTCATGAGGTTCAA |
| | | GGTGCACATGGAAGGGTCAATGAATGGCCATGAGTTCGAGATCGAGG |
| | | GTGAGGGCGAGGGCCGCCCATATGAAGGGACCCAGACCGCGAAGGT |
| | | GTGTGTGTTATGTGTGTGACGTGTGGATGTGATGTGTGCTTGAGTATA |
| | | AGTGTGAATGGCATCCGGTGATGAAGCGCGCGAAACAAGATTCTCCTT |
| | | CTTCCTCCCTTCCAGCTCAAAGTAACGAAGGGCGGGCCTCTGCCCTT |
| | | CTCTTGGGATATCCTGAGCCCGCAGTTTATGTACGGCAGCCGGGCTTT |
| | | CACCAAACACCCTGCCGATATCCCAGACTACTATAAACAGTCCTTTCC |
| | | AGAAGGATTTAAGTGGGAGCGAGTCATGAATTTCGAGGACGGAGGTG |
| | | CCGTGACGGTTACTCAGGACACCAGCCTGGAGGACGGCACCCTGATC |
| | | TACAAGGTGAAGCTGAGGGGCACCAACTTCCCCCCCGACGGCCCCGT |
| | | GATGCAGAAGAAGACCATGGGCTGGGAGGCCAGCACCGAGAGGCTG |
| | | TACCCCGAGGACGGCGTGCTGAAGGGCGACATCAAGATGGCCCTGA |
| | | GGCTGAAGGACGGCGGCAGGTACCTGGCCGACTTCAAGACCACCTAC |
| | | AAGGCCAAGAAGCCCGTGCAGATGCCCGGCGCCTACAACGTGGACA |
| | | GGAAGCTGGACATCACCAGCCACAACGAGGACTACACCGTGGTGGAG |
| | | CAGTACGAGAGGAGCGAGGGCAGGCACAGCACCGGCGGCATGGACG |
| | | AGCTGTACAAGGACTACAAGGACGATGATGACAAGTGATAAACAAATG |
| | | GTAAGGAAGGGCACATCAATCTTTGCTTAATTGTCCTTTACTCTAAAGA |
| | | TGTATTTTATCATACTGAATGCTAAACTTGATATCTCCTTTTAGGTCATT |
| | | GATGTCCTTCACCCCGGGAAGGCGACAGTGCCTAAGACAGAAATTCG |
| | | GGAAAAACTAGCCAAAATGTACAAGACCACACCGGATGTCATCTTTGT |
| | | ATTTGGATTCAGAACTCA |
| Exon encoding | 130 | ATGGCGAGAACGATGGTGGCTATGGTCTCCAAAGGCGAGGCAG |
| for first part of | | |
| mScarlet | | |
| First part of | 131 | GTAAGTCTTACCCTATTGAATGATTACTTAAATGGGGGTGTGGCTGAG |
| intronic region | | CCGATGTAGCGTGATTGCTAGCTACGAGTGCGTGTTGTATTAACAATG |
| | | GCTCCTCCGTGTGGCTGGCCACTCCAG |
| Cryptic exon | 132 | TGATAAAGGAATTCATGAGGTTCAAGGTGCACATGGAAGGGTCAATGA |
| encoding for | | ATGGCCATGAGTTCGAGATCGAGGGTGAGGGCGAGGGCCGCCCATA |
| second part of | | TGAAGGGACCCAGACCGCGAAG |
| mScarlet | | |
| Second part of | 133 | GTGTGTGTGTTATGTGTGTGACGTGTGGATGTGATGTGTGCTTGAGTA |
| intronic region | | TAAGTGTGAATGGCATCCGGTGATGAAGCGCGCGAAACAAGATTCTC |
| (TG-repeats in | | CTTCTTCCTCCCTTCCAG |
| bold) | | |
| Exon encoding | 134 | CTCAAAGTAACGAAGGGCGGGCCTCTGCCCTTCTCTTGGGATATCCT |
| for third part of | | GAGCCCGCAGTTTATGTACGGCAGCCGGGCTTTCACCAAACACCCTG |
| mScarlet | | CCGATATCCCAGACTACTATAAACAGTCCTTTCCAGAAGGATTTAAGTG |
| | | GGAGCGAGTCATGAATTTCGAGGACGGAGGTGCCGTGACGGTTACTC |
| | | AGGACACCAGCCTGGAGGACGGCACCCTGATCTACAAGGTGAAGCTG |
| | | AGGGGCACCAACTTCCCCCCCGACGGCCCCGTGATGCAGAAGAAGA |
| | | CCATGGGCTGGGAGGCCAGCACCGAGAGGCTGTACCCCGAGGACGG |
| | | CGTGCTGAAGGGCGACATCAAGATGGCCCTGAGGCTGAAGGACGGC |
| | | GGCAGGTACCTGGCCGACTTCAAGACCACCTACAAGGCCAAGAAGCC |
| | | CGTGCAGATGCCCGGCGCCTACAACGTGGACAGGAAGCTGGACATCA |
| | | CCAGCCACAACGAGGACTACACCGTGGTGGAGCAGTACGAGAGGAG |
| | | CGAGGGCAGGCACAGCACCGGCGGCATGGACGAGCTGTACAAGGAC |
| | | TACAAGGACGATGATGACAAGTGA |

### Example 2G

This Example also has a downstream TDP-43 binding domain.

| | SEQ ID NO: | Sequence |
|---|---|---|
| Construct | 135 | CGGCCGCTTCTTGGTGCCAGCTTATCATAGCGCTACCGGTCGCCACCA |
| 2G (start | | TGGCGAGGACAATGGTTGCCATGGTGTCCAAAGGAGAGGCAGGTAAGT |
| codon in | | AGCTTATGGCTTTGGGGCCGGTCCCAAATTCGTGTGACTGGCGCGGAT |
| bold) | | CTGGGTGTTTGTGAAACAAGTGTGCATGTCTTTTTCGCCTTTCGATTTCC |
| | | GGGTGCCTGTTTTTCAAAGTGATCAAAGAATTTATGAGGTTCAAGGTGC |
| | | ACATGGAAGGTAGCATGAACGGTCATGAGTTCGAGATAGAAGGCGAGG |
| | | GCGAGGGACGCCCGTACGAAGGCACTCAGACGGCAAAGGTGTGTGTG |
| | | TCCTGTGTGTGGAGTGTGCTTGCGTGGCGTGCCTGCCACCGACCTCTG |
| | | AGTGCATGCCTGCAAGCTGCCTTCGTCCACGCTTTCCGGATACCCAACT |
| | | TTCTTTTTTACAGCTCAAGGTGACAAAGGGCGGGCCTCTGCCCTTCTCT |
| | | TGGGATATCCTGAGCCCGCAGTTTATGTACGGCAGCCGGGCTTTCACC |
| | | AAACACCCTGCCGATATCCCAGACTACTATAAACAGTCCTTTCCAGAAG |
| | | GATTTAAGTGGGAGCGAGTCATGAATTTCGAGGACGGAGGTGCCGTGA |
| | | CGGTTACTCAGGACACCAGCCTGGAGGACGGCACCCTGATCTACAAGG |
| | | TGAAGCTGAGGGGCACCAACTTCCCCCCCGACGGCCCCGTGATGCAGA |
| | | AGAAGACCATGGGCTGGGAGGCCAGCACCGAGAGGCTGTACCCCGAG |
| | | GACGGCGTGCTGAAGGGCGACATCAAGATGGCCCTGAGGCTGAAGGA |
| | | CGGCGGCAGGTACCTGGCCGACTTCAAGACCACCTACAAGGCCAAGAA |
| | | GCCCGTGCAGATGCCCGGCGCCTACAACGTGGACAGGAAGCTGGACAT |
| | | CACCAGCCACAACGAGGACTACACCGTGGTGGAGCAGTACGAGAGGAG |
| | | CGAGGGCAGGCACAGCACCGGCGGCATGGACGAGCTGTACAAGGACTA |
| | | CAAGGACGATGATGACAAGTGATAAACAAATGGTAAGGAAGGGCACAT |
| | | CAATCTTTGCTTAATTGTCCTTTACTCTAAAGATGTATTTTATCATAC |
| | | TGAATGCTAAACTTGATATCTCCTTTTAGGTCATTGATGTCCTTCACC |
| | | CCGGGAAGGCGACAGTGCCTAAGACAGAAATTCGGGAAAAACTAGCCA |
| | | AAATGTACAAGACCACACCGGATGTCATCTTTGTATTTGGATTCAGAA |
| | | CTCA |
| Exon | 136 | ATGGCGAGGACAATGGTTGCCATGGTGTCCAAAGGAGAGGCAG |
| encoding | | |
| for first | | |
| part of | | |
| mScarlet | | |
| First part | 137 | GTAAGTAGCTTATGGCTTTGGGGCCGGTCCCAAATTCGTGTGACTGGC |
| of intronic | | GCGGATCTGGGTGTTTGTGAAACAAGTGTGCATGTCTTTTTCGCCTTT |
| region | | CGATTTCCGGGTGCCTGTTTTTCAAAG |
| Cryptic | 138 | TGATCAAAGAATTTATGAGGTTCAAGGTGCACATGGAAGGTAGCATGAA |
| exon | | CGGTCATGAGTTCGAGATAGAAGGCGAGGGCGAGGGACGCCCGTACG |
| encoding | | AAGGCACTCAGACGGCAAAG |
| for second | | |
| part of | | |
| mScarlet | | |
| Second | 139 | GTGTGTGTGTCCTGTGTGTGGAGTGTGCTTGCGTGGCGTGCCTGCCAC |
| part of | | CGACCTCTGAGTGCATGCCTGCAAGCTGCCTTCGTCCACGCTTTCCGG |
| intronic | | ATACCCAACTTTCTTTTTTACAG |
| region (TG | | |
| repeats in | | |
| bold)) | | |
| Exon | 140 | CTCAAGGTGACAAAGGGGGGGCCTCTGCCCTTCTCTTGGGATATCCTG |
| encoding | | AGCCCGCAGTTTATGTACGGCAGCCGGGCTTTCACCAAACACCCTGCCG |
| for third | | ATATCCCAGACTACTATAAACAGTCCTTTCCAGAAGGATTTAAGTGGGA |
| part of | | GCGAGTCATGAATTTCGAGGACGGAGGTGCCGTGACGGTTACTCAGGA |
| mScarlet | | CACCAGCCTGGAGGACGGCACCCTGATCTACAAGGTGAAGCTGAGGGG |
| | | CACCAACTTCCCCCCCGACGGCCCCGTGATGCAGAAGAAGACCATGGG |
| | | CTGGGAGGCCAGCACCGAGAGGCTGTACCCCGAGGACGGCGTGCTGA |
| | | AGGGCGACATCAAGATGGCCCTGAGGCTGAAGGACGGCGGCAGGTAC |
| | | CTGGCCGACTTCAAGACCACCTACAAGGCCAAGAAGCCCGTGCAGATG |
| | | CCCGGCGCCTACAACGTGGACAGGAAGCTGGACATCACCAGCCACAAC |
| | | GAGGACTACACCGTGGTGGAGCAGTACGAGAGGAGCGAGGGCAGGCA |
| | | CAGCACCGGCGGCATGGACGAGCTGTACAAGGACTACAAGGACGATGA |
| | | TGACAAGTGA |

### Example 2H

This Example has short TG repeats on both sides of the cryptic exon.

| | SEQ ID NO: | Sequence |
|---|---|---|
| Construct | 141 | CGGCCGCTTCTTGGTGCCAGCTTATCATAGCGCTACCGGTCGCCACCA |
| 2H (start | | TGGCCCGAACAATGGTCGCCATGGTGTCCAAGGGAGAAGCGGGTAAGT |
| codon in | | ACACCGGCCTAACTGGTCTCAGTCAGAATAAGAGTGTCTGAAATCAGGT |
| bold) | | GGAGTGGTTGGGCAATTAGCGTGCTTGATTTTCTCTGCGTGACTGGCGT |
| | | ACGTTGCTGTGGTTGTCTTGTGTGGTAGTGATCAAAGAATTTATGAGGTT |
| | | CAAAGTCCACATGGAAGGATCTATGAATGGCCACGAGTTTGAGATTGAA |
| | | GGAGAGGGAGAGGGACGGCCGTACGAAGGGACACAAACGGCCAAGGT |
| | | GTGTGTGGTGTGTTTGACCGTCCGGGTGAATGTCTCCTAATAGTGCGTG |
| | | CGTGACCCGTAGTGTGGATGCAGGGGACCGGGAAGTGTGTCTAACTGT |
| | | TCCACCCCCCTTTTACAGCTCAAAGTGACCAAGGGGGGCCTCTGCCC |
| | | TTCTCTTGGGATATCCTGAGCCCGCAGTTTATGTACGGCAGCCGGGCTT |
| | | TCACCAAACACCCTGCCGATATCCCAGACTACTATAAACAGTCCTTTCCA |
| | | GAAGGATTTAAGTGGGAGCGAGTCATGAATTTCGAGGACGGAGGTGCC |
| | | GTGACGGTTACTCAGGACACCAGCCTGGAGGACGGCACCCTGATCTAC |
| | | AAGGTGAAGCTGAGGGGCACCAACTTCCCCCCCGACGGCCCCGTGATG |
| | | CAGAAGAAGACCATGGGCTGGGAGGCCAGCACCGAGAGGCTGTACCC |
| | | CGAGGACGGCGTGCTGAAGGGCGACATCAAGATGGCCCTGAGGCTGA |
| | | AGGACGGCGGCAGGTACCTGGCCGACTTCAAGACCACCTACAAGGCCA |
| | | AGAAGCCCGTGCAGATGCCCGGCGCCTACAACGTGGACAGGAAGCTG |
| | | GACATCACCAGCCACAACGAGGACTACACCGTGGTGGAGCAGTACGAG |
| | | AGGAGCGAGGGCAGGCACAGCACCGGCGGCATGGACGAGCTGTACAAG |
| | | GACTACAAGGACGATGATGACAAGTGATAAACAAATGGTAAGGAAGGGC |
| | | ACATCAATCTTTGCTTAATTGTCCTTTACTCTAAAGATGTATTTTATCA |
| | | TACTGAATGCTAAACTTGATATCTCCTTTTAGGTCATTGATGTCCTTCAC |
| | | CCCGGGAAGGCGACAGTGCCTAAGACAGAAATTCGGGAAAAACTAGCC |
| | | AAAATGTACAAGACCACACCGGATGTCATCTTTGTATTTGGATTCAGAAC |
| | | TCA |
| Exon | 142 | ATGGCCCGAACAATGGTCGCCATGGTGTCCAAGGGAGAAGCGG |
| encoding | | |
| for first | | |
| part of | | |
| mScarlet | | |
| First part | 143 | GTAAGTACACCGGCCTAACTGGTCTCAGTCAGAATAAGAGTGTCTGAAA |
| of intronic | | TCAGGTGGAGTGGTTGGGCAATTAGCGTGCTTGATTTTCTCTGCGTGAC |
| region (TG | | TGGCGTACGTTGCTGTGGTTGTCTTGTGTGGTAG |
| repeats in | | |
| bold) | | |
| Cryptic | 144 | TGATCAAAGAATTTATGAGGTTCAAAGTCCACATGGAAGGATCTATGAAT |
| exon | | GGCCACGAGTTTGAGATTGAAGGAGAGGGAGAGGGACGGCCGTACGA |
| encoding | | AGGGACACAAACGGCCAAG |
| for second | | |
| part of | | |
| mScarlet | | |
| Second | 145 | GTGTGTGTGGTGTGTTTGACCGTCCGGGTGAATGTCTCCTAATAGTGCG |
| part of | | TGCGTGACCCGTAGTGTGGATGCAGGGGACCGGGAAGTGTGTCTAACT |
| intronic | | GTTCCACCCCCCTTTTACAG |
| region (TG | | |
| repeats in | | |
| bold) | | |
| Exon | 146 | CTCAAAGTGACCAAGGGCGGGCCTCTGCCCTTCTCTTGGGATATCCTGA |
| encoding | | GCCCGCAGTTTATGTACGGCAGCCGGGCTTTCACCAAACACCCTGCCG |
| for third | | ATATCCCAGACTACTATAAACAGTCCTTTCCAGAAGGATTTAAGTGGGAG |
| part of | | CGAGTCATGAATTTCGAGGACGGAGGTGCCGTGACGGTTACTCAGGAC |
| mScarlet | | ACCAGCCTGGAGGACGGCACCCTGATCTACAAGGTGAAGCTGAGGGGC |
| | | ACCAACTTCCCCCCCGACGGCCCCGTGATGCAGAAGAAGACCATGGGC |
| | | TGGGAGGCCAGCACCGAGAGGCTGTACCCCGAGGACGGCGTGCTGAA |
| | | GGGCGACATCAAGATGGCCCTGAGGCTGAAGGACGGCGGCAGGTACC |
| | | TGGCCGACTTCAAGACCACCTACAAGGCCAAGAAGCCCGTGCAGATGC |
| | | CCGGCGCCTACAACGTGGACAGGAAGCTGGACATCACCAGCCACAACG |
| | | AGGACTACACCGTGGTGGAGCAGTACGAGAGGAGCGAGGGCAGGCAC |
| | | AGCACCGGCGGCATGGACGAGCTGTACAAGGACTACAAGGACGATGAT |
| | | GACAAGTGA |

### Example 2I

This Example construct did not have any expended TG repeats, but instead was TG-enriched, with TGs spaced throughout the introns.

| | SEQ ID | |
|---|---|---|
| | NO: | Sequence |
| Construct | 147 | CGGCCGCTTCTTGGTGCCAGCTTATCATAGCGCTACCGGTCGCCACCATGG |
| 21 (start | | CGAGAACAATGGTCGCGATGGTATCTAAGGGCGAAGCAGGTAAGCGGCGT |
| codon in | | GCTTGTTGCGTGGTTGGGGTGTGGGTGTGAGTGGGATGGGAGAGTGGTTG |
| bold) | | TCGCGTGTGGTTGGCTCGGGTGCTTGGATGGGTGATTGTCGGCGTGTTTGA |
| | | CAGTGATAAAAGAGTTTATGAGATTCAAAGTCCACATGGAGGGATCAATGAA |
| | | CGGACACGAATTTGAAATTGAAGGCGAGGGCGAAGGAAGACCTTATGAGG |
| | | GGACACAGACCGCCAAGGTGCGTGCGTGGATCGTGTGCATGTGGGGTGGT |
| | | TGATTAGGGGTGTATGGCTGGGTGATTGAGGCGTGTATGGTGGTGTGGATG |
| | | ACAAGAGTGATTGTTGGTGTGAATGACGAGTGACTGTCTAACGTCTTGACC |
| | | GATTCTACAGTTGAAGGTTACGAAGGGCGGGCCTCTGCCCTTCTCTTGGGA |
| | | TATCCTGAGCCCGCAGTTTATGTACGGCAGCCGGGCTTTCACCAAACACCC |
| | | TGCCGATATCCCAGACTACTATAAACAGTCCTTTCCAGAAGGATTTAAGTGG |
| | | GAGCGAGTCATGAATTTCGAGGACGGAGGTGCCGTGACGGTTACTCAGGA |
| | | CACCAGCCTGGAGGACGGCACCCTGATCTACAAGGTGAAGCTGAGGGGCA |
| | | CCAACTTCCCCCCCGACGGCCCCGTGATGCAGAAGAAGACCATGGGCTGG |
| | | GAGGCCAGCACCGAGAGGCTGTACCCCGAGGACGGCGTGCTGAAGGGCG |
| | | ACATCAAGATGGCCCTGAGGCTGAAGGACGGCGGCAGGTACCTGGCCGAC |
| | | TTCAAGACCACCTACAAGGCCAAGAAGCCCGTGCAGATGCCCGGCGCCTA |
| | | CAACGTGGACAGGAAGCTGGACATCACCAGCCACAACGAGGACTACACCG |
| | | TGGTGGAGCAGTACGAGAGGAGCGAGGGCAGGCACAGCACCGGCGGCAT |
| | | GGACGAGCTGTACAAGGACTACAAGGACGATGATGACAAGTGATAAACAAA |
| | | TGGTAAGGAAGGGCACATCAATCTTTGCTTAATTGTCCTTTACTCTAAAGAT |
| | | GTATTTTATCATACTGAATGCTAAACTTGATATCTCCTTTTAGGTCATTGATG |
| | | TCCTTCACCCCGGGAAGGCGACAGTGCCTAAGACAGAAATTCGGGAAAAAC |
| | | TAGCCAAAATGTACAAGACCACACCGGATGTCATCTTTGTATTTGGATTCAG |
| | | AACTCA |
| Exon | 148 | ATGGCGAGAACAATGGTCGCGATGGTATCTAAGGGCGAAGCAG |
| encoding | | |
| for first | | |
| part of | | |
| mScarlet | | |
| First part | 149 | GTAAGCGGCGTGCTTGTTGCGTGGTTGGGGTGTGGGTGTGAGTGGGATGG |
| of intronic | | GAGAGTGGTTGTCGCGTGTGGTTGGCTCGGGTGCTTGGATGGGTGATTGT |
| region | | CGGCGTGTTTGACAG |
| (TG-rich | | |
| region | | |
| underlined) | | |
| Cryptic | 150 | TGATAAAAGAGTTTATGAGATTCAAAGTCCACATGGAGGGATCAATGAACGG |
| exon | | ACACGAATTTGAAATTGAAGGCGAGGGCGAAGGAAGACCTTATGAGGGGAC |
| encoding | | ACAGACCGCCAAG |
| for second | | |
| part of | | |
| mScarlet | | |
| Second | 151 | GTGCGTGCGTGGATCGTGTGCATGTGGGGTGGTTGATTAGGGGTGTATGG |
| part of | | CTGGGTGATTGAGGCGTGTATGGTGGTGTGGATGACAAGAGTGATTGTTGG |
| intronic | | TGTGAATGACGAGTGACTGTCTAACGTCTTGACCGATTCTACAG |
| region | | |
| (TG-rich | | |
| region | | |
| underlined) | | |
| Exon | 152 | TTGAAGGTTACGAAGGGCGGGCCTCTGCCCTTCTCTTGGGATATCCTGAGC |
| encoding | | CCGCAGTTTATGTACGGCAGCCGGGCTTTCACCAAACACCCTGCCGATATC |
| for third | | CCAGACTACTATAAACAGTCCTTTCCAGAAGGATTTAAGTGGGAGCGAGTC |
| part of | | ATGAATTTCGAGGACGGAGGTGCCGTGACGGTTACTCAGGACACCAGCCTG |
| mScarlet | | GAGGACGGCACCCTGATCTACAAGGTGAAGCTGAGGGGCACCAACTTCCC |
| | | CCCCGACGGCCCCGTGATGCAGAAGAAGACCATGGGCTGGGAGGCCAGC |
| | | ACCGAGAGGCTGTACCCCGAGGACGGCGTGCTGAAGGGCGACATCAAGAT |
| | | GGCCCTGAGGCTGAAGGACGGCGGCAGGTACCTGGCCGACTTCAAGACCA |
| | | CCTACAAGGCCAAGAAGCCCGTGCAGATGCCCGGCGCCTACAACGTGGAC |
| | | AGGAAGCTGGACATCACCAGCCACAACGAGGACTACACCGTGGTGGAGCA |
| | | GTACGAGAGGAGCGAGGGCAGGCACAGCACCGGCGGCATGGACGAGCTG |
| | | TACAAGGACTACAAGGACGATGATGACAAGTGA |

### Example 2J

Similar to Example 21, this Example construct did not have any expended TG repeats, but instead was TG-enriched, with TGs spaced throughout the introns, but had comparatively weaker cryptic splice sites.

| | SEQ ID | |
|---|---|---|
| | NO: | Sequence |
| Construct | 153 | CGGCCGCTTCTTGGTGCCAGCTTATCATAGCGCTACCGGTCGCCACCATG |
| 2J (start | | GCGCGGACGATGGTAGCAATGGTGTCTAAGGGCGAAGCAGGTAAGTAGTGT |
| codon in | | GTTTGATGAGTGTATGTGGTGTGTCTGAGAGTGTAGTGTATGAGTGATTGA |
| bold) | | CGTGAGTGTTTGTAAGGCGTGTCTGTTTGAGTGACTGGTCGTGTGATTGAC |
| | | AGTTATAAAAGAATTTATGAGGTTCAAAGTCCACATGGAAGGCTCTATGAA |
| | | CGGTCATGAGTTTGAAATTGAAGGTGAGGGTGAAGGCCGCCCTTATGAAGG |
| | | CACACAAACTGCAAAGGTGGGTGCGTGCTGGGCGTGTCTGTCGGGTGAATG |
| | | CACTGGAGTGCGTGTCTGCGTGGGTGTTGAGTGGATGTAGGTGTGACTGCC |
| | | TCGTGTGCTTGCGAGAGTGAATGGAGTGTGCTTGATGCATTTTTTTATTCT |
| | | CGTGTCAGCTGAAAGTGACGAAGGGCGGGCCTCTGCCCTTCTCTTGGGAT |
| | | ATCCTGAGCCCGCAGTTTATGTACGGCAGCCGGGCTTTCACCAAACACCC |
| | | TGCCGATATCCCAGACTACTATAAACAGTCCTTTCCAGAAGGATTTAAGT |
| | | GGGAGCGAGTCATGAATTTCGAGGACGGAGGTGCCGTGACGGTTACTCAG |
| | | GACACCAGCCTGGAGGACGGCACCCTGATCTACAAGGTGAAGCTGAGGGG |
| | | CACCAACTTCCCCCCCGACGGCCCCGTGATGCAGAAGAAGACCATGGGCT |
| | | GGGAGGCCAGCACCGAGAGGCTGTACCCCGAGGACGGCGTGCTGAAGGGC |
| | | GACATCAAGATGGCCCTGAGGCTGAAGGACGGCGGCAGGTACCTGGCCGA |
| | | CTTCAAGACCACCTACAAGGCCAAGAAGCCCGTGCAGATGCCCGGCGCCT |
| | | ACAACGTGGACAGGAAGCTGGACATCACCAGCCACAACGAGGACTACACC |
| | | GTGGTGGAGCAGTACGAGAGGAGCGAGGGCAGGCACAGCACCGGCGGCAT |
| | | GGACGAGCTGTACAAGGACTACAAGGACGATGATGACAAGTGATAAACAA |
| | | ATGGTAAGGAAGGGCACATCAATCTTTGCTTAATTGTCCTTTACTCTAAA |
| | | GATGTATTTTATCATACTGAATGCTAAACTTGATATCTCCTTTTAGGTCA |
| | | TTGATGTCCTTCACCCCGGGAAGGCGACAGTGCCTAAGACAGAAATTCGG |
| | | GAAAAACTAGCCAAAATGTACAAGACCACACCGGATGTCATCTTTGTATT |
| | | TGGATTCAGAACTCA |
| Exon | 154 | ATGGCGCGGACGATGGTAGCAATGGTGTCTAAGGGCGAAGCAG |
| encoding | | |
| for first | | |
| part of | | |
| mScarlet | | |
| First part | 155 | GTAAGTAGTGTGTTTGATGAGTGTATGTGGTGTGTCTGAGAGTGTAGTGTAT |
| of intronic | | GAGTGATTGACGTGAGTGTTTGTAAGGCGTGTCTGTTTGAGTGACTGGTCG |
| region | | TGTGATTGACAG |
| (TG-rich | | |
| region | | |
| underlined) | | |
| Cryptic | 156 | TTATAAAAGAATTTATGAGGTTCAAAGTCCACATGGAAGGCTCTATGAACGG |
| exon | | TCATGAGTTTGAAATTGAAGGTGAGGGTGAAGGCCGCCCTTATGAAGGCAC |
| encoding | | ACAAACTGCAAAG |
| for second | | |
| part of | | |
| mScarlet | | |
| Second | 157 | GTGGGTGCGTGCTGGGCGTGTCTGTCGGGTGAATGCACTGGAGTGCGTGT |
| part of | | CTGCGTGGGTGTTGAGTGGATGTAGGTGTGACTGCCTCGTGTGCTTGCGA |
| intronic | | GAGTGAATGGAGTGTGCTTGATGCATTTTTTTATTCTCGTGTCAG |
| region | | |
| (TG-rich | | |
| region | | |
| underlined) | | |
| Exon | 158 | CTGAAAGTGACGAAGGGCGGGCCTCTGCCCTTCTCTTGGGATATCCTGAGC |
| encoding | | CCGCAGTTTATGTACGGCAGCCGGGCTTTCACCAAACACCCTGCCGATATC |
| for third | | CCAGACTACTATAAACAGTCCTTTCCAGAAGGATTTAAGTGGGAGCGAGTC |
| part of | | ATGAATTTCGAGGACGGAGGTGCCGTGACGGTTACTCAGGACACCAGCCT |
| mScarlet | | GGAGGACGGCACCCTGATCTACAAGGTGAAGCTGAGGGGCACCAACTTCC |
| | | CCCCCGACGGCCCCGTGATGCAGAAGAAGACCATGGGCTGGGAGGCCAG |
| | | CACCGAGAGGCTGTACCCCGAGGACGGCGTGCTGAAGGGCGACATCAAGA |
| | | TGGCCCTGAGGCTGAAGGACGGCGGCAGGTACCTGGCCGACTTCAAGACC |
| | | ACCTACAAGGCCAAGAAGCCCGTGCAGATGCCCGGCGCCTACAACGTGGA |
| | | CAGGAAGCTGGACATCACCAGCCACAACGAGGACTACACCGTGGTGGAGC |
| | | AGTACGAGAGGAGCGAGGGCAGGCACAGCACCGGGGGCATGGACGAGCT |
| | | GTACAAGGACTACAAGGACGATGATGACAAGTGA |

### Example 3

The next example construct was also of “Design 2” but differed in that the transgene encoded for Cre recombinase with an SV40 nuclear localization signal fused to mNeonGreen (a fluorescent protein) separated by a T2A self-cleaving sequence. Different from Example 2, the intronic region (both first part and second part), TDP-43 binding domain and the further intronic sequence had the same sequences as described for Example 1A.

The construct contains (from 5′→3′):

A first exon, encoding for a first part of the transgene (here, Cre recombinase with a nuclear localisation signal derived from SV40 virus) which included a start codon

A regulatory domain comprising:
A cryptic exon sequence embedded within an intronic region, wherein the cryptic exon sequence encodes for a second part of the transgene (here, Cre recombinase). The cryptic exon sequence is defined by a splice acceptor site and splice donor site, where one of these splice sites is repressed by TDP-43 binding. The intronic region itself is defined by a second splice donor and acceptor site and is split into two parts, a first part upstream of the cryptic exon sequence and a second part downstream of the cryptic exon sequence. Here, the intronic region comprises a TDP-43 binding domain and is based on AARS1.

A third exon, encoding for a third part of the transgene (here, Cre recombinase),
a sequence comprising a T2A cleavage site,
a sequence encoding for a second transgene (mNeonGreen)
A downstream intron and exon sequence (here, derived from RPS24).

The Cre recombinase transgene was split into three portions. The first exon was upstream of the regulatory domain, the second exon was the cryptic exon sequence, and the third exon was downstream of the regulatory domain. The transgene was split into three exons that could be effectively spliced as predicted using the Splice AI algorithm. First, good splice site contexts were identified in the Cre recombinase coding sequence by searching for tandem consensus exonic splice site motifs ([C/A/G]AG-G). Next, the sequence between the tandem splice motifs, which would become the cryptic exon, was randomly mutated (using synonymous mutations only), and sequences with SpliceAI scores of ˜0.3 were selected.

| | SEQ ID | |
|---|---|---|
| | NO: | Sequence |
| Construct 3 | 86 | ATGCCCAAGAAGAAGAGGAAGGTGTCCAACCTGTTAACAGTCCACCAG |
| (intronic | | AACCTCCCGGCCCTGCCCGTGGATGCCACGTCGGACGAGGTTCGCAAG |
| regions shown | | AACCTCATGGACATGTTCCGGGACCGTCAGGCATTCTCTGAACACACC |
| underlined) | | TGGAAAATGCTGCTTAGCGTATGTCGATCATGGGGGGCCTGGTGCAAGT |
| | | TGAATAATCGTAAATGGTTCCCGGCTGAACCCGAGGACGTCAGAGACTA |
| | | CCTTTTGTACCTGCAAGCAAGGGGATTAGCCGTTAAGACTATACAGCAG |
| | | CATTTGGGACAATTAAATATGTTGCACAGGTAAGAATGCACATCACTTC |
| | | TTGAGAGTATGGAGGAGTGAAATGACACTCAGTGCCAGAGTTACTGTAT |
| | | ATCTACACTTTAAAAGTGTAGCTTTTAAAAGATAAGCAAGCACAATCTT |
| | | TTGTGTGTGTGTGTGTGAATGTGTGTGTGTGTGTGTGTCACCCAGGCGG |
| | | TCCGGGCTTCCCCGGCCTTCGGATTCGAACGCAGTGAGCCTAGTCATGC |
| | | GCCGGATTAGAAAGGAAAATGTTGACGCTGGAGAACGGGCAAAGCAAG |
| | | TATGCATCACCCCCCCAGCTAATTTTTTTTTGTATTTTTTACCGAGTCGG |
| | | GGTTTCGCAATGTTGCCCAGGCTGGTCTCAGAGTCTCGCTCTGTTGTCT |
| | | ACGCTGGAGTGCAGTAACATGAGCCACTGTGCCCGGCCAATCCTAAGA |
| | | ATTTCTTTTGCGGTGGTTGCAAGTCTGGGCAGAACTCTTGTCAGGGGCT |
| | | GTAACTGGACTTATCTTTACTCCTTTGTCAGGCTTTAGCGTTTGAGAGAA |
| | | CAGATTTTGATCAAGTGCGATCCCTTATGGAGAACTCTGACCGTTGCCA |
| | | AGACATAAGAAATCTTGCTTTCTTGGGCATCGCGTACAACACCTTACTGA |
| | | GAATTGCGGAGATTGCCCGGATTCGAGTCAAGGATATAAGCCGCACCG |
| | | ACGGAGGACGGATGCTCATCCACATTGGGAGAACGAAGACCCTAGTGT |
| | | CAACCGCCGGCGTGGAGAAAGCTCTGAGCCTTGGAGTCACAAAACTGG |
| | | TCGAGCGGTGGATCAGCGTGTCAGGCGTCGCCGACGACCCCAACAACT |
| | | ACCTGTTCTGCCGAGTCCGGAAGAACGGGGTCGCCGCACCATCAGCGA |
| | | CGTCGCAGCTCTCCACGCGGGCCCTCGAAGGCATCTTCGAAGCTACTC |
| | | ACCGACTGATCTACGGTGCGAAAGACGATTCTGGTCAGcgaTACCTTGC |
| | | TTGGAGTGGGCATAGTGCACGGGTGGGGGGGGCTAGGGATATGGCTA |
| | | GAGCTGGAGTCTCAATCCCTGAAATTATGCAAGCTGGGGGTTGGACAAA |
| | | TGTTAATATTGTAATGAACTATATAAGAAACTTGGATAGTGAGACAGGGG |
| | | CTATGGTGCGCCTGTTAGAAGATGGGGACGGCTCTGGATCTCCGGCGG |
| | | CGAAACGCGTGAAACTGGATGGCAGTGGAGAGGGCAGAGGAAGTCTG |
| | | CTAACATGCGGTGACGTCGAGGAGAATCCTGGCCCAGTAAGCAAAGGC |
| | | GAGGAAGATAATATGGCCTCATTACCCGCAACACACGAACTCCATATAT |
| | | TCGGATCCATCAACGGAGTCGATTTCGACATGGTAGGGCAGGGCACCG |
| | | GGAATCCCAACGACGGATACGAGGAGCTGAACCTGAAATCTACTAAGG |
| | | GCGATTTGCAATTTTCTCCTTGGATCCTGGTGCCGCACATCGGCTACGG |
| | | ATTTCATCAGTACCTCCCTTATCCAGACGGGATGAGTCCATTCCAGGCG |
| | | GCTATGGTCGACGGGAGCGGCTATCAGGTGCACAGGACAATGCAATTC |
| | | GAAGACGGAGCATCTCTTACCGTGAATTATCGCTATACTTACGAAGGCT |
| | | CCCATATTAAGGGCGAGGCTCAAGTTAAGGGGACTGGTTTTCCAGCCG |
| | | ATGGCCCCGTCATGACAAACTCGCTCACAGCAGCCGATTGGTGCCGGT |
| | | CCAAGAAAACTTACCCTAATGATAAGACCATTATTTCAACCTTCAAATGG |
| | | AGCTACACCACGGGAAACGGAAAGCGATACCGCAGTACTGCCAGAACC |
| | | ACATATACATTTGCCAAGCCCATGGCCGCTAACTATCTTAAGAATCAGC |
| | | CAATGTACGTCTTCAGAAAAACCGAACTGAAGCACAGCAAAACCGAGCT |
| | | GAACTTTAAGGAGTGGCAGAAAGCTTTCACGGACGTTATGGGAATGGAC |
| | | GAGCTATATAAAGGATCTGGTTACCCATACGATGTTCCAGATTACGCTT |
| | | GATAAACAAATGGTAAGGAAGGGCACATCAATCTTTGCTTAATTGTCCTT |
| | | TACTCTAAAGATGTATTTTATCATACTGAATGCTAAACTTGATATCTCC |
| | | TTTTAGGTCATTGATGTCCTTCACCCCGGGAAGGCGACAGTGCCTAAGA |
| | | CAGAAATTCGGGAAAAACTAGCCAAAATGTACAAGACCACACCGGATGT |
| | | CATCTTTGTATTTGGATTCAGAACTCA |
| First Cre | 87 | ATGCCCAAGAAGAAGAGGAAGGTGTCCAACCTGTTAACAGTCCACCAG |
| recombinase | | AACCTCCCGGCCCTGCCCGTGGATGCCACGTCGGACGAGGTTCGCAA |
| exonic | | GAACCTCATGGACATGTTCCGGGACCGTCAGGCATTCTCTGAACACACC |
| sequence and | | TGGAAAATGCTGCTTAGCGTATGTCGATCATGGGCGGCCTGGTGCAAG |
| part of | | TTGAATAATCGTAAATGGTTCCCGGCTGAACCCGAGGACGTCAGAGACT |
| transgene | | ACCTTTTGTACCTGCAAGCAAGGGGATTAGCCGTTAAGACTATACAGCA |
| | | GCATTTGGGACAATTAAATATGTTGCACAG |
| CE and | 88 | GCGGTCCGGGCTTCCCCGGCCTTCGGATTCGAACGCAGTGAGCCTAGT |
| Second Cre | | CATGCGCCGGATTAGAAAGGAAAATGTTGACGCTGGAGAACGGGCAAA |
| recombinase | | GCAA |
| exon | | |
| sequence and | | |
| part of | | |
| transgene | | |
| Third Cre | 89 | GCTTTAGCGTTTGAGAGAACAGATTTTGATCAAGTGCGATCCCTTATGG |
| recombinase | | AGAACTCTGACCGTTGCCAAGACATAAGAAATCTTGCTTTCTTGGGCAT |
| exon | | CGCGTACAACACCTTACTGAGAATTGCGGAGATTGCCCGGATTCGAGTC |
| sequence and | | AAGGATATAAGCCGCACCGACGGAGGACGGATGCTCATCCACATTGGG |
| part of | | AGAACGAAGACCCTAGTGTCAACCGCCGGCGTGGAGAAAGCTCTGAGC |
| transgene, | | CTTGGAGTCACAAAACTGGTCGAGCGGTGGATCAGCGTGTCAGGCGTC |
| and T2A- | | GCCGACGACCCCAACAACTACCTGTTCTGCCGAGTCCGGAAGAACGGG |
| mNeonGreen | | GTCGCCGCACCATCAGCGACGTCGCAGCTCTCCACGCGGGCCCTCGA |
| (T2A in italics, | | AGGCATCTTCGAAGCTACTCACCGACTGATCTACGGTGCGAAAGACGAT |
| mNeonGreen | | TCTGGTCAGCGATACCTTGCTTGGAGTGGGCATAGTGCACGGGTGGGG |
| underlined, | | GCGGCTAGGGATATGGCTAGAGCTGGAGTCTCAATCCCTGAAATTATGC |
| PTC shown in | | AAGCTGGGGGTTGGACAAATGTTAATATTGTAATGAACTATATAAGAAAC |
| bold) | | TTGGATAGTGAGACAGGGGCTATGGTGCGCCTGTTAGAAGATGGGGAC |
| | | GGCTCTGGATCTCCGGCGGCGAAACGCGTGAAACTGGATGGCAGTGG |
| | | AGAGGGCAGAGGAAGTCTGCTAACATGCGGTGACGTCGAGGAGAATCC |
| | | TGGCCCAGTAAGCAAAGGCGAGGAAGATAATATGGCCTCATTACCCGC |
| | | AACACACGAACTCCATATATTCGGATCCATCAACGGAGTCGATTTCGAC |
| | | ATGGTAGGGCAGGGCACCGGGAATCCCAACGACGGATACGAGGAGCT |
| | | GAACCTGAAATCTACTAAGGGCGATTTGCAATTTTCTCCTTGGATCCTG |
| | | GTGCCGCACATCGGCTACGGATTTCATCAGTACCTCCCTTATCCAGACG |
| | | GGATGAGTCCATTCCAGGCGGCTATGGTCGACGGGAGCGGCTATCAGG |
| | | TGCACAGGACAATGCAATTCGAAGACGGAGCATCTCTTACCGTGAATTA |
| | | TCGCTATACTTACGAAGGCTCCCATATTAAGGGCGAGGCTCAAGTTAAG |
| | | GGGACTGGTTTTCCAGCCGATGGCCCCGTCATGACAAACTCGCTCACA |
| | | GCAGCCGATTGGTGCCGGTCCAAGAAAACTTACCCTAATGATAAGACCA |
| | | TTATTTCAACCTTCAAATGGAGCTACACCACGGGAAACGGAAAGCGATA |
| | | CCGCAGTACTGCCAGAACCACATATACATTTGCCAAGCCCATGGCCGCT |
| | | AACTATCTTAAGAATCAGCCAATGTACGTCTTCAGAAAAACCGAACTGAA |
| | | GCACAGCAAAACCGAGCTGAACTTTAAGGAGTGGCAGAAAGCTTTCAC |
| | | GGACGTTATGGGAATGGACGAGCTATATAAAGGATCTGGTTACCCATAC |
| | | GATGTTCCAGATTACGCTTGA |

### Example 4

Example 4 was similar to Example 3, apart from the exons encoded for a Cas9 protein, with a nucleoplasmin nuclear localization signal, a tri-FLAG tag, and an N-terminal T2A-mCherry with a C terminal FLAG. The transgene was split into three exons that could be effectively spliced as predicted using the Splice AI algorithm. Again, good splice site contexts were identified in the Cas9 coding sequence by searching for tandem consensus exonic splice site motifs ([C/A/G]AG-G). Next, the sequence between the tandem splice motifs, which would become the cryptic exon, was randomly mutated (using synonymous mutations only).

In the selected example, the cryptic splice acceptor site (i.e., the first acceptor splice site) had a splice score of 0.06 as determined by the Splice AI algorithm and the cryptic splice donor site (i.e., the first splice donor site) had a splice score of 0.17 as determined by the Splice AI algorithm.

| | SEQ ID | |
|---|---|---|
| | NO: | Sequence |
| Construct 4 | 90 | ATGGACTATAAGGACCACGACGGAGACTACAAGGATCATGATATTGATTACAA |
| (intronic | | AGACGATGACGATAAGATGGCCCCAAAGAAGAAGCGGAAGGTCGGTATCCA |
| regions shown | | CGGAGTCCCAGCAGCCGACAAGAAGTACAGCATCGGCCTGGACATCGGCAC |
| underlined) | | CAACTCTGTGGGCTGGGCCGTGATCACCGACGAGTACAAGGTGCCCAGCAA |
| | | GAAATTCAAGGTGCTGGGCAACACCGACCGGCACAGCATCAAGAAGAACCT |
| | | GATCGGAGCCCTGCTGTTCGACAGCGGCGAAACAGCCGAGGCCACCCGGCT |
| | | GAAGAGAACCGCCAGAAGAAGATACACCAGACGGAAGAACCGGATCTGCTA |
| | | TCTGCAAGAGATCTTCAGCAACGAGATGGCCAAGGTGGACGACAGCTTCTTC |
| | | CACAGACTGGAAGAGTCCTTCCTGGTGGAAGAGGATAAGAAGCACGAGCGG |
| | | CACCCCATCTTCGGCAACATCGTGGACGAGGTGGCCTACCACGAGAAGTAC |
| | | CCCACCATCTACCACCTGAGAAAGAAACTGGTGGACAGCACCGACAAGGCC |
| | | GACCTGCGGCTGATCTATCTGGCCCTGGCCCACATGATCAAGTTCCGGGGC |
| | | CACTTCCTGATCGAGGGCGACCTGAACCCCGACAACAGCGACGTGGACAAG |
| | | CTGTTCATCCAGCTGGTGCAGACCTACAACCAGCTGTTCGAGGAAAACCCCA |
| | | TCAACGCCAGCGGCGTGGACGCCAAGGCCATCCTGTCTGCCAGACTGAGCA |
| | | AGAGCAGACGGCTGGAAAATCTGATCGCCCAGCTGCCCGGCGAGAAGAAGA |
| | | ATGGCCTGTTCGGAAACCTGATTGCCCTGAGCCTGGGCCTGACCCCCAACTT |
| | | CAAGAGCAACTTCGACCTGGCCGAGGATGCCAAACTGCAGCTGAGCAAGGA |
| | | CACCTACGACGACGACCTGGACAACCTGCTGGCCCAGATCGGCGACCAGTA |
| | | CGCCGACCTGTTTCTGGCCGCCAAGAACCTGTCCGACGCCATCCTGCTGAG |
| | | CGACATCCTGAGAGTGAACACCGAGATCACCAAGGCCCCCCTGAGCGCCTC |
| | | TATGATCAAGAGATACGACGAGCACCACCAGGACCTGACCCTGCTGAAAGCT |
| | | CTCGTGCGGCAGCAGCTGCCTGAGAAGTACAAAGAGATTTTCTTCGACCAGA |
| | | GCAAGAACGGCTACGCCGGCTACATTGACGGCGGAGCCAGCCAGGAAGAGT |
| | | TCTACAAGTTCATCAAGCCCATCCTGGAAAAGATGGACGGCACCGAGGAACT |
| | | GCTCGTGAAGCTGAACAGAGAGGACCTGCTGCGGAAGCAGCGGACCTTCGA |
| | | CAACGGCAGCATCCCCCACCAGATCCACCTGGGAGAGCTGCACGCCATTCT |
| | | GCGGCGGCAGGAAGATTTTTACCCATTCCTGAAGGACAACCGGGAAAAGATC |
| | | GAGAAGATCCTGACCTTCCGCATCCCCTACTACGTGGGCCCTCTGGCCAGG |
| | | GGAAACAGCAGATTCGCCTGGATGACCAGAAAGAGCGAGGAAACCATCACC |
| | | CCCTGGAACTTCGAGGAAGTGGTGGACAAGGGCGCTTCCGCCCAGAGCTTC |
| | | ATCGAGCGGATGACCAACTTCGATAAGAACCTGCCCAACGAGAAGGTGCTGC |
| | | CCAAGCACAGCCTGCTGTACGAGTACTTCACCGTGTATAACGAGCTGACCAA |
| | | AGTGAAATACGTGACCGAGGGAATGAGAAAGCCCGCCTTCCTGAGCGGCGA |
| | | GCAGAAAAAGGCCATCGTGGACCTGCTGTTCAAGACCAACCGGAAAGTGAC |
| | | CGTGAAGCAGCTGAAAGAGGACTACTTCAAGAAAATCGAGTGCTTCGACTCC |
| | | GTGGAAATCTCCGGCGTGGAAGATCGGTTCAACGCCTCCCTGGGCACATAC |
| | | CACGATCTGCTGAAAATTATCAAGGACAAGGACTTCCTGGACAATGAGGAAA |
| | | ACGAGGACATTCTGGAAGATATCGTGCTGACCCTGACACTGTTTGAGGACAG |
| | | AGAGATGATCGAGGAACGGCTGAAAACCTATGCCCACCTGTTCGACGACAAA |
| | | GTGATGAAGCAGCTGAAGCGGCGGAGATACACCGGCTGGGGCAGGTAAGAA |
| | | TGCACATCACTTCTTGAGAGTATGGAGGAGTGAAATGACACTCAGTGCCAGA |
| | | GTTACTGTATATCTACACTTTAAAAGTGTAGCTTTTAAAAGATAAGCAAGCACA |
| | | ATCTTTTGTGTGTGTGTGTGTGAATGTGTGTGTGTGTGTGTGTCACCCAGATT |
| | | ATCACGCAAATTGATCAATGGAATAAGAGATAAACAGTCCGGAAAAACAATCC |
| | | TTGATTTTTTAAAAAGTGATGGGTTCGCAAATAGAAATTTTATGCAACTCATAC |
| | | ATGATGACAGCTTGACATTCAAAGAGGACATTCAGAAGGCGCAGGTATGCAT |
| | | CACCCCCCCAGCTAATTTTTTTTTGTATTTTTTACCGAGTCGGGGTTTCGCAAT |
| | | GTTGCCCAGGCTGGTCTCAGAGTCTCGCTCTGTTGTCTACGCTGGAGTGCAG |
| | | TAACATGAGCCACTGTGCCCGGCCAATCCTAAGAATTTCTTTTGCGGTGGTT |
| | | GCAAGTCTGGGCAGAACTCTTGTCAGGGGCTGTAACTGGACTTATCTTTACT |
| | | CCTTTGTCAGGTATCCGGCCAGGGCGATAGCCTGCACGAGCACATTGCCAAT |
| | | CTGGCCGGCAGCCCCGCCATTAAGAAGGGCATCCTGCAGACAGTGAAGGTG |
| | | GTGGACGAGCTCGTGAAAGTGATGGGCCGGCACAAGCCCGAGAACATCGTG |
| | | ATCGAAATGGCCAGAGAGAACCAGACCACCCAGAAGGGACAGAAGAACAGC |
| | | CGCGAGAGAATGAAGCGGATCGAAGAGGGCATCAAAGAGCTGGGCAGCCAG |
| | | ATCCTGAAAGAACACCCCGTGGAAAACACCCAGCTGCAGAACGAGAAGCTGT |
| | | ACCTGTACTACCTGCAGAATGGGGGGGATATGTACGTGGACCAGGAACTGG |
| | | ACATCAACCGGCTGTCCGACTACGATGTGGACCATATCGTGCCTCAGAGCTT |
| | | TCTGAAGGACGACTCCATCGACAACAAGGTGCTGACCAGAAGCGACAAGAAC |
| | | CGGGGCAAGAGCGACAACGTGCCCTCCGAAGAGGTCGTGAAGAAGATGAAG |
| | | AACTACTGGCGGCAGCTGCTGAACGCCAAGCTGATTACCCAGAGAAAGTTCG |
| | | ACAATCTGACCAAGGCCGAGAGAGGCGGCCTGAGCGAACTGGATAAGGCCG |
| | | GCTTCATCAAGAGACAGCTGGTGGAAACCCGGCAGATCACAAAGCACGTGG |
| | | CACAGATCCTGGACTCCCGGATGAACACTAAGTACGACGAGAATGACAAGCT |
| | | GATCCGGGAAGTGAAAGTGATCACCCTGAAGTCCAAGCTGGTGTCCGATTTC |
| | | CGGAAGGATTTCCAGTTTTACAAAGTGCGCGAGATCAACAACTACCACCACG |
| | | CCCACGACGCCTACCTGAACGCCGTCGTGGGAACCGCCCTGATCAAAAAGT |
| | | ACCCTAAGCTGGAAAGCGAGTTCGTGTACGGCGACTACAAGGTGTACGACGT |
| | | GCGGAAGATGATCGCCAAGAGCGAGCAGGAAATCGGCAAGGCTACCGCCAA |
| | | GTACTTCTTCTACAGCAACATCATGAACTTTTTCAAGACCGAGATTACCCTGG |
| | | CCAACGGCGAGATCCGGAAGCGGCCTCTGATCGAGACAAACGGCGAAACCG |
| | | GGGAGATCGTGTGGGATAAGGGCCGGGATTTTGCCACCGTGCGGAAAGTGC |
| | | TGAGCATGCCCCAAGTGAATATCGTGAAAAAGACCGAGGTGCAGACAGGCG |
| | | GCTTCAGCAAAGAGTCTATCCTGCCCAAGAGGAACAGCGATAAGCTGATCGC |
| | | CAGAAAGAAGGACTGGGACCCTAAGAAGTACGGCGGCTTCGACAGCCCCAC |
| | | CGTGGCCTATTCTGTGCTGGTGGTGGCCAAAGTGGAAAAGGGCAAGTCCAA |
| | | GAAACTGAAGAGTGTGAAAGAGCTGCTGGGGATCACCATCATGGAAAGAAGC |
| | | AGCTTCGAGAAGAATCCCATCGACTTTCTGGAAGCCAAGGGCTACAAAGAAG |
| | | TGAAAAAGGACCTGATCATCAAGCTGCCTAAGTACTCCCTGTTCGAGCTGGA |
| | | AAACGGCCGGAAGAGAATGCTGGCCTCTGCCGGCGAACTGCAGAAGGGAAA |
| | | CGAACTGGCCCTGCCCTCCAAATATGTGAACTTCCTGTACCTGGCCAGCCAC |
| | | TATGAGAAGCTGAAGGGCTCCCCCGAGGATAATGAGCAGAAACAGCTGTTTG |
| | | TGGAACAGCACAAGCACTACCTGGACGAGATCATCGAGCAGATCAGCGAGTT |
| | | CTCCAAGAGAGTGATCCTGGCCGACGCTAATCTGGACAAAGTGCTGTCCGCC |
| | | TACAACAAGCACCGGGATAAGCCCATCAGAGAGCAGGCCGAGAATATCATCC |
| | | ACCTGTTTACCCTGACCAATCTGGGAGCCCCTGCCGCCTTCAAGTACTTTGA |
| | | CACCACCATCGACCGGAAGAGGTACACCAGCACCAAAGAGGTGCTGGACGC |
| | | CACCCTGATCCACCAGAGCATCACCGGCCTGTACGAGACACGGATCGACCT |
| | | GTCTCAGCTGGGAGGCGACAAAAGGCCGGCGGCCACGAAAAAGGCCGGCC |
| | | AGGCAAAAAAGAAAAAG |
| First Cas9 | 91 | ATGGACTATAAGGACCACGACGGAGACTACAAGGATCATGATATTGATTACA |
| exonic | | AAGACGATGACGATAAGATGGCCCCAAAGAAGAAGCGGAAGGTCGGTATCCA |
| sequence/part | | CGGAGTCCCAGCAGCCGACAAGAAGTACAGCATCGGCCTGGACATCGGCAC |
| of transgene | | CAACTCTGTGGGCTGGGCCGTGATCACCGACGAGTACAAGGTGCCCAGCAA |
| | | GAAATTCAAGGTGCTGGGCAACACCGACCGGCACAGCATCAAGAAGAACCT |
| | | GATCGGAGCCCTGCTGTTCGACAGCGGCGAAACAGCCGAGGCCACCCGGCT |
| | | GAAGAGAACCGCCAGAAGAAGATACACCAGACGGAAGAACCGGATCTGCTA |
| | | TCTGCAAGAGATCTTCAGCAACGAGATGGCCAAGGTGGACGACAGCTTCTTC |
| | | CACAGACTGGAAGAGTCCTTCCTGGTGGAAGAGGATAAGAAGCACGAGCGG |
| | | CACCCCATCTTCGGCAACATCGTGGACGAGGTGGCCTACCACGAGAAGTAC |
| | | CCCACCATCTACCACCTGAGAAAGAAACTGGTGGACAGCACCGACAAGGCC |
| | | GACCTGCGGCTGATCTATCTGGCCCTGGCCCACATGATCAAGTTCCGGGGC |
| | | CACTTCCTGATCGAGGGCGACCTGAACCCCGACAACAGCGACGTGGACAAG |
| | | CTGTTCATCCAGCTGGTGCAGACCTACAACCAGCTGTTCGAGGAAAACCCCA |
| | | TCAACGCCAGCGGCGTGGACGCCAAGGCCATCCTGTCTGCCAGACTGAGCA |
| | | AGAGCAGACGGCTGGAAAATCTGATCGCCCAGCTGCCCGGCGAGAAGAAGA |
| | | ATGGCCTGTTCGGAAACCTGATTGCCCTGAGCCTGGGCCTGACCCCCAACTT |
| | | CAAGAGCAACTTCGACCTGGCCGAGGATGCCAAACTGCAGCTGAGCAAGGA |
| | | CACCTACGACGACGACCTGGACAACCTGCTGGCCCAGATCGGCGACCAGTA |
| | | CGCCGACCTGTTTCTGGCCGCCAAGAACCTGTCCGACGCCATCCTGCTGAG |
| | | CGACATCCTGAGAGTGAACACCGAGATCACCAAGGCCCCCCTGAGCGCCTC |
| | | TATGATCAAGAGATACGACGAGCACCACCAGGACCTGACCCTGCTGAAAGCT |
| | | CTCGTGCGGCAGCAGCTGCCTGAGAAGTACAAAGAGATTTTCTTCGACCAGA |
| | | GCAAGAACGGCTACGCCGGCTACATTGACGGCGGAGCCAGCCAGGAAGAGT |
| | | TCTACAAGTTCATCAAGCCCATCCTGGAAAAGATGGACGGCACCGAGGAACT |
| | | GCTCGTGAAGCTGAACAGAGAGGACCTGCTGCGGAAGCAGCGGACCTTCGA |
| | | CAACGGCAGCATCCCCCACCAGATCCACCTGGGAGAGCTGCACGCCATTCT |
| | | GCGGCGGCAGGAAGATTTTTACCCATTCCTGAAGGACAACCGGGAAAAGATC |
| | | GAGAAGATCCTGACCTTCCGCATCCCCTACTACGTGGGCCCTCTGGCCAGG |
| | | GGAAACAGCAGATTCGCCTGGATGACCAGAAAGAGCGAGGAAACCATCACC |
| | | CCCTGGAACTTCGAGGAAGTGGTGGACAAGGGCGCTTCCGCCCAGAGCTTC |
| | | ATCGAGCGGATGACCAACTTCGATAAGAACCTGCCCAACGAGAAGGTGCTGC |
| | | CCAAGCACAGCCTGCTGTACGAGTACTTCACCGTGTATAACGAGCTGACCAA |
| | | AGTGAAATACGTGACCGAGGGAATGAGAAAGCCCGCCTTCCTGAGCGGCGA |
| | | GCAGAAAAAGGCCATCGTGGACCTGCTGTTCAAGACCAACCGGAAAGTGAC |
| | | CGTGAAGCAGCTGAAAGAGGACTACTTCAAGAAAATCGAGTGCTTCGACTCC |
| | | GTGGAAATCTCCGGCGTGGAAGATCGGTTCAACGCCTCCCTGGGCACATAC |
| | | CACGATCTGCTGAAAATTATCAAGGACAAGGACTTCCTGGACAATGAGGAAA |
| | | ACGAGGACATTCTGGAAGATATCGTGCTGACCCTGACACTGTTTGAGGACAG |
| | | AGAGATGATCGAGGAACGGCTGAAAACCTATGCCCACCTGTTCGACGACAAA |
| | | GTGATGAAGCAGCTGAAGCGGCGGAGATACACCGGCTGGGGCAG |
| Cryptic exon, | 92 | TTATCACGCAAATTGATCAATGGAATAAGAGATAAACAGTCCGGAAAAACAAT |
| and second | | CCTTGATTTTTTAAAAAGTGATGGGTTCGCAAATAGAAATTTTATGCAACTCAT |
| Cas9 exonic | | ACATGATGACAGCTTGACATTCAAAGAGGACATTCAGAAGGCGCA |
| sequence/part | | |
| of transgene | | |
| Third Cas9 | 93 | GTATCCGGCCAGGGCGATAGCCTGCACGAGCACATTGCCAATCTGGCCGGC |
| exonic | | AGCCCCGCCATTAAGAAGGGCATCCTGCAGACAGTGAAGGTGGTGGACGAG |
| sequence/part | | CTCGTGAAAGTGATGGGCCGGCACAAGCCCGAGAACATCGTGATCGAAATG |
| of transgene | | GCCAGAGAGAACCAGACCACCCAGAAGGGACAGAAGAACAGCCGCGAGAGA |
| andT2A | | ATGAAGCGGATCGAAGAGGGCATCAAAGAGCTGGGCAGCCAGATCCTGAAA |
| mCherry (PTC | | GAACACCCCGTGGAAAACACCCAGCTGCAGAACGAGAAGCTGTACCTGTACT |
| in bold and not | | ACCTGCAGAATGGGGGGGATATGTACGTGGACCAGGAACTGGACATCAACC |
| underlined, | | GGCTGTCCGACTACGATGTGGACCATATCGTGCCTCAGAGCTTTCTGAAGGA |
| T2A bold and | | CGACTCCATCGACAACAAGGTGCTGACCAGAAGCGACAAGAACCGGGGCAA |
| underlined, | | GAGCGACAACGTGCCCTCCGAAGAGGTCGTGAAGAAGATGAAGAACTACTG |
| mCherry Flag | | GCGGCAGCTGCTGAACGCCAAGCTGATTACCCAGAGAAAGTTCGACAATCTG |
| in italics only) | | ACCAAGGCCGAGAGAGGCGGCCTGAGCGAACTGGATAAGGCCGGCTTCATC |
| | | AAGAGACAGCTGGTGGAAACCCGGCAGATCACAAAGCACGTGGCACAGATC |
| | | CTGGACTCCCGGATGAACACTAAGTACGACGAGAATGACAAGCTGATCCGG |
| | | GAAGTGAAAGTGATCACCCTGAAGTCCAAGCTGGTGTCCGATTTCCGGAAGG |
| | | ATTTCCAGTTTTACAAAGTGCGCGAGATCAACAACTACCACCACGCCCACGA |
| | | CGCCTACCTGAACGCCGTCGTGGGAACCGCCCTGATCAAAAAGTACCCTAAG |
| | | CTGGAAAGCGAGTTCGTGTACGGCGACTACAAGGTGTACGACGTGCGGAAG |
| | | ATGATCGCCAAGAGCGAGCAGGAAATCGGCAAGGCTACCGCCAAGTACTTCT |
| | | TCTACAGCAACATCATGAACTTTTTCAAGACCGAGATTACCCTGGCCAACGGC |
| | | GAGATCCGGAAGCGGCCTCTGATCGAGACAAACGGCGAAACCGGGGAGATC |
| | | GTGTGGGATAAGGGCCGGGATTTTGCCACCGTGCGGAAAGTGCTGAGCATG |
| | | CCCCAAGTGAATATCGTGAAAAAGACCGAGGTGCAGACAGGCGGCTTCAGC |
| | | AAAGAGTCTATCCTGCCCAAGAGGAACAGCGATAAGCTGATCGCCAGAAAGA |
| | | AGGACTGGGACCCTAAGAAGTACGGCGGCTTCGACAGCCCCACCGTGGCCT |
| | | ATTCTGTGCTGGTGGTGGCCAAAGTGGAAAAGGGCAAGTCCAAGAAACTGAA |
| | | GAGTGTGAAAGAGCTGCTGGGGATCACCATCATGGAAAGAAGCAGCTTCGA |
| | | GAAGAATCCCATCGACTTTCTGGAAGCCAAGGGCTACAAAGAAGTGAAAAAG |
| | | GACCTGATCATCAAGCTGCCTAAGTACTCCCTGTTCGAGCTGGAAAACGGCC |
| | | GGAAGAGAATGCTGGCCTCTGCCGGCGAACTGCAGAAGGGAAACGAACTGG |
| | | CCCTGCCCTCCAAATATGTGAACTTCCTGTACCTGGCCAGCCACTATGAGAA |
| | | GCTGAAGGGCTCCCCCGAGGATAATGAGCAGAAACAGCTGTTTGTGGAACA |
| | | GCACAAGCACTACCTGGACGAGATCATCGAGCAGATCAGCGAGTTCTCCAAG |
| | | AGAGTGATCCTGGCCGACGCTAATCTGGACAAAGTGCTGTCCGCCTACAACA |
| | | AGCACCGGGATAAGCCCATCAGAGAGCAGGCCGAGAATATCATCCACCTGTT |
| | | TACCCTGACCAATCTGGGAGCCCCTGCCGCCTTCAAGTACTTTGACACCACC |
| | | ATCGACCGGAAGAGGTACACCAGCACCAAAGAGGTGCTGGACGCCACCCTG |
| | | ATCCACCAGAGCATCACCGGCCTGTACGAGACACGGATCGACCTGTCTCAG |
| | | CTGGGAGGCGACAAAAGGCCGGCGGCCACGAAAAAGGCCGGCCAGGCAAA |
| | | AAAGAAAAAGGAATTCGGCAGTGGAGAGGGCAGAGGAAGTCTGCTAACATG |
| | | CGGTGACGTCGAGGAGAATCCTGGCCCAGTCAGCAAAGGGGAAGAGGACAA |
| | | CATGGCCATCATTAAGGAGTTTATGCGATTCAAAGTACACATGGAGGGATCT |
| | | GTTAATGGCCATGAATTTGAGATAGAGGGGGAAGGTGAGGGTCGCCCTTAC |
| | | GAAGGCACGCAGACGGCTAAGCTGAAGGTCACGAAAGGGGGACCCTTGCCCT |
| | | TCGCATGGGACATACTCTCCCCACAGTTTATGTATGGTTCTAAGGCATATGT |
| | | TAAGCACCCTGCAGACATCCCAGACTATCTGAAGCTCTCCTTTCCTGAGGGG |
| | | TTTAAGTGGGAACGCGTTATGAACTTTGAGGATGGAGGGGTCGTGACTGTTA |
| | | CCCAGGATTCTTCCCTGCAAGATGGAGAGTTCATATACAAAGTGAAACTTCG |
| | | GGGAACGAATTTCCCATCAGACGGGCCAGTGATGCAGAAAAAGACGATGGG |
| | | GTGGGAGGCTTCATCCGAGAGGATGTATCCCGAGGACGGAGCATTGAAAGG |
| | | CGAAATAAAACAAAGGCTGAAGTTGAAGGATGGGGGCCACTACGACGCGGA |
| | | GGTTAAAACAACGTATAAAGCTAAAAAGCCAGTACAGCTCCCAGGCGCATAT |
| | | AACGTGAATATAAAGCTTGACATAACGAGTCATAACGAGGATTACACAATCG |
| | | TAGAACAGTACGAAAGAGCTGAAGGACGGCACTCCACCGGTGGGATGGATGA |
| | | ACTCTATAAAGACTACAAGGACGATGATGACAAGTAA |

The above construct was incorporated into a plasmid. In addition to the features described above, the plasmid further comprises an enhancer sequence and a promoter sequence upstream of the construct (here, a CMV enhancer and CMV promoter respectively) and a polyadenylation site downstream of the construct.

The full plasmid containing the Cas9 construct detailed above is provided by below (SEQ ID NO: 94).

| | |
|---|---|
| 1 | ATATATGGAG TTCCGCGTTA CATAACTTAC GGTAAATGGC CCGCCTGGCT GACCGCCCAA |
| 61 | CGACCCCCGC CCATTGACGT CAATAATGAC GTATGTTCCC ATAGTAACGC CAATAGGGAC |
| 121 | TTTCCATTGA CGTCAATGGG TGGAGTATTT ACGGTAAACT GCCCACTTGG CAGTACATCA |
| 181 | AGTGTATCAT ATGCCAAGTA CGCCCCCTAT TGACGTCAAT GACGGTAAAT GGCCCGCCTG |
| 241 | GCATTATGCC CAGTACATGA CCTTATGGGA CTTTCCTACT TGGCAGTACA TCTACGTATT |
| 301 | AGTCATCGCT ATTACCATGC TGATGCGGTT TTGGCAGTAC ATCAATGGGC GTGGATAGCG |
| 361 | GTTTGACTCA CGGGGATTTC CAAGTCTCCA CCCCATTGAC GTCAATGGGA GTTTGTTTTG |
| 421 | GCACCAAAAT CAACGGGACT TTCCAAAATG TCGTAACAAC TCCGCCCCAT TGACGCAAAT |
| 481 | GGGCGGTAGG CGTGTACGGT GGGAGGTCTA TATAAGCAGA GCTGGTTTAG TGAACCGTCA |
| 541 | GATCAGATCT TTGTCGATCC TACCATCCAC TCGACACACC CGCCAGCGGC CGCTTCTTGG |
| 601 | TGCCAGCTTA TCAggtgcca ccatggacta taaggaccac gacggagact acaaggatca |
| 661 | tgatattgat tacaaagacg atgacgataa gatggcccca aagaagaagc ggaaggtcgg |
| 721 | tatccacgga gtcccagcag ccgacaagaa gtacagcatc ggcctggaca tcggcaccaa |
| 781 | ctctgtgggc tgggccgtga tcaccgacga gtacaaggtg cccagcaaga aattcaaggt |
| 841 | gctgggcaac accgaccggc acagcatcaa gaagaacctg atcggagccc tgctgttcga |
| 901 | cagcggcgaa acagccgagg ccacccggct gaagagaacc gccagaagaa gatacaccag |
| 961 | acggaagaac cggatctgct atctgcaaga gatcttcagc aacgagatgg ccaaggtgga |
| 1021 | cgacagcttc ttccacagac tggaagagtc cttcctggtg gaagaggata agaagcacga |
| 1081 | gcggcacccc atcttcggca acatcgtgga cgaggtggcc taccacgaga agtaccccac |
| 1141 | catctaccac ctgagaaaga aactggtgga cagcaccgac aaggccgacc tgcggctgat |
| 1201 | ctatctggcc ctggcccaca tgatcaagtt ccggggccac ttcctgatcg agggcgacct |
| 1261 | gaaccccgac aacagcgacg tggacaagct gttcatccag ctggtgcaga cctacaacca |
| 1321 | gctgttcgag gaaaacccca tcaacgccag cggcgtggac gccaaggcca tcctgtctgc |
| 1381 | cagactgagc aagagcagac ggctggaaaa tctgategcc cagctgcccg gcgagaagaa |
| 1441 | gaatggcctg ttcggaaacc tgattgccct gagcctgggc ctgaccccca acttcaagag |
| 1501 | caacttcgac ctggccgagg atgccaaact gcagctgagc aaggacacct acgacgacga |
| 1561 | cctggacaac ctgctggccc agatcggcga ccagtacgcc gacctgtttc tggccgccaa |
| 1621 | gaacctgtcc gacgccatcc tgctgagcga catcctgaga gtgaacaccg agatcaccaa |
| 1681 | ggcccccctg agcgcctcta tgatcaagag atacgacgag caccaccagg acctgaccct |
| 1741 | gctgaaagct ctcgtgcggc agcagctgcc tgagaagtac aaagagattt tcttcgacca |
| 1801 | gagcaagaac ggctacgccg gctacattga cggcggagcc agccaggaag agttctacaa |
| 1861 | gttcatcaag cccatcctgg aaaagatgga cggcaccgag gaactgctcg tgaagctgaa |
| 1921 | cagagaggac ctgctgcgga agcagcggac cttcgacaac ggcagcatcc cccaccagat |
| 1981 | ccacctggga gagctgcacg ccattctgcg gcggcaggaa gatttttacc cattcctgaa |
| 2041 | ggacaaccgg gaaaagatcg agaagatcct gaccttccgc atcccctact acgtgggccc |
| 2101 | tctggccagg ggaaacagca gattcgcctg gatgaccaga aagagcgagg aaaccatcac |
| 2161 | cccctggaac ttcgaggaag tggtggacaa gggcgcttcc gcccagagct tcatcgagcg |
| 2221 | gatgaccaac ttcgataaga acctgcccaa cgagaaggtg ctgcccaagc acagcctgct |
| 2281 | gtacgagtac ttcaccgtgt ataacgagct gaccaaagtg aaatacgtga ccgagggaat |
| 2341 | gagaaagccc gccttcctga gcggcgagca gaaaaaggcc atcgtggacc tgctgttcaa |
| 2401 | gaccaaccgg aaagtgaccg tgaagcagct gaaagaggac tacttcaaga aaatcgagtg |
| 2461 | cttcgactcc gtggaaatct ccggcgtgga agatcggttc aacgcctccc tgggcacata |
| 2521 | ccacgatctg ctgaaaatta tcaaggacaa ggacttcctg gacaatgagg aaaacgagga |
| 2581 | cattctggaa gatatcgtgc tgaccctgac actgtttgag gacagagaga tgatcgagga |
| 2641 | acggctgaaa acctatgccc acctgttcga cgacaaagtg atgaagcagc tgaagcggcg |
| 2701 | gagatacacc ggctggggca gGTAAGAATG CACATCACTT CTTGAGAGTA TGGAGGAGTG |
| 2761 | AAATGACACT CAGTGCCAGA GTTACTGTAT ATCTACACTT TAAAAGTGTA GCTTTTAAAA |
| 2821 | GATAAGCAAG CACAATCTTT TGTGTGTGTG TGTGTGAATG TGTGTGTGTG TGTGTGTCAC |
| 2881 | CCAGATTATC ACGCAAATTG ATCAATGGAA TAAGAGATAA ACAGTCCGGA AAAACAATCC |
| 2941 | TTGATTTTTT AAAAAGTGAT GGGTTCGCAA ATAGAAATTT TATGCAACTC ATACATGATG |
| 3001 | ACAGCTTGAC ATTCAAAGAG GACATTCAGA AGGCGCAGGT ATGCATCACC CCCCCAGCTA |
| 3061 | ATTTTTTTTT GTATTTTTTA CCGAGTCGGG GTTTCGCAAT GTTGCCCAGG CTGGTCTCAG |
| 3121 | AGTCTCGCTC TGTTGTCTAC GCTGGAGTGC AGTAACATGA GCCACTGTGC CCGGCCAATC |
| 3181 | CTAAGAATTT CTTTTGCGGT GGTTGCAAGT CTGGGCAGAA CTCTTGTCAG GGGCTGTAAC |
| 3241 | TGGACTTATC TTTACTCCTT TGTCAGgtAt ccggccaggg cgatagcctg cacgagcaca |
| 3301 | ttgccaatct ggccggcagc cccgccatta agaagggcat cctgcagaca gtgaaggtgg |
| 3361 | tggacgagct cgtgaaagtg atgggccggc acaagcccga gaacatcgtg atcgaaatgg |
| 3421 | ccagagagaa ccagaccacc cagaagggac agaagaacag ccgcgagaga atgaagcgga |
| 3481 | tcgaagaggg catcaaagag ctgggcagcc agatcctgaa agaacacccc gtggaaaaca |
| 3541 | cccagctgca gaacgagaag ctgtacctgt actacctgca gaatgggcgg gatatgtacg |
| 3601 | tggaccagga actggacatc aaccggctgt ccgactacga tgtggaccat atcgtgcctc |
| 3661 | agagctttct gaaggacgac tccatcgaca acaaggtgct gaccagaagc gacaagaacc |
| 3721 | ggggcaagag cgacaacgtg ccctccgaag aggtcgtgaa gaagatgaag aactactggc |
| 3781 | ggcagctgct gaacgccaag ctgattaccc agagaaagtt cgacaatctg accaaggccg |
| 3841 | agagaggcgg cctgagcgaa ctggataagg ccggcttcat caagagacag ctggtggaaa |
| 3901 | cccggcagat cacaaagcac gtggcacaga tcctggactc ccggatgaac actaagtacg |
| 3961 | acgagaatga caagctgatc cgggaagtga aagtgatcac cctgaagtcc aagctggtgt |
| 4021 | ccgatttccg gaaggatttc cagttttaca aagtgcgcga gatcaacaac taccaccacg |
| 4081 | cccacgacgc ctacctgaac gccgtcgtgg gaaccgccct gatcaaaaag taccctaagc |
| 4141 | tggaaagcga gttcgtgtac ggcgactaca aggtgtacga cgtgcggaag atgatcgcca |
| 4201 | agagcgagca ggaaatcggc aaggctaccg ccaagtactt cttctacagc aacatcatga |
| 4261 | actttttcaa gaccgagatt accctggcca acggcgagat ccggaagcgg cctctgatcg |
| 4321 | agacaaacgg cgaaaccggg gagatcgtgt gggataaggg ccgggatttt gccaccgtgc |
| 4381 | ggaaagtgct gagcatgccc caagtgaata tcgtgaaaaa gaccgaggtg cagacaggcg |
| 4441 | gcttcagcaa agagtctatc ctgcccaaga ggaacagcga taagctgatc gccagaaaga |
| 4501 | aggactggga ccctaagaag tacggcggct tcgacagccc caccgtggcc tattctgtgc |
| 4561 | tggtggtggc caaagtggaa aagggcaagt ccaagaaact gaagagtgtg aaagagctgc |
| 4621 | tggggatcac catcatggaa agaagcagct tcgagaagaa tcccatcgac tttctggaag |
| 4681 | ccaagggcta caaagaagtg aaaaaggacc tgatcatcaa gctgcctaag tactccctgt |
| 4741 | tcgagctgga aaacggccgg aagagaatgc tggcctctgc cggcgaactg cagaagggaa |
| 4801 | acgaactggc cctgccctcc aaatatgtga acttcctgta cctggccagc cactatgaga |
| 4861 | agctgaaggg ctcccccgag gataatgagc agaaacagct gtttgtggaa cagcacaagc |
| 4921 | actacctgga cgagatcatc gagcagatca gcgagttctc caagagagtg atcctggccg |
| 4981 | acgctaatct ggacaaagtg ctgtccgcct acaacaagca ccgggataag cccatcagag |
| 5041 | agcaggccga gaatatcatc cacctgttta ccctgaccaa tctgggagcc cctgccgcct |
| 5101 | tcaagtactt tgacaccacc atcgaccgga agaggtacac cagcaccaaa gaggtgctgg |
| 5161 | acgccaccct gatccaccag agcatcaccg gcctgtacga gacacggatc gacctgtctc |
| 5221 | agctgggagg cgacaaaagg ccggcggcca cgaaaaaggc cggccaggca aaaaagaaaa |
| 5281 | aggaattcgg cagtggagag ggcagaggaa gtctgctaac atgcggtgac gtcgaggaga |
| 5341 | atcctggccc aGTCAGCAAA GGGGAAGAGG ACAACATGGC CATCATTAAG GAGTTTATGC |
| 5401 | GATTCAAAGT ACACATGGAG GGATCTGTTA ATGGCCATGA ATTTGAGATA GAGGGGGAAG |
| 5461 | GTGAGGGTCG CCCTTACGAA GGCACGCAGA CGGCTAAGCT GAAGGTCACG AAAGGGGGAC |
| 5521 | CCTTGCCCTT CGCATGGGAC ATACTCTCCC CACAGTTTAT GTATGGTTCT AAGGCATATG |
| 5581 | TTAAGCACCC TGCAGACATC CCAGACTATC TGAAGCTCTC CTTTCCTGAG GGGTTTAAGT |
| 5641 | GGGAACGCGT TATGAACTTT GAGGATGGAG GGGTCGTGAC TGTTACCCAG GATTCTTCCC |
| 5701 | TGCAAGATGG AGAGTTCATA TACAAAGTGA AACTTCGGGG AACGAATTTC CCATCAGACG |
| 5761 | GGCCAGTGAT GCAGAAAAAG ACGATGGGGT GGGAGGCTTC ATCCGAGAGG ATGTATCCCG |
| 5821 | AGGACGGAGC ATTGAAAGGC GAAATAAAAC AAAGGCTGAA GTTGAAGGAT GGGGGCCACT |
| 5881 | ACGACGCGGA GGTTAAAACA ACGTATAAAG CTAAAAAGCC AGTACAGCTC CCAGGCGCAT |
| 5941 | ATAACGTGAA TATAAAGCTT GACATAACGA GTCATAACGA GGATTACACA ATCGTAGAAC |
| 6001 | AGTACGAAAG AGCTGAAGGA CGGCACTCCA CCGGTGGGAT GGATGAACTC TATAAAGACT |
| 6061 | ACAAGGACGA TGATGACAAG TAAACAAATG GTAAGGAAGG GCACATCAAT CTTTGCTTAA |
| 6121 | TTGTCCTTTA CTCTAAAGAT GTATTTTATC ATACTGAATG CTAAACTTGA TATCTCCTTT |
| 6181 | TAGGTCATTG ATGTCCTTCA CCCCGGGAAG GCGACAGTGC CTAAGACAGA AATTCGGGAA |
| 6241 | AAACTAGCCA AAATGTACAA GACCACACCG GATGTCATCT TTGTATTTGG ATTCAGAACT |
| 6301 | CAGTAAACTG GATCCGCAGG CCTCTGCTAG CTTGACTGAC TGAGATACAG CGTACCTTCA |
| 6361 | GCTCACAGAC ATGATAAGAT ACATTGATGA GTTTGGACAA ACCACAACTA GAATGCAGTG |
| 6421 | AAAAAAATGC TTTATTTGTG AAATTTGTGA TGCTATTGCT TTATTTGTAA CCATTATAAG |
| 6481 | CTGCAATAAA CAAGTTAACA ACAACAATTG CATTCATTTT ATGTTTCAGG TTCAGGGGGA |
| 6541 | GGTGTGGGAG GTTTTTTAAA GCAAGTAAAA CCTCTACAAA TGTGGTATTG GCCCATCTCT |
| 6601 | ATCGGTATCG TAGCATAACC CCTTGGGGCC TCTAAACGGG TCTTGAGGGG TTTTTTGTGC |
| 6661 | CCCTCGGGCC GGATTGCTAT CTACCGGCAT TGGCGCAGAA AAAAATGCCT GATGCGACGC |
| 6721 | TGCGCGTCTT ATACTCCCAC ATATGCCAGA TTCAGCAACG GATACGGCTT CCCCAACTTG |
| 6781 | CCCACTTCCA TACGTGTCCT CCTTACCAGA AATTTATCCT TAAGGTCGTC AGCTATCCTG |
| 6841 | CAGGCGATCT CTCGATTTCG ATCAAGACAT TCCTTTAATG GTCTTTTCTG GACACCACTA |
| 6901 | GGGGTCAGAA GTAGTTCATC AAACTTTCTT CCCTCCCTAA TCTCATTGGT TACCTTGGGC |
| 6961 | TATCGAAACT TAATTAACCA GTCAAGTCAG CTACTTGGCG AGATCGACTT GTCTGGGTTT |
| 7021 | CGACTACGCT CAGAATTGCG TCAGTCAAGT TCGATCTGGT CCTTGCTATT GCACCCGTTC |
| 7081 | TCCGATTACG AGTTTCATTT AAATCATGTG AGCAAAAGGC CAGCAAAAGG CCAGGAACCG |
| 7141 | TAAAAAGGCC GCGTTGCTGG CGTTTTTCCA TAGGCTCCGC CCCCCTGACG AGCATCACAA |
| 7201 | AAATCGACGC TCAAGTCAGA GGTGGCGAAA CCCGACAGGA CTATAAAGAT ACCAGGCGTT |
| 7261 | TCCCCCTGGA AGCTCCCTCG TGCGCTCTCC TGTTCCGACC CTGCCGCTTA CCGGATACCT |
| 7321 | GTCCGCCTTT CTCCCTTCGG GAAGCGTGGC GCTTTCTCAT AGCTCACGCT GTAGGTATCT |
| 7381 | CAGTTCGGTG TAGGTCGTTC GCTCCAAGCT GGGCTGTGTG CACGAACCCC CCGTTCAGCC |
| 7441 | CGACCGCTGC GCCTTATCCG GTAACTATCG TCTTGAGTCC AACCCGGTAA GACACGACTT |
| 7501 | ATCGCCACTG GCAGCAGCCA CTGGTAACAG GATTAGCAGA GCGAGGTATG TAGGCGGTGC |
| 7561 | TACAGAGTTC TTGAAGTGGT GGCCTAACTA CGGCTACACT AGAAGAACAG TATTTGGTAT |
| 7621 | CTGCGCTCTG CTGAAGCCAG TTACCTTCGG AAAAAGAGTT GGTAGCTCTT GATCCGGCAA |
| 7681 | ACAAACCACC GCTGGTAGCG GTGGTTTTTT TGTTTGCAAG CAGCAGATTA CGCGCAGAAA |
| 7741 | AAAAGGATCT CAAGAAGATC CTTTGATCTT TTCTACGGGG TCTGACGCTC AGTGGAACGA |
| 7801 | AAACTCACGT TAAGGGATTT TGGTCATGAG ATTATCAAAA AGGATCTTCA CCTAGATCCT |
| 7861 | TTTAAATTAA AAATGAAGTT TTAAATCAAT CTAAAGTATA TATGAGTAAA CTTGGTCTGA |
| 7921 | CAGTTACCAA TGCTTAATCA GTGAGGCACC TATCTCAGCG ATCTGTCTAT TTCGTTCATC |
| 7981 | CATAGTTGCA TTTAAATTTC CGAACTCTCC AAGGCCCTCG TCGGAAAATC TTCAAACCTT |
| 8041 | TCGTCCGATC CATCTTGCAG GCTACCTCTC GAACGAACTA TCGCAAGTCT CTTGGCCGGC |
| 8101 | CTTGCGCCTT GGCTATTGCT TGGCAGCGCC TATCGCCAGG TATTACTCCA ATCCCGAATA |
| 8161 | TCCGAGATCG GGATCACCCG AGAGAAGTTC AACCTACATC CTCAATCCCG ATCTATCCGA |
| 8221 | GATCCGAGGA ATATCGAAAT CGGGGCGCGC CTGGTGTACC GAGAACGATC CTCTCAGTGC |
| 8281 | GAGTCTCGAC GATCCATATC GTTGCTTGGC AGTCAGCCAG TCGGAATCCA GCTTGGGACC |
| 8341 | CAGGAAGTCC AATCGTCAGA TATTGTACTC AAGCCTGGTC ACGGCAGCGT ACCGATCTGT |
| 8401 | TTAAACCTAG ATATTGATAG TCTGATCGGT CAACGTATAA TCGAGTCCTA GCTTTTGCAA |
| 8461 | ACATCTATCA AGAGACAGGA TCAGCAGGAG GCTTTCGCAT GAGTATTCAA CATTTCCGTG |
| 8521 | TCGCCCTTAT TCCCTTTTTT GCGGCATTTT GCCTTCCTGT TTTTGCTCAC CCAGAAACGC |
| 8581 | TGGTGAAAGT AAAAGATGCT GAAGATCAGT TGGGTGCGCG AGTGGGTTAC ATCGAACTGG |
| 8641 | ATCTCAACAG CGGTAAGATC CTTGAGAGTT TTCGCCCCGA AGAACGCTTT CCAATGATGA |
| 8701 | GCACTTTTAA AGTTCTGCTA TGTGGCGCGG TATTATCCCG TATTGACGCC GGGCAAGAGC |
| 8761 | AACTCGGTCG CCGCATACAC TATTCTCAGA ATGACTTGGT TGAGTATTCA CCAGTCACAG |
| 8821 | AAAAGCATCT TACGGATGGC ATGACAGTAA GAGAATTATG CAGTGCTGCC ATAACCATGA |
| 8881 | GTGATAACAC TGCGGCCAAC TTACTTCTGA CAACGATTGG AGGACCGAAG GAGCTAACCG |
| 8941 | CTTTTTTGCA CAACATGGGG GATCATGTAA CTCGCCTTGA TCGTTGGGAA CCGGAGCTGA |
| 9001 | ATGAAGCCAT ACCAAACGAC GAGCGTGACA CCACGATGCC TGTAGCAATG GCAACAACCT |
| 9061 | TGCGTAAACT ATTAACTGGC GAACTACTTA CTCTAGCTTC CCGGCAACAG TTGATAGACT |
| 9121 | GGATGGAGGC GGATAAAGTT GCAGGACCAC TTCTGCGCTC GGCCCTTCCG GCTGGCTGGT |
| 9181 | TTATTGCTGA TAAATCTGGA GCCGGTGAGC GTGGGTCTCG CGGTATCATT GCAGCACTGG |
| 9241 | GGCCAGATGG TAAGCCCTCC CGTATCGTAG TTATCTACAC GACGGGGAGT CAGGCAACTA |
| 9301 | TGGATGAACG AAATAGACAG ATCGCTGAGA TAGGTGCCTC ACTGATTAAG CATTGGTAAC |
| 9361 | CGATTCTAGG TGCATTGGCG CAGAAAAAAA TGCCTGATGC GACGCTGCGC GTCTTATACT |
| 9421 | CCCACATATG CCAGATTCAG CAACGGATAC GGCTTCCCCA ACTTGCCCAC TTCCATACGT |
| 9481 | GTCCTCCTTA CCAGAAATTT ATCCTTAAGA TCCCGAATCG TTTAAACTCG ACTCTGGCTC |
| 9541 | TATCGAATCT CCGTCGTTTC GAGCTTACGC GAACAGCCGT GGCGCTCATT TGCTCGTCGG |
| 9601 | GCATCGAATC TCGTCAGCTA TCGTCAGCTT ACCTTTTTGG CAGCGATCGC GGCTCCCGAC |
| 9661 | ATCTTGGACC ATTAGCTCCA CAGGTATCTT CTTCCCTCTA GTGGTCATAA CAGCAGCTTC |
| 9721 | AGCTACCTCT CAATTCAAAA AACCCCTCAA GACCCGTTTA GAGGCCCCAA GGGGTTATGC |
| 9781 | TATCAATCGT TGCGTTACAC ACACAAAAAA CCAACACACA TCCATCTTCG ATGGATAGCG |
| 9841 | ATTTTATTAT CTAACTGCTG ATCGAGTGTA GCCAGATCTA GTAATCAATT ACGGGGTCAT |
| 9901 | TAGTTCATAG CCC |

### Comments on Design 2 Constructs

Like Design 1, expression of the construct of Design 2 can be switched “on” or “off” depending on the presence of a splicing repressor that is either depleted or not depleted in neurodegenerative disease, e.g., TDP-43. In the presence of TDP-43, such as in healthy cells, splicing of the cryptic exon is repressed such that it is not present in the resultant transcribed mRNA. During subsequent translation, the ribosome encounters a premature termination codon within the leading to a non-functional truncated protein. Upon depletion of TDP-43, such as in diseased cells, the cryptic exon is instead retained in the resultant transcribed mRNA. Since the cryptic exon is frame-shift inducing (i.e., it has a sequence length that is not divisible by 3), the premature termination codon is no longer in frame with the start codon, allowing translation of the full-length translational protein. However, a frame shift may not be necessary if the cryptic exon encodes an essential part of the transgene such that without it the protein product is non-functional (e.g., a catalytic domain), or if the cryptic exon contains the start codon for the transgene.

A construct of Design 2 has many advantages. As compared with Design 1 the construct sequence is smaller. Additionally, and unlike Design 1 where, in diseased cells, an unwanted peptide is produced from the upstream regulatory region, which may either be an N-terminal sequence attached to the transgene protein product, or a short released peptide, in Design 2 no unwanted peptides are produced. Further, there is reduced potential for leaky expression of the full-length protein if the cryptic exon is expressed. Design 2 constructs are guaranteed to have zero leaky expression in the absence of the cryptic exon because the full, uninterrupted transgene sequence will not be present. In contrast, in Design 1 the full, uninterrupted transgene sequence is present in both healthy and diseased cells, leading to the possibility of leaky expression in healthy cells due to, for example, leaky ribosome scanning or alternative transcription initiation.

While the example Design 2 construct can comprise an intron (here together with a downstream exon sequence, derived from the RSP24 gene) downstream of the regulatory domain, this is a non-essential feature of the construct but is preferred because, similar to the Design 1 constructs, it can trigger nonsense mediated decay (NMD) of transcripts that do not include the cryptic exon sequence (i.e., those produced in healthy cells). This therefore further improves the safety of the constructs.

While the above example constructs show an exon encoding for part of the protein upstream and downstream of the cryptic exon, this need not be present if the start codon were to be included in the cryptic exon sequence itself. The cryptic exon may encode for an N-terminal, internal part or the C-terminal part of the protein.

While the above example constructs make use of a frame-shift inducing cryptic exon sequence, regulation can be obtained without requiring a frame-shift if the cryptic exon itself contains a start codon. Alternatively, a frame-shift inducing cryptic exon would not be required if the cryptic exon sequence was selected such that it encoded an essential part of the protein (e.g., a catalytic domain). In healthy cells, where the cryptic exon is not included in the mRNA product of the construct, a truncated non-functional transgene would be produced.

As described for Design 1, it is also envisaged that other TDP-43 binding domains can be used.

### Example 5

An exemplary construct was designed according to “Design 3”. The example construct comprises (from upstream to downstream)

A sequence comprising a start codon
A regulatory domain comprising
   a 3′ exonic sequence (here, based on exon 4 of AARS1)
   a splice donor site
   a single regulatory intronic region (here based on an intronic region between exon 4 and 5 of AARS1, comprising a TDP-43 binding domain)
   A splice acceptor site
   A 5′ exonic sequence (here, based on exon 5 of AARS1)
A P2A cleavage site and
A transgene for FLAG-mCherry

A further intron sequence comprising an intron in an exonic context (here, based on RPS24).

| | SEQ ID | |
|---|---|---|
| | NO: | Sequence |
| Construct | 95 | ATATATGGAGTTCCGCGTTACATAACTTACGGTAAATGGCCCGCCTGGCTGA |
| (single | | CCGCCCAACGACCCCCGCCCATTGACGTCAATAATGACGTATGTTCCCATAG |
| regulatory | | TAACGCCAATAGGGACTTTCCATTGACGTCAATGGGTGGAGTATTTACGGTAA |
| intron | | ACTGCCCACTTGGCAGTACATCAAGTGTATCATATGCCAAGTACGCCCCCTAT |
| underlined) | | TGACGTCAATGACGGTAAATGGCCCGCCTGGCATTATGCCCAGTACATGACC |
| | | TTATGGGACTTTCCTACTTGGCAGTACATCTACGTATTAGTCATCGCTATTAC |
| | | CATGCTGATGCGGTTTTGGCAGTACATCAATGGGCGTGGATAGCGGTTTGAC |
| | | TCACGGGGATTTCCAAGTCTCCACCCCATTGACGTCAATGGGAGTTTGTTTTG |
| | | GCACCAAAATCAACGGGACTTTCCAAAATGTCGTAACAACTCCGCCCCATTG |
| | | ACGCAAATGGGCGGTAGGCGTGTACGGTGGGAGGTCTATATAAGCAGAGCT |
| | | GGTTTAGTGAACCGTCAGATCAGATCTTTGTCGATCCTACCATCCACTCGACA |
| | | CACCCGCCAGCGGCCGCTTCTTGGTGCCAGCTTATCATAGCGCTACCGGTC |
| | | GCCACCATGGCGAGAACCATGGTAGCCATGGAGACCATGGGGCTCATGACA |
| | | ACAGATCTGGCAAAATTTGGGGTAAGAATGCACATCACTTCTTGAGAGTATGG |
| | | AGGAGTGAAATGACACTCAGTGCCAGAGTTACTGTATATCTACACTTTAAAAG |
| | | TGTAGCTTTTAAAAGATAAGCAAGCACAATCTTTTGTGTGTGTGTGTGTGAAT |
| | | GTGTGTGTGTGTGTGTGTCACCCAGGCTGGAGTGCAGTGGCATGATCACAG |
| | | CTCACTGCAGCCTCAAACTTCCTGGGCTCAAGTGATCCTCTCCCGAGTAGCT |
| | | GGGACTACAGGCTGGATGCCACCAAAATCCTCCCAGGCAACATACGGCAGC |
| | | GGCGCCACCAACTTTTCCCTGCTCAAGCAAGCCGGCGACGTGGAAGAGAAT |
| | | CCCGGCCCCGTCAGCAAAGGGGAAGAGGACAACATGGCCATCATTAAGGAG |
| | | TTTATGCGATTCAAAGTACACATGGAGGGATCTGTTAATGGCCATGAATTTGA |
| | | GATAGAGGGGGAAGGTGAGGGTCGCCCTTACGAAGGCACGCAGACGGCTAA |
| | | GCTGAAGGTCACGAAAGGGGGACCCTTGCCCTTCGCATGGGACATACTCTC |
| | | CCCACAGTTTATGTATGGTTCTAAGGCATATGTTAAGCACCCTGCAGACATCC |
| | | CAGACTATCTGAAGCTCTCCTTTCCTGAGGGGTTTAAGTGGGAACGCGTTAT |
| | | GAACTTTGAGGATGGAGGGGTCGTGACTGTTACCCAGGATTCTTCCCTGCAA |
| | | GATGGAGAGTTCATATACAAAGTGAAACTTCGGGGAACGAATTTCCCATCAG |
| | | ACGGGCCAGTGATGCAGAAAAAGACGATGGGGTGGGAGGCTTCATCCGAGA |
| | | GGATGTATCCCGAGGACGGAGCATTGAAAGGCGAAATAAAACAAAGGCTGAA |
| | | GTTGAAGGATGGGGGCCACTACGACGCGGAGGTTAAAACAACGTATAAAGCT |
| | | AAAAAGCCAGTACAGCTCCCAGGCGCATATAACGTGAATATAAAGCTTGACAT |
| | | AACGAGTCATAACGAGGATTACACAATCGTAGAACAGTACGAAAGAGCTGAA |
| | | GGACGGCACTCCACCGGTGGGATGGATGAACTCTATAAAGACTACAAGGAC |
| | | GATGATGACAAGTAAACAAATGGTAAGGAAGGGCACATCAATCTTTGCTTAAT |
| | | TGTCCTTTACTCTAAAGATGTATTTTATCATACTGAATGCTAAACTTGATATCT |
| | | CCTTTTAGGTCATTGATGTCCTTCACCCCGGGAAGGCGACAGTGCCTAAGAC |
| | | AGAAATTCGGGAAAAACTAGCCAAAATGTACAAGACCACACCGGATGTCATC |
| | | TTTGTATTTGGATTCAGAACTCAGTAAACTGGATCCGCAGGCCTCTGCTAGCT |
| | | TGACTGACTGAGATACAGCGTACCTTCAGCTCACAGACATGATAAGATACATT |
| | | GATGAGTTTGGACAAACCACAACTAGAATGCAGTGAAAAAAATGCTTTATTTG |
| | | TGAAATTTGTGATGCTATTGCTTTATTTGTAACCATTATAAGCTGCAATAAACA |
| | | AGTTAACAACAACAATTGCATTCATTTTATGTTTCAGGTTCAGGGGGAGGTGT |
| | | GGGAGGTTTTTTAAAGCAAGTAAAACCTCTACAAATGTGGTATTGGCCCATCT |
| | | CTATCGGTATCGTAGCATAACCCCTTGGGGCCTCTAAACGGGTCTTGAGGGG |
| | | TTTTTTGTGCCCCTCGGGCCGGATTGCTATCTACCGGCATTGGCGCAGAAAA |
| | | AAATGCCTGATGCGACGCTGCGCGTCTTATACTCCCACATATGCCAGATTCA |
| | | GCAACGGATACGGCTTCCCCAACTTGCCCACTTCCATACGTGTCCTCCTTAC |
| | | CAGAAATTTATCCTTAAGGTCGTCAGCTATCCTGCAGGCGATCTCTCGATTTC |
| | | GATCAAGACATTCCTTTAATGGTCTTTTCTGGACACCACTAGGGGTCAGAAGT |
| | | AGTTCATCAAACTTTCTTCCCTCCCTAATCTCATTGGTTACCTTGGGCTATCGA |
| | | AACTTAATTAACCAGTCAAGTCAGCTACTTGGCGAGATCGACTTGTCTGGGTT |
| | | TCGACTACGCTCAGAATTGCGTCAGTCAAGTTCGATCTGGTCCTTGCTATTGC |
| | | ACCCGTTCTCCGATTACGAGTTTCATTTAAATCATGTGAGCAAAAGGCCAGCA |
| | | AAAGGCCAGGAACCGTAAAAAGGCCGCGTTGCTGGCGTTTTTCCATAGGCTC |
| | | CGCCCCCCTGACGAGCATCACAAAAATCGACGCTCAAGTCAGAGGTGGCGA |
| | | AACCCGACAGGACTATAAAGATACCAGGCGTTTCCCCCTGGAAGCTCCCTCG |
| | | TGCGCTCTCCTGTTCCGACCCTGCCGCTTACCGGATACCTGTCCGCCTTTCT |
| | | CCCTTCGGGAAGCGTGGCGCTTTCTCATAGCTCACGCTGTAGGTATCTCAGT |
| | | TCGGTGTAGGTCGTTCGCTCCAAGCTGGGCTGTGTGCACGAACCCCCCGTT |
| | | CAGCCCGACCGCTGCGCCTTATCCGGTAACTATCGTCTTGAGTCCAACCCGG |
| | | TAAGACACGACTTATCGCCACTGGCAGCAGCCACTGGTAACAGGATTAGCAG |
| | | AGCGAGGTATGTAGGCGGTGCTACAGAGTTCTTGAAGTGGTGGCCTAACTAC |
| | | GGCTACACTAGAAGAACAGTATTTGGTATCTGCGCTCTGCTGAAGCCAGTTA |
| | | CCTTCGGAAAAAGAGTTGGTAGCTCTTGATCCGGCAAACAAACCACCGCTGG |
| | | TAGCGGTGGTTTTTTTGTTTGCAAGCAGCAGATTACGCGCAGAAAAAAAGGAT |
| | | CTCAAGAAGATCCTTTGATCTTTTCTACGGGGTCTGACGCTCAGTGGAACGA |
| | | AAACTCACGTTAAGGGATTTTGGTCATGAGATTATCAAAAAGGATCTTCACCT |
| | | AGATCCTTTTAAATTAAAAATGAAGTTTTAAATCAATCTAAAGTATATATGAGTA |
| | | AACTTGGTCTGACAGTTACCAATGCTTAATCAGTGAGGCACCTATCTCAGCGA |
| | | TCTGTCTATTTCGTTCATCCATAGTTGCATTTAAATTTCCGAACTCTCCAAGGC |
| | | CCTCGTCGGAAAATCTTCAAACCTTTCGTCCGATCCATCTTGCAGGCTACCTC |
| | | TCGAACGAACTATCGCAAGTCTCTTGGCCGGCCTTGCGCCTTGGCTATTGCT |
| | | TGGCAGCGCCTATCGCCAGGTATTACTCCAATCCCGAATATCCGAGATCGGG |
| | | ATCACCCGAGAGAAGTTCAACCTACATCCTCAATCCCGATCTATCCGAGATCC |
| | | GAGGAATATCGAAATCGGGGCGCGCCTGGTGTACCGAGAACGATCCTCTCA |
| | | GTGCGAGTCTCGACGATCCATATCGTTGCTTGGCAGTCAGCCAGTCGGAATC |
| | | CAGCTTGGGACCCAGGAAGTCCAATCGTCAGATATTGTACTCAAGCCTGGTC |
| | | ACGGCAGCGTACCGATCTGTTTAAACCTAGATATTGATAGTCTGATCGGTCAA |
| | | CGTATAATCGAGTCCTAGCTTTTGCAAACATCTATCAAGAGACAGGATCAGCA |
| | | GGAGGCTTTCGCATGAGTATTCAACATTTCCGTGTCGCCCTTATTCCCTTTTT |
| | | TGCGGCATTTTGCCTTCCTGTTTTTGCTCACCCAGAAACGCTGGTGAAAGTAA |
| | | AAGATGCTGAAGATCAGTTGGGTGCGCGAGTGGGTTACATCGAACTGGATCT |
| | | CAACAGCGGTAAGATCCTTGAGAGTTTTCGCCCCGAAGAACGCTTTCCAATG |
| | | ATGAGCACTTTTAAAGTTCTGCTATGTGGCGCGGTATTATCCCGTATTGACGC |
| | | CGGGCAAGAGCAACTCGGTCGCCGCATACACTATTCTCAGAATGACTTGGTT |
| | | GAGTATTCACCAGTCACAGAAAAGCATCTTACGGATGGCATGACAGTAAGAG |
| | | AATTATGCAGTGCTGCCATAACCATGAGTGATAACACTGCGGCCAACTTACTT |
| | | CTGACAACGATTGGAGGACCGAAGGAGCTAACCGCTTTTTTGCACAACATGG |
| | | GGGATCATGTAACTCGCCTTGATCGTTGGGAACCGGAGCTGAATGAAGCCAT |
| | | ACCAAACGACGAGCGTGACACCACGATGCCTGTAGCAATGGCAACAACCTTG |
| | | CGTAAACTATTAACTGGCGAACTACTTACTCTAGCTTCCCGGCAACAGTTGAT |
| | | AGACTGGATGGAGGCGGATAAAGTTGCAGGACCACTTCTGCGCTCGGCCCT |
| | | TCCGGCTGGCTGGTTTATTGCTGATAAATCTGGAGCCGGTGAGCGTGGGTCT |
| | | CGCGGTATCATTGCAGCACTGGGGCCAGATGGTAAGCCCTCCCGTATCGTA |
| | | GTTATCTACACGACGGGGAGTCAGGCAACTATGGATGAACGAAATAGACAGA |
| | | TCGCTGAGATAGGTGCCTCACTGATTAAGCATTGGTAACCGATTCTAGGTGC |
| | | ATTGGCGCAGAAAAAAATGCCTGATGCGACGCTGCGCGTCTTATACTCCCAC |
| | | ATATGCCAGATTCAGCAACGGATACGGCTTCCCCAACTTGCCCACTTCCATA |
| | | CGTGTCCTCCTTACCAGAAATTTATCCTTAAGATCCCGAATCGTTTAAACTCG |
| | | ACTCTGGCTCTATCGAATCTCCGTCGTTTCGAGCTTACGCGAACAGCCGTGG |
| | | CGCTCATTTGCTCGTCGGGCATCGAATCTCGTCAGCTATCGTCAGCTTACCTT |
| | | TTTGGCAGCGATCGCGGCTCCCGACATCTTGGACCATTAGCTCCACAGGTAT |
| | | CTTCTTCCCTCTAGTGGTCATAACAGCAGCTTCAGCTACCTCTCAATTCAAAA |
| | | AACCCCTCAAGACCCGTTTAGAGGCCCCAAGGGGTTATGCTATCAATCGTTG |
| | | CGTTACACACACAAAAAACCAACACACATCCATCTTCGATGGATAGCGATTTT |
| | | ATTATCTAACTGCTGATCGAGTGTAGCCAGATCTAGTAATCAATTACGGGGTC |
| | | ATTAGTTCATAGCCC |
| Regulatory | 96 | GGTTTAGTGAACCGTCAGATCAGATCTTTGTCGATCCTACCATCCACTCGACA |
| domain (single | | CACCCGCCAGCGGCCGCTTCTTGGTGCCAGCTTATCATAGCGCTACCGGTC |
| regulatory | | GCCACCATGGCGAGAACCATGGTAGCCATGGAGACCATGGGGCTCATGACA |
| intron | | ACAGATCTGGCAAAATTTGGGGTAAGAATGCACATCACTTCTTGAGAGTATGG |
| underlined and | | AGGAGTGAAATGACACTCAGTGCCAGAGTTACTGTATATCTACACTTTAAAAG |
| TDP-43 | | TGTAGCTTTTAAAAGATAAGCAAGCACAATCTTTTGTGTGTGTGTGTGTGAAT |
| binding | | GTGTGTGTGTGTGTGTGTCACCCAGGCTGGAGTGCAGTGGCATGATCACAG |
| domain in | | CTCACTGCAGCCTCAAACTTCCTGGGCTCAAGTGATCCTCTCCCGAGTAGCT |
| bold) | | GGGACTACAGGCTGGATGCCACCAAAATCCTCCCAGGCAACATAC |
| Single | 97 | GTAAGAATGCACATCACTTCTTGAGAGTATGGAGGAGTGAAATGACACTCAG |
| Regulatory | | TGCCAGAGTTACTGTATATCTACACTTTAAAAGTGTAGCTTTTAAAAGATAAGC |
| Intron | | AAGCACAATCTTTTGTGTGTGTGTGTGTGAATGTGTGTGTGTGTGTGTGTCAC |
| | | CCAG |
| mCherry- | 98 | GTCAGCAAAGGGGAAGAGGACAACATGGCCATCATTAAGGAGTTTATGCGAT |
| FLAG (FLAG- | | TCAAAGTACACATGGAGGGATCTGTTAATGGCCATGAATTTGAGATAGAGGG |
| underlined) | | GGAAGGTGAGGGTCGCCCTTACGAAGGCACGCAGACGGCTAAGCTGAAGGT |
| | | CACGAAAGGGGGACCCTTGCCCTTCGCATGGGACATACTCTCCCCACAGTTT |
| | | ATGTATGGTTCTAAGGCATATGTTAAGCACCCTGCAGACATCCCAGACTATCT |
| | | GAAGCTCTCCTTTCCTGAGGGGTTTAAGTGGGAACGCGTTATGAACTTTGAG |
| | | GATGGAGGGGTCGTGACTGTTACCCAGGATTCTTCCCTGCAAGATGGAGAGT |
| | | TCATATACAAAGTGAAACTTCGGGGAACGAATTTCCCATCAGACGGGCCAGT |
| | | GATGCAGAAAAAGACGATGGGGTGGGAGGCTTCATCCGAGAGGATGTATCC |
| | | CGAGGACGGAGCATTGAAAGGCGAAATAAAACAAAGGCTGAAGTTGAAGGAT |
| | | GGGGGCCACTACGACGCGGAGGTTAAAACAACGTATAAAGCTAAAAAGCCA |
| | | GTACAGCTCCCAGGCGCATATAACGTGAATATAAAGCTTGACATAACGAGTC |
| | | ATAACGAGGATTACACAATCGTAGAACAGTACGAAAGAGCTGAAGGACGGCA |
| | | CTCCACCGGTGGGATGGATGAACTCTATAAAGACTACAAGGACGATGATGAC |
| | | AAGTAA |
| Coding | 99 | ATGGCGAGAACCATGGTAGCCATGGAGACCATGGGGCTCATGACAACAGAT |
| sequence | | CTGGCAAAATTTGGGGTAAGAATGCACATCACTTCTTGA |
| when intron is | | |
| not retained | | |
| Amino acid | 100 | MARTMVAMETMGLMTTDLAKFGVRMHITS* |
| product when | | |
| intron is not | | |
| retained | | |
| Coding | 101 | ATGGCGAGAACCATGGTAGCCATGGAGACCATGGGGCTCATGACAACAGAT |
| sequence | | CTGGCAAAATTTGGGGCTGGAGTGCAGTGGCATGATCACAGCTCACTGCAG |
| when intron is | | CCTCAAACTTCCTGGGCTCAAGTGATCCTCTCCCGAGTAGCTGGGACTACAG |
| retained | | GCTGGATGCCACCAAAATCCTCCCAGGCAACATACGGCAGCGGCGCCACCA |
| | | ACTTTTCCCTGCTCAAGCAAGCCGGCGACGTGGAAGAGAATCCCGGCCCCG |
| | | TCAGCAAAGGGGAAGAGGACAACATGGCCATCATTAAGGAGTTTATGCGATT |
| | | CAAAGTACACATGGAGGGATCTGTTAATGGCCATGAATTTGAGATAGAGGGG |
| | | GAAGGTGAGGGTCGCCCTTACGAAGGCACGCAGACGGCTAAGCTGAAGGTC |
| | | ACGAAAGGGGGACCCTTGCCCTTCGCATGGGACATACTCTCCCCACAGTTTA |
| | | TGTATGGTTCTAAGGCATATGTTAAGCACCCTGCAGACATCCCAGACTATCTG |
| | | AAGCTCTCCTTTCCTGAGGGGTTTAAGTGGGAACGCGTTATGAACTTTGAGG |
| | | ATGGAGGGGTCGTGACTGTTACCCAGGATTCTTCCCTGCAAGATGGAGAGTT |
| | | CATATACAAAGTGAAACTTCGGGGAACGAATTTCCCATCAGACGGGCCAGTG |
| | | ATGCAGAAAAAGACGATGGGGTGGGAGGCTTCATCCGAGAGGATGTATCCC |
| | | GAGGACGGAGCATTGAAAGGCGAAATAAAACAAAGGCTGAAGTTGAAGGATG |
| | | GGGGCCACTACGACGCGGAGGTTAAAACAACGTATAAAGCTAAAAAGCCAGT |
| | | ACAGCTCCCAGGCGCATATAACGTGAATATAAAGCTTGACATAACGAGTCATA |
| | | ACGAGGATTACACAATCGTAGAACAGTACGAAAGAGCTGAAGGACGGCACTC |
| | | CACCGGTGGGATGGATGAACTCTATAAAGACTACAAGGACGATGATGACAAG |
| | | TAA |
| Amino acid | 102 | MARTMVAMETMGLMTTDLAKFGAGVQWHDHSSLQPQTSWAQVILSRVAGTTG |
| product when | | WMPPKSSQATYGSGATNFSLLKQAGDVEENPGPVSKGEEDNMAIIKEFMRFKV |
| intron is | | HMEGSVNGHEFEIEGEGEGRPYEGTQTAKLKVTKGGPLPFAWDILSPQFMYGS |
| retained | | KAYVKHPADIPDYLKLSFPEGFKWERVMNFEDGGVVTVTQDSSLQDGEFIYKVK |
| | | LRGTNFPSDGPVMQKKTMGWEASSERMYPEDGALKGEIKQRLKLKDGGHYDAE |
| | | VKTTYKAKKPVQLPGAYNVNIKLDITSHNEDYTIVEQYERAEGRHSTGGMDELY |
| | | KDYKDDDDK* |

The further intron sequence, the P2A cleavage sequence, the 3′ exonic sequence and 5′ exonic sequence were otherwise as described for Example 1A.

### Comments on Design 3

While demonstrated here with the transgene completely downstream of the regulatory domain, in other embodiments, the transgene sequence may be upstream and downstream of the single regulatory intron (i.e., as shown in FIG. 3, and in Examples 6A-D). Similarly, while shown here with the binding domain within the single regulatory intron, the binding domain may instead be upstream or downstream of the single regulatory intron. As described for Design 1, it is also envisaged that other TDP-43 binding domains can be used. As with Design 1 and Design 2 constructs, the P2A cleavage site, premature termination codon and further intronic sequence are only optional features and could be emitted.

### Results and Discussion

### Direct and Indirect TDP-43-Dependent Expression of Fluorescent Proteins

As indicated above, the present inventors generated a range of TDP-43-dependent expression vectors based on existing and novel cryptic exons, which express fluorescent proteins in response to TDP-43-knockdown. First, we generated a vector featuring an upstream, frame-shifting cryptic exon based on AARS1 (but with shorter introns, and an extra adenosine within the cryptic exon sequence), fused to mCherry with an N-terminal P2A site. This vector was transfected into SK-N-DZ cells with doxycycline-dependent TDP-43 knockdown, and the fluorescence was analysed by flow cytometry (see Methods). It was found that only minimal leaky expression was detected in untreated cells, but in cells with doxycycline treatment a large increase in mCherry signal was detected (FIG. 4 Part A; “AARS1-based Reporter”; fold-change in mean mCherry signal=8.2×).

Next, we generated three entirely synthetic cryptic exons and surrounding introns, aided by computational splicing prediction programs; the generated exonic and intronic sequences that were not derived from or based on any existing sequence (see Examples 2A-2C). In each case, the cryptic exon sequence encoded an internal part of mCherry, with the N- and C-terminal mCherry sequence encoded by the upstream and downstream exons respectively, such that only inclusion of the cryptic exon would result in a full mCherry transcript being expressed. Designs 1 and 2 featured a TDP-43 binding domain, comprising a TG-rich region upstream of the cryptic exon, whereas Design 3 featured a TDP-43 binding domain TG-rich region downstream of the cryptic exon. All three vectors exhibited increased mCherry expression upon TDP-43 knockdown, ranging from a 2.2× increase for Design 3, to a 16.1× increase for Design 2 (FIG. 4, Part A).

Next, further synthetic cryptic exons and surrounding introns were generated (see Examples 2D-2J). In each case, the cryptic exon sequence encoded an internal part of mScarlet, with the N- and C-terminal mScarlet sequence encoded by the upstream and downstream exons respectively, such that only inclusion of the cryptic exon would result in a full mScarlet transcript being expressed. Notably, these constructs either contained shorter TG repeats in the intronic regions flanking the cryptic exon, or comprised longer TG rich sequences in the intronic regions flanking the cryptic exon. These are summarised below. All example constructs showed increased expression in Dox-treated cells as compared to untreated cells.

These results are demonstrated in FIG. 12.

| | | p-value of TG- | | | |
|---|---|---|---|---|---|
| | | rich region | | | |
| | | (i.e., chance of | | Expression | |
| | | similarly TG | | in Dox- | Targeted |
| | | region by | Expression in | treated | SpliceAI |
| Example | TG position | random chance) | NT cells | cells | score |
| 2D | Short | 6E−8 | + | ++++ | 0.8 |
| | repeats both | | | | |
| | sides of | | | | |
| | cryptic | | | | |
| 2E | Repeats on | 2E−10 | − | ++++ | 0.8 |
| | both sides | | | | |
| | of cryptic | | | | |
| 2F | Downstream | <1E−6 | − | ++ | 0.3 |
| | TG | | | | |
| | repeats | | | | |
| 2G | Short | <1E−6 | + | ++ | 0.7 |
| | downstream | | | | |
| | TG repeats | | | | |
| 2H | Short | <1E−6 | − | +++ | 0.7 |
| | repeats on | | | | |
| | both sides | | | | |
| | of cryptic | | | | |
| 2I | TG-rich | <1E−6 | +++ | +++++ | 0.8 |
| | (without | | | | |
| | long | | | | |
| | repeats) on | | | | |
| | both sides | | | | |
| | of cryptic | | | | |
| 2J | TG-rich | <1E−6 | − | ++ | 0.2 |
| | (without | | | | |
| | long | | | | |
| | repeats) on | | | | |
| | both sides | | | | |
| | of cryptic | | | | |

Next, we designed a vector encoding Cre recombinase, where an internal part of the Cre recombinase sequence was encoded by a novel cryptic exon sequence (see Example 4). This was flanked by the same AARS1-derived intronic region used for Example 1A. Computational splicing prediction software was used to optimise this vector. To assess expression and activity of Cre recombinase inside cells, we cotransfected with a plasmid encoding mScarlet that featured a constitutive “poison exon” (an exon containing premature termination codons) flanked by two LoxP sites, such that Cre recombinase-mediated excision of the poison exon would be required for efficient mScarlet expression. Cells without TDP-43 knockdown, or cells in which the Cre recombinase was not transfected, exhibited minimal mScarlet expression, but cells transfected with both plasmids, and with TDP-43 knockdown, exhibited a 15.7× increase in mean mScarlet signal (FIG. 4, Part B). Furthermore, this result demonstrates that novel and different cryptic exon sequences can be inserted into the AARS1-derived intronic context and still behave as a cryptic exon.

Finally, a construct was developed comprising a single regulatory intron (i.e., according to Example 5) to provide proof of concept for a construct of “Design 3”. In such designs, the regulatory domain comprises a single regulatory intron, and transgenic expression was determined by whether intronic splicing was repressed. Cells without TDP-43 knockdown, exhibited minimal mCherry expression indicative of intron retention in the mRNA product, while cells with Dox-inducible TDP-43 knockdown, showed a marked increase in signal, indicating that the intron was effectively spliced (see FIG. 9).

TDP-43-Dependent Gaussia princeps Luciferase Expression

Gaussia princeps luciferase (GLuc) is a secreted luciferase, and is therefore suitable for use in biomarker studies, including minimally invasive biomarker studies in vivo. We designed a vector encoding GLuc (see Example 1B above). The construct was otherwise the same as described in Example 1A

As before, we transfected this vector into SK-N-DZ cells with or without TDP-43 knockdown. We then assessed the level of secreted GLuc enzyme by removing 20 ul of media from the cell culture and assessing chemiluminescence (see Methods). Supernatant from cells transfected with vectors not encoding GLuc, or cells without TDP-43 knockdown, did not give a strong signal; however, markedly raised signal was detectable from cells transfected with the cryptic Gluc vector and with TDP-43 knockdown (FIG. 5).

### TDP-43-Dependent Gene Editing

Next, it was assessed whether the cryptic exon could be used to limit gene editing via Cas9 enzyme to cells with TDP-43 depletion. Aided by computational splicing prediction software, we designed a mammalian Streptococcus pyogenes (S. pyogenes) Cas9 expression vector in which an internal part of the Cas9 coding sequence was encoded by a novel “cryptic exon”, flanked by intronic sequences derived from AARS1 (see Example 4). We then co-transfected SK-N-DZ cells with and without doxycycline-dependent TDP-43 knockdown with this vector, plus a vector encoding a single-guide RNA (sgRNA) targeting the human CDK4 gene. We then analysed expression of the FLAG-tagged Cas9 enzyme via western blotting, and analysed gene editing via amplicon Illumina sequencing.

Full-length FLAG-tagged Cas9 enzyme was only detected in cells transfected with the cryptic exon-containing vector with TDP-43 knockdown, whereas it was detected in cells transfected with a constitutive FLAG-tagged Cas9 expression plasmid in both conditions (FIG. 6, Part A). Consistent with these results, significantly raised numbers of indels were detected only in cells transfected with the constitutive Cas9 expression vector, or in cells transfected with the cryptic-exon Cas9 vector that had TDP-43 knocked down (FIG. 6, Part B).

### TDP-43 Fusion Protein Expression and Autoregulation

One approach for correcting TDP-43 nuclear loss of function is to express a splicing repressor that binds to the same target sequences as TDP-43. While this could be achieved via the transgenic expression of TDP-43; this could exacerbate cytoplasmic aggregation and toxicity. A different approach is therefore to express the RNA-binding domain of TDP-43 fused to a different splicing repressor; this avoids the risks associated with expressing the C-terminal domain of TDP-43, which is heavily implicated in cytoplasmic aggregation and toxicity. However, given that overexpression of TDP-43 can be toxic in vivo, it is expected that similar toxicity could result from expression of a TDP-43-based fusion protein, even if the toxic C-terminal domain is replaced with a safer alternative.

Instead, constructs according to the present invention presents a possible solution to this issue, because expression of the transgenic protein relies on TDP-43 loss of nuclear function. As a result, it is possible that our expression system can autoregulate if the therapeutic transgene were a TDP-43-based splicing repressor fusion protein. This is because expression of the transgene would in turn inhibit further expression of the transgene by repressing inclusion of the cryptic exon necessary for protein expression.

To test this idea, we fused the AARS1-based frameshifting system used for the Example 1A mCherry reporter, and replaced the mCherry with a TDP-43/Raver1 fusion (see Example 1C). This protein has previously shown to partially rescue TDP-43 loss of function. We also generated an RNA-binding-deficient mutant of the same construct, in which two phenylalanines in RNA-recognition domain 1 of TDP-43 were mutated to leucine (see Example 1C mutant).

We cotransfected these constructs into SK-N-DZ cells with inducible TDP-43 knockdown, combined with a minigene plasmid for a cryptic exon present in the human INSR gene [Ling et al. Science, 2015, 349 (6248); 650-5, which is incorporated herein by reference]. It was found that upon TDP-43 knockdown the inclusion of the cryptic exons increased (FIG. 7, Part A, lane 3 versus lane 6). In cells cotransfected with cryptic TDP-43-RAVER1 fusion, the percent inclusion of the cryptic exons was decreased, demonstrating that the loss of TDP-43-derived splicing repression was rescued (FIG. 7, Part A, lane 4). Rescue was not detected for the RNA-binding-deficient mutant, as expected (FIG. 7, Part A, lane 5).

INSR Minigene
SEQ ID NO: 103
ATATATGGAGTTCCGCGTTACATAACTTACGGTAAATGGCCCGCCT
GGCTGACCGCCCAACGACCCCCGCCCATTGACGTCAATAATGACG
TATGTTCCCATAGTAACGCCAATAGGGACTTTCCATTGACGTCAA
TGGGTGGAGTATTTACGGTAAACTGCCCACTTGGCAGTACATCAA
GTGTATCATATGCCAAGTACGCCCCCTATTGACGTCAATGACGGT
AAATGGCCCGCCTGGCATTATGCCCAGTACATGACCTTATGGGAC
TTTCCTACTTGGCAGTACATCTACGTATTAGTCATCGCTATTACC
ATGCTGATGCGGTTTTGGCAGTACATCAATGGGCGTGGATAGCGG
TTTGACTCACGGGGATTTCCAAGTCTCCACCCCATTGACGTCAAT
GGGAGTTTGTTTTGGCACCAAAATCAACGGGACTTTCCAAAATGT
CGTAACAACTCCGCCCCATTGACGCAAATGGGGGGTAGGCGTGTA
CGGTGGGAGGTCTATATAAGCAGAGCTGGTTTAGTGAACCGTCAG
ATCAGATCTTTGTCGATCCTACCATCCACTCGACACACCCGCCAG
CGGCCGCTTCTTGGTGCCAGCTTATCAGAACTACTCCTTCTATGC
CTTGGACAACCAGAACCTAAGGCAGCTCTGGGACTGGAGCAAACA
CAACCTCACCATCACTCAGGGGAAACTCTTCTTCCACTATAACCC
CAAACTCTGCTTGTCAGAAATCCACAAGATGGAAGAAGTTTCAGG
AACCAAGGGGCGCCAGGAGAGAAACGACATTGCCCTGAAGACCAA
TGGGGACCAGGCATCCTGTAAGTCACTGGTCCCCAACCTTTTTGG
CATGAGGGACCGGTGTAGTGGAAGATGGTTTTTCCATGGACTGGT
GGTGGGTGGGGATGGTTTCAGCATGATTCAAGTGCATTACATTTA
CTATGCACTTTATTCCTATTATGATTACATTGTAATATATAATGA
AAGAATTGTACAACTCACCATCATGTAGAATCAATGGGAACCCTG
AGCTTGTGTTCCTGCAACTAGATGGTCCCAGCTGGGGGTGATGGG
AGACAGTGACAGATCATCAGGCATTAGATTTTCATAAGGAGTGTG
CAGTCTAGGTACCTCATGTACACAGTTCACAATAGGGTTCACACC
CCTGTGAGAATCTAATGCCGCCGCTAATCTGACAGGAGGCAGAAC
TCAGGTGGTCATGCAAGCGATGGGGAGTGACTGTAAATACAGATG
AAGCTTCACTTGCTCACCTATCACTCACCTCCTGCTGTACAGCCC
TGTTCGTAACAGGCCATGGATAAGTACTGGTCTGTGGCCCAGGGG
CTGGGGACCCCTGCTGTAAGTGGTCCACAAACCAGATAATGTGGC
TGTCCTCTCTCATCCATCACAGTCACCCCCAGGGGGTATTACTTC
CCTCTAACAACTCACTGTGTGATAGGCTTTCTTACTGAGGGCAGA
TTCTGCACATTTATTAATATTATCACTATGCTTACTGTGCCATAT
AGTACCGGATACGGGATGAAGTCATACAAGCACTGAATGAATGGA
TGAATGAATGATGGATGAATGGATGACACCTTCTTATATGTGTAT
CAGGCTGATGCTGAAGACTTCAAAGTTGAGTAAAATACCTATGTC
AGTCTGCATCTCCTGGGAAGTGACTGCCAAGTTGAAGTTAGGAGT
GCAGAAAATGTATTGAGGGTAATATTCATAAAATATGAAACAGAG
GAAGAGCTTCTTTTTTTTTTTTTTTTTTTTTTGGGACAGAGTCTT
GCTCTGTCACCCAGGGCTGGAGTGCAGTGGCGTGATCTTGCCTCA
CTGCAACCTCCTTCCCCTGGGTTCAGGTAATTATCTCGCCTCAGC
CTCCAGAATAGCTGGGATTACAGGCACATGCCACCAAGCCCGGCT
AATTTTTTTTTTTGTATTTTTAGTAGAGACAGGGTTTTGCCATGT
TGGCCAGGGTGGTCTTGAACTCCTGACCTCAGGTGATCCTCCCGC
CTCGGCCTCCCAAAGTGCTGAGATTACAGGTGTGAGTCACCACGC
TCAGCCATGAAGAGCCTTTTGACAATAGCGTGTGTCTGACCTCTG
TGAACAGAGAGCGGGAAGGAGGGAGGATAGGGCTGGGAGAGTCTC
AGATGGTGATGCATCCCTGAGTCTTGGCCAAACCCAGAAAGAGAT
CAAGGCCACGGTTGTCTGCAGGGAAGTTCTGCATTGCAAAGGGAC
GGCCAGGCATCTACCAAGCTCAGTCATAGGTGGGGGCTGTCCAGG
GAGAGTCAGGTTTTGGCTGGAATGCTACAGCAGGTCCTGCAGTTT
CTGCAGCTGCAGGCTGCCTGCTGACTGCACTTCCCTGACAGATTC
TAAACAGTGAGCTGCCAAGGGCTTCTGGGATACCTTCATGGGGAG
TTAGTTACTTATGTCAAAATGTAGTGCAAGGGCTGGGCATGGTGG
CTCACGCCTGGAATCCCAGCACTCTGGGAGGCCGAGGCAGGCAGA
TCACTTGAGGTCAGGAGTTCGAGACCAGCCTGGCCAATGTGGTGA
AACTCCATCTCTACTAAAAAAAAAAAATACAAAAACTAGCTGGAC
GTGGTGGTGGGTGCCTGTAATCCCAGCTACTTGAGAGGCTGAGGC
ATGAGAATTGCTTAAACCCGGTAGGTGGACTGCACTCCAGCCTTG
GTGACAGAGCAAGACTGTCTCAAAAAAAATGTAGTGCAAGGAGAG
AGAGCGAGGTTGGGGTGAGGTTTAGGAGAGGGTTTGTCTTCTAGG
CAGAGAGAATTACTTAGATGCGTCTCTCCGATGTCTAATGATCTG
CAGGGTCTCTAAACTCACTTGGCATAGGTTTATTTGCACTGGAGT
TGCACCTCCTTCCAGGTCAGTCTTACAAGTCCATATGCGAGACAA
CGTTGTGTCAGGACAAACATCACCCTTGGAAATCCCTTCCTCCAA
TAACTATTGGCCGGTTGTCCTTCTTGCGCGGGTACAGACTGCGCT
TATTCAGTTGACTGTCTGGCTGAGTCAAGTCATTGGCTTACGTGA
GTGTGAGTGGCCAAGTTGCAAAACTGGCTCTTACCTTTGAATCTT
CCCCCATTCATACTCAGCCAGGCACATGGGGAGGAGACCCTTAAG
GGAATAGCAGCGTCACCTCTGCCTTCTCACGGTCCCTCCAGGAAG
TGTGGGGGTCCCAGGCTTTGGTCTGAAACTACACTGAAATAGCTC
ATTTTTGCCTTTTGTTTTAACTTTTCCAGGTGAAAATGAGTTACT
TAAATTTTCTTACATTCGGACATCTTTTGACAAGATCTTGCTGAG
ATGGGAGCCGTACTGGCCCCCCGACTTCCGAGACCTCTTGGGGTT
CATGCTGTTCTACAAAGAGGCGTAAACTGGATCCGCAGGCCTCTG
CTAGCTTGACTGACTGAGATACAGCGTACCTTCAGCTCACAGACA
TGATAAGATACATTGATGAGTTTGGACAAACCACAACTAGAATGC
AGTGAAAAAAATGCTTTATTTGTGAAATTTGTGATGCTATTGCTT
TATTTGTAACCATTATAAGCTGCAATAAACAAGTTAACAACAACA
ATTGCATTCATTTTATGTTTCAGGTTCAGGGGGAGGTGTGGGAGG
TTTTTTAAAGCAAGTAAAACCTCTACAAATGTGGTATTGGCCCAT
CTCTATCGGTATCGTAGCATAACCCCTTGGGGCCTCTAAACGGGT
CTTGAGGGGTTTTTTGTGCCCCTCGGGCCGGATTGCTATCTACCG
GCATTGGCGCAGAAAAAAATGCCTGATGCGACGCTGCGCGTCTTA
TACTCCCACATATGCCAGATTCAGCAACGGATACGGCTTCCCCAA
CTTGCCCACTTCCATACGTGTCCTCCTTACCAGAAATTTATCCTT
AAGGTCGTCAGCTATCCTGCAGGCGATCTCTCGATTTCGATCAAG
ACATTCCTTTAATGGTCTTTTCTGGACACCACTAGGGGTCAGAAG
TAGTTCATCAAACTTTCTTCCCTCCCTAATCTCATTGGTTACCTT
GGGCTATCGAAACTTAATTAACCAGTCAAGTCAGCTACTTGGCGA
GATCGACTTGTCTGGGTTTCGACTACGCTCAGAATTGCGTCAGTC
AAGTTCGATCTGGTCCTTGCTATTGCACCCGTTCTCCGATTACGA
GTTTCATTTAAATCATGTGAGCAAAAGGCCAGCAAAAGGCCAGGA
ACCGTAAAAAGGCCGCGTTGCTGGCGTTTTTCCATAGGCTCCGCC
CCCCTGACGAGCATCACAAAAATCGACGCTCAAGTCAGAGGTGGC
GAAACCCGACAGGACTATAAAGATACCAGGCGTTTCCCCCTGGAA
GCTCCCTCGTGCGCTCTCCTGTTCCGACCCTGCCGCTTACCGGAT
ACCTGTCCGCCTTTCTCCCTTCGGGAAGCGTGGCGCTTTCTCATA
GCTCACGCTGTAGGTATCTCAGTTCGGTGTAGGTCGTTCGCTCCA
AGCTGGGCTGTGTGCACGAACCCCCCGTTCAGCCCGACCGCTGCG
CCTTATCCGGTAACTATCGTCTTGAGTCCAACCCGGTAAGACACG
ACTTATCGCCACTGGCAGCAGCCACTGGTAACAGGATTAGCAGAG
CGAGGTATGTAGGCGGTGCTACAGAGTTCTTGAAGTGGTGGCCTA
ACTACGGCTACACTAGAAGAACAGTATTTGGTATCTGCGCTCTGC
TGAAGCCAGTTACCTTCGGAAAAAGAGTTGGTAGCTCTTGATCCG
GCAAACAAACCACCGCTGGTAGCGGTGGTTTTTTTGTTTGCAAGC
AGCAGATTACGCGCAGAAAAAAAGGATCTCAAGAAGATCCTTTGA
TCTTTTCTACGGGGTCTGACGCTCAGTGGAACGAAAACTCACGTT
AAGGGATTTTGGTCATGAGATTATCAAAAAGGATCTTCACCTAGA
TCCTTTTAAATTAAAAATGAAGTTTTAAATCAATCTAAAGTATAT
ATGAGTAAACTTGGTCTGACAGTTACCAATGCTTAATCAGTGAGG
CACCTATCTCAGCGATCTGTCTATTTCGTTCATCCATAGTTGCAT
TTAAATTTCCGAACTCTCCAAGGCCCTCGTCGGAAAATCTTCAAA
CCTTTCGTCCGATCCATCTTGCAGGCTACCTCTCGAACGAACTAT
CGCAAGTCTCTTGGCCGGCCTTGCGCCTTGGCTATTGCTTGGCAG
CGCCTATCGCCAGGTATTACTCCAATCCCGAATATCCGAGATCGG
GATCACCCGAGAGAAGTTCAACCTACATCCTCAATCCCGATCTAT
CCGAGATCCGAGGAATATCGAAATCGGGGCGCGCCTGGTGTACCG
AGAACGATCCTCTCAGTGCGAGTCTCGACGATCCATATCGTTGCT
TGGCAGTCAGCCAGTCGGAATCCAGCTTGGGACCCAGGAAGTCCA
ATCGTCAGATATTGTACTCAAGCCTGGTCACGGCAGCGTACCGAT
CTGTTTAAACCTAGATATTGATAGTCTGATCGGTCAACGTATAAT
CGAGTCCTAGCTTTTGCAAACATCTATCAAGAGACAGGATCAGCA
GGAGGCTTTCGCATGAGTATTCAACATTTCCGTGTCGCCCTTATT
CCCTTTTTTGCGGCATTTTGCCTTCCTGTTTTTGCTCACCCAGAA
ACGCTGGTGAAAGTAAAAGATGCTGAAGATCAGTTGGGTGCGCGA
GTGGGTTACATCGAACTGGATCTCAACAGCGGTAAGATCCTTGAG
AGTTTTCGCCCCGAAGAACGCTTTCCAATGATGAGCACTTTTAAA
GTTCTGCTATGTGGCGCGGTATTATCCCGTATTGACGCCGGGCAA
GAGCAACTCGGTCGCCGCATACACTATTCTCAGAATGACTTGGTT
GAGTATTCACCAGTCACAGAAAAGCATCTTACGGATGGCATGACA
GTAAGAGAATTATGCAGTGCTGCCATAACCATGAGTGATAACACT
GCGGCCAACTTACTTCTGACAACGATTGGAGGACCGAAGGAGCTA
ACCGCTTTTTTGCACAACATGGGGGATCATGTAACTCGCCTTGAT
CGTTGGGAACCGGAGCTGAATGAAGCCATACCAAACGACGAGCGT
GACACCACGATGCCTGTAGCAATGGCAACAACCTTGCGTAAACTA
TTAACTGGCGAACTACTTACTCTAGCTTCCCGGCAACAGTTGATA
GACTGGATGGAGGCGGATAAAGTTGCAGGACCACTTCTGCGCTCG
GCCCTTCCGGCTGGCTGGTTTATTGCTGATAAATCTGGAGCCGGT
GAGCGTGGGTCTCGCGGTATCATTGCAGCACTGGGGCCAGATGGT
AAGCCCTCCCGTATCGTAGTTATCTACACGACGGGGAGTCAGGCA
ACTATGGATGAACGAAATAGACAGATCGCTGAGATAGGTGCCTCA
CTGATTAAGCATTGGTAACCGATTCTAGGTGCATTGGCGCAGAAA
AAAATGCCTGATGCGACGCTGCGCGTCTTATACTCCCACATATGC
CAGATTCAGCAACGGATACGGCTTCCCCAACTTGCCCACTTCCAT
ACGTGTCCTCCTTACCAGAAATTTATCCTTAAGATCCCGAATCGT
TTAAACTCGACTCTGGCTCTATCGAATCTCCGTCGTTTCGAGCTT
ACGCGAACAGCCGTGGCGCTCATTTGCTCGTCGGGCATCGAATCT
CGTCAGCTATCGTCAGCTTACCTTTTTGGCAGCGATCGCGGCTCC
CGACATCTTGGACCATTAGCTCCACAGGTATCTTCTTCCCTCTAG
TGGTCATAACAGCAGCTTCAGCTACCTCTCAATTCAAAAAACCCC
TCAAGACCCGTTTAGAGGCCCCAAGGGGTTATGCTATCAATCGTT
GCGTTACACACACAAAAAACCAACACACATCCATCTTCGATGGAT
AGCGATTTTATTATCTAACTGCTGATCGAGTGTAGCCAGATCTAG
TAATCAATTACGGGGTCATTAGTTCATAGCCC

Next, we examined whether the construct was able to autoregulate. We found that cryptic exon inclusion of the AARS1-derived cryptic exon was reduced in cells transfected with the cryptic TDP-43-RAVER1 fusion, but not in cells transfected with the RNA-binding-deficient mutant or with a the AARS1-based TDP-43-dependent mCherry expression vector (FIG. 7, Part B). Given that expression of the fusion protein is reliant on inclusion of the AARS1 cryptic exon, this demonstrates that our system is able to autoregulate expression if the expressed transgene is a TDP-43-based splicing repressor.

Further evidence for autoregulation of similar constructs is shown in FIGS. 17 and 18.

### Materials and Methods

### Cell Culture

SK-N-DZ cells, with a doxycycline-inducible shRNA targeting TDP-43, were grown in 24 well dishes in DMEM/F12 media supplemented with Glutamax and 10% FBS. TDP-43 knockdown was achieved via treatment with 1 μg/ml doxycycline treatment for five days. Transfections were performed on Day 3 of treatment, using Lipofectamine 3000 (Thermo Scientific), using 500 ng of DNA total per well. Equivalent transfections for untreated and doxycycline treated cells were performed using the same transfection master mixes to limit variation in transfection between conditions. For smaller samples (i.e., cells grown in a 96-well), DNA amounts and Lipofectamine amounts were scaled according to vessel surface area.

### Flow Cytometry Analysis of Cells Expressing Fluorescent Proteins

Mammalian expression vectors for fluorescent proteins were co-transfected with a mammalian 100 ng of HaloTag expression vector (Promega) into SK-N-DZ cells. 48 hours after transfection, and following overnight incubation with a HaloTag-compatible far-red JaneliaFluor 646 dye (Promega), cells were washed in PBS, then analysed with BD LSRFortessa™ X-20 Cell Analyzer. Transfected cells were selected for analysis by gating for cells with high JaneliaFluor 646 signal; untransfected cells which were incubated with the JaneliaFluor 646 dye in parallel were used as a negative control for gating. 4′,6-diamidino-2-phenylindol (DAPI) staining was used to filter dead cells. mCherry signal was quantified for transfected cells, and background subtraction was performed by analysing the level of mCherry signal from equivalent untransfected cells of similar size (as assessed by forward and side scatter height, width and area values).

| Fluorophore | Laser | Filter |
|---|---|---|
| DAPI | 355 nm | 450/50 nm bandpass |
| mCherry/mScarlet | 561 nm | 600 nm longpass and 610/20 nm bandpass |
| JaneliaFluor 646 | 631 nm | 670/14 nm bandpass |

### Luciferase Analysis

Cells were grown and transfected as described above. 48 hours after transfection, 20 ul of media was removed and luminescence was assessed using the Pierce™ Gaussia Luciferase Glow Assay Kit (Thermo Scientific) as described in the manual.

### Cas9 Transfection, Western Blotting and Indel Analysis

Cells were grown and transfected as described above; each well was transfected with 300 ng of Cas9 expression vector and 200 ng of sgRNA expression vector. Western blots were prepared using NuPage 4-12% gels (Thermo Scientific). Antibodies used were 10782-2-AP (Proteintech) for TDP-43, FLAG M2 antibody for FLAG (Sigma Aldrich), and A11126 (Thermo Scientific) for tubulin. The Cas9 guide sequence used was 5′-CACTCTTGAGGGCCACAAAG-3′ (SEQ ID NO: 104). Genomic DNA was amplified using primers

SEQ ID NO: 105
5′-TCGTCGGCAGCGTCAGATGTGTATAAGAGACAGACGAACTGT
GCTGATGGGA-3′
and
SEQ ID NO: 106
5′-GTCTCGTGGGCTCGGAGATGTGTATAAGAGACAGGGTGCCTA
TGGGACAGTGTA-3′

and sequenced on an Illumina MiSeq machine using PE250.

### RT-PCR Analysis of Splicing

Cells were grown and transfected as described above; 100 ng of minigene plasmid was mixed with 400 ng of TDP-43/RAVER1 plasmid. 48 hours after transfection, RNA was extracted via the RNeasy Plus kit (Qiagen) following the manufacturer protocol. Random hexamer reverse transcription was performed with Superscript IV (Thermo Scientific), then PCR was performed using primers SEQ ID NO: 107 5′-CGATCCTACCATCCACTCG-3′ and SEQ ID NO: 108 5′-TTAATGATGGCCATGTTGTC-3′ for AARS1, or SEQ ID NO: 109 5′-CTTCTTGGTGCCAGCTTATCAGAACTACTCCTTCTATGCCTTGG-3′ and SEQ ID NO: 110 5′-GGCCTGCGGATCCAGTTTACGCCTCTTTGTAGAACAGCATG-3′ for INSR.

### Determination of STMN2 Cryptic Splicing Event

SH-SY5Y cells were grown in DMEM/F12 containing Glutamax supplemented with 10% FBS. For induction of shRNA against TDP-43, cells were treated with concentrations of 12.5 ng/ml, 18.75 ng/ml, 21 ng/ml, 25 ng/ml, and 75 ng/ml Doxycyline Hyclate (Sigma D9891). After 10 days, cells were harvested for RNA sequencing. To isolate RNA, the QIAGEN RNeasy mini kit was used, following manufacturer's instructions including the optional DNAse step. Sequencing libraries were prepared with polyA enrichment using a TruSeq Stranded mRNA Prep Kit (Illumina) and sequenced (2×150 bp) on an Illumina HiSeq 2500 machine.

Samples were quality trimmed using Fastp with the parameter “qualified_quality_phred: 10”, and aligned to the GRCh38 genome build using STAR (v2.7.0f) with gene models from GENCODE v31. STAR aligned BAMs were used as input to MAJIQ (v2.1) for splicing analysis using the GRCh38 reference genome. The results of the PSI module were then parsed using custom R scripts to obtain a PSI and probability of change for each junction. Cryptic splicing was defined as junctions with PSI<5% in control samples, PSI>10% in the 25 ng/mL condition, provided the junction was unannotated in GENCODE v31.”

TDP-43 protein levels were assessed as indicated in Brown, AL., Wilkins, O. G., Keuss, M. J. et al. TDP-43 loss and ALS-risk SNPs drive mis-splicing and depletion of UNC13A. Nature 603, 131-137 (2022), the contents of which are incorporated herein by reference.

Sequence of mScarlet-Encoding Plasmid Containing a “Poison Exon” Flanked by LoxP Sites (Used in Example 4A)

SEQ ID NO: 111
ATGGCGAGAACAATGGTTGCTATGGTGTCCAAAGGTGAGGCAGTC
ATAAAGGAGTTTATGAGGTTCAAGGTGCACATGGAAGGGTCAATG
AACGGACATGAGTTCGAAATTGAAGGTGAGGGCGAGGGCCGCCCC
TATGAAGGGACACAAACTGCCAAGCTCAAAGTGACCAAGGGCGGG
CCTCTGCCCTTCTCTTGGGATATCCTGAGCCCGCAGTTTATGTAC
GGCAGCCGGGCTTTCACCAAACACCCTGCCGATATCCCAGACTAC
TATAAACAGTCCTTTCCAGAAGGATTTAAGTGGGAGCGAGTCATG
AATTTCGAGGACGGAGGTGCCGTGACGGTTACTCAGGTAAGTCGT
GGACTAGAGTTTTGACTCGGCGATCACTTCCCATTTATAACTTCG
TATAGCATACATTATACGAAGTTATAACAATTTCTCCTTCCCCTC
GCTTTCCTCTACCTTCTCAGGTTTACCCTGACTTGAGTTGATTT
GGTCGTGCGCGAGAAATTCAGACTGGGACGCGACCTTCAGGTAAG
GACCTGAGTCTCCATCCCCGCACGCCCGAAACTCTGGGTAATAAC
TTCGTATAGCATACATTATACGAAGTTATGCAACCCTTTCCTTTC
CTCTTTCGACTTTTCTTTTTCCAGGACACCAGCCTGGAGGACGGC
ACCCTGATCTACAAGGTGAAGCTGAGGGGCACCAACTTCCCCCCC
GACGGCCCCGTGATGCAGAAGAAGACCATGGGCTGGGAGGCCAGC
ACCGAGAGGCTGTACCCCGAGGACGGCGTGCTGAAGGGCGACATC
AAGATGGCCCTGAGGCTGAAGGACGGCGGCAGGTACCTGGCCGAC
TTCAAGACCACCTACAAGGCCAAGAAGCCCGTGCAGATGCCCGGC
GCCTACAACGTGGACAGGAAGCTGGACATCACCAGCCACAACGAG
GACTACACCGTGGTGGAGCAGTACGAGAGGAGCGAGGGCAGGCAC
AGCACCGGCGGCATGGACGAGCTGTACAAGGACTACAAGGACGAT
GATGACAAGTGA

### Generation and Analysis of Barcoded Cas9 Cryptic Variants (i.e., Used in Example 1E)

To generate cryptic exons encoding part of Cas9 enzyme with a range of different synonymous mutations, oligos containing degenerate bases in the wobble position (i.e., third position) of relevant codons were ordered; these were then introduced in plasmids featuring 12 nt barcodes (produced via whole plasmid PCR with partially degenerate primers) via Gibson assembly.

The resulting plasmids were then transfected into SK-N-DZ cells as described above. Following RNA extraction, reverse transcription was performed with Superscript IV (Thermo Scientific) using a specific reverse transcription primer against the construct RNA, followed by PCR to amplify the relevant cDNA and add Illumina-compatible overhangs. Following sequencing using an Illumina MiSeq machine (Paired End 250), reads were analysed via a custom R script.

### Fluorescence Microscopy

Cells were imaged using an Olympus CKX53 microscope at 20× magnification with Green illumination, filtering for excitation in the red channel. Relevant settings (exposure, illumination level, objective lens) were kept consistent between images.

### “Algorithm 1” for Designing a Synthetic Cryptic Exon (i.e., as Used in Example 2C)

```
from keras.models import load model
from pkg resources import resource filename
from spliceai.utils import one hot encode
import numpy as np
import pandas as pd
import gzip
import random
paths =(‘models/spliceai{ }.h5’.format(x) for x in range(1, 6))
models = [load model(resource filename(‘spliceai’, x)) for x in paths]
def get_probs(input_sequence):
  context = 10000
  x = one hot encode(‘N’ *(context/ /2) + input sequence + ‘N’*(context//2)) [None,:]
  y = np.mean([models [m].predict(x) for m in range(5)], axis=0)
  acceptor_prob = y[0, :, 1]
  donor_prob = y[0, :, 2]
  return acceptor prob, donor prob
def make_nt_seq(aa_seq) :
  d = {“A”: [“GCT”, “GCC”, “GCA”, “GCG”], “I”: [“ATT”, “ATC”, “ATA”],
  “R”: [“CGT”, “CGC”, “CGA”, “CGG”, “AGA”, “AGG”],
  “L”: [“CTT”, “CTC”, “CTA”, “CTG”, “TTA”, “TTG”],
  “N”: [“AAT”, “AAC”], “K”: [“AAA”, “AAG”], “D”: [“GAT”, “GAC”], “M”: [“ATG”],
  “F”: [“TTT”, “TTC”], “C”: [“TGT”, “TGC”], “P”: [“CCT”, “CCC”, “CCA”, “CCG”],
  “Q”: [“CAA”, “CAG”], “S”: [“TCT”, “TCC”, “TCA”, “TCG”, “AGT”, “AGC”],
  “E”: [“GAA”, “GAG”], “T”: [“ACT”, “ACC”, “ACA”, “ACG”],
  “W”: [“TGG”],
  “G”: [“GGT”, “GGC”, “GGA”, “GGG”], “Y”: [“TAT”, “TAC”],
  “H”: [“CAT”, “CAC”], “V”: [“GTT”, “GTC”, “GTA”, “GTG”]}
  seq = “”
  for aa in aa seq:
    seq += random.choice(d [aa])
  return seq
def make random_seq(1):
  nts = [“A”, “C”, “G”, “T”]
  return ‘’.join(random.choices(nts, k=1))
def make ppt(1, frac) :
  s = “”
  for _ in range(1) :
  if random.uniform(0,1) <= frac:
    s += random.choice([“C”, “T”])
  else:
    s += random.choice([“A”, “G”])
    return s
def random_mut(seq, rate) :
  out = []
  nts = [“A”, “C”, “G”, “T”]
  for s in seq:
    if random.uniform(0,1) <= rate:
      out.append(random.choice(nts))
    else:
      out.append(s)
  return ‘’.join(out)
def main():
      upstream =
″TAGTGAACCGTCAGATCAGATCTTTGTCGATCCTACCATCCACTCGACACACCCGCCAGCGGCCGCTTCTTGGTGCCAGCTT
ATCAtagcgctaccggtcgccaccatggCgagaACCATGGTAGCCATGGAGaccATGgggctcATGGTCAGCAAAGGGGAAGA
GGACAACATGGCCATCATTAAGGAGTTTATGCGATTCAAAGTACACATGGAGGGATCTGTTAATGGCCATGAATTTGAGATAG
AGGGGGAAGGTGAGGGTCGCCCTTACGAAGGCACGCAGACGGCTAAGCTGAAGGTCACGAAAGGGGGACCCTTGCCCTTCGCA
TGGGACATACTCTCCCCACAG″
      downstream =
″ACTATCTGAAGCTCTCCTTTCCTGAGGGGTTTAAGTGGGAACGCGTTATGAACTTTGAGGATGGAGGGGTCGTGACTGTTAC
CCAGGATTCTTCCCTGCAAGATGGAGAGTTCATATACAAAGTGAAACTTCGGGGAACGAATTTCCCATCAGACGGGCCAGTGA
TGCAGAAAAAGACGATGGGGTGGGAGGCTTCATCCGAGAGGATGTATCCCGAGGACGGAGCATTGAAAGGCGAAATAAAACAA
AGGCTGAAGTTGAAGGATGGGGGCCACTACGACGCGGAGGTTAAAACAACGTATAAAGCTAAAAAGCCAGTACAGCTCCCAGG
CGCATATAACGTGAATATAAAGCTTGACATAACGAGTCATAACGAGGATTACACAATCGTAGAACAGTACGAAAGAGCTGAAG
GACGGCACTCCACCGGTGGGATGGATGAACTCTATAAAGACTACAAGGACGATGATGACAAGTAAACAAATGGTAAGGAAGGG
CACATCAATCTTTGCTTAATTGTCCTTTACTCTAAAGATGTATTTTATCATACTGAATGCTAAACTTGATATCTCCTTTTAGG
TCATTGATGTCCTTCACCCCGGGAAGGCGACAGTGCCTAAGACAGAAATTCGGGAAAAACTAGCCAAAATGTACAAGACCACA
CCGGATGTCATCTTTGTATTTGGATTCAGAACTCAGTAAACTGGATCCGCAGGCCTCTGCTAGCTTGACTGACTGAGATACAG
CGTACCT”
    cryptic_aa = “FMYGSKAYVKHPADIP”
    to_add_3p = “G”
    output_file = “synthetics” + make random seq(10) + “.csv”
    n = 20
    steps = 500
    early_stop = 50
    ideal_cryptic_score = 0.8
    l = 120
    with open(“output_downstreamUG/” + output_file, ‘w’) as file:
      file.write(“score,seq\n”)
      for iteration_number in range(n):
        to_add = “TGTGTGTGTGTGTGTGTTTGTGTGTGTGTGTGTGTG″
        intron1 = “GTAAG” + make_random_seq(1) + make ppt(30, 0.8) + “AG”
        cryptic = make_nt_seq(cryptic aa) + to add 3p
        intron2 = “GTAAG” + to_add + make_random_seq(1) + make_ppt(30, 0.8) + “CAG”
        intron1_start = len(upstream)
        intron1_end = len(upstream+intron1)
        intron2_start = len(upstream+intron1+cryptic)
        intron2_end = len(upstream+intron1+cryptic+intron2)
        stuck = 0
        print(“number “ + str(iteration_number))
        for j in range(steps):
          print(j)
          if j > 0:
            new_intron1 = “GT” + random_mut(intron1, 0.03) [2: len(intron1)−2] + “AG”
            new_intron2 = random_mut(intron2 [0:5], 0.03) + to add +
random_mut(intron2 [len(to_add)+5:len(intron2)−3], 0.03) + “CAG”
            if random.uniform(0,1) < 0.2:
              new_cryptic = make_nt_seq(cryptic aa) + to_add_3p
            else:
              new_cryptic = cryptic
              seq = upstream + new_intron1 + new_cryptic + new_intron2 + downstream
          else:
            seq = upstream + intron1 + cryptic + intron2 + downstream
            acceptor_prob, donor_prob = get probs(seq)
          # Is it good?
          Const_donor = donor_prob [intron1_start−1]
          ce_acceptor = acceptor_prob [intron1_end]
          ce_donor = donor_prob [intron2_start−1]
          const_acceptor = acceptor prob [intron2_end]
          score = const donor + const acceptor − abs(ce donor−
ideal_cryptic_score)*2 − abs(ce acceptor−ideal cryptic score)*2
          score += −2*(max(acceptor prob [intron1_start+2 : intron1_end−2]) + \
            max(donor_prob [intron1_start+2:intron1_end−2])+ \
            max(acceptor_prob [intron2_start+2:intron2_end−2]) + \
            max(donor_prob [intron2_start+2:intron2_end−2]))
          if j == 0:
            best score = score
            best seq = seq
            print(score)
            continue
          if score > best_score:
            intron1 = new_intron1
            intron2 = new_intron2
            cryptic = new_cryptic
            best score = score
            best seq = seq
            stuck = 0
            print(score)
            print(‘ ‘.join([str(const_donor), str(ce_acceptor), str(ce_donor), str(const_acceptor)]))
        else:
          stuck +=1
          if stuck > early stop:
            break
            file.write(‘,’.join([str(best_score), best seq]) + “\n″)
    df = pd.DataFrame.from_dict({‘don’ : donor prob, ‘acc’:acceptor prob,
‘pos’:range(0,len(acceptor_prob))})
if__name__ == “__main__″
    main()
```

### Supplementary Exemplification

### Fluorescence Microscopy and Nanopore Sequencing of Example 1A

Example 1A is a Design 1 construct that fuses an upstream cryptic exon based on AARS1 to a downstream mCherry sequence (see above for further details of design and sequence).

Fluorescence microscopy and nanopore sequencing was performed on SK-N-DZ cells that were transfected with a constitutively expressing mCherry vector or Example 1A, both with or without TDP-43 knockdown. FIG. 13A shows the fluorescence microscopy images. FIG. 13B shows the quantification of the images shown in FIG. 13A, where the numbers correspond to the log 2-fold-change in fluorescence signal upon in cells with TDP-43 knockdown. FIG. 13C shows the nanopore analysis of the splicing of the cells in FIG. 13A. A “Productive Transcript” is defined as one that enables expression of the transgene, in this case mCherry. For Example 1A (“Cryptic mCherry”) this means transcripts that have the upstream cryptic exon included.

### Fluorescence Microscopy and Nanopore Sequencing of Examples 2D-2J

Fluorescence microscopy and nanopore sequencing was performed on SK-N-DZ cells that were transfected with various Design 2 constructs (Examples 2D-2J) encoding mScarlet, both with or without TDP-43 knockdown. FIG. 14A shows the quantification of fluorescence microscopy images where the numbers refer to the log 2-fold change in fluorescence upon TDP-43 knockdown (e.g. 7.2 corresponds to 2\hat{ }7.2=147× increase in fluorescence). FIG. 14B shows nanopore analysis of the splicing of the cells in FIG. 14A. A “Productive Transcript” is defined as one that enables expression of the transgene mScarlet (i.e., with the cryptic exon included and spliced as expected)

### Example 6: Further Design 3 Vectors

Further Design 3 vectors comprising a single TG rich intron were designed.

It was found that the single intron designs were very successful when they comprised a “decoy” or alternative splice site, in addition to the first or cryptic splice site. The alternative or decoy splice site is the splice site that is used preferentially in normal cells (i.e., cells with TDP-43), whereas upon TDP-43 depletion, the first or cryptic splice site (i.e., flanked by TG-rich sequences) competes with the decoy or alternative splice site (i.e., not flanked by TG-rich sequences). Such designs can be generated computationally using a very similar approach to that described above.

### Example 6A

This Design 3 construct features a single intron with a cryptic acceptor splice site. It has a TG-rich region at the 3′ end of this intron (i.e., flanking the acceptor splice site) and a decoy splice site further downstream. When spliced productively it encodes mScarlet. The SpliceAI predicted splice strength of the cryptic acceptor splice site to be 25%.

| | | |
|---|---|---|
| Whole construct | SEQ ID | CTTCTTGGTGCCAGCTTATCATAGCGCTACCGGTCGCCACC |
| (start codon | NO: 161 | ATGGCGAGAACAATGGTTGCTATGGTGTCCAAAGGTGAGG |
| highlighted) | | CAGTCATAAAGGAGTTTATGAGGTTCAAGGTGCACATGGAA |
| | | GGGTCAATGAACGGACATGAGTTCGAGATCGAAGGTGAAG |
| | | GCGAAGGTCGGCCATACGAGGGAACGCAAACTGCAAAGCT |
| | | CAAGGTGACGAAAGGCGGACCACTACCCTTCAGCTGGGAT |
| | | ATCCTCAGTCCTCAGGTAAGTATGCATCTGTTGTTGTAAAGC |
| | | AGGCTAACAGCTTTGTCTTCATCGCCCCCACTCATTTTTGGT |
| | | TAATCTTACGGATGCCTGTAGAGGGGAATGTTGATTGTGTG |
| | | TGTGTGTGTGTGTGAATGTGTGTGTGTGTGTGTGACAGTTC |
| | | ATGTACGGATCGCGTGCCTTCACTAAGCACCCCGCTGATAT |
| | | CCCTGACTATTACAAACAGTCCTTCCCTGAGGGCTTCAAAT |
| | | GGGAGCGAGTAATGAATTTTGAGGACGGTGGAGCCGTGAC |
| | | GGTCACTCAGGATACTTCTCTTGAAGACGGTACCTTGATCT |
| | | ATAAAGTTAAGTTACGCGGCACTAATTTCCCACCTGATGGG |
| | | CCGGTCATGCAAAAGAAGACTATGGGGTGGGAGGCGTCGA |
| | | CAGAGCGCCTCTATCCCGAGGACGGCGTTTTGAAGGGGGA |
| | | TATTAAGATGGCACTAAGACTCAAAGATGGGGGAAGGTACC |
| | | TGGCCGACTTCAAGACCACCTACAAGGCCAAGAAGCCCGT |
| | | GCAGATGCCCGGCGCCTACAACGTGGACAGGAAGCTGGAC |
| | | ATCACCAGCCACAACGAGGACTACACCGTGGTGGAGCAGT |
| | | ACGAGAGGAGCGAGGGCAGGCACAGCACCGGCGGCATGG |
| | | ACGAGCTGTACAAGGACTACAAGGACGATGATGACAAGTGA |
| | | TAAACAAATGGTAAGGAAGGGCACATCAATCTTTGCTTAATT |
| | | GTCCTTTACTCTAAAGATGTATTTTATCATACTGAATGCTAAA |
| | | CTTGATATCTCCTTTTAGGTCATTGATGTCCTTCACCCCGGG |
| | | AAGGCGACAGTGCCTAAGACAGAAATTCGGGAAAAACTAGC |
| | | CAAAATGTACAAGACCACACCGGATGTCATCTTTGTATTTGG |
| | | ATTCAGAACTCAGTAAACTGGATCCGCAGGCC |
| Single intron | SEQ ID | GTAAGTATGCATCTGTTGTTGTAAAGCAGGCTAACAGCTTT |
| when spliced | NO: 162 | GTCTTCATCGCCCCCACTCATTTTTGGTTAATCTTACGGATG |
| productively | | CCTGTAGAGGGGAATGTTGATTGTGTGTGTGTGTGTGTGTG |
| (UG-rich region | | AATGTGTGTGTGTGTGTGTGACAG |
| underlined) | | |
| Single intron | SEQ ID | GTAAGTATGCATCTGTTGTTGTAAAGCAGGCTAACAGCTTT |
| when spliced | NO: 163 | GTCTTCATCGCCCCCACTCATTTTTGGTTAATCTTACGGATG |
| using the decoy | | CCTGTAGAGGGGAATGTTGATTGTGTGTGTGTGTGTGTGTG |
| splice site | | AATGTGTGTGTGTGTGTGTGACAGTTCATGTACGGATCGCG |
| | | TGCCTTCACTAAGCACCCCGCTGATATCCCTGACTATTACA |
| | | AACAG |
| Coding | SEQ ID | ATGGCGAGAACAATGGTTGCTATGGTGTCCAAAGGTGAGG |
| sequence when | NO: 164 | CAGTCATAAAGGAGTTTATGAGGTTCAAGGTGCACATGGAA |
| spliced | | GGGTCAATGAACGGACATGAGTTCGAGATCGAAGGTGAAG |
| productively | | GCGAAGGTCGGCCATACGAGGGAACGCAAACTGCAAAGCT |
| | | CAAGGTGACGAAAGGCGGACCACTACCCTTCAGCTGGGAT |
| | | ATCCTCAGTCCTCAGTTCATGTACGGATCGCGTGCCTTCAC |
| | | TAAGCACCCCGCTGATATCCCTGACTATTACAAACAGTCCTT |
| | | CCCTGAGGGCTTCAAATGGGAGCGAGTAATGAATTTTGAGG |
| | | ACGGTGGAGCCGTGACGGTCACTCAGGATACTTCTCTTGAA |
| | | GACGGTACCTTGATCTATAAAGTTAAGTTACGCGGCACTAA |
| | | TTTCCCACCTGATGGGCCGGTCATGCAAAAGAAGACTATGG |
| | | GGTGGGAGGCGTCGACAGAGCGCCTCTATCCCGAGGACG |
| | | GCGTTTTGAAGGGGGATATTAAGATGGCACTAAGACTCAAA |
| | | GATGGGGGAAGGTACCTGGCCGACTTCAAGACCACCTACA |
| | | AGGCCAAGAAGCCCGTGCAGATGCCCGGCGCCTACAACGT |
| | | GGACAGGAAGCTGGACATCACCAGCCACAACGAGGACTAC |
| | | ACCGTGGTGGAGCAGTACGAGAGGAGCGAGGGCAGGCAC |
| | | AGCACCGGCGGCATGGACGAGCTGTACAAGGACTACAAGG |
| | | ACGATGATGACAAGTGA |

### Example 6B

This Design 3 construct features a single intron with a cryptic acceptor splice site. It has a TG-rich region at the 3′ end of this intron (i.e., flanking the acceptor splice site). There is a decoy splice site further downstream and when spliced productively it encodes mScarlet. The SpliceAI predicted splice strength of the cryptic acceptor splice site is 75%.

| | | |
|---|---|---|
| Whole construct | SEQ ID | CTTCTTGGTGCCAGCTTATCATAGCGCTACCGGTCGCCACCAT |
| (start codon | NO: 165 | GGCGAGAACAATGGTTGCTATGGTGTCCAAAGGTGAGGCAGT |
| highlighted) | | CATAAAGGAGTTTATGAGGTTCAAGGTGCACATGGAAGGGTCA |
| | | ATGAACGGACATGAGTTCGAGATCGAAGGTGAAGGAGAGGGC |
| | | CGACCCTACGAAGGGACGCAGACGGCCAAGCTGAAGGTGACG |
| | | AAAGGCGGACCGTTGCCATTCTCGTGGGATATTCTGTCACCTC |
| | | AGGTAAGTATCGGTTTTTGTTGTTTGCAAGAGTGAAAAAGTGTA |
| | | GAAAACTAAATTATTAGATGTCGGGTGGTGCTGCTCGTTAGTCT |
| | | AACTCTAGCCGTTAACTTTGTGTGTGTGTGTGTGTGTGAATGTG |
| | | TGTGTGTGTGTGTGTTAGTTCATGTACGGCAGTCGTGCGTTCA |
| | | CGAAACACCCTGCTGACATCCCTGACTACTACAAACAGTCCTT |
| | | TCCCGAGGGCTTCAAATGGGAACGTGTCATGAATTTTGAAGAC |
| | | GGAGGGGCCGTGACCGTCACTCAAGATACGTCGTTGGAAGAC |
| | | GGAACGTTGATATATAAGGTCAAATTGCGCGGAACAAACTTCC |
| | | CACCCGATGGTCCAGTAATGCAAAAGAAGACTATGGGTTGGGA |
| | | GGCAAGTACAGAGAGGCTGTACCCGGAAGATGGTGTTCTAAAA |
| | | GGAGATATCAAGATGGCCCTGAGGCTTAAGGACGGTGGGCGA |
| | | TACCTGGCCGACTTCAAGACCACCTACAAGGCCAAGAAGCCC |
| | | GTGCAGATGCCCGGCGCCTACAACGTGGACAGGAAGCTGGAC |
| | | ATCACCAGCCACAACGAGGACTACACCGTGGTGGAGCAGTAC |
| | | GAGAGGAGCGAGGGCAGGCACAGCACCGGCGGCATGGACGA |
| | | GCTGTACAAGGACTACAAGGACGATGATGACAAGTGATAAACA |
| | | AATGGTAAGGAAGGGCACATCAATCTTTGCTTAATTGTCCTTTA |
| | | CTCTAAAGATGTATTTTATCATACTGAATGCTAAACTTGATATC |
| | | TCCTTTTAGGTCATTGATGTCCTTCACCCCGGGAAGGCGACAGT |
| | | GCCTAAGACAGAAATTCGGGAAAAACTAGCCAAAATGTACAAG |
| | | ACCACACCGGATGTCATCTTTGTATTTGGATTCAGAACTCAGTA |
| | | AACTGGATCCGCAGGCC |
| Single intron | SEQ ID | GTAAGTATCGGTTTTTGTTGTTTGCAAGAGTGAAAAAGTGTAGA |
| when spliced | NO: 166 | AAACTAAATTATTAGATGTCGGGTGGTGCTGCTCGTTAGTCTAA |
| productively | | CTCTAGCCGTTAACTTTGTGTGTGTGTGTGTGTGTGAATGTGT |
| (UG-rich region | | GTGTGTGTGTGTGTTAG |
| underlined) | | |
| Single intron | SEQ ID | GTAAGTATCGGTTTTTGTTGTTTGCAAGAGTGAAAAAGTGTAGA |
| when spliced | NO: 167 | AAACTAAATTATTAGATGTCGGGTGGTGCTGCTCGTTAGTCTAA |
| using the decoy | | CTCTAGCCGTTAACTTTGTGTGTGTGTGTGTGTGTGAATGTGT |
| splice site | | GTGTGTGTGTGTGTTAGTTCATGTACGGCAGTCGTGCGTTCAC |
| | | GAAACACCCTGCTGACATCCCTGACTACTACAAACAG |
| Coding | SEQ ID | ATGGCGAGAACAATGGTTGCTATGGTGTCCAAAGGTGAGGCA |
| sequence when | NO: 168 | GTCATAAAGGAGTTTATGAGGTTCAAGGTGCACATGGAAGGGT |
| spliced | | CAATGAACGGACATGAGTTCGAGATCGAAGGTGAAGGAGAGG |
| productively | | GCCGACCCTACGAAGGGACGCAGACGGCCAAGCTGAAGGTGA |
| | | CGAAAGGCGGACCGTTGCCATTCTCGTGGGATATTCTGTCACC |
| | | TCAGTTCATGTACGGCAGTCGTGCGTTCACGAAACACCCTGCT |
| | | GACATCCCTGACTACTACAAACAGTCCTTTCCCGAGGGCTTCA |
| | | AATGGGAACGTGTCATGAATTTTGAAGACGGAGGGGCCGTGA |
| | | CCGTCACTCAAGATACGTCGTTGGAAGACGGAACGTTGATATA |
| | | TAAGGTCAAATTGCGCGGAACAAACTTCCCACCCGATGGTCCA |
| | | GTAATGCAAAAGAAGACTATGGGTTGGGAGGCAAGTACAGAGA |
| | | GGCTGTACCCGGAAGATGGTGTTCTAAAAGGAGATATCAAGAT |
| | | GGCCCTGAGGCTTAAGGACGGTGGGCGATACCTGGCCGACTT |
| | | CAAGACCACCTACAAGGCCAAGAAGCCCGTGCAGATGCCCGG |
| | | CGCCTACAACGTGGACAGGAAGCTGGACATCACCAGCCACAA |
| | | CGAGGACTACACCGTGGTGGAGCAGTACGAGAGGAGCGAGG |
| | | GCAGGCACAGCACCGGCGGCATGGACGAGCTGTACAAGGACT |
| | | ACAAGGACGATGATGACAAGTGA |

### Example 6C

This further Design 3 construct features a single intron with a cryptic acceptor splice site. It has a TG-rich region spread throughout the intron, with no long pure TG repeats. There is a decoy splice site further downstream and when spliced productively it encodes mScarlet. The SpliceAI predicted splice strength of the cryptic acceptor splice site is 75%.

| | | |
|---|---|---|
| Whole | SEQ ID | CTTCTTGGTGCCAGCTTATCATAGCGCTACCGGTCGCCACCATGG |
| construct | NO: 169 | CGAGAACAATGGTTGCTATGGTGTCCAAAGGTGAGGCAGTCATAA |
| (start | | AGGAGTTTATGAGGTTCAAGGTGCACATGGAAGGGTCAATGAACG |
| codon | | GACATGAGTTCGAGATTGAAGGTGAAGGCGAAGGACGCCCCTAT |
| highlighted) | | GAAGGAACCCAAACGGCCAAGCTGAAAGTGACTAAAGGTGGACC |
| | | ACTGCCGTTCTCTTGGGACATCCTGAGCCCTCAGGTAAGTGTGTG |
| | | ATGGGTGAATGGGGAGTGCTTGAGTTGTGGCGTGCTTGAACTGG |
| | | GTGATTGTGTGGAAGTGTGGGTGACGCGTGTATGGGTGCATGTG |
| | | AGAGTGTCTGTCTTTGTGTGCTAGTTTATGTACGGTTCCCGCGCG |
| | | TTCACCAAACACCCAGCTGACATCCCGGACTACTACAAGCAATCC |
| | | TTCCCTGAGGGCTTTAAGTGGGAACGCGTGATGAACTTTGAGGAC |
| | | GGTGGTGCTGTTACAGTCACGCAAGATACTTCCTTAGAAGACGGT |
| | | ACCCTTATATACAAGGTTAAACTACGGGGCACCAATTTCCCTCCT |
| | | GACGGGCCGGTTATGCAGAAAAAAACAATGGGGTGGGAAGCCAGT |
| | | ACCGAGCGGCTCTACCCAGAAGATGGCGTACTCAAAGGTGATATC |
| | | AAAATGGCTCTCCGCCTTAAAGACGGCGGTCGGTACCTGGCCGA |
| | | CTTCAAGACCACCTACAAGGCCAAGAAGCCCGTGCAGATGCCCG |
| | | GCGCCTACAACGTGGACAGGAAGCTGGACATCACCAGCCACAAC |
| | | GAGGACTACACCGTGGTGGAGCAGTACGAGAGGAGCGAGGGCA |
| | | GGCACAGCACCGGCGGCATGGACGAGCTGTACAAGGACTACAAG |
| | | GACGATGATGACAAGTGATAAACAAATGGTAAGGAAGGGCACATC |
| | | AATCTTTGCTTAATTGTCCTTTACTCTAAAGATGTATTTTATCAT |
| | | ACTGAATGCTAAACTTGATATCTCCTTTTAGGTCATTGATGTCCT |
| | | TCACCCCGGGAAGGCGACAGTGCCTAAGACAGAAATTCGGGAAAA |
| | | ACTAGCCAAAATGTACAAGACCACACCGGATGTCATCTTTGTATT |
| | | TGGATTCAGAACTCAGTAAACTGGATCCGCAGGCC |
| Single | SEQ ID | GTAAGTGTGTGATGGGTGAATGGGGAGTGCTTGAGTTGTGGCGT |
| intron when | NO: 170 | GCTTGAACTGGGTGATTGTGTGGAAGTGTGGGTGACGCGTGTAT |
| spliced | | GGGTGCATGTGAGAGTGTCTGTCTTTGTGTGCTAG |
| productively | | |
| (TG-rich | | |
| region | | |
| underlined) | | |
| Single | SEQ ID | GTAAGTGTGTGATGGGTGAATGGGGAGTGCTTGAGTTGTGGCGT |
| intron when | NO: 171 | GCTTGAACTGGGTGATTGTGTGGAAGTGTGGGTGACGCGTGTAT |
| spliced | | GGGTGCATGTGAGAGTGTCTGTCTTTGTGTGCTAGTTTATGTACG |
| using the | | GTTCCCGCGCGTTCACCAAACACCCAGCTGACATCCCGGACTACT |
| decoy | | ACAAGCAATCCTTCCCTGAGGGCTTTAAGTGGGAACGCGTGATGA |
| splice site | | ACTTTGAGGACGGTGGTGCTGTTACAGTCACGCAAGATACTTCCT |
| | | TAGAAGACGGTACCCTTATATACAAGGTTAAACTACGGGGCACCA |
| | | ATTTCCCTCCTGACGGGCCGGTTATGCAGAAAAAAACAATGGGGT |
| | | GGGAAGCCAGTACCGAGCGGCTCTACCCAGAAGATGGCGTACTC |
| | | AAAGGTGATATCAAAATGGCTCTCCGCCTTAAAG |
| Coding | SEQ ID | ATGGCGAGAACAATGGTTGCTATGGTGTCCAAAGGTGAGGCAGT |
| sequence | NO: 172 | CATAAAGGAGTTTATGAGGTTCAAGGTGCACATGGAAGGGTCAAT |
| when | | GAACGGACATGAGTTCGAGATTGAAGGTGAAGGCGAAGGACGCC |
| spliced | | CCTATGAAGGAACCCAAACGGCCAAGCTGAAAGTGACTAAAGGTG |
| productively | | GACCACTGCCGTTCTCTTGGGACATCCTGAGCCCTCAGTTTATGT |
| | | ACGGTTCCCGCGCGTTCACCAAACACCCAGCTGACATCCCGGAC |
| | | TACTACAAGCAATCCTTCCCTGAGGGCTTTAAGTGGGAACGCGTG |
| | | ATGAACTTTGAGGACGGTGGTGCTGTTACAGTCACGCAAGATACT |
| | | TCCTTAGAAGACGGTACCCTTATATACAAGGTTAAACTACGGGGC |
| | | ACCAATTTCCCTCCTGACGGGCCGGTTATGCAGAAAAAAACAATG |
| | | GGGTGGGAAGCCAGTACCGAGCGGCTCTACCCAGAAGATGGCGT |
| | | ACTCAAAGGTGATATCAAAATGGCTCTCCGCCTTAAAGACGGCGG |
| | | TCGGTACCTGGCCGACTTCAAGACCACCTACAAGGCCAAGAAGC |
| | | CCGTGCAGATGCCCGGCGCCTACAACGTGGACAGGAAGCTGGAC |
| | | ATCACCAGCCACAACGAGGACTACACCGTGGTGGAGCAGTACGA |
| | | GAGGAGCGAGGGCAGGCACAGCACCGGCGGCATGGACGAGCTG |
| | | TACAAGGACTACAAGGACGATGATGACAAGTGA |

### Example 6D

This further Design 3 construct features a single intron with a cryptic donor splice site. It has a TG-rich region spread throughout the 5′ end of the intron. There is a decoy splice site upstream of the cryptic donor splice site and when spliced productively it encodes mScarlet. The SpliceAI predicted splice strength of the cryptic acceptor splice site is 20%.

| | | |
|---|---|---|
| Whole | SEQ ID NO: | CTTCTTGGTGCCAGCTTATCATAGCGCTACCGGTCGCCACCA |
| construct | 173 | TGGCGAGAACAATGGTTGCAATGGTTTCTAAAGGTGAGGCAG |
| (start codon | | TCATAAAGGAGTTTATGAGGTTCAAAGTTCACATGGAAGGGT |
| highlighted) | | CAATGAACGGACATGAGTTCGAAATTGAAGGTGAGGGGGAG |
| | | GGCCGCCCCTATGAAGGGACACAAACTGCCAAGCTCAAGGT |
| | | GACCAAGGGCGGGCCATTGCCCTTCTCTTGGGATATCCTGTC |
| | | TCCGCAAGTGAGTGTGTGTGTGAATGAGTGTGTATGTGTGTG |
| | | TCTGGGGGTGCTTGTGAGTGGGTGGGAGTGTATGCGGGTGT |
| | | TTGAAGTGGGGCTCCGATCATCTCTGTCCAGCCGCTTAGTTT |
| | | CCCGTTCCCCACTTATTTCCTGGTAGTTTATGTACGGCAGCC |
| | | GGGCTTTCACCAAACACCCTGCCGATATCCCGGACTACTATA |
| | | AACAGTCCTTTCCAGAAGGTTTTAAGTGGGAGCGCGTCATGA |
| | | ATTTCGAGGACGGAGGTGCCGTCACGGTTACTCAGGACACC |
| | | AGCCTGGAGGACGGCACCCTGATCTACAAGGTGAAGCTGAG |
| | | GGGCACCAACTTCCCCCCCGACGGCCCCGTGATGCAGAAGA |
| | | AGACCATGGGCTGGGAGGCCAGCACCGAGAGGCTGTACCC |
| | | CGAGGACGGCGTGCTGAAGGGCGACATCAAGATGGCCCTGA |
| | | GGCTGAAGGACGGCGGCAGGTACCTGGCCGACTTCAAGACC |
| | | ACCTACAAGGCCAAGAAGCCCGTGCAGATGCCCGGCGCCTA |
| | | CAACGTGGACAGGAAGCTGGACATCACCAGCCACAACGAGG |
| | | ACTACACCGTGGTGGAGCAGTACGAGAGGAGCGAGGGCAG |
| | | GCACAGCACCGGCGGCATGGACGAGCTGTACAAGGACTACA |
| | | AGGACGATGATGACAAGTGATAAACAAATGGTAAGGAAGGGC |
| | | ACATCAATCTTTGCTTAATTGTCCTTTACTCTAAAGATGTAT |
| | | TTTATCATACTGAATGCTAAACTTGATATCTCCTTTTAGGTC |
| | | ATTGATGTCCTTCACCCCGGGAAGGCGACAGTGCCTAAGACA |
| | | GAAATTCGGGAAAAACTAGCCAAAATGTACAAGACCACACCG |
| | | GATGTCATCTTTGTATTTGGATTCAGAACTCAGTAAACTGGA |
| | | TCCGCAGGCC |
| Single intron | SEQ ID NO: | GTGAGTGTGTGTGTGAATGAGTGTGTATGTGTGTGTCTGGG |
| when spliced | 174 | GGTGCTTGTGAGTGGGTGGGAGTGTATGCGGGTGTTTGAAG |
| productively | | TGGGGCTCCGATCATCTCTGTCCAGCCGCTTAGTTTCCCGT |
| (UG-rich | | TCCCCACTTATTTCCTGGTAG |
| region | | |
| underlined) | | |
| Single intron | SEQ ID NO: | GTGAGGCAGTCATAAAGGAGTTTATGAGGTTCAAAGTTCACA |
| when spliced | 175 | TGGAAGGGTCAATGAACGGACATGAGTTCGAAATTGAAGGTG |
| using the | | AGGGGGAGGGCCGCCCCTATGAAGGGACACAAACTGCCAAG |
| decoy splice | | CTCAAGGTGACCAAGGGCGGGCCATTGCCCTTCTCTTGGGA |
| site | | TATCCTGTCTCCGCAAGTGAGTGTGTGTGTGAATGAGTGTGT |
| | | ATGTGTGTGTCTGGGGGTGCTTGTGAGTGGGTGGGAGTGTA |
| | | TGCGGGTGTTTGAAGTGGGGCTCCGATCATCTCTGTCCAGC |
| | | CGCTTAGTTTCCCGTTCCCCACTTATTTCCTGGTAG |
| Coding | SEQ ID NO: | ATGGCGAGAACAATGGTTGCAATGGTTTCTAAAGGTGAGGCA |
| sequence | 176 | GTCATAAAGGAGTTTATGAGGTTCAAAGTTCACATGGAAGGG |
| when spliced | | TCAATGAACGGACATGAGTTCGAAATTGAAGGTGAGGGGGA |
| productively | | GGGCCGCCCCTATGAAGGGACACAAACTGCCAAGCTCAAGG |
| | | TGACCAAGGGCGGGCCATTGCCCTTCTCTTGGGATATCCTGT |
| | | CTCCGCAATTTATGTACGGCAGCCGGGCTTTCACCAAACACC |
| | | CTGCCGATATCCCGGACTACTATAAACAGTCCTTTCCAGAAG |
| | | GTTTTAAGTGGGAGCGCGTCATGAATTTCGAGGACGGAGGT |
| | | GCCGTCACGGTTACTCAGGACACCAGCCTGGAGGACGGCAC |
| | | CCTGATCTACAAGGTGAAGCTGAGGGGCACCAACTTCCCCC |
| | | CCGACGGCCCCGTGATGCAGAAGAAGACCATGGGCTGGGA |
| | | GGCCAGCACCGAGAGGCTGTACCCCGAGGACGGCGTGCTG |
| | | AAGGGCGACATCAAGATGGCCCTGAGGCTGAAGGACGGCG |
| | | GCAGGTACCTGGCCGACTTCAAGACCACCTACAAGGCCAAG |
| | | AAGCCCGTGCAGATGCCCGGCGCCTACAACGTGGACAGGAA |
| | | GCTGGACATCACCAGCCACAACGAGGACTACACCGTGGTGG |
| | | AGCAGTACGAGAGGAGCGAGGGCAGGCACAGCACCGGCGGC |
| | | ATGGACGAGCTGTACAAGGACTACAAGGACGATGATGACAA |
| | | GTGA |

### Fluorescence Microscopy and Nanopore Sequencing of Examples 6A-6D

Fluorescence microscopy and nanopore sequencing was performed on SK-N-DZ cells that were transfected with the above Example 6A-6D constructs with or without TDP-43 knockdown. FIG. 15A shows the fluorescence intensity in normal SK-N-DZ cells or those with TDP-43 knockdown where the numbers refer to the log 2-fold change in fluorescence upon TPD-43 knockdown (performed in triplicate) FIG. 15B shows nanopore analysis of the splicing from the cells. Error bars show standard deviation across three experimental replicates.

### Nanopore Sequencing Traces

Nanopore sequencing provides an excellent demonstration that splicing has occurred as expected. This is because it can provide the sequence of an entire mRNA transcript in a single read from a single molecule. Quantifications from nanopore sequencing are demonstrated in FIGS. 13C, 14B and 15B for Design 1, 2 and 3 constructs respectively, and FIG. 16 additionally shows four example nanopore traces for mScarlet for Examples 2E, 6A, 6B and 6D.
A. mScarlet vector “A11” [Example 2E], a “Design 2” construct. The cryptic exon is shown. Knockdown of TDP-43 results in inclusion of the cryptic exon, and an increase in full or partial intron retention.
B. mScarlet vector “B11” [Example 6A], a “Design 3” construct with a cryptic acceptor. In normal cells the decoy splice site is used. The cryptic acceptor is only used at a detectable level upon TDP-43 knockdown.
C. mScarlet vector “B12” [Example 6B], a “Design 3” construct with a cryptic acceptor. In normal cells the decoy splice site is mostly used. The cryptic acceptor is used at a very high level upon TDP-43 knockdown.
D. mScarlet vector “C3” [Example 6D], a “Design 3” construct with a cryptic donor. In normal cells the decoy splice site is used. The cryptic donor is only used at a detectable level upon TDP-43 knockdown.

In each case, the top part shows the predicted splicing pattern as designed. For In FIG. 16B-D, the alternative exon crated by the decoy splice site is shown in the second row and the cryptic exons or cryptic splice sites are highlighted (i.e., those that are expected to be used upon TDP-43 knockdown).

In each case, usage of the cryptic exon or cryptic splice site is required for expression of mScarlet. In each case, the usage of the cryptic splice site(s) is highlighted with a STAR symbol. In each case the constitutively spliced intron from RPS24 is visibly spliced correctly at the 3′ end of the transcript.

### Example 7: Design 2 TDP-43/Raver 1 Fusion Vectors

Additional “Design 2”-style vectors (i.e., feature an internal cryptic exon encoding part of the transgene) were designed that encode the TDP-43/Raver1 fusion protein. These vectors only express high levels of TDP-43/Raver1 fusion protein upon depletion of TDP-43.

Importantly, because these vectors express TDP-43/Raver1 fusion, they are able to rescue cryptic splicing events (i.e. compensate for the loss of endogenous TDP-43). This includes themselves, in other words, they are able to autoregulate, (i.e. they block their own cryptic splicing).

As such, to measure their cryptic splicing characteristics dependent on endogenous TDP-43, a vector expressing a mutant protein is used such that the transgenic protein does not interfere with the splicing regulation. The mutant we use has two phenylalanine to leucine mutations (F2L) in the TDP-43 RNA binding domain, which prevents the vector-expressed protein from repressing splicing and causing underestimation of cryptic exon inclusion. This was achieved with two T to C point mutations, which are highlighted in the sequences below.

For therapeutic applications, the non-mutant protein is used so that splicing can be rescued. As such, our experiments below analyse both the F2L mutant and wild-type versions of each example, to analyse cryptic splicing of the vector, or to analyse autoregulation and splicing rescue, respectively.

The sequences of the Example 7 constructs are shown below, where CDS stands for coding sequence. Example 7A has slightly weaker cryptic exon expression meaning there is a lower risk of leaky expression. Example 7B has stronger expression, designed such that there is better rescue of splicing as it should have higher maximal protein expression levels.

| | | |
|---|---|---|
| Example 7A | | |
| Whole Transcript | SEQ ID NO: | CTGGAGACGCCATCCACGCTGTTTTGACCTCCATAGA |
| (start codon in bold; | 177 | AGAATCTTTGTCGATCCTACCATCCACTCGACACACC |
| T signifies position | | CGCCAGCGGCCGCTTCTTGGTGCCAGCTTATCATAG |
| of dual T to C | | CGCTACCGGTCGCCACCATGGGCCCAAAAAAGAAGA |
| mutation in F2L | | GAAAGGTAGAAGACCCCGGCGGACCGGCGGCGAAA |
| mutant) | | CGCGTGAAACTGGATGGAGGTTACCCATACGATGTTC |
| | | CAGATTACGCTGGTGGTATGTCAGAATATATTCGGGT |
| | | CACCGAGGACGAGAACGACGAGCCTATCGAGATACC |
| | | ATCCGAAGACGACGGAACAGTCCTCCTGAGTACCGT |
| | | GACAGCACAATTCCCAGGGGCCTGCGGCCTCCGTTA |
| | | CAGAAACCCTGTTAGCCAGTGTATGAGGGGTGTGCG |
| | | GCTCGTGGAAGGCATACTCCACGCTCCGGACGCCGG |
| | | GTGGGGTAACTTGGTTTATGTCGTAAATTACCCTAAG |
| | | GACAATAAACGAAAGATGGACGAAACCGACGCTAGTA |
| | | GCGCCGTGAAAGTAAAACGGGCAGTGCAGAAGACAT |
| | | CTGACCTCATCGTCTTAGGTCTGCCTTGGAAGACCAC |
| | | AGAGCAGGATCTGAAAGAATATTTCTCTACTTTTGGC |
| | | GAAGTCCTGATGGTGCAGGTGAAAAAGGATCTGAAG |
| | | ACAGGGCATAGCAAAGGGTTCGGATTTGTCAGGTTCA |
| | | CTGAGTATGAGACCCAGGTGAAAGTGATGTCCCAGC |
| | | GACATATGATCGATGGGCGGTGGTGCGATTGTAAGC |
| | | TGCCTAATAGCAAGCAGTCTCAGGACGAACCCTTAAG |
| | | ATCCCGCAAGGTGTTCGTGGGTCGCTGCACGGAGGA |
| | | CATGACCGAGGATGAGCTAAGGTAAGTGGTGTGCCT |
| | | GTTGTGTGGTTGGGGTGTGGGTGTGTGTGGGATGGG |
| | | AGTGTGGTTGTCGCGTGTGATTGGCTCGGGTGCTTG |
| | | GATGGGTGATTGTCGGCGTGTTTGACAGGGAATTCTT |
| | | TTCGCAATACGGAGACGTAATGGACGTATTTATACCC |
| | | AAGCCATTCCGGGCCTTTGCCTTCGTTACTTTTGCTG |
| | | ATGACCAGGTGCGTGCATGAATCGTGTGCATGTTGTG |
| | | TGGTTGATTAGGGGTGTATGACCGGGTGATTGAGGC |
| | | GTGTATGGTGGTGTGGATGACAAGAGTGATTGTTGGT |
| | | GTGAATGAAGAGTGACTGACTAACGTCTTGACCGATT |
| | | CTACAGATCGCGCAATCCTTGTGCGGCGAGGACCTT |
| | | ATTATTAAGGGCATCTCTGTACACATCAGCAATGCAG |
| | | AGCCCAAGCATAATTCTAACCTGCCACCTTTACTGGG |
| | | CCCCTCTGGCGGCGACCGGGAGCCAATGGGACTAG |
| | | GCCCACCAGCAACGCAGCTGACTCCACCACCCGCCC |
| | | CAGTTGGCTTGCGTGGATCCAACCACCGTGGACTTC |
| | | CCAAAGATAGTGGCCCCTTGCCTACGCCACCCGGCG |
| | | TGAGCCTGCTAGGCGAGCCACCAAAGGATTACAGGA |
| | | TACCCCTGAACCCTTACCTTAATCTCCACTCACTGCT |
| | | GCCCTCTAGCAATCTTGCGGGAAAAGAGACCAGGGG |
| | | CTGGGGCGGAAGCGGGAGAGGGCGAAGACCAGCTG |
| | | AGCCGCCACTGCCTTCGCCAGCAGTTCCTGGAGGAG |
| | | GGTCAGGCAGTAACAATGGCAACAAAGCGTTCCAAAT |
| | | GAAAAGTCGACTCTTGTCTCCCATTGCCTCTAACCGC |
| | | CTGCCTCCCGAACCCGGGCTGCCAGACTCCTATGGA |
| | | TTTGATTACCCGACAGATGTGGGTCCTCGCCGCTTGT |
| | | TCAGCCATCCCAGAGAACCTACTCTAGGAGCCCACG |
| | | GGCCGAGTAGGCACAAAATGTCGCCTCCGCCGTCCT |
| | | CATTCAACGAGCCTAGATCCGGCGGTGGGTCCGGAG |
| | | GCCCACTTTCGCACTTCTGATAAACAAATGGTAAGGA |
| | | AGGGCACATCAATCTTTGCTTAATTGTCCTTTACTCTA |
| | | AAGATGTATTTTATCATACTGAATGCTAAACTTGATAT |
| | | CTCCTTTTAGGTCATTGATGTCCTTCACCCCGGGAAG |
| | | GCGACAGTGCCTAAGACAGAAATTCGGGAAAAACTAG |
| | | CCAAAATGTACAAGACCACACCGGATGTCATCTTTGT |
| | | ATTTGGATTCAGAACTCAGTAAACTGGATCCGCAGGC |
| | | C |
| Upstream CDS | SEQ ID NO: | ATGGGCCCAAAAAAGAAGAGAAAGGTAGAAGACCCC |
| | 178 | GGCGGACCGGCGGCGAAACGCGTGAAACTGGATGG |
| | | AGGTTACCCATACGATGTTCCAGATTACGCTGGTGGT |
| | | ATGTCAGAATATATTCGGGTCACCGAGGACGAGAACG |
| | | ACGAGCCTATCGAGATACCATCCGAAGACGACGGAA |
| | | CAGTCCTCCTGAGTACCGTGACAGCACAATTCCCAGG |
| | | GGCCTGCGGCCTCCGTTACAGAAACCCTGTTAGCCA |
| | | GTGTATGAGGGGTGTGCGGCTCGTGGAAGGCATACT |
| | | CCACGCTCCGGACGCCGGGTGGGGTAACTTGGTTTA |
| | | TGTCGTAAATTACCCTAAGGACAATAAACGAAAGATG |
| | | GACGAAACCGACGCTAGTAGCGCCGTGAAAGTAAAA |
| | | CGGGCAGTGCAGAAGACATCTGACCTCATCGTCTTAG |
| | | GTCTGCCTTGGAAGACCACAGAGCAGGATCTGAAAG |
| | | AATATTTCTCTACTTTTGGCGAAGTCCTGATGGTGCA |
| | | GGTGAAAAAGGATCTGAAGACAGGGCATAGCAAAGG |
| | | GTTCGGATTTGTCAGGTTCACTGAGTATGAGACCCAG |
| | | GTGAAAGTGATGTCCCAGCGACATATGATCGATGGG |
| | | CGGTGGTGCGATTGTAAGCTGCCTAATAGCAAGCAGT |
| | | CTCAGGACGAACCCTTAAGATCCCGCAAGGTGTTCGT |
| | | GGGTCGCTGCACGGAGGACATGACCGAGGATGAGCT |
| | | AAG |
| First part of intron | SEQ ID NO: | GTAAGTGGTGTGCCTGTTGTGTGGTTGGGGTGTGGG |
| | 179 | TGTGTGTGGGATGGGAGTGTGGTTGTCGCGTGTGAT |
| | | TGGCTCGGGTGCTTGGATGGGTGATTGTCGGCGTGT |
| | | TTGACAG |
| Cryptic exon | SEQ ID NO: | GGAATTCTTTTCGCAATACGGAGACGTAATGGACGTA |
| | 180 | TTTATACCCAAGCCATTCCGGGCCTTTGCCTTCGTTA |
| | | CTTTTGCTGATGACCAG |
| Second part of | SEQ ID NO: | GTGCGTGCATGAATCGTGTGCATGTTGTGTGGTTGAT |
| intron | 181 | TAGGGGTGTATGACCGGGTGATTGAGGCGTGTATGG |
| | | TGGTGTGGATGACAAGAGTGATTGTTGGTGTGAATGA |
| | | AGAGTGACTGACTAACGTCTTGACCGATTCTACAG |
| Downstream CDS | SEQ ID NO: | ATCGCGCAATCCTTGTGCGGCGAGGACCTTATTATTA |
| | 182 | AGGGCATCTCTGTACACATCAGCAATGCAGAGCCCAA |
| | | GCATAATTCTAACCTGCCACCTTTACTGGGCCCCTCT |
| | | GGCGGCGACCGGGAGCCAATGGGACTAGGCCCACC |
| | | AGCAACGCAGCTGACTCCACCACCCGCCCCAGTTGG |
| | | CTTGCGTGGATCCAACCACCGTGGACTTCCCAAAGAT |
| | | AGTGGCCCCTTGCCTACGCCACCCGGCGTGAGCCTG |
| | | CTAGGCGAGCCACCAAAGGATTACAGGATACCCCTG |
| | | AACCCTTACCTTAATCTCCACTCACTGCTGCCCTCTA |
| | | GCAATCTTGCGGGAAAAGAGACCAGGGGCTGGGGC |
| | | GGAAGCGGGAGAGGGCGAAGACCAGCTGAGCCGCC |
| | | ACTGCCTTCGCCAGCAGTTCCTGGAGGAGGGTCAGG |
| | | CAGTAACAATGGCAACAAAGCGTTCCAAATGAAAAGT |
| | | CGACTCTTGTCTCCCATTGCCTCTAACCGCCTGCCTC |
| | | CCGAACCCGGGCTGCCAGACTCCTATGGATTTGATTA |
| | | CCCGACAGATGTGGGTCCTCGCCGCTTGTTCAGCCA |
| | | TCCCAGAGAACCTACTCTAGGAGCCCACGGGCCGAG |
| | | TAGGCACAAAATGTCGCCTCCGCCGTCCTCATTCAAC |
| | | GAGCCTAGATCCGGCGGTGGGTCCGGAGGCCCACTT |
| | | TCGCACTTCTGA |
| Example 7B | | |
| Whole Transcript | SEQ ID NO: | CTGGAGACGCCATCCACGCTGTTTTGACCTCCATAGAA |
| (start codon in bold; | 183 | GAATCTTTGTCGATCCTACCATCCACTCGACACACCCGC |
| T signifies position | | CAGCGGCCGCTTCTTGGTGCCAGCTTATCATAGCGCTA |
| of dual T to C | | CCGGTCGCCACCATGGGCCCAAAAAAGAAGAGAAAGGT |
| mutation in F2L | | AGAAGACCCCGGCGGACCGGCGGCGAAACGCGTGAAA |
| mutant) | | CTGGATGGAGGTTACCCATACGATGTTCCAGATTACGCT |
| | | GGTGGTATGTCAGAATATATTCGGGTCACCGAGGACGA |
| | | GAACGACGAGCCTATCGAGATACCATCCGAAGACGACG |
| | | GAACAGTCCTCCTGAGTACCGTGACAGCACAATTCCCA |
| | | GGGGCCTGCGGCCTCCGTTACAGAAACCCTGTTAGCCA |
| | | GTGTATGAGGGGTGTGCGGCTCGTGGAAGGCATACTCC |
| | | ACGCTCCGGACGCCGGGTGGGGTAACTTGGTTTATGTC |
| | | GTAAATTACCCTAAGGACAATAAACGAAAGATGGACGAA |
| | | ACCGACGCTAGTAGCGCCGTGAAAGTAAAACGGGCAGT |
| | | GCAGAAGACATCTGACCTCATCGTCTTAGGTCTGCCTTG |
| | | GAAGACCACAGAGCAGGATCTGAAAGAATATTTCTCTAC |
| | | TTTTGGCGAAGTCCTGATGGTGCAGGTGAAAAAGGATC |
| | | TGAAGACAGGGCATAGCAAAGGGTTCGGATTTGTCAGG |
| | | TTCACTGAGTATGAGACCCAGGTGAAAGTGATGTCCCA |
| | | GCGACATATGATCGATGGGCGGTGGTGCGATTGTAAGC |
| | | TGCCTAATAGCAAGCAGTCTCAGGACGAACCCTTAAGAT |
| | | CCCGCAAGGTGTTCGTGGGTCGCTGCACGGAGGACAT |
| | | GACGGAGGACGAACTAAGGTAAGCGGAGTGCTTGTTGC |
| | | GTGGTTGGGGTGTGGGTGTGAGTGGGATGGGAGAGTG |
| | | GTTGTTGCGTGTGGTTGGCTCGGGTGCATGGATGAGTG |
| | | ATTGTCGGCGTGTTTGTGTGTGACAGGGAATTTTTCTCG |
| | | CAATACGGCGACGTAATGGACGTGTTTATCCCGAAGCC |
| | | CTTCCGAGCCTTCGCGTTCGTTACCTTCGCAGATGATCA |
| | | GGTGCTGTGTGTGTGCGAGTGCATGTGGGGTGGTTGAA |
| | | CAGGGGTGTATGGCTGGGTGTTTGAGGCGTGGATGATT |
| | | GTGTGCATGACAAGAGTGAATGTTGGTGTGAATGACGA |
| | | GTGACTGTCTAACGTCTTGACCGATTCTACAGATCGCGC |
| | | AATCGCTGTGCGGAGAAGACCTTATTATTAAGGGCATCT |
| | | CTGTACACATCAGCAATGCAGAGCCCAAGCATAATTCTA |
| | | ACCTGCCACCTTTACTGGGCCCCTCTGGCGGCGACCGG |
| | | GAGCCAATGGGACTAGGCCCACCAGCAACGCAGCTGA |
| | | CTCCACCACCCGCCCCAGTTGGCTTGCGTGGATCCAAC |
| | | CACCGTGGACTTCCCAAAGATAGTGGCCCCTTGCCTAC |
| | | GCCACCCGGCGTGAGCCTGCTAGGCGAGCCACCAAAG |
| | | GATTACAGGATACCCCTGAACCCTTACCTTAATCTCCAC |
| | | TCACTGCTGCCCTCTAGCAATCTTGCGGGAAAAGAGAC |
| | | CAGGGGCTGGGGCGGAAGCGGGAGAGGGCGAAGACC |
| | | AGCTGAGCCGCCACTGCCTTCGCCAGCAGTTCCTGGAG |
| | | GAGGGTCAGGCAGTAACAATGGCAACAAAGCGTTCCAA |
| | | ATGAAAAGTCGACTCTTGTCTCCCATTGCCTCTAACCGC |
| | | CTGCCTCCCGAACCCGGGCTGCCAGACTCCTATGGATT |
| | | TGATTACCCGACAGATGTGGGTCCTCGCCGCTTGTTCA |
| | | GCCATCCCAGAGAACCTACTCTAGGAGCCCACGGGCCG |
| | | AGTAGGCACAAAATGTCGCCTCCGCCGTCCTCATTCAA |
| | | CGAGCCTAGATCCGGCGGTGGGTCCGGAGGCCCACTT |
| | | TCGCACTTCTGATAAACAAATGGTAAGGAAGGGCACATCA |
| | | ATCTTTGCTTAATTGTCCTTTACTCTAAAGATGTATTTTA |
| | | TCATACTGAATGCTAAACTTGATATCTCCTTTTAGGTCAT |
| | | TGATGTCCTTCACCCCGGGAAGGCGACAGTGCCTAAGA |
| | | CAGAAATTCGGGAAAAACTAGCCAAAATGTACAAGACCA |
| | | CACCGGATGTCATCTTTGTATTTGGATTCAGAACTCAGT |
| | | AAACTGGATCCGCAGGCC |
| Upstream CDS | SEQ ID NO: | ATGGGCCCAAAAAAGAAGAGAAAGGTAGAAGACCCCGG |
| | 184 | CGGACCGGCGGCGAAACGCGTGAAACTGGATGGAGGT |
| | | TACCCATACGATGTTCCAGATTACGCTGGTGGTATGTCA |
| | | GAATATATTCGGGTCACCGAGGACGAGAACGACGAGCC |
| | | TATCGAGATACCATCCGAAGACGACGGAACAGTCCTCC |
| | | TGAGTACCGTGACAGCACAATTCCCAGGGGCCTGCGGC |
| | | CTCCGTTACAGAAACCCTGTTAGCCAGTGTATGAGGGG |
| | | TGTGCGGCTCGTGGAAGGCATACTCCACGCTCCGGACG |
| | | CCGGGTGGGGTAACTTGGTTTATGTCGTAAATTACCCTA |
| | | AGGACAATAAACGAAAGATGGACGAAACCGACGCTAGT |
| | | AGCGCCGTGAAAGTAAAACGGGCAGTGCAGAAGACATC |
| | | TGACCTCATCGTCTTAGGTCTGCCTTGGAAGACCACAGA |
| | | GCAGGATCTGAAAGAATATTTCTCTACTTTTGGCGAAGT |
| | | CCTGATGGTGCAGGTGAAAAAGGATCTGAAGACAGGGC |
| | | ATAGCAAAGGGTTCGGATTTGTCAGGTTCACTGAGTATG |
| | | AGACCCAGGTGAAAGTGATGTCCCAGCGACATATGATC |
| | | GATGGGCGGTGGTGCGATTGTAAGCTGCCTAATAGCAA |
| | | GCAGTCTCAGGACGAACCCTTAAGATCCCGCAAGGTGT |
| | | TCGTGGGTCGCTGCACGGAGGACATGACGGAGGACGA |
| | | ACTAAG |
| First part of intron | SEQ ID NO: | GTAAGCGGAGTGCTTGTTGCGTGGTTGGGGTGTGGGTG |
| | 185 | TGAGTGGGATGGGAGAGTGGTTGTTGCGTGTGGTTGGC |
| | | TCGGGTGCATGGATGAGTGATTGTCGGCGTGTTTGTGT |
| | | GTGACAG |
| Cryptic exon | SEQ ID NO: | GGAATTTTTCTCGCAATACGGCGACGTAATGGACGTGTT |
| | 186 | TATCCCGAAGCCCTTCCGAGCCTTCGCGTTCGTTACCTT |
| | | CGCAGATGATCAG |
| Second part of | SEQ ID NO: | GTGCTGTGTGTGTGCGAGTGCATGTGGGGTGGTTGAAC |
| intron | 187 | AGGGGTGTATGGCTGGGTGTTTGAGGCGTGGATGATTG |
| | | TGTGCATGACAAGAGTGAATGTTGGTGTGAATGACGAG |
| | | TGACTGTCTAACGTCTTGACCGATTCTACAG |
| Downstream CDS | SEQ ID NO: | ATCGCGCAATCGCTGTGCGGAGAAGACCTTATTATTAAG |
| | 188 | GGCATCTCTGTACACATCAGCAATGCAGAGCCCAAGCA |
| | | TAATTCTAACCTGCCACCTTTACTGGGCCCCTCTGGCGG |
| | | CGACCGGGAGCCAATGGGACTAGGCCCACCAGCAACG |
| | | CAGCTGACTCCACCACCCGCCCCAGTTGGCTTGCGTGG |
| | | ATCCAACCACCGTGGACTTCCCAAAGATAGTGGCCCCT |
| | | TGCCTACGCCACCCGGCGTGAGCCTGCTAGGCGAGCC |
| | | ACCAAAGGATTACAGGATACCCCTGAACCCTTACCTTAA |
| | | TCTCCACTCACTGCTGCCCTCTAGCAATCTTGCGGGAAA |
| | | AGAGACCAGGGGCTGGGGCGGAAGCGGGAGAGGGCG |
| | | AAGACCAGCTGAGCCGCCACTGCCTTCGCCAGCAGTTC |
| | | CTGGAGGAGGGTCAGGCAGTAACAATGGCAACAAAGCG |
| | | TTCCAAATGAAAAGTCGACTCTTGTCTCCCATTGCCTCT |
| | | AACCGCCTGCCTCCCGAACCCGGGCTGCCAGACTCCTA |
| | | TGGATTTGATTACCCGACAGATGTGGGTCCTCGCCGCT |
| | | TGTTCAGCCATCCCAGAGAACCTACTCTAGGAGCCCAC |
| | | GGGCCGAGTAGGCACAAAATGTCGCCTCCGCCGTCCTC |
| | | ATTCAACGAGCCTAGATCCGGCGGTGGGTCCGGAGGC |
| | | CCACTTTCGCACTTCTGA |

### Results and Discussion of Design 2 TDP-43/Ravel 1 Fusion Vectors (i.e., Example 7A and 7B).

### RT-PCR Analysis of F2L Mutants

First, we analysed the splicing with the F2L mutants of each example (to avoid underestimating cryptic exon inclusion due to autoregulation). We transfected SK-N-DZ cells with either vector, with or without TDP-43 depletion, then used reverse transcription PCR (RT-PCR) to analyse the splicing of the cryptic exon.

FIG. 17 shows that in cells without TDP-43 depletion, the cryptic exon is almost undetectable in Example 7A and only weakly expressed in Example 7B. However, upon TDP-43 depletion, the cryptic exon is expressed strongly for both examples (with stronger overall expression for Example 7B). It should be noted that the shRNA against TDP-43 (shTDP) only targets endogenous TDP-43.

### RT-PCR Analysis of Wildtype Sequences

Next, we performed the same experiment, but with functional TDP-43 sequences (i.e., without the F2L mutation). The results are demonstrated in FIG. 18. In this case, the TDP-43/Raver1 constructs suppress their own cryptic splicing, resulting in minimal cryptic exon inclusion even upon depletion of endogenous TDP-43. As such, these vectors are said to “autoregulate”.

### Splicing Rescue of Endogenous Transcripts

Next, we measured whether the constructs, in addition to autoregulation (thus “rescuing” their own cryptic splicing”), were also able to rescue the cryptic splicing of endogenous transcripts.

To this end, we performed RT-PCR against endogenous UNC13A and ELAVL3 in cells expressing Example constructs 7A or 7B, or a constitutive expression vector for TDP-43/Raver1 fusion protein (+ve control) or mScarlet (−ve control).

As expected, cryptic exon inclusion is essentially undetectable in normal cells. For cells expressing mScarlet, knockdown of endogenous TDP-43 results in high levels of cryptic exon inclusion. However, cells expressing TDP-43/Raver1 constructs featured much reduced levels of cryptic exon inclusion, demonstrating a rescue of cryptic splicing.

These results are shown in FIG. 19. FIG. 19A shows the RT-PCR of the endogenously expressed UNC13A transcript for cells expressing the indicated constructs. FIG. 19B shows the quantification of the above RT-PCRs against UNC13A, and equivalent RT-PCRs (not shown) performed against the ELAVL3 cryptic exon, with or without knockdown of endogenous TDP-43.

### Example 8: Design 2 Prime-Editing Vectors

A further Design 2 construct expressing Cas9 was designed with some improvements. This particular construct contained a different cryptic exon that demonstrated higher expression upon TDP-43 depletion, enabling more efficient genome editing. Further this sequence was placed within a “prime editing” vector. This enabled us to more clearly demonstrate its activity. (Note that Prime Editing uses a H840A mutant Cas9.)

The intron used in this case is a modified version of the truncated AARS1 cryptic exon-containing intron. This features higher downstream TG density to improve binding of TDP-43. The sequence of this construct is detailed below.

| | | |
|---|---|---|
| Example 8 | | |
| Sequence | | |
| Whole | SEQ ID | TGAACCGTCAGATCCGCTAGAGATCCGCGGCCGCTAATACGAC |
| Transcript | NO: 189 | TCACTATAGGGAGAGCCGCCACCATGAAACGGACAGCCGACGG |
| (start codon in | | AAGCGAGTTCGAGTCACCAAAGAAGAAGCGGAAAGTCGACAAG |
| bold) | | AAGTACAGCATCGGCCTGGACATCGGCACCAACTCTGTGGGCT |
| | | GGGCCGTGATCACCGACGAGTACAAGGTGCCCAGCAAGAAATT |
| | | CAAGGTGCTGGGCAACACCGACCGGCACAGCATCAAGAAGAAC |
| | | CTGATCGGAGCCCTGCTGTTCGACAGCGGCGAAACAGCCGAGG |
| | | CCACCCGGCTGAAGAGAACCGCCAGAAGAAGATACACCAGACG |
| | | GAAGAACCGGATCTGCTATCTGCAAGAGATCTTCAGCAACGAGA |
| | | TGGCCAAGGTGGACGACAGCTTCTTCCACAGACTGGAAGAGTC |
| | | CTTCCTGGTGGAAGAGGATAAGAAGCACGAGCGGCACCCCATC |
| | | TTCGGCAACATCGTGGACGAGGTGGCCTACCACGAGAAGTACC |
| | | CCACCATCTACCACCTGAGAAAGAAACTGGTGGACAGCACCGA |
| | | CAAGGCCGACCTGCGGCTGATCTATCTGGCCCTGGCCCACATG |
| | | ATCAAGTTCCGGGGCCACTTCCTGATCGAGGGCGACCTGAACC |
| | | CCGACAACAGCGACGTGGACAAGCTGTTCATCCAGCTGGTGCA |
| | | GACCTACAACCAGCTGTTCGAGGAAAACCCCATCAACGCCAGC |
| | | GGCGTGGACGCCAAGGCCATCCTGTCTGCCAGACTGAGCAAGA |
| | | GCAGAAAGCTGGAAAATCTGATCGCCCAGCTGCCCGGCGAGAA |
| | | GAAGAATGGCCTGTTCGGAAACCTGATTGCCCTGAGCCTGGGC |
| | | CTGACCCCCAACTTCAAGAGCAACTTCGACCTGGCCGAGGATG |
| | | CCAAACTGCAGCTGAGCAAGGACACCTACGACGACGACCTGGA |
| | | CAACCTGCTGGCCCAGATCGGCGACCAGTACGCCGACCTGTTT |
| | | CTGGCCGCCAAGAACCTGTCCGACGCCATCCTGCTGAGCGACA |
| | | TCCTGAGAGTGAACACCGAGATCACCAAGGCCCCCCTGAGCGC |
| | | CTCTATGATCAAGAGATACGACGAGCACCACCAGGACCTGACCC |
| | | TGCTGAAAGCTCTCGTGCGGCAGCAGCTGCCTGAGAAGTACAA |
| | | AGAGATTTTCTTCGACCAGAGCAAGAACGGCTACGCCGGCTACA |
| | | TTGACGGCGGAGCCAGCCAGGAAGAGTTCTACAAGTTCATCAA |
| | | GCCCATCCTGGAAAAGATGGACGGCACCGAGGAACTGCTCGTG |
| | | AAGCTGAAGAGAGAGGACCTGCTGCGGAAGCAGCGGACCTTCG |
| | | ACAACGGCAGCATCCCCCACCAGATCCACCTGGGAGAGCTGCA |
| | | CGCCATTCTGCGGCGGCAGGAAGATTTTTACCCATTCCTGAAGG |
| | | ACAACCGGGAAAAGATCGAGAAGATCCTGACCTTCCGCATCCCC |
| | | TACTACGTGGGCCCTCTGGCCAGGGGAAACAGCAGATTCGCCT |
| | | GGATGACCAGAAAGAGCGAGGAAACCATCACCCCCTGGAACTT |
| | | CGAGGAAGTGGTGGACAAGGGCGCTTCCGCCCAGAGCTTCATC |
| | | GAGCGGATGACCAACTTCGATAAGAACCTGCCCAACGAGAAGG |
| | | TGCTGCCCAAGCACAGCCTGCTGTACGAGTACTTCACCGTGTAT |
| | | AACGAGCTGACCAAAGTGAAATACGTGACCGAGGGAATGAGAA |
| | | AGCCCGCCTTCCTGAGCGGCGAGCAGAAAAAGGCCATCGTGGA |
| | | CCTGCTGTTCAAGACCAACCGGAAAGTGACCGTGAAGCAGCTG |
| | | AAAGAGGACTACTTCAAGAAAATCGAGTGCTTCGACTCCGTGGA |
| | | AATCTCCGGCGTGGAAGATCGGTTCAACGCCTCCCTGGGCACA |
| | | TACCACGATCTGCTGAAAATTATCAAGGACAAGGACTTCCTGGA |
| | | CAATGAGGAAAACGAGGACATTCTGGAAGATATCGTGCTGACCC |
| | | TGACACTGTTTGAGGACAGAGAGATGATCGAGGAACGGCTGAA |
| | | AACCTATGCCCACCTGTTCGACGACAAAGTGATGAAGCAGCTGA |
| | | AGCGGCGGAGATACACCGGCTGGGGCAGGTAAGAATGCACATC |
| | | ACTTCTTGAGAGTATGGAGGAGTGAAATGACACTCAGTGCCAGAG |
| | | TTACTGTATATCTACACTTTAAAAGTGTAGCTTTTAAAAGATAAG |
| | | CAAGCACAATCTTTTGTGTGTGTGTGTGTGAATGTGTGTGTGTGT |
| | | GTGTGTCACCCAGACTGTCCCGTAAGCTCATCAATGGCATACGC |
| | | GATAAACAATCGGGGAAGACAATCCTGGATTTTCTTAAATCTGAT |
| | | GGATTTGCGAATCGTAACTTTATGCAGCTGATCCATGATGACTCC |
| | | CTGACGTTCAAAGAGGATATTCAGAAGGCGCAGGTAAGTATCGT |
| | | GTGTGTGTTTTTTTTTGTATTTTTTACCGAGTCGGGGTTTCGCAA |
| | | TGTTGCCCAGGCTGGTCTCAGAGTCTCGCTCTGTTGTCTACGCT |
| | | GGAGTGCAGTAACATGAGCCACTGTGCCCGGCCAATCCTAAGA |
| | | ATTTCTTTTGCGGTGGTTGCAAGTCTGGGCAGAACTCTTGTCAG |
| | | GGGCTGTAACTGGACTTATCTTTACTCCTTTGTCAGGTATCCGG |
| | | CCAGGGCGATAGCCTGCACGAGCACATTGCCAATCTGGCCGGC |
| | | AGCCCCGCCATTAAGAAGGGCATCCTGCAGACAGTGAAGGTGG |
| | | TGGACGAGCTCGTGAAAGTGATGGGCCGGCACAAGCCCGAGAA |
| | | CATCGTGATCGAAATGGCCAGAGAGAACCAGACCACCCAGAAG |
| | | GGACAGAAGAACAGCCGCGAGAGAATGAAGCGGATCGAAGAGG |
| | | GCATCAAAGAGCTGGGCAGCCAGATCCTGAAAGAACACCCCGT |
| | | GGAAAACACCCAGCTGCAGAACGAGAAGCTGTACCTGTACTACC |
| | | TGCAGAATGGGCGGGATATGTACGTGGACCAGGAACTGGACAT |
| | | CAACCGGCTGTCCGACTACGATGTGGACGCTATCGTGCCTCAG |
| | | AGCTTTCTGAAGGACGACTCCATCGACAACAAGGTGCTGACCAG |
| | | AAGCGACAAGAACCGGGGCAAGAGCGACAACGTGCCCTCCGAA |
| | | GAGGTCGTGAAGAAGATGAAGAACTACTGGCGGCAGCTGCTGA |
| | | ACGCCAAGCTGATTACCCAGAGAAAGTTCGACAATCTGACCAAG |
| | | GCCGAGAGAGGCGGCCTGAGCGAACTGGATAAGGCCGGCTTC |
| | | ATCAAGAGACAGCTGGTGGAAACCCGGCAGATCACAAAGCACG |
| | | TGGCACAGATCCTGGACTCCCGGATGAACACTAAGTACGACGA |
| | | GAATGACAAGCTGATCCGGGAAGTGAAAGTGATCACCCTGAAGT |
| | | CCAAGCTGGTGTCCGATTTCCGGAAGGATTTCCAGTTTTACAAA |
| | | GTGCGCGAGATCAACAACTACCACCACGCCCACGACGCCTACC |
| | | TGAACGCCGTCGTGGGAACCGCCCTGATCAAAAAGTACCCTAA |
| | | GCTGGAAAGCGAGTTCGTGTACGGCGACTACAAGGTGTACGAC |
| | | GTGCGGAAGATGATCGCCAAGAGCGAGCAGGAAATCGGCAAGG |
| | | CTACCGCCAAGTACTTCTTCTACAGCAACATCATGAACTTTTTC |
| | | AAGACCGAGATTACCCTGGCCAACGGCGAGATCCGGAAGCGGCC |
| | | TCTGATCGAGACAAACGGCGAAACCGGGGAGATCGTGTGGGAT |
| | | AAGGGCCGGGATTTTGCCACCGTGCGGAAAGTGCTGAGCATGC |
| | | CCCAAGTGAATATCGTGAAAAAGACCGAGGTGCAGACAGGCGG |
| | | CTTCAGCAAAGAGTCTATCCTGCCCAAGAGGAACAGCGATAAGC |
| | | TGATCGCCAGAAAGAAGGACTGGGACCCTAAGAAGTACGGCGG |
| | | CTTCGACAGCCCCACCGTGGCCTATTCTGTGCTGGTGGTGGCC |
| | | AAAGTGGAAAAGGGCAAGTCCAAGAAACTGAAGAGTGTGAAAGA |
| | | GCTGCTGGGGATCACCATCATGGAAAGAAGCAGCTTCGAGAAG |
| | | AATCCCATCGACTTTCTGGAAGCCAAGGGCTACAAAGAAGTGAA |
| | | AAAGGACCTGATCATCAAGCTGCCTAAGTACTCCCTGTTCGAGC |
| | | TGGAAAACGGCCGGAAGAGAATGCTGGCCTCTGCCGGCGAACT |
| | | GCAGAAGGGAAACGAACTGGCCCTGCCCTCCAAATATGTGAACT |
| | | TCCTGTACCTGGCCAGCCACTATGAGAAGCTGAAGGGCTCCCC |
| | | CGAGGATAATGAGCAGAAACAGCTGTTTGTGGAACAGCACAAGC |
| | | ACTACCTGGACGAGATCATCGAGCAGATCAGCGAGTTCTCCAAG |
| | | AGAGTGATCCTGGCCGACGCTAATCTGGACAAAGTGCTGTCCG |
| | | CCTACAACAAGCACCGGGATAAGCCCATCAGAGAGCAGGCCGA |
| | | GAATATCATCCACCTGTTTACCCTGACCAATCTGGGAGCCCCTG |
| | | CCGCCTTCAAGTACTTTGACACCACCATCGACCGGAAGAGGTAC |
| | | ACCAGCACCAAAGAGGTGCTGGACGCCACCCTGATCCACCAGA |
| | | GCATCACCGGCCTGTACGAGACACGGATCGACCTGTCTCAGCT |
| | | GGGAGGTGACTCCGGCGGAAGCTCTGGTGGCAGCAAGCGGAC |
| | | CGCCGACGGCTCTGAATTCGAGAGCCCTAAGAAGAAAAGAAAG |
| | | GTGAGCGGAGGCTCTAGCGGCGGAAGCACCCTGAACATTGAAG |
| | | ACGAGTATAGACTGCATGAAACAAGCAAGGAACCCGACGTGTCC |
| | | CTGGGCTCCACCTGGCTGTCCGACTTTCCCCAGGCCTGGGCCG |
| | | AGACAGGAGGAATGGGCCTGGCCGTGCGGCAGGCACCCCTGA |
| | | TCATCCCTCTGAAGGCCACCTCTACACCCGTGAGCATCAAGCAG |
| | | TACCCTATGTCTCAGGAGGCCAGACTGGGCATCAAGCCTCACAT |
| | | CCAGAGGCTGCTGGACCAGGGCATCCTGGTGCCATGCCAGAGC |
| | | CCCTGGAACACACCACTGCTGCCCGTGAAGAAGCCAGGCACCA |
| | | ATGACTATAGACCCGTGCAGGATCTGAGAGAGGTGAACAAGAG |
| | | GGTGGAGGATATCCACCCCACCGTGCCCAACCCTTACAATCTGC |
| | | TGTCCGGCCTGCCCCCTTCTCACCAGTGGTATACAGTGCTGGAC |
| | | CTGAAGGATGCCTTCTTTTGTCTGAGACTGCACCCTACCAGCCA |
| | | GCCACTGTTCGCCTTTGAGTGGAGGGACCCTGAGATGGGCATC |
| | | TCTGGCCAGCTGACCTGGACACGCCTGCCTCAGGGCTTCAAGA |
| | | ATAGCCCAACACTGTTTAACGAGGCCCTGCACCGCGACCTGGC |
| | | AGATTTCCGGATCCAGCACCCAGATCTGATCCTGCTGCAGTACG |
| | | TGGACGATCTGCTGCTGGCCGCCACCAGCGAGCTGGATTGCCA |
| | | GCAGGGAACACGCGCCCTGCTGCAGACCCTGGGAAACCTGGG |
| | | ATATAGGGCATCCGCCAAGAAGGCCCAGATCTGTCAGAAGCAG |
| | | GTGAAGTACCTGGGCTATCTGCTGAAGGAGGGCCAGAGATGGC |
| | | TGACAGAGGCCAGGAAGGAGACAGTGATGGGCCAGCCAACACC |
| | | CAAGACCCCAAGACAGCTGAGGGAGTTCCTGGGCAAAGCAGGA |
| | | TTTTGCAGGCTGTTCATCCCAGGATTCGCAGAGATGGCAGCACC |
| | | TCTGTACCCACTGACCAAGCCGGGCACCCTGTTTAATTGGGGCC |
| | | CTGACCAGCAGAAGGCCTATCAGGAGATCAAGCAGGCCCTGCT |
| | | GACAGCACCAGCCCTGGGCCTGCCAGACCTGACCAAGCCTTTC |
| | | GAGCTGTTTGTGGATGAGAAGCAGGGCTACGCCAAGGGCGTGC |
| | | TGACCCAGAAGCTGGGACCATGGAGACGGCCCGTGGCCTATCT |
| | | GTCCAAGAAGCTGGACCCAGTGGCAGCAGGATGGCCACCATGC |
| | | CTGAGGATGGTGGCAGCAATCGCCGTGCTGACAAAGGATGCCG |
| | | GCAAGCTGACCATGGGACAGCCACTGGTCATCCTGGCACCACA |
| | | CGCAGTGGAGGCCCTGGTGAAGCAGCCTCCAGATCGCTGGCTG |
| | | TCTAACGCCCGGATGACACACTACCAGGCCCTGCTGCTGGACA |
| | | CCGATCGCGTGCAGTTTGGCCCTGTGGTGGCCCTGAATCCAGC |
| | | CACCCTGCTGCCTCTGCCAGAGGAGGGCCTGCAGCACAACTGT |
| | | CTGGACATCCTGGCAGAGGCACACGGAACAAGGCCAGACCTGA |
| | | CCGATCAGCCCCTGCCTGACGCCGATCACACATGGTATACCGAT |
| | | GGAAGCTCCCTGCTGCAGGAGGGCCAGAGGAAGGCAGGAGCA |
| | | GCAGTGACCACAGAGACAGAAGTGATCTGGGCCAAGGCCCTGC |
| | | CAGCAGGCACATCCGCCCAGCGGGCCGAGCTGATCGCCCTGA |
| | | CCCAGGCCCTGAAGATGGCCGAGGGCAAGAAGCTGAACGTGTA |
| | | CACAGACTCCAGATATGCCTTCGCCACCGCACACATCCACGGA |
| | | GAGATCTACAGGCGCCGGGGCTGGCTGACCTCTGAGGGCAAG |
| | | GAGATCAAGAACAAGGATGAGATCCTGGCCCTGCTGAAGGCCC |
| | | TGTTTCTGCCCAAGCGGCTGAGCATCATCCACTGTCCTGGACAC |
| | | CAGAAGGGACACTCCGCCGAGGCAAGGGGCAATCGGATGGCC |
| | | GACCAGGCCGCCAGAAAGGCTGCTATTACTGAAACTCCCGACA |
| | | CTTCCACTCTGCTGATTGAAAACTCCTCCCCTTCTGGCGGCTCA |
| | | AAAAGAACCGCCGACGGCAGCGAATTCGAGTCTCCCAAGAAGA |
| | | AGAGGAAAGTCGGCTCTGGCCCTGCCGCTAAGAGAGTGAAGCT |
| | | GGACTAACCGGTCAGCGGCCGC |
| Upstream | SEQ ID | ATGAAACGGACAGCCGACGGAAGCGAGTTCGAGTCACCAAAGA |
| CDS | NO: 190 | AGAAGCGGAAAGTCGACAAGAAGTACAGCATCGGCCTGGACAT |
| | | CGGCACCAACTCTGTGGGCTGGGCCGTGATCACCGACGAGTAC |
| | | AAGGTGCCCAGCAAGAAATTCAAGGTGCTGGGCAACACCGACC |
| | | GGCACAGCATCAAGAAGAACCTGATCGGAGCCCTGCTGTTCGA |
| | | CAGCGGCGAAACAGCCGAGGCCACCCGGCTGAAGAGAACCGC |
| | | CAGAAGAAGATACACCAGACGGAAGAACCGGATCTGCTATCTGC |
| | | AAGAGATCTTCAGCAACGAGATGGCCAAGGTGGACGACAGCTT |
| | | CTTCCACAGACTGGAAGAGTCCTTCCTGGTGGAAGAGGATAAGA |
| | | AGCACGAGCGGCACCCCATCTTCGGCAACATCGTGGACGAGGT |
| | | GGCCTACCACGAGAAGTACCCCACCATCTACCACCTGAGAAAGA |
| | | AACTGGTGGACAGCACCGACAAGGCCGACCTGCGGCTGATCTA |
| | | TCTGGCCCTGGCCCACATGATCAAGTTCCGGGGCCACTTCCTGA |
| | | TCGAGGGCGACCTGAACCCCGACAACAGCGACGTGGACAAGCT |
| | | GTTCATCCAGCTGGTGCAGACCTACAACCAGCTGTTCGAGGAAA |
| | | ACCCCATCAACGCCAGCGGCGTGGACGCCAAGGCCATCCTGTC |
| | | TGCCAGACTGAGCAAGAGCAGAAAGCTGGAAAATCTGATCGCC |
| | | CAGCTGCCCGGCGAGAAGAAGAATGGCCTGTTCGGAAACCTGA |
| | | TTGCCCTGAGCCTGGGCCTGACCCCCAACTTCAAGAGCAACTTC |
| | | GACCTGGCCGAGGATGCCAAACTGCAGCTGAGCAAGGACACCT |
| | | ACGACGACGACCTGGACAACCTGCTGGCCCAGATCGGCGACCA |
| | | GTACGCCGACCTGTTTCTGGCCGCCAAGAACCTGTCCGACGCC |
| | | ATCCTGCTGAGCGACATCCTGAGAGTGAACACCGAGATCACCAA |
| | | GGCCCCCCTGAGCGCCTCTATGATCAAGAGATACGACGAGCAC |
| | | CACCAGGACCTGACCCTGCTGAAAGCTCTCGTGCGGCAGCAGC |
| | | TGCCTGAGAAGTACAAAGAGATTTTCTTCGACCAGAGCAAGAAC |
| | | GGCTACGCCGGCTACATTGACGGCGGAGCCAGCCAGGAAGAGT |
| | | TCTACAAGTTCATCAAGCCCATCCTGGAAAAGATGGACGGCACC |
| | | GAGGAACTGCTCGTGAAGCTGAAGAGAGAGGACCTGCTGCGGA |
| | | AGCAGCGGACCTTCGACAACGGCAGCATCCCCCACCAGATCCA |
| | | CCTGGGAGAGCTGCACGCCATTCTGCGGCGGCAGGAAGATTTT |
| | | TACCCATTCCTGAAGGACAACCGGGAAAAGATCGAGAAGATCCT |
| | | GACCTTCCGCATCCCCTACTACGTGGGCCCTCTGGCCAGGGGA |
| | | AACAGCAGATTCGCCTGGATGACCAGAAAGAGCGAGGAAACCA |
| | | TCACCCCCTGGAACTTCGAGGAAGTGGTGGACAAGGGCGCTTC |
| | | CGCCCAGAGCTTCATCGAGCGGATGACCAACTTCGATAAGAACC |
| | | TGCCCAACGAGAAGGTGCTGCCCAAGCACAGCCTGCTGTACGA |
| | | GTACTTCACCGTGTATAACGAGCTGACCAAAGTGAAATACGTGA |
| | | CCGAGGGAATGAGAAAGCCCGCCTTCCTGAGCGGCGAGCAGAA |
| | | AAAGGCCATCGTGGACCTGCTGTTCAAGACCAACCGGAAAGTG |
| | | ACCGTGAAGCAGCTGAAAGAGGACTACTTCAAGAAAATCGAGTG |
| | | CTTCGACTCCGTGGAAATCTCCGGCGTGGAAGATCGGTTCAACG |
| | | CCTCCCTGGGCACATACCACGATCTGCTGAAAATTATCAAGGAC |
| | | AAGGACTTCCTGGACAATGAGGAAAACGAGGACATTCTGGAAGA |
| | | TATCGTGCTGACCCTGACACTGTTTGAGGACAGAGAGATGATCG |
| | | AGGAACGGCTGAAAACCTATGCCCACCTGTTCGACGACAAAGTG |
| | | ATGAAGCAGCTGAAGCGGCGGAGATACACCGGCTGGGGCAG |
| First part of | SEQ ID | GTAAGAATGCACATCACTTCTTGAGAGTATGGAGGAGTGAAATG |
| intron | NO: 191 | ACACTCAGTGCCAGAGTTACTGTATATCTACACTTTAAAAGTGTA |
| | | GCTTTTAAAAGATAAGCAAGCACAATCTTTTGTGTGTGTGTGTGT |
| | | GAATGTGTGTGTGTGTGTGTGTCACCCAG |
| Cryptic exon | SEQ ID | ACTGTCCCGTAAGCTCATCAATGGCATACGCGATAAACAATCGG |
| | NO: 192 | GGAAGACAATCCTGGATTTTCTTAAATCTGATGGATTTGCGAATC |
| | | GTAACTTTATGCAGCTGATCCATGATGACTCCCTGACGTTCAAAG |
| | | AGGATATTCAGAAGGCGCAG |
| Second part of | SEQ ID | GTAAGTATCGTGTGTGTGTTTTTTTTTGTATTTTTTACCGAGTC |
| intron | NO: 193 | GGGGTTTCGCAATGTTGCCCAGGCTGGTCTCAGAGTCTCGCTCT |
| | | GTTGTCTACGCTGGAGTGCAGTAACATGAGCCACTGTGCCCGGC |
| | | CAATCCTAAGAATTTCTTTTGCGGTGGTTGCAAGTCTGGGCAGA |
| | | ACTCTTGTCAGGGGCTGTAACTGGACTTATCTTTACTCCTTTGT |
| | | CAG |
| Downstream | SEQ ID | GTATCCGGCCAGGGCGATAGCCTGCACGAGCACATTGCCAATC |
| CDS | NO: 194 | TGGCCGGCAGCCCCGCCATTAAGAAGGGCATCCTGCAGACAGT |
| | | GAAGGTGGTGGACGAGCTCGTGAAAGTGATGGGCCGGCACAAG |
| | | CCCGAGAACATCGTGATCGAAATGGCCAGAGAGAACCAGACCA |
| | | CCCAGAAGGGACAGAAGAACAGCCGCGAGAGAATGAAGCGGAT |
| | | CGAAGAGGGCATCAAAGAGCTGGGCAGCCAGATCCTGAAAGAA |
| | | CACCCCGTGGAAAACACCCAGCTGCAGAACGAGAAGCTGTACC |
| | | TGTACTACCTGCAGAATGGGCGGGATATGTACGTGGACCAGGA |
| | | ACTGGACATCAACCGGCTGTCCGACTACGATGTGGACGCTATCG |
| | | TGCCTCAGAGCTTTCTGAAGGACGACTCCATCGACAACAAGGTG |
| | | CTGACCAGAAGCGACAAGAACCGGGGCAAGAGCGACAACGTGC |
| | | CCTCCGAAGAGGTCGTGAAGAAGATGAAGAACTACTGGCGGCA |
| | | GCTGCTGAACGCCAAGCTGATTACCCAGAGAAAGTTCGACAATC |
| | | TGACCAAGGCCGAGAGAGGCGGCCTGAGCGAACTGGATAAGG |
| | | CCGGCTTCATCAAGAGACAGCTGGTGGAAACCCGGCAGATCAC |
| | | AAAGCACGTGGCACAGATCCTGGACTCCCGGATGAACACTAAGT |
| | | ACGACGAGAATGACAAGCTGATCCGGGAAGTGAAAGTGATCAC |
| | | CCTGAAGTCCAAGCTGGTGTCCGATTTCCGGAAGGATTTCCAGT |
| | | TTTACAAAGTGCGCGAGATCAACAACTACCACCACGCCCACGAC |
| | | GCCTACCTGAACGCCGTCGTGGGAACCGCCCTGATCAAAAAGT |
| | | ACCCTAAGCTGGAAAGCGAGTTCGTGTACGGCGACTACAAGGT |
| | | GTACGACGTGCGGAAGATGATCGCCAAGAGCGAGCAGGAAATC |
| | | GGCAAGGCTACCGCCAAGTACTTCTTCTACAGCAACATCATGAA |
| | | CTTTTTCAAGACCGAGATTACCCTGGCCAACGGCGAGATCCGGA |
| | | AGCGGCCTCTGATCGAGACAAACGGCGAAACCGGGGAGATCGT |
| | | GTGGGATAAGGGCCGGGATTTTGCCACCGTGCGGAAAGTGCTG |
| | | AGCATGCCCCAAGTGAATATCGTGAAAAAGACCGAGGTGCAGA |
| | | CAGGCGGCTTCAGCAAAGAGTCTATCCTGCCCAAGAGGAACAG |
| | | CGATAAGCTGATCGCCAGAAAGAAGGACTGGGACCCTAAGAAG |
| | | TACGGCGGCTTCGACAGCCCCACCGTGGCCTATTCTGTGCTGG |
| | | TGGTGGCCAAAGTGGAAAAGGGCAAGTCCAAGAAACTGAAGAG |
| | | TGTGAAAGAGCTGCTGGGGATCACCATCATGGAAAGAAGCAGC |
| | | TTCGAGAAGAATCCCATCGACTTTCTGGAAGCCAAGGGCTACAA |
| | | AGAAGTGAAAAAGGACCTGATCATCAAGCTGCCTAAGTACTCCC |
| | | TGTTCGAGCTGGAAAACGGCCGGAAGAGAATGCTGGCCTCTGC |
| | | CGGCGAACTGCAGAAGGGAAACGAACTGGCCCTGCCCTCCAAA |
| | | TATGTGAACTTCCTGTACCTGGCCAGCCACTATGAGAAGCTGAA |
| | | GGGCTCCCCCGAGGATAATGAGCAGAAACAGCTGTTTGTGGAA |
| | | CAGCACAAGCACTACCTGGACGAGATCATCGAGCAGATCAGCG |
| | | AGTTCTCCAAGAGAGTGATCCTGGCCGACGCTAATCTGGACAAA |
| | | GTGCTGTCCGCCTACAACAAGCACCGGGATAAGCCCATCAGAG |
| | | AGCAGGCCGAGAATATCATCCACCTGTTTACCCTGACCAATCTG |
| | | GGAGCCCCTGCCGCCTTCAAGTACTTTGACACCACCATCGACCG |
| | | GAAGAGGTACACCAGCACCAAAGAGGTGCTGGACGCCACCCTG |
| | | ATCCACCAGAGCATCACCGGCCTGTACGAGACACGGATCGACC |
| | | TGTCTCAGCTGGGAGGTGACTCCGGCGGAAGCTCTGGTGGCAG |
| | | CAAGCGGACCGCCGACGGCTCTGAATTCGAGAGCCCTAAGAAG |
| | | AAAAGAAAGGTGAGCGGAGGCTCTAGCGGCGGAAGCACCCTGA |
| | | ACATTGAAGACGAGTATAGACTGCATGAAACAAGCAAGGAACCC |
| | | GACGTGTCCCTGGGCTCCACCTGGCTGTCCGACTTTCCCCAGG |
| | | CCTGGGCCGAGACAGGAGGAATGGGCCTGGCCGTGCGGCAGG |
| | | CACCCCTGATCATCCCTCTGAAGGCCACCTCTACACCCGTGAGC |
| | | ATCAAGCAGTACCCTATGTCTCAGGAGGCCAGACTGGGCATCAA |
| | | GCCTCACATCCAGAGGCTGCTGGACCAGGGCATCCTGGTGCCA |
| | | TGCCAGAGCCCCTGGAACACACCACTGCTGCCCGTGAAGAAGC |
| | | CAGGCACCAATGACTATAGACCCGTGCAGGATCTGAGAGAGGT |
| | | GAACAAGAGGGTGGAGGATATCCACCCCACCGTGCCCAACCCT |
| | | TACAATCTGCTGTCCGGCCTGCCCCCTTCTCACCAGTGGTATAC |
| | | AGTGCTGGACCTGAAGGATGCCTTCTTTTGTCTGAGACTGCACC |
| | | CTACCAGCCAGCCACTGTTCGCCTTTGAGTGGAGGGACCCTGA |
| | | GATGGGCATCTCTGGCCAGCTGACCTGGACACGCCTGCCTCAG |
| | | GGCTTCAAGAATAGCCCAACACTGTTTAACGAGGCCCTGCACCG |
| | | CGACCTGGCAGATTTCCGGATCCAGCACCCAGATCTGATCCTGC |
| | | TGCAGTACGTGGACGATCTGCTGCTGGCCGCCACCAGCGAGCT |
| | | GGATTGCCAGCAGGGAACACGCGCCCTGCTGCAGACCCTGGGA |
| | | AACCTGGGATATAGGGCATCCGCCAAGAAGGCCCAGATCTGTC |
| | | AGAAGCAGGTGAAGTACCTGGGCTATCTGCTGAAGGAGGGCCA |
| | | GAGATGGCTGACAGAGGCCAGGAAGGAGACAGTGATGGGCCA |
| | | GCCAACACCCAAGACCCCAAGACAGCTGAGGGAGTTCCTGGGC |
| | | AAAGCAGGATTTTGCAGGCTGTTCATCCCAGGATTCGCAGAGAT |
| | | GGCAGCACCTCTGTACCCACTGACCAAGCCGGGCACCCTGTTT |
| | | AATTGGGGCCCTGACCAGCAGAAGGCCTATCAGGAGATCAAGC |
| | | AGGCCCTGCTGACAGCACCAGCCCTGGGCCTGCCAGACCTGAC |
| | | CAAGCCTTTCGAGCTGTTTGTGGATGAGAAGCAGGGCTACGCCA |
| | | AGGGCGTGCTGACCCAGAAGCTGGGACCATGGAGACGGCCCG |
| | | TGGCCTATCTGTCCAAGAAGCTGGACCCAGTGGCAGCAGGATG |
| | | GCCACCATGCCTGAGGATGGTGGCAGCAATCGCCGTGCTGACA |
| | | AAGGATGCCGGCAAGCTGACCATGGGACAGCCACTGGTCATCC |
| | | TGGCACCACACGCAGTGGAGGCCCTGGTGAAGCAGCCTCCAGA |
| | | TCGCTGGCTGTCTAACGCCCGGATGACACACTACCAGGCCCTG |
| | | CTGCTGGACACCGATCGCGTGCAGTTTGGCCCTGTGGTGGCCC |
| | | TGAATCCAGCCACCCTGCTGCCTCTGCCAGAGGAGGGCCTGCA |
| | | GCACAACTGTCTGGACATCCTGGCAGAGGCACACGGAACAAGG |
| | | CCAGACCTGACCGATCAGCCCCTGCCTGACGCCGATCACACAT |
| | | GGTATACCGATGGAAGCTCCCTGCTGCAGGAGGGCCAGAGGAA |
| | | GGCAGGAGCAGCAGTGACCACAGAGACAGAAGTGATCTGGGCC |
| | | AAGGCCCTGCCAGCAGGCACATCCGCCCAGCGGGCCGAGCTG |
| | | ATCGCCCTGACCCAGGCCCTGAAGATGGCCGAGGGCAAGAAGC |
| | | TGAACGTGTACACAGACTCCAGATATGCCTTCGCCACCGCACAC |
| | | ATCCACGGAGAGATCTACAGGCGCCGGGGCTGGCTGACCTCTG |
| | | AGGGCAAGGAGATCAAGAACAAGGATGAGATCCTGGCCCTGCT |
| | | GAAGGCCCTGTTTCTGCCCAAGCGGCTGAGCATCATCCACTGTC |
| | | CTGGACACCAGAAGGGACACTCCGCCGAGGCAAGGGGCAATC |
| | | GGATGGCCGACCAGGCCGCCAGAAAGGCTGCTATTACTGAAAC |
| | | TCCCGACACTTCCACTCTGCTGATTGAAAACTCCTCCCCTTCTG |
| | | GCGGCTCAAAAAGAACCGCCGACGGCAGCGAATTCGAGTCTCC |
| | | CAAGAAGAAGAGGAAAGTCGGCTCTGGCCCTGCCGCTAAGAGA |
| | | GTGAAGCTGGACTAA |

As an example genome editing event, the UNC13A cryptic exon donor splice site was targeted and nanopore sequencing was used to measure editing efficiency of this locus.

### Results and Discussion

We transfected either constitutive or cryptic PE-Max (“PE”=Prime Editing) vectors into SK-N-DZ cells, with or without TDP-43 knockdown. It was found that the cryptic exon was only expressed at a detectable level upon TDP-43 knockdown. High levels of genome editing were seen at the expected locus for both conditions when using the constitutive PE-Max vector, but only upon TDP-43 knockdown for the cryptic vector. FIG. 20 shows A) diagrams of the vectors used, B) RT-PCR analysis of splicing with and without TDP-43 knockdown and C) Analysis of the genome editing at the expected locus via Nanopore amplicon sequencing.

### Example 9—Luciferase Vector

A further Design 2 construct was designed which encoded Luciferase. FIG. 21 shows A) Luciferase activity from media of SK-N-DZ cells with or without TDP-43 knockdown. Error bars show standard deviation of three technical replicates and B) Nanopore traces from these cells

The sequence of this construct is detailed below.

| | | |
|---|---|---|
| Example 9 | | |
| Whole | SEQ | CTTCTTGGTGCCAGCTTATCATAGCGCTACCGGTCGCCACCATGGGAGT |
| construct | ID NO: | CAAAGTTCTGTTTGCCCTGATCTGCATCGCTGTGGCCGAGGCCAAGCCC |
| (start | 195 | ACCGAGAACAACGAAGACTTCAACATCGTGGCCGTGGCCAGCAACTTC |
| codon in | | GCGACCACGGATCTCGATGCTGACCGCGGGAAGTTGCCCGGCAAGAAG |
| bold) | | CTGCCGCTGGAGGTGCTCAAAGAGTTGGAGGCAAACGCGAGGAAGGCC |
| | | GGCTGCACCAGGTAAGAATGCACATCACTTCTTGAGAGTATGGAGGAGT |
| | | GAAATGACACTCATGCCAGAGTTACTGTATGTCTGCACTTTAAAAGTGTA |
| | | GCTTTTAAAAGATGATGAATGACTGTCTGTTTGTGTGTGTGTGTGTGAAT |
| | | GTGTGTGTGTGTGTGTGTCACCCAGGGGTTGCCTGATCTGCTTAAGCCA |
| | | CATCAAGTGTACGCCAAAAATGAAGAAGTTCATCCCAGGTATGCATGAC |
| | | TGCCTGCATGCTTGTTTGTTTGTTTGTATGTTTGTATGGAGTCGGGGTTT |
| | | CTGAATGTATGCCTGAGGCTGGTTGCAGAGTCTCGCTCTGGATGTCTAC |
| | | GCTGGATGTGCAGTAACATGAGCCACTGTGCCCGGCCAATCCTAAGAAT |
| | | TTCTTTTGCGGTGGTTGCAAGTCTGGGCAGAACTCTTGTCAGGGGCTGT |
| | | AACTGGACTTATCTTTACTCCTTTGTCAGGACGGTGTCATACGTACGAAG |
| | | GCGATAAAGAGTCCGCACAGGGCGGCATAGGCGAGGCGATCGTCGACA |
| | | TTCCTGAGATTCCTGGGTTCAAGGACTTGGAGCCCTTGGAGCAGTTCAT |
| | | CGCACAGGTCGATCTGTGTGTGGACTGCACAACTGGCTGCCTCAAAGG |
| | | GCTTGCCAACGTGCAGTGTTCTGACCTGCTCAAGAAGTGGCTGCCGCAA |
| | | CGCTGTGCGACCTTTGCCAGCAAGATCCAGGGCCAGGTGGACAAGATC |
| | | AAGGGGGCCGGTGGTGACTAAACAAATGGTAAGGAAGGGCACATCAAT |
| | | CTTTGCTTAATTGTCCTTTACTCTAAAGATGTATTTTATCATACTGAATGC |
| | | TAAACTTGATATCTCCTTTTAGGTCATTGATGTCCTTCACCCCGGGAAGG |
| | | CGACAGTGCCTAAGACAGAAATTCGGGAAAAACTAGCCAAAATGTACAA |
| | | GACCACACCGGATGTCATCTTTGTATTTGGATTCAGAACTCAGTAAACTG |
| | | GATCCGCAGGCC |
| Upstream | SEQ | ATGGGAGTCAAAGTTCTGTTTGCCCTGATCTGCATCGCTGTGGCCGAGG |
| CDS | ID NO: | CCAAGCCCACCGAGAACAACGAAGACTTCAACATCGTGGCCGTGGCCA |
| | 196 | GCAACTTCGCGACCACGGATCTCGATGCTGACCGCGGGAAGTTGCCCG |
| | | GCAAGAAGCTGCCGCTGGAGGTGCTCAAAGAGTTGGAGGCAAACGCGA |
| | | GGAAGGCCGGCTGCACCAG |
| Upstream | SEQ | GTAAGAATGCACATCACTTCTTGAGAGTATGGAGGAGTGAAATGACACT |
| intron | ID NO: | CATGCCAGAGTTACTGTATGTCTGCACTTTAAAAGTGTAGCTTTTAAAAG |
| | 197 | ATGATGAATGACTGTCTGTTTGTGTGTGTGTGTGTGAATGTGTGTGTGTG |
| | | TGTGTGTCACCCAG |
| Cryptic | SEQ | GGGTTGCCTGATCTGCTTAAGCCACATCAAGTGTACGCCAAAAATGAAG |
| exon | ID NO: | AAGTTCATCCCAG |
| | 198 | |
| Downstream | SEQ | GTATGCATGACTGCCTGCATGCTTGTTTGTTTGTTTGTATGTTTGTATGG |
| intron | ID NO: | AGTCGGGGTTTCTGAATGTATGCCTGAGGCTGGTTGCAGAGTCTCGCTC |
| | 199 | TGGATGTCTACGCTGGATGTGCAGTAACATGAGCCACTGTGCCCGGCC |
| | | AATCCTAAGAATTTCTTTTGCGGTGGTTGCAAGTCTGGGCAGAACTCTTG |
| | | TCAGGGGCTGTAACTGGACTTATCTTTACTCCTTTGTCAG |
| Downstream | SEQ | GACGGTGTCATACGTACGAAGGCGATAAAGAGTCCGCACAGGGCGGCA |
| CDS | ID NO: | TAGGCGAGGCGATCGTCGACATTCCTGAGATTCCTGGGTTCAAGGACTT |
| | 200 | GGAGCCCTTGGAGCAGTTCATCGCACAGGTCGATCTGTGTGTGGACTG |
| | | CACAACTGGCTGCCTCAAAGGGCTTGCCAACGTGCAGTGTTCTGACCTG |
| | | CTCAAGAAGTGGCTGCCGCAACGCTGTGCGACCTTTGCCAGCAAGATC |
| | | CAGGGCCAGGTGGACAAGATCAAGGGGGCCGGTGGTGACTAA |

### Example 10: Combining Multiple Cryptic Exons in a Single Vector

In this Example, it is demonstrated that multiple cryptic exons can be contained within a single vector. In the following Example 10 construct, the coding sequence for Cre recombinase is split across seven exons, three of which are flanked by TG-rich sequences, giving four constitutive exons and three cryptic exons.

| | | |
|---|---|---|
| Example 10 | | |
| Sequence | | |
| Whole Transcript | SEQ ID NO: | CTTCTTGGTGCCAGCTTATCATAGCGCTACCGGTCGCC |
| (start codon in | 201 | ACCATGCCCAAGAAGAAGAGGAAGGTGTCCAACCTGT |
| bold, constitutive | | TAACAGTCCACCAGAACCTACCTGCCCTTCCCGTCGAC |
| exons underlined, | | GCTACGTCGGACGAGGTTCGGAAGAACCTGATGGACA |
| cryptic exons | | TGTTCCGGGACCGACAGGTAAGAATGCACATCACTTCT |
| underlined and in | | TGAGAGTATGGAGGAGTGAAATGACACTCAGTGCCAGA |
| italics) | | GTTACTGTATATCTACACTTTAAAAGTGTAGCTTTTAAA |
| | | AGATAAGCAAGCACAATCTTTTGTGTGTGTGTGTGTGA |
| | | ATGTGTGTGTGTGTGTGTGTCACCCAGGCATTTAGCGA |
| | | ACACACGTGGAAGATGCTGCTTTCCGTCTGTCGGAGTT |
| | | GGGCTGCGTGGTGCAAGTTGAACAATCGTAAATGGTTT |
| | | CCAGCCGGTATGCATCACCCCCCCAGCTAATTTTTTTTT |
| | | GTATTTTTTACCGAGTCGGGGTTTCGCAATGTTGCCCA |
| | | GGCTGGTCTCAGAGTCTCGCTCTGTTGTCTACGCTGGA |
| | | GTGCAGTAACATGAGCCACTGTGCCCGGCCAATCCTAA |
| | | GAATTTCTTTTGCGGTGGTTGCAAGTCTGGGCAGAACT |
| | | CTTGTCAGGGGCTGTAACTGGACTTATCTTTACTCCTTT |
| | | GTCAGAACCGGAGGACGTCAGAGACTATTTGCTCTACC |
| | | TGCAAGCCCGCGGACTAGCCGTTAAGACTATACAGCA |
| | | GCATTTGGGACAATTAAATATGTTGCACAGGTAAGAAT |
| | | GCACATCACTTCTTGAGAGTATGGAGGAGTGAAATGAC |
| | | ACTCAGTGCCAGAGTTACTGTATATCTACACTTTAAAAG |
| | | TGTAGCTTTTAAAAGATAAGCAAGCACAATCTTTTGTGT |
| | | GTGTGTGTGTGAATGTGTGTGTGTGTGTGTGTCACCCA |
| | | GGCGGTCCGGGCTTCCCCGGCCTTCGGATTCGAACGC |
| | | AGTGAGCCTAGTCATGCGCCGGATTAGAAAGGAAAATG |
| | | TTGACGCTGGAGAACGGGCAAAGCAAGTATGCATCAC |
| | | CCCCCCAGCTAATTTTTTTTTGTATTTTTTACCGAGTCG |
| | | GGGTTTCGCAATGTTGCCCAGGCTGGTCTCAGAGTCTC |
| | | GCTCTGTTGTCTACGCTGGAGTGCAGTAACATGAGCCA |
| | | CTGTGCCCGGCCAATCCTAAGAATTTCTTTTGCGGTGG |
| | | TTGCAAGTCTGGGCAGAACTCTTGTCAGGGGCTGTAAC |
| | | TGGACTTATCTTTACTCCTTTGTCAGGCTTTAGCGTTTG |
| | | AGAGAACAGATTTTGATCAAGTGCGATCCCTTATGGAG |
| | | AACTCTGACCGTTGCCAAGACATAAGAAATCTTGCTTTC |
| | | TTGGGCATCGCGTACAACACCTTACTGAGAATTGCGGA |
| | | GATTGCCCGGATTCGAGTCAAGGATATAAGCCGCACC |
| | | GACGGAGGACGGATGCTCATCCACATTGGGAGAACGA |
| | | AAACCCTAGTGTCAACCGCCGGTGTGGAGAAAGCTCT |
| | | GAGCCTTGGAGTCACAAAATTGGTCGAGCGATGGATCA |
| | | GGTAAGAATGCACATCACTTCTTGAGAGTATGGAGGAG |
| | | TGAAATGACACTCAGTGCCAGAGTTACTGTATATCTACA |
| | | CTTTAAAAGTGTAGCTTTTAAAAGATAAGCAAGCACAAT |
| | | CTTTTGTGTGTGTGTGTGTGAATGTGTGTGTGTGTGTG |
| | | TGTCACCCAGCGTTTCCGGTGTGGCCGACGATCCAAA |
| | | CAACTATCTATTCTGTCGGGTCCGTAAAAATGGGGTTG |
| | | CGGCACCAAGCGCAACCTCCCAGTTGAGCACGAGGGC |
| | | TCTGGAAGGTATGCATCACCCCCCCAGCTAATTTTTTTT |
| | | TGTATTTTTTACCGAGTCGGGGTTTCGCAATGTTGCCC |
| | | AGGCTGGTCTCAGAGTCTCGCTCTGTTGTCTACGCTGG |
| | | AGTGCAGTAACATGAGCCACTGTGCCCGGCCAATCCTA |
| | | AGAATTTCTTTTGCGGTGGTTGCAAGTCTGGGCAGAAC |
| | | TCTTGTCAGGGGCTGTAACTGGACTTATCTTTACTCCTT |
| | | TGTCAGGTATCTTCGAAGCCACTCACCGACTGATCTAC |
| | | GGCGCAAAGGACGACTCCGGACAGCGATACCTTGCTT |
| | | GGAGTGGGCATAGTGCACGGGTGGGGGCGGCTAGGG |
| | | ATATGGCTAGAGCTGGAGTCTCAATCCCTGAAATTATG |
| | | CAAGCTGGGGGTTGGACAAATGTTAATATTGTAATGAA |
| | | CTATATAAGAAACTTGGATAGTGAGACAGGGGCTATGG |
| | | TGCGCCTGTTAGAAGATGGGGACGGCTCTGGATCTCC |
| | | GGCGGCGAAACGCGTGAAACTGGATTAA |

Via nanopore sequencing, it is found that that while occasionally one or two cryptic exons are included in normal cells, inclusion of all three cryptic exons (which is necessary for Cre recombinase expression) only occurs at detectable levels in cells with TDP-43 depletion.

FIG. 22 shows A) a schematic of the triple cryptic exon Cre-recombinase vector. Exons 2, 4 and 6 are “cryptic”, and B) Quantification of Nanopore reads for the number of cryptic exons included in each transcript for SK-N-DZ cells without (NT) or with doxycycline-induced knockdown of TDP-43. Error bars show standard error across three replicates.

We tested this vector in a different cell line with inducible TDP-43 knockdown, in this case i3 iPSCs with a halo-tagged endogenous TDP-43, enabling depletion of TDP-43 by addition of a halo-tag-compatible proteolysis targeting chimera (protac) molecule. We analysed the splicing of this construct in these cells using Nanopore, with two experimental replicates of untreated and protac treated each. As shown in FIG. 23, the three cryptic exons were undetected in untreated cells, but detected at a high level in protac-treated cells.

### Material and Methods for Supplementary Exemplification

### Cloning Methods

Gene fragments were ordered from IDT as eBlock or gBlock gene fragments. All cloning was performed using NEB HiFi assembly master mix and Stbl3 competent E. coli. Sequences were verified via Sanger or Nanopore sequencing.

### Automated Fluorescence Microscopy

Transfections of the relevant plasmids and preparation of SK-N-DZ cells was as described above but in 96 well dishes. Red fluorescence was imaged using an Incucyte S3 machine. Fluorescence intensity was integrated using CellProfiler.

### Nanopore Sequencing

RNA was extracted and reverse transcribed using Superscript IV, with oligo (dT) primer. Amplicons were produced via PCR with Q5 polymerase using primers specific to the 5′ and 3′ ends of the construct; primers had 20 nt 5′ barcodes to enable identification of individual samples. PCR products were purified and Nanopore libraries were prepared using the LSK-109 kit, following the manufacturer instructions, and sequenced on Minion or Flongle machines. Guppy was used for basecalling, using the high-accuracy mode. Reads were demultiplexed using the primer barcodes with a custom script, then reads were aligned to the reference sequences using Minimap2. It was ensured that reads were primarily aligned to the expected sequence given the barcode. Splice junctions were then extracted using pysam and a custom python script, and the level of productive splicing for each sample was quantified using R.

### Example 7—TDP-43/Raver1 Materials and Methods

Transfections of the relevant plasmids and preparation of SK-N-DZ cells was as described above. RNA was extracted from cells and reverse transcribed with Superscript IV. RT-PCR was performed using One-Taq polymerase (NEB) and visualized on a Qiaxcel automated capillary electrophoresis machine (Qiagen); primers targeting a region containing the cryptic exon within each plasmid were used.

For rescue of endogenous transcripts, piggybac expression vectors for the relevant sequences were generated via Gibson assembly, then stable SK-N-DZ polyclonal lines were generated by co-transfection of these plasmids with a piggybac transposase expression vector followed by 4 weeks of blasticidin selection (10 μg/ml), then followed by doxycycline treatment for five days. RT-PCRs were performed as described above, but using PCR primers against human UNC13A and ELAVL3 sequences.

### Example 8—Prime Editing Materials and Methods

Prime editing vectors were cloned as above. SK-N-DZ cells were co-transfected with the relevant prime editing vector plus a prime editing guide RNA expressing plasmid (spacer sequence: SEQ ID NO: 202 “TAAAAGCATGGATGGAGAGA”, extension sequence: SEQ ID NO: 203 “ATGgACTCACgCATCTCTCCATCCATGC”) and a plasmid expressing mScarlet and the blasticidin resistance gene. Transfected cells were selected with 10 μg/ml blasticidin for 5 days, with or without 1 μg/ml doxycycline to induce TDP-43 depletion.

RT-PCRs were performed as described above but with primers targeting the prime editing vector. Genome editing was assessed using genomic DNA PCR using primers targeting the UNC13A cryptic exon locus, followed by Nanopore sequencing (as described above).

### Example 9—Luciferase Experiment Materials and Methods

Transfections of the relevant plasmids and preparation of SK-N-DZ cells was as described above. Luciferase activity from the media of SK-N-DZ cells was assessed using the Pierce Gaussia Luciferase Glow Assay kit, with three technical replicates of each. Nanopore sequencing was performed as described above.

### Example 10—Cre Recombinase Materials and Methods

SK-N-DZ cells were used with or without TDP-43 knockdown and transfected with the relevant plasmid. Nanopore sequencing was performed as described above, with three technical replicates.

i3 iPSCs with halo-tagged TDP-43 were electroporated using a P3 Primary Cell 4D-Nucleofector. Halo-tag-compatible protac molecule (HaloPROTAC3, Promega) was added 24 hours after electroporation at a final concentration of 300 nM. After three days of treatment, RNA was extracted and targeted Nanopore sequencing was performed as described above.

What is claimed is:

1. A construct comprising
a start codon,
a regulatory domain comprising
a first splice acceptor site and a first splice donor site,
a binding domain for a splicing factor of the hnRNP family, located within 150 nucleotides of the first splice donor site and/or first splice acceptor site; and/or
located between the first splice donor site and first splice acceptor site, and
a transgene sequence,
wherein the construct is configured such that
(i) when placed in a cell with nuclear depletion of the splicing factor, splicing of the first splice acceptor site and first donor site is not repressed, such that a functional protein is produced from the transgene sequence, and
(ii) when placed in a cell without nuclear depletion of the splicing factor, splicing of the first splice acceptor site and/or first donor site is repressed such that no functional protein is produced from the transgene sequence.

2. The construct of claim 1, wherein the binding domain for a splicing factor is a TDP-43 binding domain, and wherein the splicing factor of the hnRNP family is TDP-43.

3. The construct according to claim 2, wherein the TDP-43 binding domain comprises a region of at least 6 nucleotides with a statistically significant enrichment of TG dinucleotides and/or TGNNTG hexanucleotides, wherein N is A, T, C or G, and wherein statistically significant enrichment is defined as a probability of less than 0.2% that a random sequence of nucleotides of equal length would feature an equal number of TG dinucleotides and/or TGNNTG hexanucleotides.

4. The construct according to claim 2, wherein the TDP-43 binding domain comprises the sequence TGTGTG, or wherein the TDP-43 binding domain comprises the sequence TGTGTGTG, or wherein the TDP-43 binding domain comprises the sequence TGTGTGTG.

5. The construct according to claim 1, wherein the binding domain for the splicing factor of the hnRNP family is located within 150 nucleotides of the first splice donor site and/or first splice acceptor site, or wherein the binding domain for the splicing factor of the hnRNP family is located within 100 nucleotides of the first splice donor site and/or first splice acceptor site, or wherein the binding domain for the splicing factor of the hnRNP family is located within 50 nucleotides of the first splice donor site and/or first splice acceptor site.

6. The construct according to claim 1, wherein
the binding domain for the splicing factor of the hnRNP family is
(i) upstream of the first splice acceptor site and first splice donor site,
(ii) between the first splice acceptor site and first splice donor site, or
(iii) downstream of the first splice acceptor site and first splice donor site.

7. The construct according to claim 1, wherein the transgene is for a diagnostic protein, or wherein the transgene is for a diagnostic protein, which is a fluorescent protein, a luminescent protein, or a protein with a detectable antibody-binding tag.

8. The construct according to claim 1, wherein the transgene is for a therapeutic protein, or wherein the transgene is for a therapeutic protein, which is a nuclease, a chaperone, a proteasomal protein, a recombinase protein, a splicing regulator, or a transcription factor, or wherein the transgene is for a chaperone, which is a heat shock protein or a foldase.

9. The construct according to claim 1, wherein the first acceptor splice site and the first donor splice site have a splice score of 0.01 or above as determined by the Splice AI algorithm, more preferably a splice score of 0.05 or above as determined by the Splice AI algorithm.

10. The construct according to claim 1, further comprising a premature termination codon (PTC) downstream of the regulatory domain, configured such that
(i) when placed in a cell with nuclear depletion of the splicing factor, the PTC is out of frame with the start codon in the mRNA product of the construct
(ii) when placed in a cell without nuclear depletion of the splicing factor, the PTC is in frame with the start codon in the mRNA product of the construct.

11. The construct according to claim 10, further comprising a further intronic sequence downstream of the regulatory domain, and wherein the PTC is at least 40 nucleotides upstream of the further intronic sequence.

12. The construct according to claim 1, wherein the start codon is upstream of the regulatory domain.

13. The construct according to claim 1, wherein the first splice acceptor site and first splice donor site define a cryptic exon sequence, and wherein
the regulatory domain further comprises:
an intronic region, wherein the cryptic exon sequence is located within said intronic region, configured such that
(i) when placed in a cell with nuclear depletion of the splicing repressor protein, the cryptic exon sequence is present in the mRNA product of the construct, and
(ii) when placed in a cell without nuclear depletion of the splicing repressor protein the cryptic exon sequence is absent in the mRNA product of the construct

14. The construct according to claim 13, wherein the cryptic exon is frame-shifting cryptic exon sequence with a length of nucleotides that is not divisible by 3, configured such that
(i) when placed in a cell with nuclear depletion of the splicing factor, the complete transgene sequence is in frame with the start codon, and
(ii) when placed in a cell without nuclear depletion of the splicing factor, at least part of the transgene sequence is out of frame with the start codon.

15. The construct according to claim 13, wherein the intronic region is formed of a first part which is upstream of the first splice acceptor site, and a second part which is downstream of the first splice donor site, and wherein the first part and second part are derived from AARS1, or wherein the intronic region is formed of a first part that has a sequence that is at least 80% identical to SEQ ID NO: 30 and wherein a second part, which is downstream of the first splice donor site, has a sequence that is at least 80% identical to SEQ ID NO: 32.

16. The construct according to claim 13, the transgene sequence is completely downstream of the regulatory domain.

17. The construct according to claim 16, further comprising a cleavage site comprising a self-cleaving site or a protease cleavage site between the regulatory domain and the transgene sequence, or comprising a cleavage site selected from P2A, T2A, F2A, E2A, furin, PCSK1, PCSK6, PCSK7, cathepsin B, granzyme B, factor XA, enterokinase, genenase, sortase, precission protease, thrombin, TEV protease or elastase 1.

18. The construct according to claim 13, wherein at least part of the transgene sequence is encoded by the cryptic exon sequence.

19. The construct according to claim 18, wherein the cryptic exon sequence encodes for an N-terminal part, internal part, C-terminal part of the transgene sequence, or any combination thereof.

20. The construct according to claim 1, wherein the regulatory domain comprises a single regulatory intron between the first splice donor site and the first splice acceptor site, configured such that
(i) when placed in a cell that is depleted of splicing factor, the single regulatory intron is spliced, and
(ii) when placed in a cell that is not depleted of splicing factor, the single regulatory intron is (i) not spliced or (ii) incorrectly spliced.

21. A vector comprising the construct of claim 1.

22. A system comprising a cell and the construct of claim 1, or a vector comprising the construct, wherein the system is configured such that:
(i) upon depletion of the splicing factor of the hnRNP family from the cell nucleus, the system produces a functional protein, and
(ii) wherein upon no depletion of the splicing factor of the hnRNP family, the system does not produce a functional protein.

23. The construct of claim 1, or a vector comprising the construct, for use in therapy.

24. The construct of claim 1, or a vector comprising the construct, for use in the treatment for a disease associated with depletion of a splicing factor of the hnRNP family, wherein the treatment comprises contacting a cell with the construct or vector such that
(i) in a cell with nuclear depletion of the splicing factor of the hnRNP family, the cell produces a functional protein, and
in a cell without nuclear depletion of the splicing factor of the hnRNP family, the cell does not produce a functional protein.

25. Use of the construct of claim 1, or use of a vector comprising the construct, in a method of selectively producing functional protein in a diseased cell that has nuclear depletion of a splicing factor of the hnRNP family.

26. The construct of claim 24, or a vector comprising the construct of claim 24, for use in the treatment for a disease associated with depletion of a splicing factor of the hnRNP family, wherein the disease is a neurodegenerative disease or muscle disease, or wherein the disease is amyotrophic lateral sclerosis (ALS) or frontotemporal dementia (FTD).


SEQUENCE LISTING (203 sequences)

SEQ ID NO: 1 - DNA, 101 bp, synthetic construct
  source 1..101: mol_type = other DNA
tagataaaaggatggatggagagatgggtgagtacatggatggatagatggatgagttggtgggtagattcgtggctagatggatgatggatggatggaca

SEQ ID NO: 2 - DNA, 36 bp, synthetic construct
  source 1..36: mol_type = other DNA
tgtgtgtgtgtgtgtgaatgtgtgtgtgtgtgtgtg

SEQ ID NO: 3 - DNA, 10 bp, synthetic construct
  source 1..10: mol_type = other DNA
tgaatgaatg

SEQ ID NO: 4 - DNA, 14 bp, synthetic construct
  source 1..14: mol_type = other DNA
tgaatgaaatgatg

SEQ ID NO: 5 - DNA, 10 bp, synthetic construct
  source 1..10: mol_type = other DNA
tgtgtgtgtg

SEQ ID NO: 6 - DNA, 12 bp, synthetic construct
  source 1..12: mol_type = other DNA
tgtgtgtgtgtg

SEQ ID NO: 7 - DNA, 14 bp, synthetic construct
  source 1..14: mol_type = other DNA
tgtgtgtgtgtgtg

SEQ ID NO: 8 - DNA, 16 bp, synthetic construct
  source 1..16: mol_type = other DNA
tgtgtgtgtgtgtgtg

SEQ ID NO: 9 - DNA, 18 bp, synthetic construct
  source 1..18: mol_type = other DNA
tgtgtgtgtgtgtgtgtg

SEQ ID NO: 10 - protein, 8 aa, synthetic construct
DYKDDDDK

SEQ ID NO: 11 - protein, 5 aa, synthetic construct
DDDDK

SEQ ID NO: 12 - protein, 6 aa, synthetic construct
HHHHHH

SEQ ID NO: 13 - protein, 9 aa, synthetic construct
YPYDVPDYA

SEQ ID NO: 14 - protein, 10 aa, synthetic construct
EQKLISEEDL

SEQ ID NO: 15 - protein, 14 aa, synthetic construct
GKPIPNPLLGLDST

SEQ ID NO: 16 - protein, 15 aa, synthetic construct
KETAAAKFERQHMDS

SEQ ID NO: 17 - protein, 13 aa, synthetic construct
GAPVPYPDPLEPR

SEQ ID NO: 18 - protein, 10 aa, unidentified
MASMTGQQMG

SEQ ID NO: 19 - protein, 11 aa, synthetic construct
YTDIEMNRLGK

SEQ ID NO: 20 - protein, 9 aa, synthetic construct
EEEEYMPME

SEQ ID NO: 21 - protein, 8 aa, synthetic construct
WSHPQFEK

SEQ ID NO: 22 - protein, 11 aa, synthetic construct
QPELAPEDPED

SEQ ID NO: 23 - protein, 29 aa, synthetic construct
TTNPGVSAWQVNTAYTAGQLVIYNGKTYK

SEQ ID NO: 24 - protein, 26 aa, synthetic construct
KRRWKKNFIAVSAANRFKKISSSGAL

SEQ ID NO: 25 - DNA, 1683 bp, synthetic construct
  source 1..1683: mol_type = other DNA
ggtttagtgaaccgtcagatcagatctttgtcgatcctaccatccactcgacacacccgccagcggccgcttcttggtgccagcttatcatagcgctaccggtcgccaccatggcgagaaccatggtagccatggagaccatggggctcatgacaacagatctggcaaaatttggggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggctggagtgcagtggcatgatcacagctcactgcagcctcaaacttcctgggctcaagtgatcctctcccgagtagctgggactacaggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggctggatgccaccaaaatcctcccaggcaacatacggcagcggcgccaccaacttttccctgctcaagcaagccggcgacgtggaagagaatcccggccccgtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 26 - DNA, 541 bp, synthetic construct
  source 1..541: mol_type = other DNA
atgacaacagatctggcaaaatttggggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggctggagtgcagtggcatgatcacagctcactgcagcctcaaacttcctgggctcaagtgatcctctcccgagtagctgggactacaggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggctggatgccaccaaaatcctcccaggcaacatac

SEQ ID NO: 27 - DNA, 479 bp, synthetic construct
  source 1..479: mol_type = other DNA
gtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggctggagtgcagtggcatgatcacagctcactgcagcctcaaacttcctgggctcaagtgatcctctcccgagtagctgggactacaggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcag

SEQ ID NO: 28 - DNA, 155 bp, synthetic construct
  source 1..155: mol_type = other DNA
ggtttagtgaaccgtcagatcagatctttgtcgatcctaccatccactcgacacacccgccagcggccgcttcttggtgccagcttatcatagcgctaccggtcgccaccatggcgagaaccatggtagccatggagaccatggggctcatgaca

SEQ ID NO: 29 - DNA, 21 bp, synthetic construct
  source 1..21: mol_type = other DNA
acagatctggcaaaatttggg

SEQ ID NO: 30 - DNA, 163 bp, synthetic construct
  source 1..163: mol_type = other DNA
gtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccag

SEQ ID NO: 31 - DNA, 88 bp, synthetic construct
  source 1..88: mol_type = other DNA
gctggagtgcagtggcatgatcacagctcactgcagcctcaaacttcctgggctcaagtgatcctctcccgagtagctgggactacag

SEQ ID NO: 32 - DNA, 228 bp, synthetic construct
  source 1..228: mol_type = other DNA
gtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcag

SEQ ID NO: 33 - DNA, 33 bp, synthetic construct
  source 1..33: mol_type = other DNA
gctggatgccaccaaaatcctcccaggcaacat

SEQ ID NO: 34 - DNA, 66 bp, synthetic construct
  source 1..66: mol_type = other DNA
ggcagcggcgccaccaacttttccctgctcaagcaagccggcgacgtggaagagaatcccggcccc

SEQ ID NO: 35 - DNA, 705 bp, synthetic construct
  source 1..705: mol_type = other DNA
gtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaa

SEQ ID NO: 36 - DNA, 219 bp, synthetic construct
  source 1..219: mol_type = other DNA
acaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 37 - DNA, 267 bp, synthetic construct
  source 1..267: mol_type = other DNA
atggcgagaaccatggtagccatggagaccatggggctcatgacaacagatctggcaaaatttggggctggatgccaccaaaatcctcccaggcaacatacggcagcggcgccaccaacttttccctgctcaagcaagccggcgacgtggaagagaatcccggccccgtcagcaaaggggaagaggacaacatggccatcattaaggagtttatgcgattcaaagtacacatggagggatctgttaatggccatgaatttgagatag

SEQ ID NO: 38 - protein, 88 aa, synthetic construct
MARTMVAMETMGLMTTDLAKFGAGCHQNPPRQHTAAAPPTFPCSSKPATWKRIPAPSAKGKRTTWPSLRSLCDSKYTWRDLLMAMNLR

SEQ ID NO: 39 - DNA, 963 bp, synthetic construct
  source 1..963: mol_type = other DNA
atggcgagaaccatggtagccatggagaccatggggctcatgacaacagatctggcaaaatttggggctggagtgcagtggcatgatcacagctcactgcagcctcaaacttcctgggctcaagtgatcctctcccgagtagctgggactacaggctggatgccaccaaaatcctcccaggcaacatacggcagcggcgccaccaacttttccctgctcaagcaagccggcgacgtggaagagaatcccggccccgtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaataa

SEQ ID NO: 40 - protein, 320 aa, synthetic construct
MARTMVAMETMGLMTTDLAKFGAGVQWHDHSSLQPQTSWAQVILSRVAGTTGWMPPKSSQATYGSGATNFSLLKQAGDVEENPGPVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 41 - DNA, 5817 bp, synthetic construct
  source 1..5817: mol_type = other DNA
atatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcttcttggtgccagcttatcatagcgctaccggtcgccaccatggcgagaaccatggtagccatggagaccatggggctcatgacaacagatctggcaaaatttggggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggctggagtgcagtggcatgatcacagctcactgcagcctcaaacttcctgggctcaagtgatcctctcccgagtagctgggactacaggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggctggatgccaccaaaatcctcccaggcaacatacggcagcggcgccaccaacttttccctgctcaagcaagccggcgacgtggaagagaatcccggccccgtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagccc

SEQ ID NO: 42 - DNA, 555 bp, synthetic construct
  source 1..555: mol_type = other DNA
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgac

SEQ ID NO: 43 - DNA, 132 bp, synthetic construct
  source 1..132: mol_type = other DNA
atggcgagaaccatggtagccatggagaccatggggctcatgacaacagatctggcaaaatttggggctggatgccaccaaaatcctcccaggcaacatacggcagcggcgagggcagaggaagtctgctaa

SEQ ID NO: 44 - DNA, 810 bp, synthetic construct
  source 1..810: mol_type = other DNA
atggcgagaaccatggtagccatggagaccatggggctcatgacaacagatctggcaaaatttggggctggagtgcagtggcatgatcacagctcactgcagcctcaaacttcctgggctcaagtgatcctctcccgagtagctgggactacaggctggatgccaccaaaatcctcccaggcaacatacggcagcggcgagggcagaggaagtctgctaacatgcggtgacgtcgaggagaatcctggcccaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 45 - DNA, 1536 bp, synthetic construct
  source 1..1536: mol_type = other DNA
gtcagcaaaggggaagagccaaaaaagaagagaaaggtagaagaccccggcggaccggcggcgaaacgcgtgaaactggatggaggttacccatacgatgttccagattacgctggtggtatgtcagaatatattcgggtcaccgaggacgagaacgacgagcctatcgagataccatccgaagacgacggaacagtcctcctgagtaccgtgacagcacaattcccaggggcctgcggcctccgttacagaaaccctgttagccagtgtatgaggggtgtgcggctcgtggaaggcatactccacgctccggacgccgggtggggtaacttggtttatgtcgtaaattaccctaaggacaataaacgaaagatggacgaaaccgacgctagtagcgccgtgaaagtaaaacgggcagtgcagaagacatctgacctcatcgtcttaggtctgccttggaagaccacagagcaggatctgaaagaatatttctctacttttggcgaagtcctgatggtgcaggtgaaaaaggatctgaagacagggcatagcaaagggttcggatttgtcaggttcactgagtatgagacccaggtgaaagtgatgtcccagcgacatatgatcgatgggcggtggtgcgattgtaagctgcctaatagcaagcagtctcaggacgaacccttaagatcccgcaaggtgttcgtgggtcgctgcacggaggatatgaccgaggacgaactcagggaatttttttcacaatacggagacgtaatggacgtctttatccccaagccttttcgggcctttgccttcgttactttcgctgatgatcagattgctcaatccttgtgcggcgaggatcttattattaagggcatctctgtacacatcagcaatgcagagcccaagcataattctaacctgccacctttactgggcccctcaggcggcgaccgggagccaatgggactaggcccaccagcaacgcagctgactccaccacccgccccagttggcttgcgtggatccaaccaccgtggacttcccaaagatagtggccccttgcctacgccacccggcgtgagcctgctaggcgagccaccaaaggattacaggatacccctgaacccttaccttaatctccacagcctgctgccctctagcaatcttgcgggaaaagagaccaggggctggggcggaagcgggagagggcgaagaccagctgagccgccactgccttcgccagcagttcctggaggagggtcaggcagtaacaatggcaacaaagcgttccaaatgaaaagtcgactcttgtctcccattgcctctaaccgcctgcctcccgaacccgggctgccagactcctatggatttgattacccgacagatgtgggtcctcgccgcttgttcagccatcccagagaacctactctaggagcccacgggccgagtaggcacaaaatgtcgcctccgccgtcctcattcaacgagcctagatccggcggtgggtccggaggcccactttcgcacttctga

SEQ ID NO: 46 - DNA, 1536 bp, synthetic construct
  source 1..1536: mol_type = other DNA
gtcagcaaaggggaagagccaaaaaagaagagaaaggtagaagaccccggcggaccggcggcgaaacgcgtgaaactggatggaggttacccatacgatgttccagattacgctggtggtatgtcagaatatattcgggtcaccgaggacgagaacgacgagcctatcgagataccatccgaagacgacggaacagtcctcctgagtaccgtgacagcacaattcccaggggcctgcggcctccgttacagaaaccctgttagccagtgtatgaggggtgtgcggctcgtggaaggcatactccacgctccggacgccgggtggggtaacttggtttatgtcgtaaattaccctaaggacaataaacgaaagatggacgaaaccgacgctagtagcgccgtgaaagtaaaacgggcagtgcagaagacatctgacctcatcgtcttaggtctgccttggaagaccacagagcaggatctgaaagaatatttctctacttttggcgaagtcctgatggtgcaggtgaaaaaggatctgaagacagggcatagcaaagggctcggacttgtcaggttcactgagtatgagacccaggtgaaagtgatgtcccagcgacatatgatcgatgggcggtggtgcgattgtaagctgcctaatagcaagcagtctcaggacgaacccttaagatcccgcaaggtgttcgtgggtcgctgcacggaggatatgaccgaggacgaactcagggaatttttttcacaatacggagacgtaatggacgtctttatccccaagccttttcgggcctttgccttcgttactttcgctgatgatcagattgctcaatccttgtgcggcgaggatcttattattaagggcatctctgtacacatcagcaatgcagagcccaagcataattctaacctgccacctttactgggcccctcaggcggcgaccgggagccaatgggactaggcccaccagcaacgcagctgactccaccacccgccccagttggcttgcgtggatccaaccaccgtggacttcccaaagatagtggccccttgcctacgccacccggcgtgagcctgctaggcgagccaccaaaggattacaggatacccctgaacccttaccttaatctccacagcctgctgccctctagcaatcttgcgggaaaagagaccaggggctggggcggaagcgggagagggcgaagaccagctgagccgccactgccttcgccagcagttcctggaggagggtcaggcagtaacaatggcaacaaagcgttccaaatgaaaagtcgactcttgtctcccattgcctctaaccgcctgcctcccgaacccgggctgccagactcctatggatttgattacccgacagatgtgggtcctcgccgcttgttcagccatcccagagaacctactctaggagcccacgggccgagtaggcacaaaatgtcgcctccgccgtcctcattcaacgagcctagatccggcggtgggtccggaggcccactttcgcacttctga

SEQ ID NO: 47 - DNA, 1803 bp, synthetic construct
  source 1..1803: mol_type = other DNA
ggtttagtgaaccgtcagatcagatctttgtcgatcctaccatccactcgacacacccgccagcggccgcttcttggtgccagcttatcatagcgctaccggtcgccaccatggcgagaaccatggtagccatggagaccatggggctcatgacaacagatctggcaaaatttgggagatacaccggctggggcaggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccagattatcacgcaaattgatcaatggaataagagataaacagtccggaaaaacaatccttgattttttaaaaagtgatgggttcgcaaatagaaattttatgcaactcatacatgatgacagcttgacattcaaagaggacattcagaaggcgcaggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggtatccggccagggcgatagcctgcaatcctcccaggcaacatacggcagcggcgccaccaacttttccctgctcaagcaagccggcgacgtggaagagaatcccggccccgtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 48 - DNA, 20 bp, synthetic construct
  source 1..20: mol_type = other DNA
agatacaccggctggggcag

SEQ ID NO: 49 - DNA, 154 bp, synthetic construct
  source 1..154: mol_type = other DNA
attatcacgcaaattgatcaatggaataagagataaacagtccggaaaaacaatccttgattttttaaaaagtgatgggttcgcaaatagaaattttatgcaactcatacatgatgacagcttgacattcaaagaggacattcagaaggcgcag

SEQ ID NO: 50 - DNA, 25 bp, synthetic construct
  source 1..25: mol_type = other DNA
gtatccggccagggcgatagcctgc

SEQ ID NO: 51 - DNA, 154 bp, synthetic construct
  source 1..154: mol_type = other DNA
gctatcgcgtaaacttattaatggcatccgggataagcagtccgggaagactattctcgatttcctgaagtctgatggctttgcgaaccggaacttcatgcagctgatccatgacgactctctaacgttcaaggaggacattcagaaggcgcag

SEQ ID NO: 52 - DNA, 154 bp, synthetic construct
  source 1..154: mol_type = other DNA
actctctcgaaagctgatcaatggaatacgggataaacaatcggggaaaacaattctagattttctcaagtcggatggctttgcgaatcgcaatttcatgcaacttattcatgatgattcgcttacatttaaggaggatatacagaaggctcag

SEQ ID NO: 53 - DNA, 154 bp, synthetic construct
  source 1..154: mol_type = other DNA
actttctcgaaagctgattaacggtatacgcgataagcagtctggaaaaacgattctggatttcctgaagtccgatgggtttgcgaaccgcaattttatgcaacttatacacgatgattcactgacatttaaggaggatatacagaaagcgcag

SEQ ID NO: 54 - DNA, 154 bp, synthetic construct
  source 1..154: mol_type = other DNA
actgtctcgaaagctcattaatggtatccgcgacaagcaatctgggaaaactatccttgatttcctcaagtccgatggctttgcaaatcggaactttatgcaactcattcatgacgactcgctaacttttaaagaagatattcaaaaggcgcag

SEQ ID NO: 55 - DNA, 154 bp, synthetic construct
  source 1..154: mol_type = other DNA
actatctcgcaagctcattaacggtatacgagacaaacagtcggggaaaacgatactcgatttcctcaagtctgacggcttcgctaatcgtaatttcatgcaactgattcacgacgactctctcacattcaaagaagacatacaaaaggcacaa

SEQ ID NO: 56 - DNA, 154 bp, synthetic construct
  source 1..154: mol_type = other DNA
gctgtctcgtaagctaatcaacggaatccgtgacaagcaatctgggaaaacaatacttgacttcctaaagtcagatggtttcgctaaccgtaatttcatgcagctaattcatgacgactcacttacgtttaaggaagatatccagaaggcgcag

SEQ ID NO: 57 - DNA, 154 bp, synthetic construct
  source 1..154: mol_type = other DNA
gctttcgcgaaaactaatcaatcggattcgcgataagcaatcgggaaaaacaatacttgattttctaaagtctgatgggtttgcaaatcggaattttatgcaactgattcatgatgattcgctgactttcaaagaggatattcagaaggcacag

SEQ ID NO: 58 - DNA, 154 bp, synthetic construct
  source 1..154: mol_type = other DNA
actatctcgtaaactgattaatgggatacgagataagcaatcgggaaaaacgatcctggacttcctgaaatcagacgggtttgctaatcgaaatttcatgcaacttatccacgacgattcgcttacgtttaaggaggatattcaaaaagcgcaa

SEQ ID NO: 59 - DNA, 154 bp, synthetic construct
  source 1..154: mol_type = other DNA
gctttcccgtaaacttataaatggtattcgtgataaacagtctggcaagactattcttgatttcctaaagtcagatggtttcgctaaccggaactttatgcaacttattcatgatgactctctaacctttaaggaggacatacagaaagcgcag

SEQ ID NO: 60 - DNA, 154 bp, synthetic construct
  source 1..154: mol_type = other DNA
actctcacgtaaactgatcaacgggatacgggataaacagtcgggcaaaactatactagatttcctgaagtcagatgggtttgcgaaccgtaattttatgcagcttattcatgacgattccctaacttttaaggaagacatacagaaagcacag

SEQ ID NO: 61 - DNA, 154 bp, synthetic construct
  source 1..154: mol_type = other DNA
gctgtctcgaaaactgataaatggtatccgcgacaagcaatcagggaagacgattcttgattttcttaaatctgatggctttgctaatcgtaactttatgcagcttatccacgacgattccctgaccttcaaagaagatatacagaaggcccaa

SEQ ID NO: 62 - DNA, 154 bp, synthetic construct
  source 1..154: mol_type = other DNA
actttcacgaaaactgataaacggtattcgagataagcaatccggtaagaccatactggatttccttaaatctgatggttttgcgaatcgcaattttatgcagctaatccatgacgattctctgacctttaaagaagatatccagaaggcccag

SEQ ID NO: 63 - DNA, 154 bp, synthetic construct
  source 1..154: mol_type = other DNA
actttctcggaagcttatcaatgggatccgagataagcaatcaggcaaaacgatccttgattttcttaagtccgatggatttgctaaccggaattttatgcaacttatccacgatgactctctcacttttaaagaggatatccaaaaggcacaa

SEQ ID NO: 64 - DNA, 154 bp, synthetic construct
  source 1..154: mol_type = other DNA
gctctcccgcaagcttataaatggaattcgggacaaacagtctgggaagacaatcctggactttctaaagtctgatggctttgctaatcgtaactttatgcaactaatacatgacgattctcttacgtttaaagaagacatacaaaaggcacag

SEQ ID NO: 65 - DNA, 12 bp, synthetic construct
  source 1..12: mol_type = other DNA
gtgtgaatgaat

SEQ ID NO: 66 - DNA, 1288 bp, synthetic construct
  source 1..1288: mol_type = other DNA
gtgagggtcattgctcggcccctcccatgccacttccactcaccattcctgcctgcccagctcttcctctttctggccacaccatccacactctcctggccctctgagactgcccgccatgccattccctttacctggaaaactcctccctatccatcaaagtccagattcagggtcacctcctctgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatgaattaatgaataaacaggcagatggatgatgtaagctgccccagaccctgggacctctgacccccggcgaccccttgcactctccatgacactttctctcccatggtggcag

SEQ ID NO: 67 - DNA, 797 bp, synthetic construct
  source 1..797: mol_type = other DNA
tgctggtgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatg

SEQ ID NO: 68 - DNA, 1239 bp, synthetic construct
  source 1..1239: mol_type = other DNA
atggtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacaggtaagagcgttcggccttatttactgctgcctgggctcaagcactcgatagtaccgtaatattggttagacagttacacggtagtgagctggaagattgtaaatgtgtgtgtgtgtgtgtgagtgtgtgtgtgtgtgtgtgtttctagttcatgtacgggagcaaggcctacgttaaacatccggccgacattccaggtaagttcaactcactgcacatgatcgcatagcgtaataggcctcacttcttttgagctagggatagagacgcttaagttatatgttgaggcgctaagtaccgatggatgcttttcactttgatctctcctccccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 69 - DNA, 207 bp, synthetic construct
  source 1..207: mol_type = other DNA
atggtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacag

SEQ ID NO: 70 - DNA, 148 bp, synthetic construct
  source 1..148: mol_type = other DNA
gtaagagcgttcggccttatttactgctgcctgggctcaagcactcgatagtaccgtaatattggttagacagttacacggtagtgagctggaagattgtaaatgtgtgtgtgtgtgtgtgagtgtgtgtgtgtgtgtgtgtttctag

SEQ ID NO: 71 - DNA, 49 bp, synthetic construct
  source 1..49: mol_type = other DNA
ttcatgtacgggagcaaggcctacgttaaacatccggccgacattccag

SEQ ID NO: 72 - DNA, 137 bp, synthetic construct
  source 1..137: mol_type = other DNA
gtaagttcaactcactgcacatgatcgcatagcgtaataggcctcacttcttttgagctagggatagagacgcttaagttatatgttgaggcgctaagtaccgatggatgcttttcactttgatctctcctccccag

SEQ ID NO: 73 - DNA, 479 bp, synthetic construct
  source 1..479: mol_type = other DNA
actatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaa

SEQ ID NO: 74 - DNA, 1339 bp, synthetic construct
  source 1..1339: mol_type = other DNA
atggtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacaggtaagtattgactttctcgccatctcctcctcccatcgtgtgccgttatagatcatagggtctgggcttctgcgtcgaggacatccaatctgtcgagttactaaggctcatgagtctgtgttgggtcagccctgcgcgacccgtaaaatgtccattgtgtgtgtgtgtgtgtttgtgtgtgtgtgtgtgtgctgtcagttcatgtacggatcgaaggcctacgtgaagcatccggcggacataccaggtaagcatgttgcggggattcaaagcagttactgatcagtaccgcccaactttggttactggcgtgaactctcggctcagttatctattgaaacctcgcaccttatagatatcaatgcgttgttagtatcccatatcgaggatgcgtagtgtagggcgaaagctaattgcttctctttatcctgtagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 75 - DNA, 207 bp, synthetic construct; identical to SEQ ID NO: 69
  source 1..207: mol_type = other DNA

SEQ ID NO: 76 - DNA, 198 bp, synthetic construct
  source 1..198: mol_type = other DNA
gtaagtattgactttctcgccatctcctcctcccatcgtgtgccgttatagatcatagggtctgggcttctgcgtcgaggacatccaatctgtcgagttactaaggctcatgagtctgtgttgggtcagccctgcgcgacccgtaaaatgtccattgtgtgtgtgtgtgtgtttgtgtgtgtgtgtgtgtgctgtcag

SEQ ID NO: 77 - DNA, 49 bp, synthetic construct
  source 1..49: mol_type = other DNA
ttcatgtacggatcgaaggcctacgtgaagcatccggcggacataccag

SEQ ID NO: 78 - DNA, 187 bp, synthetic construct
  source 1..187: mol_type = other DNA
gtaagcatgttgcggggattcaaagcagttactgatcagtaccgcccaactttggttactggcgtgaactctcggctcagttatctattgaaacctcgcaccttatagatatcaatgcgttgttagtatcccatatcgaggatgcgtagtgtagggcgaaagctaattgcttctctttatcctgtag

SEQ ID NO: 79 - DNA, 479 bp, synthetic construct; identical to SEQ ID NO: 73
  source 1..479: mol_type = other DNA

SEQ ID NO: 80 - DNA, 1305 bp, synthetic construct
  source 1..1305: mol_type = other DNA
atggtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacaggtaagagcggggtgataagagcctcagggttatttcccagactttgaatttgctaattatctcatacgcaacctagcgaatctcataggggtccgggctacttgtctgagcttcttctcttgtgccctatgctctgttcctcttttgacgcccttagttcatgtacggttcgaaggcttacgtcaaacatcccgccgacattccgggtaagtgtgtgtgtgtgtgtgtttgtgtgtgtgtgtgtgtgagtaactccagggcctggcccctctggatccgtgaagtagcatggggttaaggcacggcggaagcgcattatctatgaatttagggccaatgcgagtcctgttagttcaaagccttctgtttacccttttccgtttccttcttatctacgcagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 81 - DNA, 207 bp, synthetic construct; identical to SEQ ID NO: 69
  source 1..207: mol_type = other DNA

SEQ ID NO: 82 - DNA, 157 bp, synthetic construct
  source 1..157: mol_type = other DNA
gtaagagcggggtgataagagcctcagggttatttcccagactttgaatttgctaattatctcatacgcaacctagcgaatctcataggggtccgggctacttgtctgagcttcttctcttgtgccctatgctctgttcctcttttgacgcccttag

SEQ ID NO: 83 - DNA, 49 bp, synthetic construct
  source 1..49: mol_type = other DNA
ttcatgtacggttcgaaggcttacgtcaaacatcccgccgacattccgg

SEQ ID NO: 84 - DNA, 194 bp, Synthetic construct
  source 1..194: mol_type = other DNA
gtaagtgtgtgtgtgtgtgtgtttgtgtgtgtgtgtgtgtgagtaactccagggcctggcccctctggatccgtgaagtagcatggggttaaggcacggcggaagcgcattatctatgaatttagggccaatgcgagtcctgttagttcaaagccttctgtttacccttttccgtttccttcttatctacgcag

SEQ ID NO: 85 - DNA, 479 bp, synthetic construct; identical to SEQ ID NO: 73
  source 1..479: mol_type = other DNA

SEQ ID NO: 86 - DNA, 2509 bp, synthetic construct
  source 1..2509: mol_type = other DNA
atgcccaagaagaagaggaaggtgtccaacctgttaacagtccaccagaacctcccggccctgcccgtggatgccacgtcggacgaggttcgcaagaacctcatggacatgttccgggaccgtcaggcattctctgaacacacctggaaaatgctgcttagcgtatgtcgatcatgggcggcctggtgcaagttgaataatcgtaaatggttcccggctgaacccgaggacgtcagagactaccttttgtacctgcaagcaaggggattagccgttaagactatacagcagcatttgggacaattaaatatgttgcacaggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggcggtccgggcttccccggccttcggattcgaacgcagtgagcctagtcatgcgccggattagaaaggaaaatgttgacgctggagaacgggcaaagcaagtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggctttagcgtttgagagaacagattttgatcaagtgcgatcccttatggagaactctgaccgttgccaagacataagaaatcttgctttcttgggcatcgcgtacaacaccttactgagaattgcggagattgcccggattcgagtcaaggatataagccgcaccgacggaggacggatgctcatccacattgggagaacgaagaccctagtgtcaaccgccggcgtggagaaagctctgagccttggagtcacaaaactggtcgagcggtggatcagcgtgtcaggcgtcgccgacgaccccaacaactacctgttctgccgagtccggaagaacggggtcgccgcaccatcagcgacgtcgcagctctccacgcgggccctcgaaggcatcttcgaagctactcaccgactgatctacggtgcgaaagacgattctggtcagcgataccttgcttggagtgggcatagtgcacgggtgggggcggctagggatatggctagagctggagtctcaatccctgaaattatgcaagctgggggttggacaaatgttaatattgtaatgaactatataagaaacttggatagtgagacaggggctatggtgcgcctgttagaagatggggacggctctggatctccggcggcgaaacgcgtgaaactggatggcagtggagagggcagaggaagtctgctaacatgcggtgacgtcgaggagaatcctggcccagtaagcaaaggcgaggaagataatatggcctcattacccgcaacacacgaactccatatattcggatccatcaacggagtcgatttcgacatggtagggcagggcaccgggaatcccaacgacggatacgaggagctgaacctgaaatctactaagggcgatttgcaattttctccttggatcctggtgccgcacatcggctacggatttcatcagtacctcccttatccagacgggatgagtccattccaggcggctatggtcgacgggagcggctatcaggtgcacaggacaatgcaattcgaagacggagcatctcttaccgtgaattatcgctatacttacgaaggctcccatattaagggcgaggctcaagttaaggggactggttttccagccgatggccccgtcatgacaaactcgctcacagcagccgattggtgccggtccaagaaaacttaccctaatgataagaccattatttcaaccttcaaatggagctacaccacgggaaacggaaagcgataccgcagtactgccagaaccacatatacatttgccaagcccatggccgctaactatcttaagaatcagccaatgtacgtcttcagaaaaaccgaactgaagcacagcaaaaccgagctgaactttaaggagtggcagaaagctttcacggacgttatgggaatggacgagctatataaaggatctggttacccatacgatgttccagattacgcttgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 87 - DNA, 320 bp, synthetic construct
  source 1..320: mol_type = other DNA
atgcccaagaagaagaggaaggtgtccaacctgttaacagtccaccagaacctcccggccctgcccgtggatgccacgtcggacgaggttcgcaagaacctcatggacatgttccgggaccgtcaggcattctctgaacacacctggaaaatgctgcttagcgtatgtcgatcatgggcggcctggtgcaagttgaataatcgtaaatggttcccggctgaacccgaggacgtcagagactaccttttgtacctgcaagcaaggggattagccgttaagactatacagcagcatttgggacaattaaatatgttgcacag

SEQ ID NO: 88 - DNA, 100 bp, synthetic construct
  source 1..100: mol_type = other DNA
gcggtccgggcttccccggccttcggattcgaacgcagtgagcctagtcatgcgccggattagaaaggaaaatgttgacgctggagaacgggcaaagcaa

SEQ ID NO: 89 - DNA, 1476 bp, synthetic construct
  source 1..1476: mol_type = other DNA
gctttagcgtttgagagaacagattttgatcaagtgcgatcccttatggagaactctgaccgttgccaagacataagaaatcttgctttcttgggcatcgcgtacaacaccttactgagaattgcggagattgcccggattcgagtcaaggatataagccgcaccgacggaggacggatgctcatccacattgggagaacgaagaccctagtgtcaaccgccggcgtggagaaagctctgagccttggagtcacaaaactggtcgagcggtggatcagcgtgtcaggcgtcgccgacgaccccaacaactacctgttctgccgagtccggaagaacggggtcgccgcaccatcagcgacgtcgcagctctccacgcgggccctcgaaggcatcttcgaagctactcaccgactgatctacggtgcgaaagacgattctggtcagcgataccttgcttggagtgggcatagtgcacgggtgggggcggctagggatatggctagagctggagtctcaatccctgaaattatgcaagctgggggttggacaaatgttaatattgtaatgaactatataagaaacttggatagtgagacaggggctatggtgcgcctgttagaagatggggacggctctggatctccggcggcgaaacgcgtgaaactggatggcagtggagagggcagaggaagtctgctaacatgcggtgacgtcgaggagaatcctggcccagtaagcaaaggcgaggaagataatatggcctcattacccgcaacacacgaactccatatattcggatccatcaacggagtcgatttcgacatggtagggcagggcaccgggaatcccaacgacggatacgaggagctgaacctgaaatctactaagggcgatttgcaattttctccttggatcctggtgccgcacatcggctacggatttcatcagtacctcccttatccagacgggatgagtccattccaggcggctatggtcgacgggagcggctatcaggtgcacaggacaatgcaattcgaagacggagcatctcttaccgtgaattatcgctatacttacgaaggctcccatattaagggcgaggctcaagttaaggggactggttttccagccgatggccccgtcatgacaaactcgctcacagcagccgattggtgccggtccaagaaaacttaccctaatgataagaccattatttcaaccttcaaatggagctacaccacgggaaacggaaagcgataccgcagtactgccagaaccacatatacatttgccaagcccatggccgctaactatcttaagaatcagccaatgtacgtcttcagaaaaaccgaactgaagcacagcaaaaccgagctgaactttaaggagtggcagaaagctttcacggacgttatgggaatggacgagctatataaaggatctggttacccatacgatgttccagattacgcttga

SEQ ID NO: 90 - DNA, 4660 bp, synthetic construct
  source 1..4660: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccagattatcacgcaaattgatcaatggaataagagataaacagtccggaaaaacaatccttgattttttaaaaagtgatgggttcgcaaatagaaattttatgcaactcatacatgatgacagcttgacattcaaagaggacattcagaaggcgcaggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggtatccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaag

SEQ ID NO: 91 - DNA, 2099 bp, synthetic construct
  source 1..2099: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcag

SEQ ID NO: 92 - DNA, 152 bp, synthetic construct
  source 1..152: mol_type = other DNA
ttatcacgcaaattgatcaatggaataagagataaacagtccggaaaaacaatccttgattttttaaaaagtgatgggttcgcaaatagaaattttatgcaactcatacatgatgacagcttgacattcaaagaggacattcagaaggcgca

SEQ ID NO: 93 - DNA, 2817 bp, synthetic construct
  source 1..2817: mol_type = other DNA
gtatccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaa

SEQ ID NO: 94 - DNA, 9913 bp, synthetic construct
  source 1..9913: mol_type = other DNA
atatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcttcttggtgccagcttatcaggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccagattatcacgcaaattgatcaatggaataagagataaacagtccggaaaaacaatccttgattttttaaaaagtgatgggttcgcaaatagaaattttatgcaactcatacatgatgacagcttgacattcaaagaggacattcagaaggcgcaggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggtatccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagccc

SEQ ID NO: 95 - DNA, 5613 bp, synthetic construct
  source 1..5613: mol_type = other DNA
atatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcttcttggtgccagcttatcatagcgctaccggtcgccaccatggcgagaaccatggtagccatggagaccatggggctcatgacaacagatctggcaaaatttggggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggctggagtgcagtggcatgatcacagctcactgcagcctcaaacttcctgggctcaagtgatcctctcccgagtagctgggactacaggctggatgccaccaaaatcctcccaggcaacatacggcagcggcgccaccaacttttccctgctcaagcaagccggcgacgtggaagagaatcccggccccgtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagccc

SEQ ID NO: 96 - DNA, 462 bp, synthetic construct
  source 1..462: mol_type = other DNA
ggtttagtgaaccgtcagatcagatctttgtcgatcctaccatccactcgacacacccgccagcggccgcttcttggtgccagcttatcatagcgctaccggtcgccaccatggcgagaaccatggtagccatggagaccatggggctcatgacaacagatctggcaaaatttggggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggctggagtgcagtggcatgatcacagctcactgcagcctcaaacttcctgggctcaagtgatcctctcccgagtagctgggactacaggctggatgccaccaaaatcctcccaggcaacatac

SEQ ID NO: 97 - DNA, 163 bp, synthetic construct; identical to SEQ ID NO: 30
  source 1..163: mol_type = other DNA

SEQ ID NO: 98 - DNA, 732 bp, synthetic construct
  source 1..732: mol_type = other DNA
gtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaa

SEQ ID NO: 99 - DNA, 90 bp, synthetic construct
  source 1..90: mol_type = other DNA
atggcgagaaccatggtagccatggagaccatggggctcatgacaacagatctggcaaaatttggggtaagaatgcacatcacttcttga

SEQ ID NO: 100 - protein, 29 aa, synthetic construct
MARTMVAMETMGLMTTDLAKFGVRMHITS

SEQ ID NO: 101 - DNA, 987 bp, synthetic construct
  source 1..987: mol_type = other DNA
atggcgagaaccatggtagccatggagaccatggggctcatgacaacagatctggcaaaatttggggctggagtgcagtggcatgatcacagctcactgcagcctcaaacttcctgggctcaagtgatcctctcccgagtagctgggactacaggctggatgccaccaaaatcctcccaggcaacatacggcagcggcgccaccaacttttccctgctcaagcaagccggcgacgtggaagagaatcccggccccgtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacagtttatgtatggttctaaggcatatgttaagcaccctgcagacatcccagactatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaa

SEQ ID NO: 102 - protein, 328 aa, synthetic construct
MARTMVAMETMGLMTTDLAKFGAGVQWHDHSSLQPQTSWAQVILSRVAGTTGWMPPKSSQATYGSGATNFSLLKQAGDVEENPGPVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKDYKDDDDK

SEQ ID NO: 103 - DNA, 7008 bp, synthetic construct
  source 1..7008: mol_type = other DNA
atatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcttcttggtgccagcttatcagaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtaagtcactggtccccaacctttttggcatgagggaccggtgtagtggaagatggtttttccatggactggtggtgggtggggatggtttcagcatgattcaagtgcattacatttactatgcactttattcctattatgattacattgtaatatataatgaaagaattgtacaactcaccatcatgtagaatcaatgggaaccctgagcttgtgttcctgcaactagatggtcccagctgggggtgatgggagacagtgacagatcatcaggcattagattttcataaggagtgtgcagtctaggtacctcatgtacacagttcacaatagggttcacacccctgtgagaatctaatgccgccgctaatctgacaggaggcagaactcaggtggtcatgcaagcgatggggagtgactgtaaatacagatgaagcttcacttgctcacctatcactcacctcctgctgtacagccctgttcgtaacaggccatggataagtactggtctgtggcccaggggctggggacccctgctgtaagtggtccacaaaccagataatgtggctgtcctctctcatccatcacagtcacccccagggggtattacttccctctaacaactcactgtgtgataggctttcttactgagggcagattctgcacatttattaatattatcactatgcttactgtgccatatagtaccggatacgggatgaagtcatacaagcactgaatgaatggatgaatgaatgatggatgaatggatgacaccttcttatatgtgtatcaggctgatgctgaagacttcaaagttgagtaaaatacctatgtcagtctgcatctcctgggaagtgactgccaagttgaagttaggagtgcagaaaatgtattgagggtaatattcataaaatatgaaacagaggaagagcttcttttttttttttttttttttttgggacagagtcttgctctgtcacccagggctggagtgcagtggcgtgatcttgcctcactgcaacctccttcccctgggttcaggtaattatctcgcctcagcctccagaatagctgggattacaggcacatgccaccaagcccggctaatttttttttttgtatttttagtagagacagggttttgccatgttggccagggtggtcttgaactcctgacctcaggtgatcctcccgcctcggcctcccaaagtgctgagattacaggtgtgagtcaccacgctcagccatgaagagccttttgacaatagcgtgtgtctgacctctgtgaacagagagcgggaaggagggaggatagggctgggagagtctcagatggtgatgcatccctgagtcttggccaaacccagaaagagatcaaggccacggttgtctgcagggaagttctgcattgcaaagggacggccaggcatctaccaagctcagtcataggtgggggctgtccagggagagtcaggttttggctggaatgctacagcaggtcctgcagtttctgcagctgcaggctgcctgctgactgcacttccctgacagattctaaacagtgagctgccaagggcttctgggataccttcatggggagttagttacttatgtcaaaatgtagtgcaagggctgggcatggtggctcacgcctggaatcccagcactctgggaggccgaggcaggcagatcacttgaggtcaggagttcgagaccagcctggccaatgtggtgaaactccatctctactaaaaaaaaaaaatacaaaaactagctggacgtggtggtgggtgcctgtaatcccagctacttgagaggctgaggcatgagaattgcttaaacccggtaggtggactgcactccagccttggtgacagagcaagactgtctcaaaaaaaatgtagtgcaaggagagagagcgaggttggggtgaggtttaggagagggtttgtcttctaggcagagagaattacttagatgcgtctctccgatgtctaatgatctgcagggtctctaaactcacttggcataggtttatttgcactggagttgcacctccttccaggtcagtcttacaagtccatatgcgagacaacgttgtgtcaggacaaacatcacccttggaaatcccttcctccaataactattggccggttgtccttcttgcgcgggtacagactgcgcttattcagttgactgtctggctgagtcaagtcattggcttacgtgagtgtgagtggccaagttgcaaaactggctcttacctttgaatcttcccccattcatactcagccaggcacatggggaggagacccttaagggaatagcagcgtcacctctgccttctcacggtccctccaggaagtgtgggggtcccaggctttggtctgaaactacactgaaatagctcatttttgccttttgttttaacttttccaggtgaaaatgagttacttaaattttcttacattcggacatcttttgacaagatcttgctgagatgggagccgtactggccccccgacttccgagacctcttggggttcatgctgttctacaaagaggcgtaaactggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagccc

SEQ ID NO: 104 - RNA, 20 nt, synthetic construct
  source 1..20: mol_type = other RNA
cactcttgagggccacaaag

SEQ ID NO: 105 - DNA, 52 bp, synthetic construct
  source 1..52: mol_type = other DNA
tcgtcggcagcgtcagatgtgtataagagacagacgaactgtgctgatggga

SEQ ID NO: 106 - DNA, 54 bp, synthetic construct
  source 1..54: mol_type = other DNA
gtctcgtgggctcggagatgtgtataagagacagggtgcctatgggacagtgta

SEQ ID NO: 107 - DNA, 19 bp, synthetic construct
  source 1..19: mol_type = other DNA
cgatcctaccatccactcg

SEQ ID NO: 108 - DNA, 20 bp, synthetic construct
  source 1..20: mol_type = other DNA
ttaatgatggccatgttgtc

SEQ ID NO: 109 - DNA, 44 bp, synthetic construct
  source 1..44: mol_type = other DNA
cttcttggtgccagcttatcagaactactccttctatgccttgg

SEQ ID NO: 110 - DNA, 41 bp, synthetic construct
  source 1..41: mol_type = other DNA
ggcctgcggatccagtttacgcctctttgtagaacagcatg

SEQ ID NO: 111 - DNA, 1046 bp, synthetic construct
  source 1..1046: mol_type = other DNA
atggcgagaacaatggttgctatggtgtccaaaggtgaggcagtcataaaggagtttatgaggttcaaggtgcacatggaagggtcaatgaacggacatgagttcgaaattgaaggtgagggcgagggccgcccctatgaagggacacaaactgccaagctcaaagtgaccaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggtaagtcgtggactagagttttgactcggcgatcacttcccatttataacttcgtatagcatacattatacgaagttataacaatttctccttcccctcgctttcctctaccttctcaggtttaccctgacttgagttgatttggtcgtgcgcgagaaattcagactgggacgcgaccttcaggtaaggacctgagtctccatccccgcacgcccgaaactctgggtaataacttcgtatagcatacattatacgaagttatgcaaccctttcctttcctctttcgacttttctttttccaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 112 - DNA, 352 bp, synthetic construct
  source 1..352: mol_type = other DNA
tagtgaaccgtcagatcagatctttgtcgatcctaccatccactcgacacacccgccagcggccgcttcttggtgccagcttatcatagcgctaccggtcgccaccatggcgagaaccatggtagccatggagaccatggggctcatggtcagcaaaggggaagaggacaacatggccatcattaaggagtttatgcgattcaaagtacacatggagggatctgttaatggccatgaatttgagatagagggggaaggtgagggtcgcccttacgaaggcacgcagacggctaagctgaaggtcacgaaagggggacccttgcccttcgcatgggacatactctccccacag

SEQ ID NO: 113 - DNA, 753 bp, synthetic construct
  source 1..753: mol_type = other DNA
actatctgaagctctcctttcctgaggggtttaagtgggaacgcgttatgaactttgaggatggaggggtcgtgactgttacccaggattcttccctgcaagatggagagttcatatacaaagtgaaacttcggggaacgaatttcccatcagacgggccagtgatgcagaaaaagacgatggggtgggaggcttcatccgagaggatgtatcccgaggacggagcattgaaaggcgaaataaaacaaaggctgaagttgaaggatgggggccactacgacgcggaggttaaaacaacgtataaagctaaaaagccagtacagctcccaggcgcatataacgtgaatataaagcttgacataacgagtcataacgaggattacacaatcgtagaacagtacgaaagagctgaaggacggcactccaccggtgggatggatgaactctataaagactacaaggacgatgatgacaagtaaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcctctgctagcttgactgactgagatacagcgtacct

SEQ ID NO: 114 - protein, 16 aa, synthetic construct
FMYGSKAYVKHPADIP

SEQ ID NO: 115 - DNA, 36 bp, synthetic construct
  source 1..36: mol_type = other DNA
tgtgtgtgtgtgtgtgtttgtgtgtgtgtgtgtgtg

SEQ ID NO: 116 - DNA, 24 bp, synthetic construct
  source 1..24: mol_type = other DNA
gactacaaggacgatgatgacaag

SEQ ID NO: 117 - DNA, 1235 bp, synthetic construct
  source 1..1235: mol_type = other DNA
cggccgcttcttggtgccagcttatcatagcgctaccggtcgccaccatggcgcggacaatggttgctatggtgtccaaaggcgaagcaggtaagagagatctgttgctctggaggggtgtgaatgctgcggcatgagtgaatgtctcgatgattgactgaatggatgcttgcgtgtgtgtgtgtggtctagttatcaaggaattcatgaggttcaaagtccacatggaaggttcaatgaacggccatgaattcgagattgaaggcgagggtgaaggccgaccttacgaaggaacacaaactgcaaaggtggttgtgtgtgtgtgcatgaatgcatgtttgtgtgattaaagcgtgcctggtttatcgacgtgtgtatgaacgatgggtgcctgccttcgccgttgtttctttctttcccgcctccagctcaaggtgacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 118 - DNA, 43 bp, synthetic construct
  source 1..43: mol_type = other DNA
atggcgcggacaatggttgctatggtgtccaaaggcgaagcag

SEQ ID NO: 119 - DNA, 102 bp, synthetic construct
  source 1..102: mol_type = other DNA
gtaagagagatctgttgctctggaggggtgtgaatgctgcggcatgagtgaatgtctcgatgattgactgaatggatgcttgcgtgtgtgtgtgtggtctag

SEQ ID NO: 120 - DNA, 116 bp, synthetic construct
  source 1..116: mol_type = other DNA
ttatcaaggaattcatgaggttcaaagtccacatggaaggttcaatgaacggccatgaattcgagattgaaggcgagggtgaaggccgaccttacgaaggaacacaaactgcaaag

SEQ ID NO: 121 - DNA, 120 bp, synthetic construct
  source 1..120: mol_type = other DNA
gtggttgtgtgtgtgtgcatgaatgcatgtttgtgtgattaaagcgtgcctggtttatcgacgtgtgtatgaacgatgggtgcctgccttcgccgttgtttctttctttcccgcctccag

SEQ ID NO: 122 - DNA, 585 bp, synthetic construct
  source 1..585: mol_type = other DNA
ctcaaggtgacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 123 - DNA, 1286 bp, synthetic construct
  source 1..1286: mol_type = other DNA
cggccgcttcttggtgccagcttatcatagcgctaccggtcgccaccatggcgagaacaatggttgctatggtgtccaagggtgaggcaggtaagaatcgtagcatacaaaattataggagtggctgtgtgaattggtcactggcaatgtccgtgcgtgagtggtgcgatcagtggtggttgaatgcctggatgactgagtgtgtgtgtgtgcttcagtcatcaaggagtttatgcgcttcaaggtgcacatggaaggatcaatgaatggccacgagttcgaaattgaaggcgagggcgagggccgcccctatgaagggacacagactgccaaggtgtgtgtgtgtgtgtgagtgtgtggttgattgtctgacaggcaggtgattagtgagtgcttgaagacgttatcaagcgtgattgttccttgggagactgaagtgtggttggaaaacgaattatcattgttcttccccgctacagctcaaggtgacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 124 - DNA, 43 bp, synthetic construct
  source 1..43: mol_type = other DNA
atggcgagaacaatggttgctatggtgtccaagggtgaggcag

SEQ ID NO: 125 - DNA, 128 bp, synthetic construct
  source 1..128: mol_type = other DNA
gtaagaatcgtagcatacaaaattataggagtggctgtgtgaattggtcactggcaatgtccgtgcgtgagtggtgcgatcagtggtggttgaatgcctggatgactgagtgtgtgtgtgtgcttcag

SEQ ID NO: 126 - DNA, 116 bp, synthetic construct
  source 1..116: mol_type = other DNA
tcatcaaggagtttatgcgcttcaaggtgcacatggaaggatcaatgaatggccacgagttcgaaattgaaggcgagggcgagggccgcccctatgaagggacacagactgccaag

SEQ ID NO: 127 - DNA, 145 bp, synthetic construct
  source 1..145: mol_type = other DNA
gtgtgtgtgtgtgtgtgagtgtgtggttgattgtctgacaggcaggtgattagtgagtgcttgaagacgttatcaagcgtgattgttccttgggagactgaagtgtggttggaaaacgaattatcattgttcttccccgctacag

SEQ ID NO: 128 - DNA, 585 bp, synthetic construct; identical to SEQ ID NO: 122
  source 1..585: mol_type = other DNA

SEQ ID NO: 129 - DNA, 1249 bp, synthetic construct
  source 1..1249: mol_type = other DNA
cggccgcttcttggtgccagcttatcatagcgctaccggtcgccaccatggcgagaacgatggtggctatggtctccaaaggcgaggcaggtaagtcttaccctattgaatgattacttaaatgggggtgtggctgagccgatgtagcgtgattgctagctacgagtgcgtgttgtattaacaatggctcctccgtgtggctggccactccagtgataaaggaattcatgaggttcaaggtgcacatggaagggtcaatgaatggccatgagttcgagatcgagggtgagggcgagggccgcccatatgaagggacccagaccgcgaaggtgtgtgtgttatgtgtgtgacgtgtggatgtgatgtgtgcttgagtataagtgtgaatggcatccggtgatgaagcgcgcgaaacaagattctccttcttcctcccttccagctcaaagtaacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 130 - DNA, 43 bp, synthetic construct
  source 1..43: mol_type = other DNA
atggcgagaacgatggtggctatggtctccaaaggcgaggcag

SEQ ID NO: 131 - DNA, 123 bp, synthetic construct
  source 1..123: mol_type = other DNA
gtaagtcttaccctattgaatgattacttaaatgggggtgtggctgagccgatgtagcgtgattgctagctacgagtgcgtgttgtattaacaatggctcctccgtgtggctggccactccag

SEQ ID NO: 132 - DNA, 116 bp, synthetic construct
  source 1..116: mol_type = other DNA
tgataaaggaattcatgaggttcaaggtgcacatggaagggtcaatgaatggccatgagttcgagatcgagggtgagggcgagggccgcccatatgaagggacccagaccgcgaag

SEQ ID NO: 133 - DNA, 113 bp, synthetic construct
  source 1..113: mol_type = other DNA
gtgtgtgtgttatgtgtgtgacgtgtggatgtgatgtgtgcttgagtataagtgtgaatggcatccggtgatgaagcgcgcgaaacaagattctccttcttcctcccttccag

SEQ ID NO: 134 - DNA, 585 bp, synthetic construct
  source 1..585: mol_type = other DNA
ctcaaagtaacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 135 - DNA, 1255 bp, synthetic construct
  source 1..1255: mol_type = other DNA
cggccgcttcttggtgccagcttatcatagcgctaccggtcgccaccatggcgaggacaatggttgccatggtgtccaaaggagaggcaggtaagtagcttatggctttggggccggtcccaaattcgtgtgactggcgcggatctgggtgtttgtgaaacaagtgtgcatgtctttttcgcctttcgatttccgggtgcctgtttttcaaagtgatcaaagaatttatgaggttcaaggtgcacatggaaggtagcatgaacggtcatgagttcgagatagaaggcgagggcgagggacgcccgtacgaaggcactcagacggcaaaggtgtgtgtgtcctgtgtgtggagtgtgcttgcgtggcgtgcctgccaccgacctctgagtgcatgcctgcaagctgccttcgtccacgctttccggatacccaactttcttttttacagctcaaggtgacaaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 136 - DNA, 43 bp, synthetic construct
  source 1..43: mol_type = other DNA
atggcgaggacaatggttgccatggtgtccaaaggagaggcag

SEQ ID NO: 137 - DNA, 123 bp, synthetic construct
  source 1..123: mol_type = other DNA
gtaagtagcttatggctttggggccggtcccaaattcgtgtgactggcgcggatctgggtgtttgtgaaacaagtgtgcatgtctttttcgcctttcgatttccgggtgcctgtttttcaaag

SEQ ID NO: 138 - DNA, 116 bp, synthetic construct
  source 1..116: mol_type = other DNA
tgatcaaagaatttatgaggttcaaggtgcacatggaaggtagcatgaacggtcatgagttcgagatagaaggcgagggcgagggacgcccgtacgaaggcactcagacggcaaag

SEQ ID NO: 139 - DNA, 119 bp, synthetic construct
  source 1..119: mol_type = other DNA
gtgtgtgtgtcctgtgtgtggagtgtgcttgcgtggcgtgcctgccaccgacctctgagtgcatgcctgcaagctgccttcgtccacgctttccggatacccaactttcttttttacag

SEQ ID NO: 140 - DNA, 585 bp, synthetic construct
  source 1..585: mol_type = other DNA
ctcaaggtgacaaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 141 - DNA, 1262 bp, synthetic construct
  source 1..1262: mol_type = other DNA
cggccgcttcttggtgccagcttatcatagcgctaccggtcgccaccatggcccgaacaatggtcgccatggtgtccaagggagaagcgggtaagtacaccggcctaactggtctcagtcagaataagagtgtctgaaatcaggtggagtggttgggcaattagcgtgcttgattttctctgcgtgactggcgtacgttgctgtggttgtcttgtgtggtagtgatcaaagaatttatgaggttcaaagtccacatggaaggatctatgaatggccacgagtttgagattgaaggagagggagagggacggccgtacgaagggacacaaacggccaaggtgtgtgtggtgtgtttgaccgtccgggtgaatgtctcctaatagtgcgtgcgtgacccgtagtgtggatgcaggggaccgggaagtgtgtctaactgttccaccccccttttacagctcaaagtgaccaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 142 - DNA, 43 bp, synthetic construct
  source 1..43: mol_type = other DNA
atggcccgaacaatggtcgccatggtgtccaagggagaagcgg

SEQ ID NO: 143 - DNA, 132 bp, synthetic construct
  source 1..132: mol_type = other DNA
gtaagtacaccggcctaactggtctcagtcagaataagagtgtctgaaatcaggtggagtggttgggcaattagcgtgcttgattttctctgcgtgactggcgtacgttgctgtggttgtcttgtgtggtag

SEQ ID NO: 144 - DNA, 116 bp, synthetic construct
  source 1..116: mol_type = other DNA
tgatcaaagaatttatgaggttcaaagtccacatggaaggatctatgaatggccacgagtttgagattgaaggagagggagagggacggccgtacgaagggacacaaacggccaag

SEQ ID NO: 145 - DNA, 117 bp, synthetic construct
  source 1..117: mol_type = other DNA
gtgtgtgtggtgtgtttgaccgtccgggtgaatgtctcctaatagtgcgtgcgtgacccgtagtgtggatgcaggggaccgggaagtgtgtctaactgttccaccccccttttacag

SEQ ID NO: 146 - DNA, 585 bp, synthetic construct
  source 1..585: mol_type = other DNA
ctcaaagtgaccaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 147 - DNA, 1273 bp, synthetic construct
  source 1..1273: mol_type = other DNA
cggccgcttcttggtgccagcttatcatagcgctaccggtcgccaccatggcgagaacaatggtcgcgatggtatctaagggcgaagcaggtaagcggcgtgcttgttgcgtggttggggtgtgggtgtgagtgggatgggagagtggttgtcgcgtgtggttggctcgggtgcttggatgggtgattgtcggcgtgtttgacagtgataaaagagtttatgagattcaaagtccacatggagggatcaatgaacggacacgaatttgaaattgaaggcgagggcgaaggaagaccttatgaggggacacagaccgccaaggtgcgtgcgtggatcgtgtgcatgtggggtggttgattaggggtgtatggctgggtgattgaggcgtgtatggtggtgtggatgacaagagtgattgttggtgtgaatgacgagtgactgtctaacgtcttgaccgattctacagttgaaggttacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 148 - DNA, 43 bp, synthetic construct
  source 1..43: mol_type = other DNA
atggcgagaacaatggtcgcgatggtatctaagggcgaagcag

SEQ ID NO: 149 - DNA, 115 bp, synthetic construct
  source 1..115: mol_type = other DNA
gtaagcggcgtgcttgttgcgtggttggggtgtgggtgtgagtgggatgggagagtggttgtcgcgtgtggttggctcgggtgcttggatgggtgattgtcggcgtgtttgacag

SEQ ID NO: 150 - DNA, 116 bp, synthetic construct
  source 1..116: mol_type = other DNA
tgataaaagagtttatgagattcaaagtccacatggagggatcaatgaacggacacgaatttgaaattgaaggcgagggcgaaggaagaccttatgaggggacacagaccgccaag

SEQ ID NO: 151 - DNA, 145 bp, synthetic construct
  source 1..145: mol_type = other DNA
gtgcgtgcgtggatcgtgtgcatgtggggtggttgattaggggtgtatggctgggtgattgaggcgtgtatggtggtgtggatgacaagagtgattgttggtgtgaatgacgagtgactgtctaacgtcttgaccgattctacag

SEQ ID NO: 152 - DNA, 585 bp, synthetic construct
  source 1..585: mol_type = other DNA
ttgaaggttacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 153 - DNA, 1273 bp, synthetic construct
  source 1..1273: mol_type = other DNA
cggccgcttcttggtgccagcttatcatagcgctaccggtcgccaccatggcgcggacgatggtagcaatggtgtctaagggcgaagcaggtaagtagtgtgtttgatgagtgtatgtggtgtgtctgagagtgtagtgtatgagtgattgacgtgagtgtttgtaaggcgtgtctgtttgagtgactggtcgtgtgattgacagttataaaagaatttatgaggttcaaagtccacatggaaggctctatgaacggtcatgagtttgaaattgaaggtgagggtgaaggccgcccttatgaaggcacacaaactgcaaaggtgggtgcgtgctgggcgtgtctgtcgggtgaatgcactggagtgcgtgtctgcgtgggtgttgagtggatgtaggtgtgactgcctcgtgtgcttgcgagagtgaatggagtgtgcttgatgcatttttttattctcgtgtcagctgaaagtgacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactca

SEQ ID NO: 154 - DNA, 43 bp, synthetic construct
  source 1..43: mol_type = other DNA
atggcgcggacgatggtagcaatggtgtctaagggcgaagcag

SEQ ID NO: 155 - DNA, 115 bp, synthetic construct
  source 1..115: mol_type = other DNA
gtaagtagtgtgtttgatgagtgtatgtggtgtgtctgagagtgtagtgtatgagtgattgacgtgagtgtttgtaaggcgtgtctgtttgagtgactggtcgtgtgattgacag

SEQ ID NO: 156 - DNA, 116 bp, synthetic construct
  source 1..116: mol_type = other DNA
ttataaaagaatttatgaggttcaaagtccacatggaaggctctatgaacggtcatgagtttgaaattgaaggtgagggtgaaggccgcccttatgaaggcacacaaactgcaaag

SEQ ID NO: 157 - DNA, 145 bp, synthetic construct
  source 1..145: mol_type = other DNA
gtgggtgcgtgctgggcgtgtctgtcgggtgaatgcactggagtgcgtgtctgcgtgggtgttgagtggatgtaggtgtgactgcctcgtgtgcttgcgagagtgaatggagtgtgcttgatgcatttttttattctcgtgtcag

SEQ ID NO: 158 - DNA, 585 bp, synthetic construct
  source 1..585: mol_type = other DNA
ctgaaagtgacgaagggcgggcctctgcccttctcttgggatatcctgagcccgcagtttatgtacggcagccgggctttcaccaaacaccctgccgatatcccagactactataaacagtcctttccagaaggatttaagtgggagcgagtcatgaatttcgaggacggaggtgccgtgacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 159 - DNA, 103 bp, synthetic construct
  source 1..103: mol_type = other DNA
tgtgtttgatgagtgtatgtggtgtgtctgagagtgtagtgtatgagtgattgacgtgagtgtttgtaaggcgtgtctgtttgagtgactggtcgtgtgattg

SEQ ID NO: 160 - DNA, 122 bp, synthetic construct
  source 1..122: mol_type = other DNA
tgggtgcgtgctgggcgtgtctgtcgggtgaatgcactggagtgcgtgtctgcgtgggtgttgagtggatgtaggtgtgactgcctcgtgtgcttgcgagagtgaatggagtgtgcttgatg

SEQ ID NO: 161 - DNA, 1175 bp, synthetic construct
  source 1..1175: mol_type = other DNA
cttcttggtgccagcttatcatagcgctaccggtcgccaccatggcgagaacaatggttgctatggtgtccaaaggtgaggcagtcataaaggagtttatgaggttcaaggtgcacatggaagggtcaatgaacggacatgagttcgagatcgaaggtgaaggcgaaggtcggccatacgagggaacgcaaactgcaaagctcaaggtgacgaaaggcggaccactacccttcagctgggatatcctcagtcctcaggtaagtatgcatctgttgttgtaaagcaggctaacagctttgtcttcatcgcccccactcatttttggttaatcttacggatgcctgtagaggggaatgttgattgtgtgtgtgtgtgtgtgtgaatgtgtgtgtgtgtgtgtgacagttcatgtacggatcgcgtgccttcactaagcaccccgctgatatccctgactattacaaacagtccttccctgagggcttcaaatgggagcgagtaatgaattttgaggacggtggagccgtgacggtcactcaggatacttctcttgaagacggtaccttgatctataaagttaagttacgcggcactaatttcccacctgatgggccggtcatgcaaaagaagactatggggtgggaggcgtcgacagagcgcctctatcccgaggacggcgttttgaagggggatattaagatggcactaagactcaaagatgggggaaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcc

SEQ ID NO: 162 - DNA, 148 bp, synthetic construct
  source 1..148: mol_type = other DNA
gtaagtatgcatctgttgttgtaaagcaggctaacagctttgtcttcatcgcccccactcatttttggttaatcttacggatgcctgtagaggggaatgttgattgtgtgtgtgtgtgtgtgtgaatgtgtgtgtgtgtgtgtgacag

SEQ ID NO: 163 - DNA, 211 bp, synthetic construct
  source 1..211: mol_type = other DNA
gtaagtatgcatctgttgttgtaaagcaggctaacagctttgtcttcatcgcccccactcatttttggttaatcttacggatgcctgtagaggggaatgttgattgtgtgtgtgtgtgtgtgtgaatgtgtgtgtgtgtgtgtgacagttcatgtacggatcgcgtgccttcactaagcaccccgctgatatccctgactattacaaacag

SEQ ID NO: 164 - DNA, 744 bp, synthetic construct
  source 1..744: mol_type = other DNA
atggcgagaacaatggttgctatggtgtccaaaggtgaggcagtcataaaggagtttatgaggttcaaggtgcacatggaagggtcaatgaacggacatgagttcgagatcgaaggtgaaggcgaaggtcggccatacgagggaacgcaaactgcaaagctcaaggtgacgaaaggcggaccactacccttcagctgggatatcctcagtcctcagttcatgtacggatcgcgtgccttcactaagcaccccgctgatatccctgactattacaaacagtccttccctgagggcttcaaatgggagcgagtaatgaattttgaggacggtggagccgtgacggtcactcaggatacttctcttgaagacggtaccttgatctataaagttaagttacgcggcactaatttcccacctgatgggccggtcatgcaaaagaagactatggggtgggaggcgtcgacagagcgcctctatcccgaggacggcgttttgaagggggatattaagatggcactaagactcaaagatgggggaaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 165 - DNA, 1175 bp, synthetic construct
  source 1..1175: mol_type = other DNA
cttcttggtgccagcttatcatagcgctaccggtcgccaccatggcgagaacaatggttgctatggtgtccaaaggtgaggcagtcataaaggagtttatgaggttcaaggtgcacatggaagggtcaatgaacggacatgagttcgagatcgaaggtgaaggagagggccgaccctacgaagggacgcagacggccaagctgaaggtgacgaaaggcggaccgttgccattctcgtgggatattctgtcacctcaggtaagtatcggtttttgttgtttgcaagagtgaaaaagtgtagaaaactaaattattagatgtcgggtggtgctgctcgttagtctaactctagccgttaactttgtgtgtgtgtgtgtgtgtgaatgtgtgtgtgtgtgtgtgttagttcatgtacggcagtcgtgcgttcacgaaacaccctgctgacatccctgactactacaaacagtcctttcccgagggcttcaaatgggaacgtgtcatgaattttgaagacggaggggccgtgaccgtcactcaagatacgtcgttggaagacggaacgttgatatataaggtcaaattgcgcggaacaaacttcccacccgatggtccagtaatgcaaaagaagactatgggttgggaggcaagtacagagaggctgtacccggaagatggtgttctaaaaggagatatcaagatggccctgaggcttaaggacggtgggcgatacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcc

SEQ ID NO: 166 - DNA, 148 bp, synthetic construct
  source 1..148: mol_type = other DNA
gtaagtatcggtttttgttgtttgcaagagtgaaaaagtgtagaaaactaaattattagatgtcgggtggtgctgctcgttagtctaactctagccgttaactttgtgtgtgtgtgtgtgtgtgaatgtgtgtgtgtgtgtgtgttag

SEQ ID NO: 167 - DNA, 211 bp, synthetic construct
  source 1..211: mol_type = other DNA
gtaagtatcggtttttgttgtttgcaagagtgaaaaagtgtagaaaactaaattattagatgtcgggtggtgctgctcgttagtctaactctagccgttaactttgtgtgtgtgtgtgtgtgtgaatgtgtgtgtgtgtgtgtgttagttcatgtacggcagtcgtgcgttcacgaaacaccctgctgacatccctgactactacaaacag

SEQ ID NO: 168 - DNA, 744 bp, synthetic construct
  source 1..744: mol_type = other DNA
atggcgagaacaatggttgctatggtgtccaaaggtgaggcagtcataaaggagtttatgaggttcaaggtgcacatggaagggtcaatgaacggacatgagttcgagatcgaaggtgaaggagagggccgaccctacgaagggacgcagacggccaagctgaaggtgacgaaaggcggaccgttgccattctcgtgggatattctgtcacctcagttcatgtacggcagtcgtgcgttcacgaaacaccctgctgacatccctgactactacaaacagtcctttcccgagggcttcaaatgggaacgtgtcatgaattttgaagacggaggggccgtgaccgtcactcaagatacgtcgttggaagacggaacgttgatatataaggtcaaattgcgcggaacaaacttcccacccgatggtccagtaatgcaaaagaagactatgggttgggaggcaagtacagagaggctgtacccggaagatggtgttctaaaaggagatatcaagatggccctgaggcttaaggacggtgggcgatacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 169 - DNA, 1150 bp, synthetic construct
  source 1..1150: mol_type = other DNA
cttcttggtgccagcttatcatagcgctaccggtcgccaccatggcgagaacaatggttgctatggtgtccaaaggtgaggcagtcataaaggagtttatgaggttcaaggtgcacatggaagggtcaatgaacggacatgagttcgagattgaaggtgaaggcgaaggacgcccctatgaaggaacccaaacggccaagctgaaagtgactaaaggtggaccactgccgttctcttgggacatcctgagccctcaggtaagtgtgtgatgggtgaatggggagtgcttgagttgtggcgtgcttgaactgggtgattgtgtggaagtgtgggtgacgcgtgtatgggtgcatgtgagagtgtctgtctttgtgtgctagtttatgtacggttcccgcgcgttcaccaaacacccagctgacatcccggactactacaagcaatccttccctgagggctttaagtgggaacgcgtgatgaactttgaggacggtggtgctgttacagtcacgcaagatacttccttagaagacggtacccttatatacaaggttaaactacggggcaccaatttccctcctgacgggccggttatgcagaaaaaaacaatggggtgggaagccagtaccgagcggctctacccagaagatggcgtactcaaaggtgatatcaaaatggctctccgccttaaagacggcggtcggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcc

SEQ ID NO: 170 - DNA, 123 bp, synthetic construct
  source 1..123: mol_type = other DNA
gtaagtgtgtgatgggtgaatggggagtgcttgagttgtggcgtgcttgaactgggtgattgtgtggaagtgtgggtgacgcgtgtatgggtgcatgtgagagtgtctgtctttgtgtgctag

SEQ ID NO: 171 - DNA, 436 bp, synthetic construct
  source 1..436: mol_type = other DNA
gtaagtgtgtgatgggtgaatggggagtgcttgagttgtggcgtgcttgaactgggtgattgtgtggaagtgtgggtgacgcgtgtatgggtgcatgtgagagtgtctgtctttgtgtgctagtttatgtacggttcccgcgcgttcaccaaacacccagctgacatcccggactactacaagcaatccttccctgagggctttaagtgggaacgcgtgatgaactttgaggacggtggtgctgttacagtcacgcaagatacttccttagaagacggtacccttatatacaaggttaaactacggggcaccaatttccctcctgacgggccggttatgcagaaaaaaacaatggggtgggaagccagtaccgagcggctctacccagaagatggcgtactcaaaggtgatatcaaaatggctctccgccttaaag

SEQ ID NO: 172 - DNA, 744 bp, synthetic construct
  source 1..744: mol_type = other DNA
atggcgagaacaatggttgctatggtgtccaaaggtgaggcagtcataaaggagtttatgaggttcaaggtgcacatggaagggtcaatgaacggacatgagttcgagattgaaggtgaaggcgaaggacgcccctatgaaggaacccaaacggccaagctgaaagtgactaaaggtggaccactgccgttctcttgggacatcctgagccctcagtttatgtacggttcccgcgcgttcaccaaacacccagctgacatcccggactactacaagcaatccttccctgagggctttaagtgggaacgcgtgatgaactttgaggacggtggtgctgttacagtcacgcaagatacttccttagaagacggtacccttatatacaaggttaaactacggggcaccaatttccctcctgacgggccggttatgcagaaaaaaacaatggggtgggaagccagtaccgagcggctctacccagaagatggcgtactcaaaggtgatatcaaaatggctctccgccttaaagacggcggtcggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 173 - DNA, 1171 bp, synthetic construct
  source 1..1171: mol_type = other DNA
cttcttggtgccagcttatcatagcgctaccggtcgccaccatggcgagaacaatggttgcaatggtttctaaaggtgaggcagtcataaaggagtttatgaggttcaaagttcacatggaagggtcaatgaacggacatgagttcgaaattgaaggtgagggggagggccgcccctatgaagggacacaaactgccaagctcaaggtgaccaagggcgggccattgcccttctcttgggatatcctgtctccgcaagtgagtgtgtgtgtgaatgagtgtgtatgtgtgtgtctgggggtgcttgtgagtgggtgggagtgtatgcgggtgtttgaagtggggctccgatcatctctgtccagccgcttagtttcccgttccccacttatttcctggtagtttatgtacggcagccgggctttcaccaaacaccctgccgatatcccggactactataaacagtcctttccagaaggttttaagtgggagcgcgtcatgaatttcgaggacggaggtgccgtcacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcc

SEQ ID NO: 174 - DNA, 144 bp, synthetic construct
  source 1..144: mol_type = other DNA
gtgagtgtgtgtgtgaatgagtgtgtatgtgtgtgtctgggggtgcttgtgagtgggtgggagtgtatgcgggtgtttgaagtggggctccgatcatctctgtccagccgcttagtttcccgttccccacttatttcctggtag

SEQ ID NO: 175 - DNA, 326 bp, synthetic construct
  source 1..326: mol_type = other DNA
gtgaggcagtcataaaggagtttatgaggttcaaagttcacatggaagggtcaatgaacggacatgagttcgaaattgaaggtgagggggagggccgcccctatgaagggacacaaactgccaagctcaaggtgaccaagggcgggccattgcccttctcttgggatatcctgtctccgcaagtgagtgtgtgtgtgaatgagtgtgtatgtgtgtgtctgggggtgcttgtgagtgggtgggagtgtatgcgggtgtttgaagtggggctccgatcatctctgtccagccgcttagtttcccgttccccacttatttcctggtag

SEQ ID NO: 176 - DNA, 744 bp, synthetic construct
  source 1..744: mol_type = other DNA
atggcgagaacaatggttgcaatggtttctaaaggtgaggcagtcataaaggagtttatgaggttcaaagttcacatggaagggtcaatgaacggacatgagttcgaaattgaaggtgagggggagggccgcccctatgaagggacacaaactgccaagctcaaggtgaccaagggcgggccattgcccttctcttgggatatcctgtctccgcaatttatgtacggcagccgggctttcaccaaacaccctgccgatatcccggactactataaacagtcctttccagaaggttttaagtgggagcgcgtcatgaatttcgaggacggaggtgccgtcacggttactcaggacaccagcctggaggacggcaccctgatctacaaggtgaagctgaggggcaccaacttcccccccgacggccccgtgatgcagaagaagaccatgggctgggaggccagcaccgagaggctgtaccccgaggacggcgtgctgaagggcgacatcaagatggccctgaggctgaaggacggcggcaggtacctggccgacttcaagaccacctacaaggccaagaagcccgtgcagatgcccggcgcctacaacgtggacaggaagctggacatcaccagccacaacgaggactacaccgtggtggagcagtacgagaggagcgagggcaggcacagcaccggcggcatggacgagctgtacaaggactacaaggacgatgatgacaagtga

SEQ ID NO: 177 - DNA, 2153 bp, synthetic construct
  source 1..2153: mol_type = other DNA
ctggagacgccatccacgctgttttgacctccatagaagaatctttgtcgatcctaccatccactcgacacacccgccagcggccgcttcttggtgccagcttatcatagcgctaccggtcgccaccatgggcccaaaaaagaagagaaaggtagaagaccccggcggaccggcggcgaaacgcgtgaaactggatggaggttacccatacgatgttccagattacgctggtggtatgtcagaatatattcgggtcaccgaggacgagaacgacgagcctatcgagataccatccgaagacgacggaacagtcctcctgagtaccgtgacagcacaattcccaggggcctgcggcctccgttacagaaaccctgttagccagtgtatgaggggtgtgcggctcgtggaaggcatactccacgctccggacgccgggtggggtaacttggtttatgtcgtaaattaccctaaggacaataaacgaaagatggacgaaaccgacgctagtagcgccgtgaaagtaaaacgggcagtgcagaagacatctgacctcatcgtcttaggtctgccttggaagaccacagagcaggatctgaaagaatatttctctacttttggcgaagtcctgatggtgcaggtgaaaaaggatctgaagacagggcatagcaaagggttcggatttgtcaggttcactgagtatgagacccaggtgaaagtgatgtcccagcgacatatgatcgatgggcggtggtgcgattgtaagctgcctaatagcaagcagtctcaggacgaacccttaagatcccgcaaggtgttcgtgggtcgctgcacggaggacatgaccgaggatgagctaaggtaagtggtgtgcctgttgtgtggttggggtgtgggtgtgtgtgggatgggagtgtggttgtcgcgtgtgattggctcgggtgcttggatgggtgattgtcggcgtgtttgacagggaattcttttcgcaatacggagacgtaatggacgtatttatacccaagccattccgggcctttgccttcgttacttttgctgatgaccaggtgcgtgcatgaatcgtgtgcatgttgtgtggttgattaggggtgtatgaccgggtgattgaggcgtgtatggtggtgtggatgacaagagtgattgttggtgtgaatgaagagtgactgactaacgtcttgaccgattctacagatcgcgcaatccttgtgcggcgaggaccttattattaagggcatctctgtacacatcagcaatgcagagcccaagcataattctaacctgccacctttactgggcccctctggcggcgaccgggagccaatgggactaggcccaccagcaacgcagctgactccaccacccgccccagttggcttgcgtggatccaaccaccgtggacttcccaaagatagtggccccttgcctacgccacccggcgtgagcctgctaggcgagccaccaaaggattacaggatacccctgaacccttaccttaatctccactcactgctgccctctagcaatcttgcgggaaaagagaccaggggctggggcggaagcgggagagggcgaagaccagctgagccgccactgccttcgccagcagttcctggaggagggtcaggcagtaacaatggcaacaaagcgttccaaatgaaaagtcgactcttgtctcccattgcctctaaccgcctgcctcccgaacccgggctgccagactcctatggatttgattacccgacagatgtgggtcctcgccgcttgttcagccatcccagagaacctactctaggagcccacgggccgagtaggcacaaaatgtcgcctccgccgtcctcattcaacgagcctagatccggcggtgggtccggaggcccactttcgcacttctgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcc

SEQ ID NO: 178 - DNA, 731 bp, synthetic construct
  source 1..731: mol_type = other DNA
atgggcccaaaaaagaagagaaaggtagaagaccccggcggaccggcggcgaaacgcgtgaaactggatggaggttacccatacgatgttccagattacgctggtggtatgtcagaatatattcgggtcaccgaggacgagaacgacgagcctatcgagataccatccgaagacgacggaacagtcctcctgagtaccgtgacagcacaattcccaggggcctgcggcctccgttacagaaaccctgttagccagtgtatgaggggtgtgcggctcgtggaaggcatactccacgctccggacgccgggtggggtaacttggtttatgtcgtaaattaccctaaggacaataaacgaaagatggacgaaaccgacgctagtagcgccgtgaaagtaaaacgggcagtgcagaagacatctgacctcatcgtcttaggtctgccttggaagaccacagagcaggatctgaaagaatatttctctacttttggcgaagtcctgatggtgcaggtgaaaaaggatctgaagacagggcatagcaaagggttcggatttgtcaggttcactgagtatgagacccaggtgaaagtgatgtcccagcgacatatgatcgatgggcggtggtgcgattgtaagctgcctaatagcaagcagtctcaggacgaacccttaagatcccgcaaggtgttcgtgggtcgctgcacggaggacatgaccgaggatgagctaag

SEQ ID NO: 179 - DNA, 115 bp, synthetic construct
  source 1..115: mol_type = other DNA
gtaagtggtgtgcctgttgtgtggttggggtgtgggtgtgtgtgggatgggagtgtggttgtcgcgtgtgattggctcgggtgcttggatgggtgattgtcggcgtgtttgacag

SEQ ID NO: 180 - DNA, 91 bp, synthetic construct
  source 1..91: mol_type = other DNA
ggaattcttttcgcaatacggagacgtaatggacgtatttatacccaagccattccgggcctttgccttcgttacttttgctgatgaccag

SEQ ID NO: 181 - DNA, 145 bp, synthetic construct
  source 1..145: mol_type = other DNA
gtgcgtgcatgaatcgtgtgcatgttgtgtggttgattaggggtgtatgaccgggtgattgaggcgtgtatggtggtgtggatgacaagagtgattgttggtgtgaatgaagagtgactgactaacgtcttgaccgattctacag

SEQ ID NO: 182 - DNA, 702 bp, synthetic construct
  source 1..702: mol_type = other DNA
atcgcgcaatccttgtgcggcgaggaccttattattaagggcatctctgtacacatcagcaatgcagagcccaagcataattctaacctgccacctttactgggcccctctggcggcgaccgggagccaatgggactaggcccaccagcaacgcagctgactccaccacccgccccagttggcttgcgtggatccaaccaccgtggacttcccaaagatagtggccccttgcctacgccacccggcgtgagcctgctaggcgagccaccaaaggattacaggatacccctgaacccttaccttaatctccactcactgctgccctctagcaatcttgcgggaaaagagaccaggggctggggcggaagcgggagagggcgaagaccagctgagccgccactgccttcgccagcagttcctggaggagggtcaggcagtaacaatggcaacaaagcgttccaaatgaaaagtcgactcttgtctcccattgcctctaaccgcctgcctcccgaacccgggctgccagactcctatggatttgattacccgacagatgtgggtcctcgccgcttgttcagccatcccagagaacctactctaggagcccacgggccgagtaggcacaaaatgtcgcctccgccgtcctcattcaacgagcctagatccggcggtgggtccggaggcccactttcgcacttctga

SEQ ID NO: 183 - DNA, 2159 bp, synthetic construct
  source 1..2159: mol_type = other DNA
ctggagacgccatccacgctgttttgacctccatagaagaatctttgtcgatcctaccatccactcgacacacccgccagcggccgcttcttggtgccagcttatcatagcgctaccggtcgccaccatgggcccaaaaaagaagagaaaggtagaagaccccggcggaccggcggcgaaacgcgtgaaactggatggaggttacccatacgatgttccagattacgctggtggtatgtcagaatatattcgggtcaccgaggacgagaacgacgagcctatcgagataccatccgaagacgacggaacagtcctcctgagtaccgtgacagcacaattcccaggggcctgcggcctccgttacagaaaccctgttagccagtgtatgaggggtgtgcggctcgtggaaggcatactccacgctccggacgccgggtggggtaacttggtttatgtcgtaaattaccctaaggacaataaacgaaagatggacgaaaccgacgctagtagcgccgtgaaagtaaaacgggcagtgcagaagacatctgacctcatcgtcttaggtctgccttggaagaccacagagcaggatctgaaagaatatttctctacttttggcgaagtcctgatggtgcaggtgaaaaaggatctgaagacagggcatagcaaagggttcggatttgtcaggttcactgagtatgagacccaggtgaaagtgatgtcccagcgacatatgatcgatgggcggtggtgcgattgtaagctgcctaatagcaagcagtctcaggacgaacccttaagatcccgcaaggtgttcgtgggtcgctgcacggaggacatgacggaggacgaactaaggtaagcggagtgcttgttgcgtggttggggtgtgggtgtgagtgggatgggagagtggttgttgcgtgtggttggctcgggtgcatggatgagtgattgtcggcgtgtttgtgtgtgacagggaatttttctcgcaatacggcgacgtaatggacgtgtttatcccgaagcccttccgagccttcgcgttcgttaccttcgcagatgatcaggtgctgtgtgtgtgcgagtgcatgtggggtggttgaacaggggtgtatggctgggtgtttgaggcgtggatgattgtgtgcatgacaagagtgaatgttggtgtgaatgacgagtgactgtctaacgtcttgaccgattctacagatcgcgcaatcgctgtgcggagaagaccttattattaagggcatctctgtacacatcagcaatgcagagcccaagcataattctaacctgccacctttactgggcccctctggcggcgaccgggagccaatgggactaggcccaccagcaacgcagctgactccaccacccgccccagttggcttgcgtggatccaaccaccgtggacttcccaaagatagtggccccttgcctacgccacccggcgtgagcctgctaggcgagccaccaaaggattacaggatacccctgaacccttaccttaatctccactcactgctgccctctagcaatcttgcgggaaaagagaccaggggctggggcggaagcgggagagggcgaagaccagctgagccgccactgccttcgccagcagttcctggaggagggtcaggcagtaacaatggcaacaaagcgttccaaatgaaaagtcgactcttgtctcccattgcctctaaccgcctgcctcccgaacccgggctgccagactcctatggatttgattacccgacagatgtgggtcctcgccgcttgttcagccatcccagagaacctactctaggagcccacgggccgagtaggcacaaaatgtcgcctccgccgtcctcattcaacgagcctagatccggcggtgggtccggaggcccactttcgcacttctgataaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcc

SEQ ID NO: 184 - DNA, 731 bp, synthetic construct
  source 1..731: mol_type = other DNA
atgggcccaaaaaagaagagaaaggtagaagaccccggcggaccggcggcgaaacgcgtgaaactggatggaggttacccatacgatgttccagattacgctggtggtatgtcagaatatattcgggtcaccgaggacgagaacgacgagcctatcgagataccatccgaagacgacggaacagtcctcctgagtaccgtgacagcacaattcccaggggcctgcggcctccgttacagaaaccctgttagccagtgtatgaggggtgtgcggctcgtggaaggcatactccacgctccggacgccgggtggggtaacttggtttatgtcgtaaattaccctaaggacaataaacgaaagatggacgaaaccgacgctagtagcgccgtgaaagtaaaacgggcagtgcagaagacatctgacctcatcgtcttaggtctgccttggaagaccacagagcaggatctgaaagaatatttctctacttttggcgaagtcctgatggtgcaggtgaaaaaggatctgaagacagggcatagcaaagggttcggatttgtcaggttcactgagtatgagacccaggtgaaagtgatgtcccagcgacatatgatcgatgggcggtggtgcgattgtaagctgcctaatagcaagcagtctcaggacgaacccttaagatcccgcaaggtgttcgtgggtcgctgcacggaggacatgacggaggacgaactaag

SEQ ID NO: 185 - DNA, 121 bp, synthetic construct
  source 1..121: mol_type = other DNA
gtaagcggagtgcttgttgcgtggttggggtgtgggtgtgagtgggatgggagagtggttgttgcgtgtggttggctcgggtgcatggatgagtgattgtcggcgtgtttgtgtgtgacag

SEQ ID NO: 186 - DNA, 91 bp, synthetic construct
  source 1..91: mol_type = other DNA
ggaatttttctcgcaatacggcgacgtaatggacgtgtttatcccgaagcccttccgagccttcgcgttcgttaccttcgcagatgatcag

SEQ ID NO: 187 - DNA, 145 bp, synthetic construct
  source 1..145: mol_type = other DNA
gtgctgtgtgtgtgcgagtgcatgtggggtggttgaacaggggtgtatggctgggtgtttgaggcgtggatgattgtgtgcatgacaagagtgaatgttggtgtgaatgacgagtgactgtctaacgtcttgaccgattctacag

SEQ ID NO: 188 - DNA, 702 bp, synthetic construct
  source 1..702: mol_type = other DNA
atcgcgcaatcgctgtgcggagaagaccttattattaagggcatctctgtacacatcagcaatgcagagcccaagcataattctaacctgccacctttactgggcccctctggcggcgaccgggagccaatgggactaggcccaccagcaacgcagctgactccaccacccgccccagttggcttgcgtggatccaaccaccgtggacttcccaaagatagtggccccttgcctacgccacccggcgtgagcctgctaggcgagccaccaaaggattacaggatacccctgaacccttaccttaatctccactcactgctgccctctagcaatcttgcgggaaaagagaccaggggctggggcggaagcgggagagggcgaagaccagctgagccgccactgccttcgccagcagttcctggaggagggtcaggcagtaacaatggcaacaaagcgttccaaatgaaaagtcgactcttgtctcccattgcctctaaccgcctgcctcccgaacccgggctgccagactcctatggatttgattacccgacagatgtgggtcctcgccgcttgttcagccatcccagagaacctactctaggagcccacgggccgagtaggcacaaaatgtcgcctccgccgtcctcattcaacgagcctagatccggcggtgggtccggaggcccactttcgcacttctga

SEQ ID NO: 189 - DNA, 6863 bp, synthetic construct
  source 1..6863: mol_type = other DNA
tgaaccgtcagatccgctagagatccgcggccgctaatacgactcactatagggagagccgccaccatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagaaagctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaagagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccagactgtcccgtaagctcatcaatggcatacgcgataaacaatcggggaagacaatcctggattttcttaaatctgatggatttgcgaatcgtaactttatgcagctgatccatgatgactccctgacgttcaaagaggatattcagaaggcgcaggtaagtatcgtgtgtgtgtttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggtatccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcaccctgaacattgaagacgagtatagactgcatgaaacaagcaaggaacccgacgtgtccctgggctccacctggctgtccgactttccccaggcctgggccgagacaggaggaatgggcctggccgtgcggcaggcacccctgatcatccctctgaaggccacctctacacccgtgagcatcaagcagtaccctatgtctcaggaggccagactgggcatcaagcctcacatccagaggctgctggaccagggcatcctggtgccatgccagagcccctggaacacaccactgctgcccgtgaagaagccaggcaccaatgactatagacccgtgcaggatctgagagaggtgaacaagagggtggaggatatccaccccaccgtgcccaacccttacaatctgctgtccggcctgcccccttctcaccagtggtatacagtgctggacctgaaggatgccttcttttgtctgagactgcaccctaccagccagccactgttcgcctttgagtggagggaccctgagatgggcatctctggccagctgacctggacacgcctgcctcagggcttcaagaatagcccaacactgtttaacgaggccctgcaccgcgacctggcagatttccggatccagcacccagatctgatcctgctgcagtacgtggacgatctgctgctggccgccaccagcgagctggattgccagcagggaacacgcgccctgctgcagaccctgggaaacctgggatatagggcatccgccaagaaggcccagatctgtcagaagcaggtgaagtacctgggctatctgctgaaggagggccagagatggctgacagaggccaggaaggagacagtgatgggccagccaacacccaagaccccaagacagctgagggagttcctgggcaaagcaggattttgcaggctgttcatcccaggattcgcagagatggcagcacctctgtacccactgaccaagccgggcaccctgtttaattggggccctgaccagcagaaggcctatcaggagatcaagcaggccctgctgacagcaccagccctgggcctgccagacctgaccaagcctttcgagctgtttgtggatgagaagcagggctacgccaagggcgtgctgacccagaagctgggaccatggagacggcccgtggcctatctgtccaagaagctggacccagtggcagcaggatggccaccatgcctgaggatggtggcagcaatcgccgtgctgacaaaggatgccggcaagctgaccatgggacagccactggtcatcctggcaccacacgcagtggaggccctggtgaagcagcctccagatcgctggctgtctaacgcccggatgacacactaccaggccctgctgctggacaccgatcgcgtgcagtttggccctgtggtggccctgaatccagccaccctgctgcctctgccagaggagggcctgcagcacaactgtctggacatcctggcagaggcacacggaacaaggccagacctgaccgatcagcccctgcctgacgccgatcacacatggtataccgatggaagctccctgctgcaggagggccagaggaaggcaggagcagcagtgaccacagagacagaagtgatctgggccaaggccctgccagcaggcacatccgcccagcgggccgagctgatcgccctgacccaggccctgaagatggccgagggcaagaagctgaacgtgtacacagactccagatatgccttcgccaccgcacacatccacggagagatctacaggcgccggggctggctgacctctgagggcaaggagatcaagaacaaggatgagatcctggccctgctgaaggccctgtttctgcccaagcggctgagcatcatccactgtcctggacaccagaagggacactccgccgaggcaaggggcaatcggatggccgaccaggccgccagaaaggctgctattactgaaactcccgacacttccactctgctgattgaaaactcctccccttctggcggctcaaaaagaaccgccgacggcagcgaattcgagtctcccaagaagaagaggaaagtcggctctggccctgccgctaagagagtgaagctggactaaccggtcagcggccgc

SEQ ID NO: 190 - DNA, 2036 bp, synthetic construct
  source 1..2036: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagaaagctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaagagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcag

SEQ ID NO: 191 - DNA, 163 bp, synthetic construct; identical to SEQ ID NO: 30
  source 1..163: mol_type = other DNA

SEQ ID NO: 192 - DNA, 154 bp, synthetic construct
  source 1..154: mol_type = other DNA
actgtcccgtaagctcatcaatggcatacgcgataaacaatcggggaagacaatcctggattttcttaaatctgatggatttgcgaatcgtaactttatgcagctgatccatgatgactccctgacgttcaaagaggatattcagaaggcgcag

SEQ ID NO: 193 - DNA, 223 bp, synthetic construct
  source 1..223: mol_type = other DNA
gtaagtatcgtgtgtgtgtttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcag

SEQ ID NO: 194 - DNA, 4206 bp, synthetic construct
  source 1..4206: mol_type = other DNA
gtatccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcaccctgaacattgaagacgagtatagactgcatgaaacaagcaaggaacccgacgtgtccctgggctccacctggctgtccgactttccccaggcctgggccgagacaggaggaatgggcctggccgtgcggcaggcacccctgatcatccctctgaaggccacctctacacccgtgagcatcaagcagtaccctatgtctcaggaggccagactgggcatcaagcctcacatccagaggctgctggaccagggcatcctggtgccatgccagagcccctggaacacaccactgctgcccgtgaagaagccaggcaccaatgactatagacccgtgcaggatctgagagaggtgaacaagagggtggaggatatccaccccaccgtgcccaacccttacaatctgctgtccggcctgcccccttctcaccagtggtatacagtgctggacctgaaggatgccttcttttgtctgagactgcaccctaccagccagccactgttcgcctttgagtggagggaccctgagatgggcatctctggccagctgacctggacacgcctgcctcagggcttcaagaatagcccaacactgtttaacgaggccctgcaccgcgacctggcagatttccggatccagcacccagatctgatcctgctgcagtacgtggacgatctgctgctggccgccaccagcgagctggattgccagcagggaacacgcgccctgctgcagaccctgggaaacctgggatatagggcatccgccaagaaggcccagatctgtcagaagcaggtgaagtacctgggctatctgctgaaggagggccagagatggctgacagaggccaggaaggagacagtgatgggccagccaacacccaagaccccaagacagctgagggagttcctgggcaaagcaggattttgcaggctgttcatcccaggattcgcagagatggcagcacctctgtacccactgaccaagccgggcaccctgtttaattggggccctgaccagcagaaggcctatcaggagatcaagcaggccctgctgacagcaccagccctgggcctgccagacctgaccaagcctttcgagctgtttgtggatgagaagcagggctacgccaagggcgtgctgacccagaagctgggaccatggagacggcccgtggcctatctgtccaagaagctggacccagtggcagcaggatggccaccatgcctgaggatggtggcagcaatcgccgtgctgacaaaggatgccggcaagctgaccatgggacagccactggtcatcctggcaccacacgcagtggaggccctggtgaagcagcctccagatcgctggctgtctaacgcccggatgacacactaccaggccctgctgctggacaccgatcgcgtgcagtttggccctgtggtggccctgaatccagccaccctgctgcctctgccagaggagggcctgcagcacaactgtctggacatcctggcagaggcacacggaacaaggccagacctgaccgatcagcccctgcctgacgccgatcacacatggtataccgatggaagctccctgctgcaggagggccagaggaaggcaggagcagcagtgaccacagagacagaagtgatctgggccaaggccctgccagcaggcacatccgcccagcgggccgagctgatcgccctgacccaggccctgaagatggccgagggcaagaagctgaacgtgtacacagactccagatatgccttcgccaccgcacacatccacggagagatctacaggcgccggggctggctgacctctgagggcaaggagatcaagaacaaggatgagatcctggccctgctgaaggccctgtttctgcccaagcggctgagcatcatccactgtcctggacaccagaagggacactccgccgaggcaaggggcaatcggatggccgaccaggccgccagaaaggctgctattactgaaactcccgacacttccactctgctgattgaaaactcctccccttctggcggctcaaaaagaaccgccgacggcagcgaattcgagtctcccaagaagaagaggaaagtcggctctggccctgccgctaagagagtgaagctggactaa

SEQ ID NO: 195 - DNA, 1238 bp, synthetic construct
  source 1..1238: mol_type = other DNA
cttcttggtgccagcttatcatagcgctaccggtcgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaggcaaacgcgaggaaggccggctgcaccaggtaagaatgcacatcacttcttgagagtatggaggagtgaaatgacactcatgccagagttactgtatgtctgcactttaaaagtgtagcttttaaaagatgatgaatgactgtctgtttgtgtgtgtgtgtgtgaatgtgtgtgtgtgtgtgtgtcacccaggggttgcctgatctgcttaagccacatcaagtgtacgccaaaaatgaagaagttcatcccaggtatgcatgactgcctgcatgcttgtttgtttgtttgtatgtttgtatggagtcggggtttctgaatgtatgcctgaggctggttgcagagtctcgctctggatgtctacgctggatgtgcagtaacatgagccactgtgcccggccaatcctaagaatttcttttgcggtggttgcaagtctgggcagaactcttgtcaggggctgtaactggacttatctttactcctttgtcaggacggtgtcatacgtacgaaggcgataaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaacaaatggtaaggaagggcacatcaatctttgcttaattgtcctttactctaaagatgtattttatcatactgaatgctaaacttgatatctccttttaggtcattgatgtccttcaccccgggaaggcgacagtgcctaagacagaaattcgggaaaaactagccaaaatgtacaagaccacaccggatgtcatctttgtatttggattcagaactcagtaaactggatccgcaggcc

SEQ ID NO: 196 - DNA, 212 bp, synthetic construct
  source 1..212: mol_type = other DNA
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaggcaaacgcgaggaaggccggctgcaccag

SEQ ID NO: 197 - DNA, 163 bp, synthetic construct
  source 1..163: mol_type = other DNA
gtaagaatgcacatcacttcttgagagtatggaggagtgaaatgacactcatgccagagttactgtatgtctgcactttaaaagtgtagcttttaaaagatgatgaatgactgtctgtttgtgtgtgtgtgtgtgaatgtgtgtgtgtgtgtgtgtcacccag

SEQ ID NO: 198 - DNA, 62 bp, synthetic construct
  source 1..62: mol_type = other DNA
gggttgcctgatctgcttaagccacatcaagtgtacgccaaaaatgaagaagttcatcccag

SEQ ID NO: 199 - DNA, 237 bp, synthetic construct
  source 1..237: mol_type = other DNA
gtatgcatgactgcctgcatgcttgtttgtttgtttgtatgtttgtatggagtcggggtttctgaatgtatgcctgaggctggttgcagagtctcgctctggatgtctacgctggatgtgcagtaacatgagccactgtgcccggccaatcctaagaatttcttttgcggtggttgcaagtctgggcagaactcttgtcaggggctgtaactggacttatctttactcctttgtcag

SEQ ID NO: 200 - DNA, 284 bp, synthetic construct
  source 1..284: mol_type = other DNA
gacggtgtcatacgtacgaaggcgataaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 201 - DNA, 2306 bp, synthetic construct
  source 1..2306: mol_type = other DNA
cttcttggtgccagcttatcatagcgctaccggtcgccaccatgcccaagaagaagaggaaggtgtccaacctgttaacagtccaccagaacctacctgcccttcccgtcgacgctacgtcggacgaggttcggaagaacctgatggacatgttccgggaccgacaggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggcatttagcgaacacacgtggaagatgctgctttccgtctgtcggagttgggctgcgtggtgcaagttgaacaatcgtaaatggtttccagccggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcagaaccggaggacgtcagagactatttgctctacctgcaagcccgcggactagccgttaagactatacagcagcatttgggacaattaaatatgttgcacaggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccaggcggtccgggcttccccggccttcggattcgaacgcagtgagcctagtcatgcgccggattagaaaggaaaatgttgacgctggagaacgggcaaagcaagtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggctttagcgtttgagagaacagattttgatcaagtgcgatcccttatggagaactctgaccgttgccaagacataagaaatcttgctttcttgggcatcgcgtacaacaccttactgagaattgcggagattgcccggattcgagtcaaggatataagccgcaccgacggaggacggatgctcatccacattgggagaacgaaaaccctagtgtcaaccgccggtgtggagaaagctctgagccttggagtcacaaaattggtcgagcgatggatcaggtaagaatgcacatcacttcttgagagtatggaggagtgaaatgacactcagtgccagagttactgtatatctacactttaaaagtgtagcttttaaaagataagcaagcacaatcttttgtgtgtgtgtgtgtgaatgtgtgtgtgtgtgtgtgtcacccagcgtttccggtgtggccgacgatccaaacaactatctattctgtcgggtccgtaaaaatggggttgcggcaccaagcgcaacctcccagttgagcacgagggctctggaaggtatgcatcacccccccagctaatttttttttgtattttttaccgagtcggggtttcgcaatgttgcccaggctggtctcagagtctcgctctgttgtctacgctggagtgcagtaacatgagccactgtgcccggccaatcctaagaatttcttttgcggtggttgcaagtctgggcagaactcttgtcaggggctgtaactggacttatctttactcctttgtcaggtatcttcgaagccactcaccgactgatctacggcgcaaaggacgactccggacagcgataccttgcttggagtgggcatagtgcacgggtgggggcggctagggatatggctagagctggagtctcaatccctgaaattatgcaagctgggggttggacaaatgttaatattgtaatgaactatataagaaacttggatagtgagacaggggctatggtgcgcctgttagaagatggggacggctctggatctccggcggcgaaacgcgtgaaactggattaa

SEQ ID NO: 202 - RNA, 20 nt, synthetic construct
  source 1..20: mol_type = other RNA
taaaagcatggatggagaga

SEQ ID NO: 203 - RNA, 28 nt, synthetic construct
  source 1..28: mol_type = other RNA
atggactcacgcatctctccatccatgc